(19) United States
(12) Patent Application Publication
(10) Pub. No.: US 2025/0367308 A1
(43) Pub. Date: Dec. 4, 2025

(54) LIGAND-DRUG CONJUGATE OF CAMPTOTHECIN ANALOGS, INTERMEDIATES, PREPARATION METHOD THEREFOR, PHARMACEUTICAL COMPOSITION AND APPLICATION THEREOF

(71) Applicant: SHANGHAI MICURX PHARMACEUTICAL CO.,LTD., Shanghai (CN)

(72) Inventors: Hongyu YANG, Shanghai (CN); Xinghai WANG, Shanghai (CN)

(73) Assignee: SHANGHAI MICURX PHARMACEUTICAL CO.,LTD., Shanghai (CN)

(21) Appl. No.: 18/861,184

(22) PCT Filed: Apr. 21, 2023
(86) PCT No.: PCT/CN2023/089679

§ 371 Date: Oct. 28, 2024

Related U.S. Application Data
(60) Provisional application No. 63/336,995, filed on Apr. 29, 2022.

(51) Int. Cl. A61K 47/68; A61P 35/00; C07D 491/22

(52) U.S. Cl. CPC A61K 47/68037; A61K 47/6851; A61P 35/00; C07D 491/22

(57) ABSTRACT

Provided are conjugates of novel camptothecin analogs with a cell binding molecule of formula (I). It also provides methods of making the conjugates of camptothecin analogs to a cell-binding agent, as well as methods of application the conjugates in targeted treating of cancer.

### CROSS-REFERENCE TO RELATED APPLICATIONS

This application claims the benefit of, and priority to, U.S. Provisional Application No. 63/336,995 (filed Apr. 29, 2022), the contents of which are incorporated herein by reference in their entirety.

### REFERENCE TO SEQUENCE LISTING SUBMITTED VIA EFS-WEB

This application includes an electronically submitted sequence listing in .XML format. The .XML file contains a sequence listing entitled “MRX-22-PCT-US” created on Jun. 19, 2025, and is 9 kilobytes in size. The sequence listing contained in this .XML file is part of the specification and is incorporated herein by reference in its entirety.

### FIELD

Provided are conjugates of camptothecin analogs with a cell-surface receptor-binding molecule for targeted therapy, as well as pharmaceutical compositions comprising such a conjugate. Also provided are camptothecin analogs, intermediates of conjugates of camptothecin analogs, preparation methods therefor. Also provided are uses of a camptothecin analog, a conjugate of a camptothecin analog, a pharmaceutical composition comprising a camptothecin analog, and a pharmaceutical composition comprising a conjugate of the camptothecin analog to a cell-binding molecule for targeted treatment of cancer.

### BACKGROUND

Cancer is a leading cause of death worldwide. Surgery, chemotherapy, radiotherapy and targeted therapy are the standard of care therapies. Although chemotherapy is widely applied, the use of most chemotherapies is limited by undesired side effects, mostly through action on cells beyond the tumor and its environment, resulting in systemic toxicity and a narrow therapeutic window. The discovery of the unique composition of cancer cell surfaces combined with the understanding of the strong and selective interaction between antibodies and cell-surface antigens opened the way to exploit antibodies as targeted delivery agents for chemotherapies, including highly toxic drugs (Drago, J. Z. et al., Nat. Rev. Clin. Oncol. 2021; Khongorzul, P. et al., Mol. Cancer Res. 2020, 18, 3-19; Joubert, N. et al; The Last Decade. Pharmaceuticals 2020, 13, 245; Ravi V. J. Chari et al., Angew. Chem. Int. Ed. 2014, 53, 3796-3827.). The resulting molecular entities, also known as antibody-drug conjugates (ADC) consist of three main parts: the antibody responsible for the selective recognition of the cancer cell surface antigen capable of internalizing the ADC, the drug payload responsible for killing the cancer cell once released inside it, and the linker connecting the antibody and payload parts.

Antibody-drug conjugates (ADCs), combining the selective targeting of tumor cells through antigen-directed recognition and potent cell-killing by cytotoxic payloads, have emerged in recent years as an efficient therapeutic approach for the treatment of various cancers (Nature review Drug Discovery, 2013, 12, 329-332). The first ADC (Mylotarg) was approved in 2000 (and following withdrawal in 2010, reapproved in 2017), and the second ADC (Adcetris) received accelerated approval in 2011 and full approval in 2015. The third (Kadcyla) and fourth (Besponsa) ADCs were approved in 2013 and 2017, respectively. Kadcyla is the first ADC approved for solid tumor treatment. Since 2019, more than ten ADCs have been approved, and there are more than 100 ADCs in clinical development.

It has been known that the payload-linker component in the ADC critically contribute to ADC homogeneity, circulation stability, pharmacokinetic profiles, tolerability and overall treatment efficacy (Acchionea, M. et al., mAbs. 2012, 4, 362; Zhao, R. Y. et al., J. Med. Chem. 2011, 54, 3606;). Despite extensive study to improve these profiles, most payload used so far include DNA damaging agents (such as calicheamicins, PBD, and duocarmysins), microtubule disrupting agents (such as maytansins, like DM1 or DM4; auruistatins like MMAE or MMAF; tubulysins) and topoisomerase inhibitors (such as camptothecins like Dxd or SN-38). (Leung, D., et al., Antibodies (Basel). 2020, 9, 2; Khongorzul, P., et al., Mol. Cancer. Res., 2020, 18, 3; Chau, C. H., et al., Lancet. 2019, 394, 793.) Among these payloads, the camptothecins have proved a promising choice with a wider therapeutic index than many other payloads for ADC construction. Two of the approved ADCs, Enhertu and Trodelvy, which employ the camptothecin payloads Dxd and SN-38 respectively, have demonstrated significant clinical benefits (Progression-Free-Survival, PFS and Overall-Survival, OS) for solid tumors in many clinical trials (Pondé, N., et al., Curr Treat Options Oncol. 2019, 20, 37; Kaplon, H., et al., Mabs. 2020, 12, 1703531.). By interacting with DNA enzyme topoisomerase I and then accumulating reversible enzyme-camptothecin-DNA ternary complexes, camptothecin can induce cell death.

Camptothecin is a potent antitumor antibiotic isolated in 1958 from extracts of Camptotheca acuminata, wherein the plant has been extensively used in traditional Chinese medicine for hundreds of years. Many camptothecin analogs have been disclosed, such as those shown below:

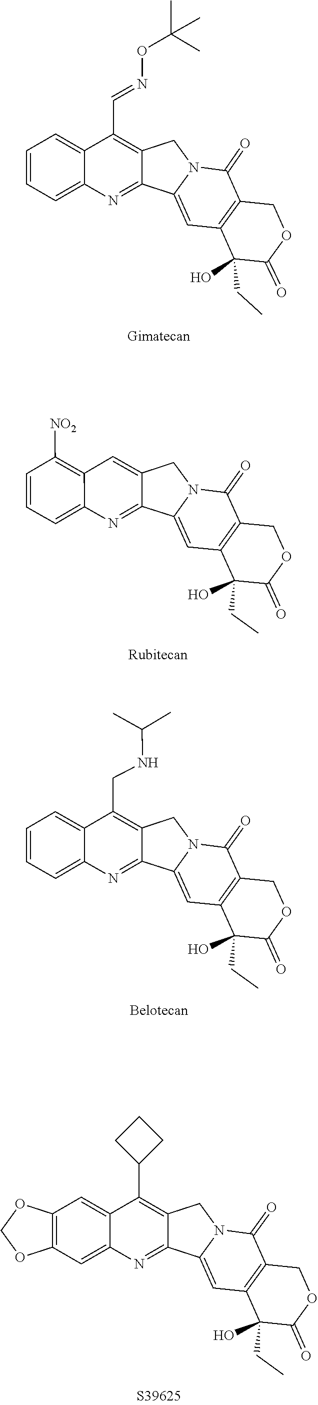 [SMILES: CC[C@@]1(O)C(=O)OCc2c1cc1n(c2=O)Cc2c-1nc1ccccc1c2/C=N/OC(C)(C)C ; CC[C@@]1(O)C(=O)OCc2c1cc1n(c2=O)Cc2cc3c([N+](=O)[O-])cccc3nc2-1 ; CC[C@@]1(O)C(=O)OCc2c1cc1n(c2=O)Cc2c-1nc1ccccc1c2CNC(C)C ; CC[C@@]1(O)C(=O)OCc2c1cc1n(c2=O)Cc2c-1nc1cc3c(cc1c2C1CCC1)OCO3]

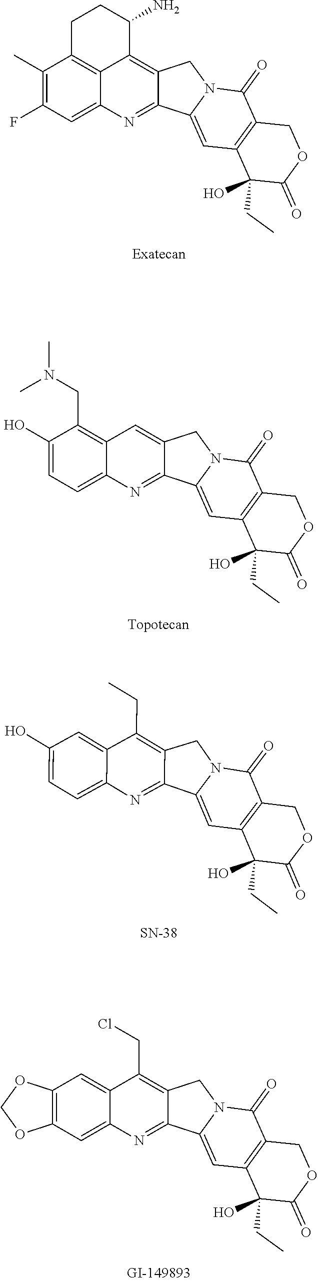 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2cc3c(CN(C)C)c(O)ccc3nc2-1 ; CCc1c2c(nc3ccc(O)cc13)-c1cc3c(c(=O)n1C2)COC(=O)[C@]3(O)CC ; "SN-38" ; CC[C@@]1(O)C(=O)OCc2c1cc1n(c2=O)Cc2c-1nc1cc3c(cc1c2CCl)OCO3]

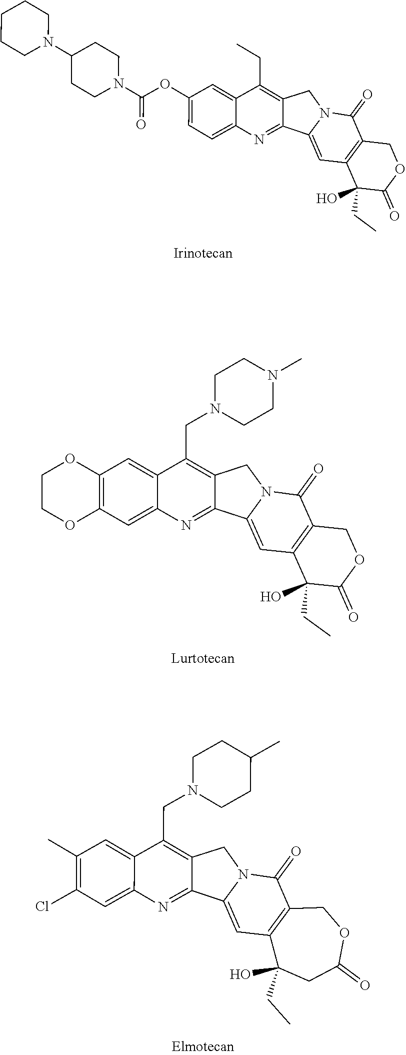 [SMILES: CCc1c2c(nc3ccc(OC(=O)N4CCC(N5CCCCC5)CC4)cc13)-c1cc3c(c(=O)n1C2)COC(=O)[C@]3(O)CC ; CC[C@@]1(O)C(=O)OCc2c1cc1n(c2=O)Cc2c-1nc1cc3c(cc1c2CN1CCN(C)CC1)OCCO3 ; CC[C@@]1(O)CC(=O)OCc2c1cc1n(c2=O)Cc2c-1nc1cc(Cl)c(C)cc1c2CN1CCC(C)CC1]

Camptothecin and most of its analogs are extremely insoluble in physiological buffer and have demonstrated high adverse drug reaction in the preliminary clinical trial since 1970s. The low solubility of camptothecin can cause their ADC conjugates to aggregate (Burke, P., et al. Bioconjugate Chem. 2009, 20, 6, 1242) which is problematic for scale-up manufacturing production and may cause systematic side-effects resulting from aggregation. So far the US FDA has only approved three water-soluble camptothecin analogs: topotecan, irinotecan and belotecan in cancer therapy (Palakurthi, S., Expert Opin Drug Deliv. 2015, 12(12), 1911). Most of the camptothecin payloads employed to date for ADC development suffer from low solubility, which further limits the Drug-to-Antibody Ratio and results in low potency.

Provided herein is a series of ligand-drug conjugates of camptothecin analogs.

### SUMMARY

Provided are conjugates of camptothecin analogs linked to a cell-binding molecule, camptothecin analog-linker compounds and camptothecin analogs, methods to prepare and to use them, and intermediates useful in the preparation thereof. The camptothecin analog conjugates of the present disclosure are water-soluble and stable in circulation, as well as providing high cytotoxicity once the free camptothecin analog or a metabolite of the camptothecin analog-linker compound is released from the conjugate in the vicinity of or within disordered cells.

Aspect 1: These compounds have the general formula I:

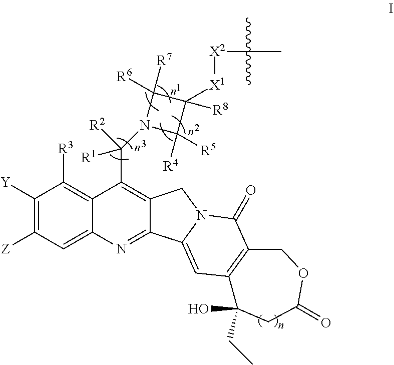

or pharmaceutically acceptable salt or solvate thereof,
wherein:
Y and Z are independently selected from H and a group consisting of halo, hydroxy, cyano, C₁-C₈ alkyl, C₁-C₈ alkoxy, (C₁-C₈ alkoxy)-C₁-C₈ alkyl, amino, (C₁-C₈ alkyl)NHC(O)O—, (C₃-C₆ cycloalkyl)NHC(O)O—, (C₁-C₈ alkyl)NH—, azetidin-1-yl, pyrrolidin-1-yl, piperidin-1-yl, azepane-1-yl, (C₁-C₈ alkyl)C(O)O—, and (C₁-C₈ alkyl)C(O)NH—; or Y and Z taken together with the atom(s) to which they are attached form a 5- to 8-membered heterocycloalkyl or 5- to 8-membered heteroaryl comprise one to three heteroatoms independently selected from N, O, S, S(O), and S(O)₂; wherein azetidin-1-yl, pyrrolidin-1-yl, piperidin-1-yl, azepane-1-yl, 5- to 8-membered heterocycloalkyl, 5- to 8-membered heteroaryl, and each “C₁-C₈ alkyl” are independently optionally substituted with one to three R⁹;
R³ is selected from H and a group consisting of halo, hydroxy, cyano, C₁-C₈ alkyl, C₁-C₈ alkoxy, (C₁-C₈ alkoxy)-C₁-C₈ alkyl, amino, (C₁-C₈ alkyl)amino, (C₁-C₆ alkyl)NHC(O)O—, (C₃-C₆ cycloalkyl)NHC(O)O—, (C₁-C₆ alkyl)C(O)O—, (C₁-C₆ alkyl)C(O)NH—, aryl, heteroaryl, and nitro; wherein each “C₁-C₈ alkyl” cycloalkyl, aryl, and heteroaryl are independently optionally substituted with one to three R⁹;
each R¹ and each R² are independently selected from H, C₁-C₆ alkyl, C₃-C₆ cycloalkyl, aryl, and heteroaryl; or each pair of R¹ and R² taken together with the atom(s) to which they are attached form 3- to 6-membered cycloalkyl, or 4- to 8-membered heterocycloalkyl; wherein each R¹, R², and R¹/R² ring group is independently optionally substituted with one to four R⁹;
n³ is an integer selected from 0 to 6;
X¹ is absent; or X¹ and R⁸ taken together with the atom(s) to which they are attached form 3- to 6-membered cycloalkyl or 4- to 8-membered heterocycloalkyl; or X¹ and R⁵ taken together with the atom(s) to which they are attached form a 3- to 6-membered cycloalkyl or 4- to 8-membered heterocycloalkyl; each of the 3- to 6-membered cycloalkyl and 4- to 8-membered heterocycloalkyl is optionally substituted with one to three R⁹;
X² comprises one or more groups independently selected from the group consisting of —O—, —S—, —NH—, C₁-C₆ alkylene, C₃-C₆ cycloalkylene, arylene, and heteroarylene; wherein the terminal group in X², connected to the [symbol]
in Formula (I), is O or S; each of C₁-C₆ alkylene, C₃-C₆ cycloalkylene, arylene, and heteroarylene is independently optionally substituted with one to three R⁹;
R⁴, R⁵, R⁶, R⁷, and R⁸ at each occurrence are independently selected from H and a group consisting of halo, hydroxy, C₁-C₆ alkyl, C₃-C₆ cycloalkyl, aryl, and heteroaryl and wherein each of C₁-C₆ alkyl, C₃-C₆ cycloalkyl, aryl, and heteroaryl is independently optionally substituted with one to four R⁹; or
R⁴ and R⁵ taken together with the atom(s) to which they are attached form 3- to 6-membered cycloalkyl, or 4- to 8-membered heterocycloalkyl; or R⁴ and R⁷ taken together with the atom(s) to which they are attached form 3- to 6-membered cycloalkyl, or 4- to 8-membered heterocycloalkyl; or R⁶ and R⁷ taken together with the atom(s) to which they are attached form 3- to 6-membered cycloalkyl, or 4- to 8-membered heterocycloalkyl; or R⁷ and R⁸ taken together with the atom(s) to which they are attached form oxo, 3- to 6-membered cycloalkyl, or 4- to 8-membered heterocycloalkyl; and wherein each of the 3- to 6-membered cycloalkyl and 4- to 8-membered heterocycloalkyl are independently optionally substituted with one to four R⁹; and the remaining of R⁴, R⁵, R⁶, R⁷, and R⁸ at each occurrence are independently selected from H and the group consisting of halo, hydroxy, C₁-C₆ alkyl, C₃-C₆ cycloalkyl, aryl, and heteroaryl, wherein the C₁-C₆ alkyl, C₃-C₆ cycloalkyl, aryl, and heteroaryl are independently optionally substituted with one to four R⁹;
R⁹ at each occurrence is independently selected from the group consisting of halogen, oxo, hydroxy, cyano, C₁-C₆ alkyl, C₃-C₆ cycloalkyl, C₁-C₆ alkoxy, aryl, and heteroaryl; or two R⁹ groups when attached to adjacent carbons and taken together with the carbons to which they are attached form a fused C₃-C₆ cycloalkyl; or two R⁹ groups when attached to the same carbon and taken together with the carbon to which they are attached form a spiro C₃-C₆ cycloalkyl; wherein each C₁-C₆ alkyl, C₃-C₆ cycloalkyl, C₁-C₆ alkoxy, aryl, heteroaryl, fused C₃-C₆ cycloalkyl, and spiro C₃-C₆ cycloalkyl is optionally independently substituted with one to three fluoro or hydroxy, and C₁-C₃ alkyl;
n¹ and n² are each an integer independently selected from 0, 1, 2, 3, and 4, provided that n¹+n² is 2, 3, 4, or 5; and
n is an integer selected from 0 and 1. In some embodiments, when one R⁴ and one R⁵ are taken together with the atom(s) to which they are attached to form 3- to 6-membered cycloalkyl, or 4- to 8-membered heterocycloalkyl, the R⁴ and R⁵ are on different, but adjacent, carbon atoms; and when one R⁶ and one R⁷ are taken together with the atom(s) to which they are attached to form 3- to 6-membered cycloalkyl, or 4- to 8-membered heterocycloalkyl, the R⁶ and R⁷ are on different, but adjacent, carbon atoms; and wherein each of the 3- to 6-membered cycloalkyl and 4- to 8-membered heterocycloalkyl are independently optionally substituted with one to four R⁹.

In some embodiments or aspects of the present invention, the composition or method has the proviso that the claim does not include any of the following substructures:

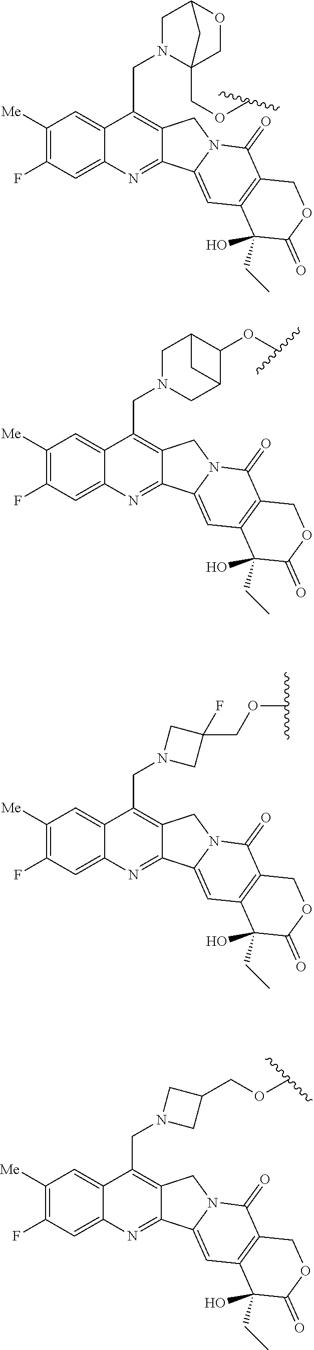

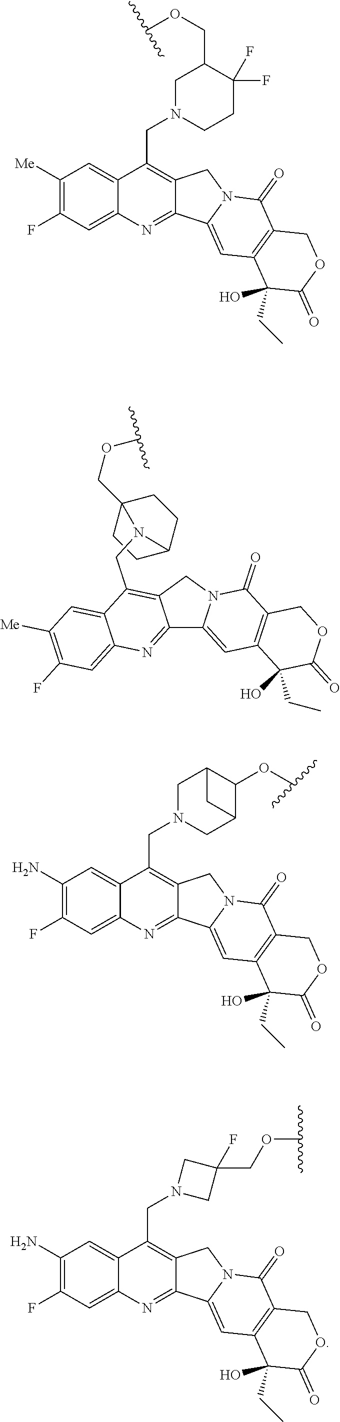 [SMILES: *OCC1CN(Cc2c3c(nc4cc(F)c(C)cc24)-c2cc4c(c(=O)n2C3)COC(=O)[C@]4(O)CC)CCC1(F)F ; *OCC12CCC(CC1)N2Cc1c2c(nc3cc(F)c(C)cc13)-c1cc3c(c(=O)n1C2)COC(=O)[C@]3(O)CC ; *OC1C2CC1CN(Cc1c3c(nc4cc(F)c(N)cc14)-c1cc4c(c(=O)n1C3)COC(=O)[C@]4(O)CC)C2 ; *OCC1(F)CN(Cc2c3c(nc4cc(F)c(N)cc24)-c2cc4c(c(=O)n2C3)COC(=O)[C@]4(O)CC)C1]

Embodiment A: In some embodiments of the present disclosure, the compound of formula (I), or pharmaceutically acceptable salt or solvate thereof, is wherein n¹ and n² are integer independently selected from 0, 1, and 2 and provided that n¹+n² is ≥2; and all other variables are as defined in Aspect 1.

Aspect 2: In some embodiments of the present disclosure, the provided ligand-drug conjugate or the pharmaceutically acceptable salt or solvate thereof is a compound with formula (II) or ligand-drug conjugate thereof or a pharmaceutically acceptable salt or solvate thereof:

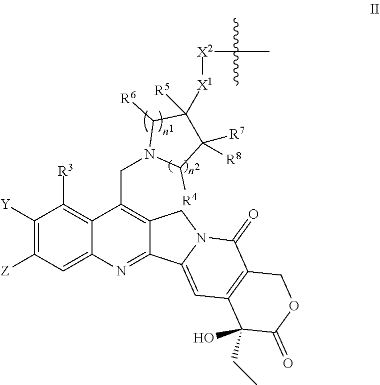

wherein: Y, Z, R³, R⁴, RV, R⁶, R⁷, R⁸, X¹, X², n¹, and n² are defined as in formula (I) in the Aspect 1 or as defined in Embodiment A.

Aspect 2-A: In some embodiments of the present disclosure, the provided ligand-drug conjugate or the pharmaceutically acceptable salt or solvate thereof is a compound with formula (IIa) or ligand-drug conjugate thereof or a pharmaceutically acceptable salt or solvate thereof:

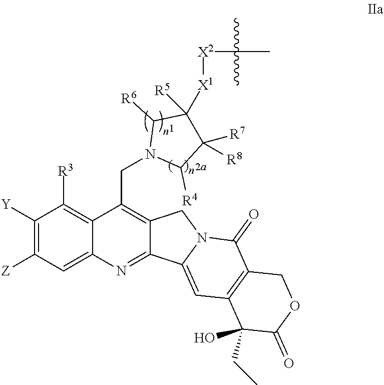

wherein: Y, Z, R³, each R⁴ (independently), each R⁶ (independently), R⁵, R⁷, R⁸, X¹, and X² are as defined as in formula (I) in Aspect 1; and n¹ and n^{2a} are each an integer independently selected from 0, 1, 2, 3, and 4, provided that n¹+n^{2a} is 2, 3, or 4. In some embodiments, Y, Z, R³, each R⁴ (independently), each R⁶ (independently), R⁷, R⁹, and X² are as defined as in formula (I) in Aspect 1; and wherein X¹ is absent; or X¹ and R⁵ taken together with the atom(s) to which they are attached form 3- to 6-membered cycloalkyl or 4- to 8-membered heterocycloalkyl, provided that R⁴ and R⁵ do not also form a ring, where the 3- to 6-membered cycloalkyl and 4- to 8-membered heterocycloalkyl is optionally substituted with one to three R⁹; R⁸ is selected from H and a group consisting of halo, hydroxy, C₁-C₆ alkyl, C₃-C₆ cycloalkyl, aryl, and heteroaryl and wherein each of C₁-C₆ alkyl, C₃-C₆ cycloalkyl, aryl, and heteroaryl is independently optionally substituted with one to four R⁹; and n¹ and n^{2a} are each an integer independently selected from 0, 1, 2, 3, and 4, provided that n¹+n^{2a} is 2, 3, or 4.

Aspect 3: In some embodiments of the present disclosure, the provided ligand-drug conjugate or the pharmaceutically acceptable salt or solvate thereof is according to formula (III):

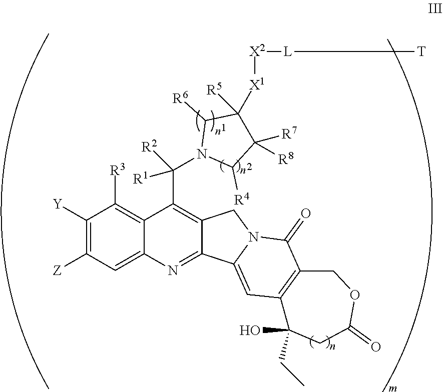

wherein:
Y and Z are independently selected from H and a group consisting of halo, hydroxy, cyano, C₁-C₈ alkyl, C₁-C₈ alkoxy, (C₁-C₈ alkoxy)-C₁-C₈ alkyl, amino, (C₁-C₈ alkyl)NHC(O)O—, (C₃-C₆ cycloalkyl)NHC(O)O—, (C₁-C₈ alkyl)NH—, azetidin-1-yl, pyrrolidin-1-yl, piperidin-1-yl, azepane-1-yl, (C₁-C₈ alkyl)C(O)O—, and (C₁-C₈ alkyl)C(O)NH—; or Y and Z taken together with the atom(s) to which they are attached form a 5- to 8-membered heterocycloalkyl or 5- to 8-membered heteroaryl comprise one to three heteroatoms independently selected from N, O, S, S(O), and S(O)₂; wherein azetidin-1-yl, pyrrolidin-1-yl, piperidin-1-yl, azepane-1-yl, 5- to 8-membered heterocycloalkyl, 5- to 8-membered heteroaryl, and each “C₁-C₈ alkyl” are independently optionally substituted with one to three R⁹;
R³ is selected from H and a group consisting of halo, hydroxy, cyano, C₁-C₈ alkyl, C₁-C₈ alkoxy, (C₁-C₈ alkoxy)-C₁-C₈ alkyl, amino, (C₁-C₈ alkyl)amino, (C₁-C₈ alkyl)NHC(O)O—, (C₃-C₆ cycloalkyl)NHC(O)O—, (C₁-C₈ alkyl)C(O)O—, (C₁-C₈ alkyl)C(O)NH—, aryl, heteroaryl, and nitro; wherein each “C₁-C₈ alkyl” cycloalkyl, aryl, and heteroaryl are independently optionally substituted with one to three R⁹;
each R¹ and each R² are independently selected from H, C₁-C₈ alkyl, C₃-C₆ cycloalkyl, aryl, and heteroaryl; or each pair of R¹ and R² taken together with the atom(s) to which they are attached form 3- to 6-membered cycloalkyl, or 4- to 8-membered heterocycloalkyl; wherein each R¹, R², and R¹/R² ring group is independently optionally substituted with one to four R⁹;
n³ is an integer selected from 0 to 6;
X¹ is absent; or X¹ and R⁸ taken together with the atom(s) to which they are attached form 3- to 6-membered cycloalkyl or 4- to 8-membered heterocycloalkyl; or X¹ and R⁵ taken together with the atom(s) to which they are attached form a 3- to 6-membered cycloalkyl or 4- to 8-membered heterocycloalkyl; each of the 3- to 6-membered cycloalkyl and 4- to 8-membered heterocycloalkyl is optionally substituted with one to three R⁹;
X² comprises one or more groups independently selected from the group consisting of —O—, —S—, —NH—, C₁-C₈ alkylene, C₃-C₆ cycloalkylene, arylene, and heteroarylene; wherein the terminal group in X², connected to L, is O or S; each of C₁-C₈ alkylene, C₃-C₆ cycloalkylene, arylene, and heteroarylene is independently optionally substituted with one to three R⁹;
R⁴, R⁵, R⁶, R⁷, and R⁸ at each occurrence are independently selected from H and a group consisting of halo, hydroxy, C₁-C₈ alkyl, C₃-C₆ cycloalkyl, aryl, and heteroaryl and wherein each of C₁-C₈ alkyl, C₃-C₆ cycloalkyl, aryl, and heteroaryl is independently optionally substituted with one to four R⁹; or
R⁴ and R⁵ taken together with the atom(s) to which they are attached form 3- to 6-membered cycloalkyl, or 4- to 8-membered heterocycloalkyl; or R⁴ and R⁷ taken together with the atom(s) to which they are attached form 3- to 6-membered cycloalkyl, or 4- to 8-membered heterocycloalkyl; or R⁶ and R⁷ taken together with the atom(s) to which they are attached form 3- to 6-membered cycloalkyl, or 4- to 8-membered heterocycloalkyl; or R⁷ and R⁸ taken together with the atom(s) to which they are attached form oxo, 3- to 6-membered cycloalkyl, or 4- to 8-membered heterocycloalkyl; and wherein each of the 3- to 6-membered cycloalkyl and 4- to 8-membered heterocycloalkyl are independently optionally substituted with one to four R⁹; and the remaining of R⁴, R⁵, R⁶, R⁷, and R⁸ at each occurrence are independently selected from H and the group consisting of halo, hydroxy, C₁-C₈ alkyl, C₃-C₆ cycloalkyl, aryl, and heteroaryl, wherein the C₁-C₈ alkyl, C₃-C₆ cycloalkyl, aryl, and heteroaryl are independently optionally substituted with one to four R⁹;
R⁹ at each occurrence is independently selected from the group consisting of halogen, oxo, hydroxy, cyano, C₁-C₈ alkyl, C₃-C₆ cycloalkyl, C₁-C₈ alkoxy, aryl, and heteroaryl; or two R⁹ groups when attached to adjacent carbons and taken together with the carbons to which they are attached form a fused C₃-C₆ cycloalkyl; or two R⁹ groups when attached to the same carbon and taken together with the carbon to which they are attached form a spiro C₃-C₆ cycloalkyl; wherein each C₁-C₈ alkyl, C₃-C₆ cycloalkyl, C₁-C₈ alkoxy, aryl, heteroaryl, fused C₃-C₆ cycloalkyl, and spiro C₃-C₆ cycloalkyl is optionally independently substituted with one to three fluoro or hydroxy, and C₁-C₃ alkyl;
n¹ and n² are each an integer independently selected from 0, 1, 2, 3, and 4, provided that n¹+n² is 2, 3, or 4; and
n is an integer selected from 0 and 1;
T is a targeting or binding ligand;
L is a releasable linker;
m is an integer or fraction of integer selected from 1 to 10.

Embodiment B: In some embodiments of the present disclosure, the compound of formula III, or pharmaceutically acceptable salt or solvate thereof, is wherein n¹ and n² are integer independently selected from 0, 1, and 2 and provided that n¹+n² is ≥2; and all other variables are as defined in Aspect 3.

Aspect 3-A: In some embodiments of the present disclosure, the provided ligand-drug conjugate or the pharmaceutically acceptable salt or solvate thereof is according to formula (IIIa):

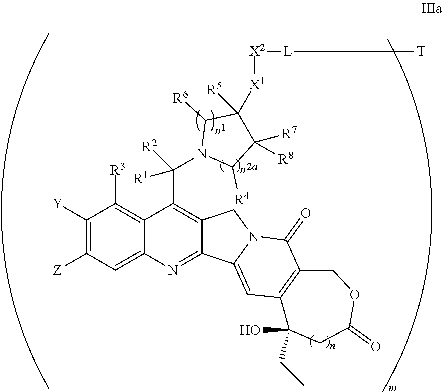

wherein: Y, Z, R¹, R², R³, each R⁴ (independently), each R⁶ (independently), R⁵, R⁷, R⁸, X¹, X², L, T, m, and n are as defined as in formula (III) in Aspect 3; and n¹ and n^{2a} are each an integer independently selected from 0, 1, 2, 3, and 4, provided that n¹+n^{2a} is 2, 3, or 4. In some embodiments, Y, Z, R¹, R², R³, each R⁴ (independently), R⁵, each R⁶ (independently), R⁷, X², L, T, m, and n are as defined as in formula (III) in Aspect 3; and wherein X¹ is absent; or X¹ and R⁵ taken together with the atom(s) to which they are attached form a 3- to 6-membered cycloalkyl or 4- to 8-membered heterocycloalkyl, provided that R⁴ and R⁵ do not also form a ring, where the 3- to 6-membered cycloalkyl and 4- to 8-membered heterocycloalkyl is optionally substituted with one to three R⁹; R⁸ is selected from H and a group consisting of halo, hydroxy, C₁-C₆ alkyl, C₃-C₆ cycloalkyl, aryl, and heteroaryl and wherein each of C₁-C₆ alkyl, C₃-C₆ cycloalkyl, aryl, and heteroaryl is independently optionally substituted with one to four R⁹; and n¹ and n^{2a} are each an integer independently selected from 0, 1, 2, 3, and 4, provided that n¹+n^{2a} is 2, 3, or 4. In some embodiments, R¹ and R² are each hydrogen.

Aspect 3-B: In some embodiments of the present disclosure, the provided ligand-drug conjugate or the pharmaceutically acceptable salt or solvate thereof is according to formula (IIIb):

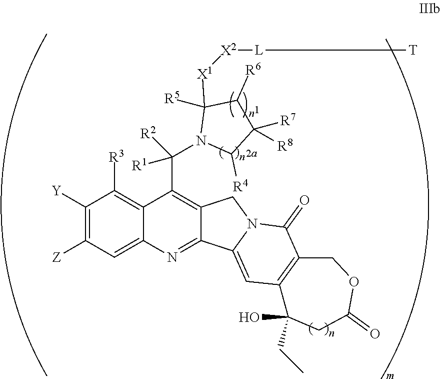

wherein: Y, Z, R¹, R², R³, X², L, T, m, and n are as defined as in formula (IIIa) in Aspect 3-A and any embodiments thereof, R⁴, R⁵, R⁶, R⁷, and R⁸ at each occurrence are independently selected from H and a group consisting of halo, hydroxy, C₁-C₈ alkyl, C₃-C₆ cycloalkyl, aryl, and heteroaryl and wherein each of C₁-C₈ alkyl, C₃-C₆ cycloalkyl, aryl, and heteroaryl is independently optionally substituted with one to four R⁹; X¹ is absent; and n¹ and n^{2a} are each an integer independently selected from 0, 1, 2, 3, and 4, provided that n¹+n^{2a} is 2, 3, or 4. In some embodiments, R¹ and R² are each hydrogen and

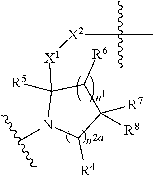

is

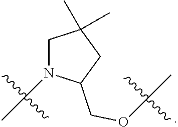 [SMILES: *OCC1CC(C)(C)CN1*]

In some embodiments of the present disclosure, the provided ligand-drug conjugate, or a pharmaceutically acceptable salt or solvate thereof, of any of the formula (I), (II), (IIa), (III), and (IIIa), including as defined in the Aspect 1, Embodiment A, Aspect 2, Aspect 2-A, Aspect 3, Aspect 3-A, and Embodiment B, wherein:

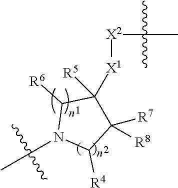

in formulas I, II, and III and

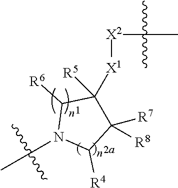

in formulas IIa, and IIIa are a structure selected from the following structures below

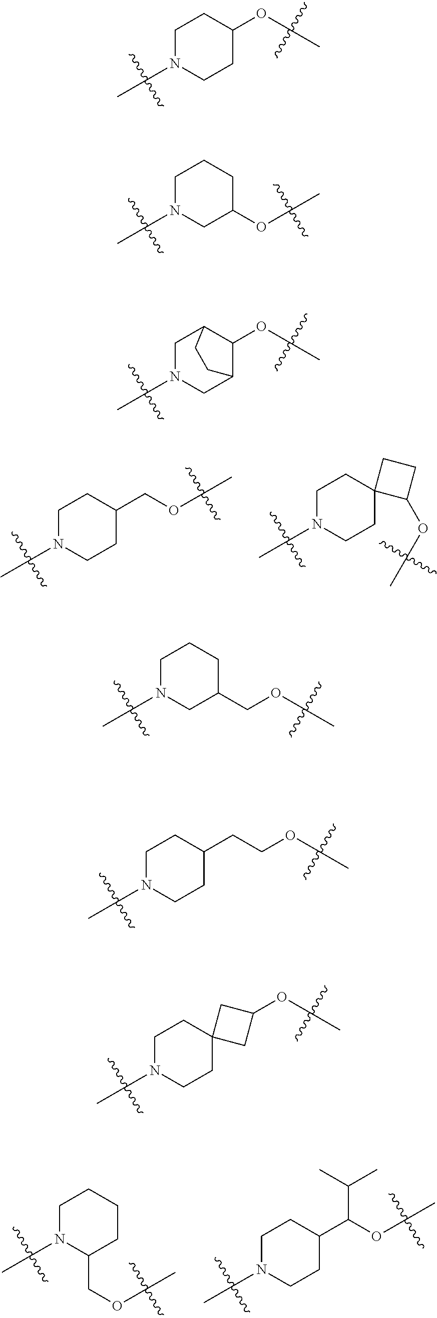

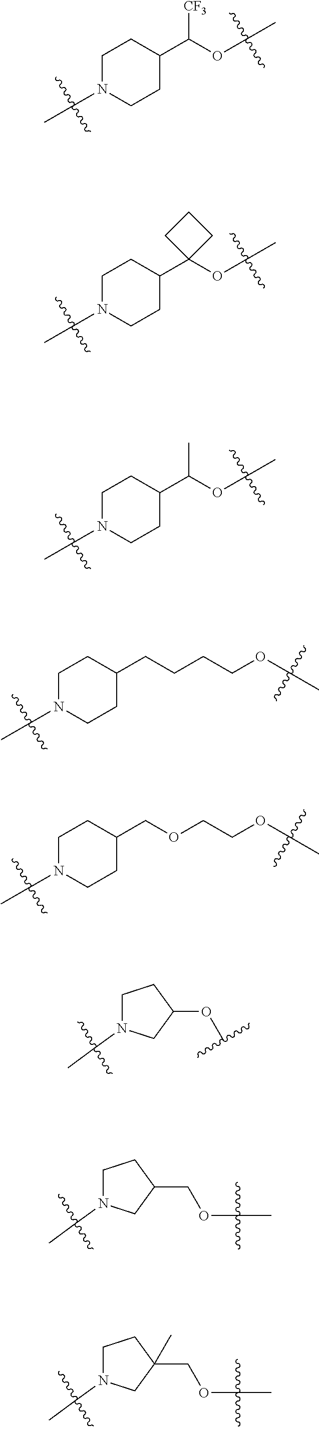

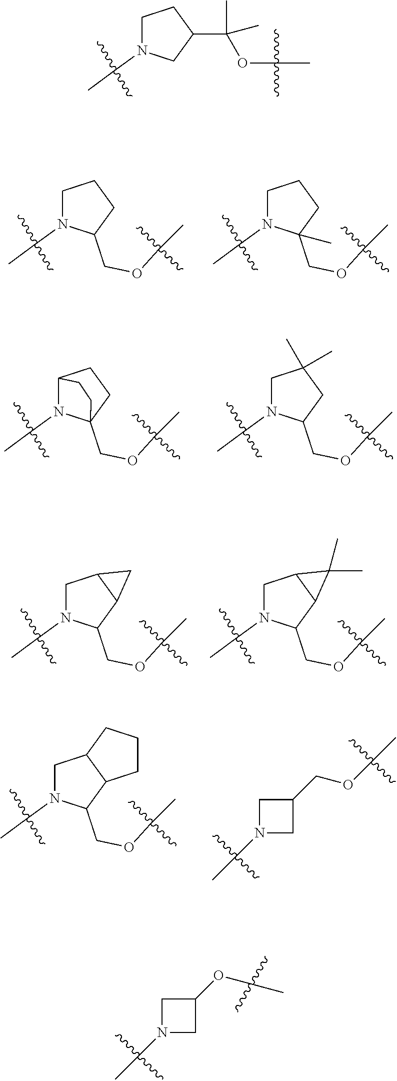

In some embodiments, the structure of any of the formula (I), (II), (IIa), (III), and (IIIa), or a pharmaceutically acceptable salt or solvate thereof, including as defined in the Aspect 1, Embodiment A, Aspect 2, Aspect 2-A, Aspect 3, Aspect 3-A, and Embodiment B, wherein

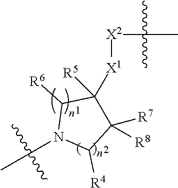

in formulas I, II, and III and

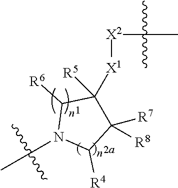

in formulas IIa, and IIIa are selected from the following structures below,

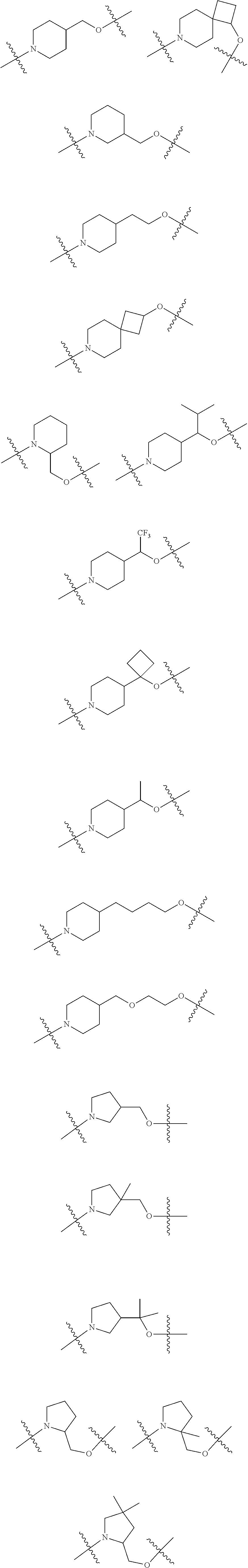

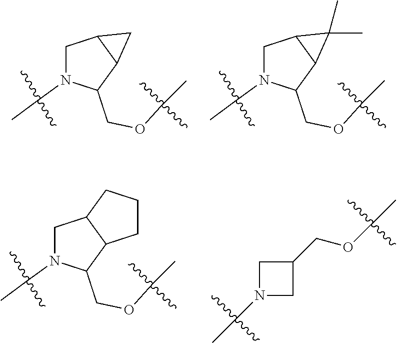 [SMILES: *OCC1C2CC2CN1* ; *OCC1C2C(CN1*)C2(C)C ; *OCC1C2CCCC2CN1* ; *OCC1CN(*)C1]

In some embodiments, the structure of any of the formula (I), (II), (IIa), (III), and (IIIa), or a pharmaceutically acceptable salt or solvate thereof, including as defined in the Aspect 1, Embodiment A, Aspect 2, Aspect 2-A, Aspect 3, Aspect 3-A, and Embodiment B, wherein

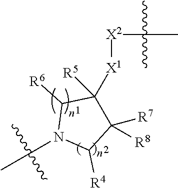

in formulas I, II, and III and

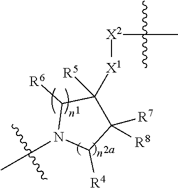

in formulas IIa, and IIIa are selected from the following structures below,

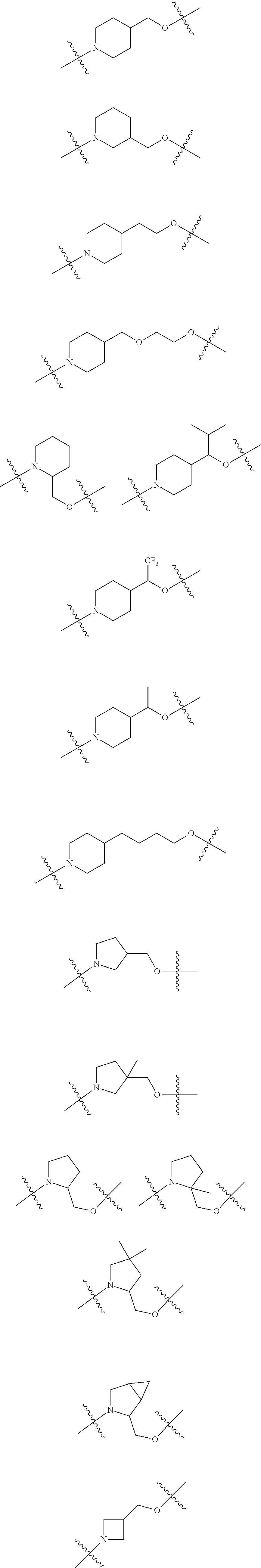

In some embodiments, the structure of any of the formula (I), (II), (IIa), (III), and (IIIa), or a pharmaceutically acceptable salt or solvate thereof, including as defined in the Aspect 1, Embodiment A, Aspect 2, Aspect 2-A, Aspect 3, Aspect 3-A, and Embodiment B, wherein

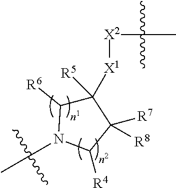

in formulas I, II, and III and

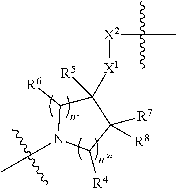

in formulas IIa, and IIIa are selected from the following structures below,

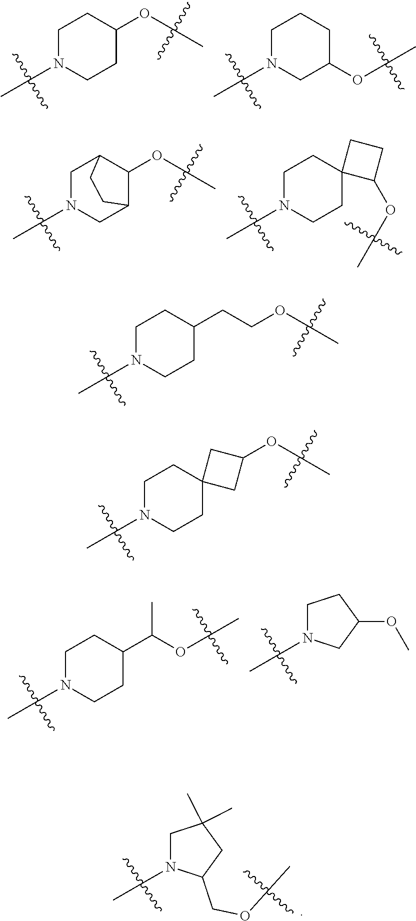

In some embodiments, the structure of any of the formula (I), (II), (IIa), (III), and (IIa), or a pharmaceutically acceptable salt or solvate thereof, including as defined in the Aspect 1, Embodiment A, Aspect 2, Aspect 2-A, Aspect 3, Aspect 3-A, and Embodiment B, wherein

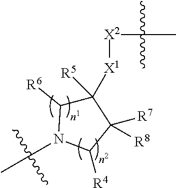

in formulas I, II, and III and

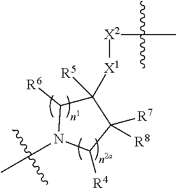

in formulas IIa, and IIIa are selected from the following structures below,

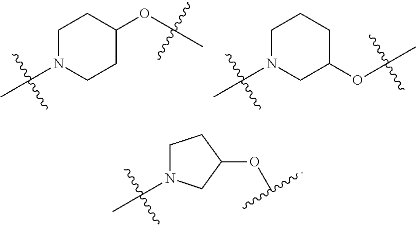 [SMILES: *OC1CCN(*)CC1 ; *OC1CCCN(*)C1 ; *OC1CCN(*)C1]

In another preferred embodiment of the present disclosure, in the ligand-drug conjugate or the pharmaceutically acceptable salt or solvate thereof according to the present disclosure, including of any of the formula (I), (II), (IIa), (III), (IIIa), and (IIIb) and any embodiments thereof, including as defined in the Aspect 2, Aspect 2-A, Aspect 3, Aspect 3-A, and Embodiment B, the linker unit L is -L¹-L²-L³-L⁴-, wherein
L¹ is selected from the group consisting of

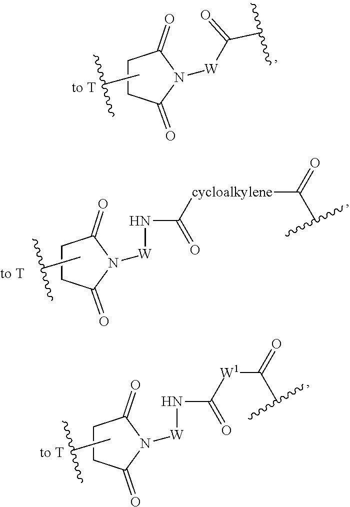

—CH₂—C(O)—NR¹⁰—W—C(O)— and —C(O)—W—C(O)—, —W—, wherein W and W¹ are independently selected from the group consisting of C₁-C₈ alkylene, —(C₁-C₈ alkylene)-cycloalkylene-, arylene, heteroarylene, and linear heteroalkylene, wherein the linear heteroalkylene comprise 1 to 8 carbon atom(s), and 1 to 3 heteroatom(s) selected from the group consisting of N, O, S, SO, and SO₂, and wherein the |—(C₁-C₈ alkylene)-cycloalkylene-linear heteroalkylene, arylene, and heteroarylene are each independently optionally further substituted by one or more substituent(s) selected from the group consisting of halogen, hydroxy, cyano, amino, alkyl, chloroalkyl, deuterated alkyl, alkoxy and cycloalkyl; wherein the left side of each of the L¹ groups provided above is attached to T;
L² is selected from the group consisting of —NR¹¹(CH₂CH₂O)ₚ¹CH₂CH₂C(O)—, —NR¹¹(CH₂CH₂O)ₚ¹CH₂CH₂—, —NR¹¹(CH₂CH₂O)ₚ¹CH₂C(O)—, —S(CH₂)ₚ¹C(O)— and a chemical bond, wherein p¹ at each occurrence is an integer selected from 1 to 20; and L² is preferably a chemical bond; wherein the left side of each of the L² groups provided above is attached to L¹;
L³ is a peptide residue composed of 2 to 7 amino acids, wherein the amino acids are optionally further substituted by one or more substituent(s) selected from the group consisting of halogen, hydroxy, cyano, amino, alkyl, chloroalkyl, deuterated alkyl, alkoxy and cycloalkyl; wherein the left side of each of the L³ groups provided above is attached to L²;
L⁴ is selected from the group consisting of —NR¹²(CR¹³R¹⁴)—, —C(O)NR¹²—, —C(O)NR¹²(CH)ₜ—,

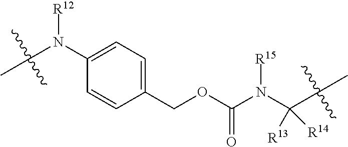

and a chemical bond, wherein t at each occurrence is an integer selected from 1 to 6; and L⁴ is preferably —NR¹²(CR¹³R¹⁴)ₜ⁻; wherein the left side of each of the L⁴ groups provided above is attached to the right side of L³ and the right side of each of the L⁴ groups is attached to X²;
R¹⁰, R¹¹ and R¹² are each independently selected from the group consisting of H, alkyl, haloalkyl, deuterated alkyl and hydroxyalkyl;
R¹³ and R¹⁴ are each independently selected from the group consisting of H, halogen, alkyl, haloalkyl, deuterated alkyl, and hydroxyalkyl; and
R¹⁵ is selected from —CH₂CH₂SO₂CH₃, and —CH₂CH₂N(CH₃)₂.

In another preferred embodiment of the present disclosure, including of any of the formula (I), (II), (IIa), (III), (IIIa), and (IIIb) and any embodiments thereof, including as defined in the Aspect 1, Embodiment A, Aspect 2, Aspect 2-A, Aspect 3, Aspect 3-A, and Embodiment B, in the ligand-drug conjugate, or the pharmaceutically acceptable salt or solvate thereof, the linker unit L¹ is selected from the group consisting of

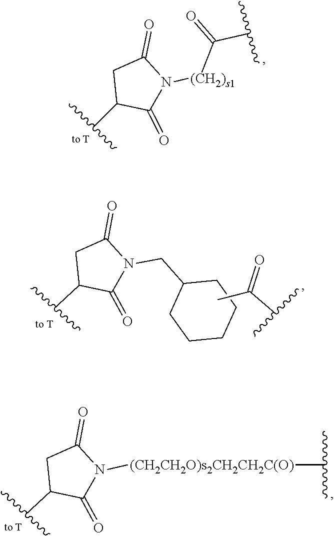

—CH₂—C(O)—NR¹⁰—(CH₂)s³-C(O)— (wherein the left hand side of this group is attached to T), —C(O)—(CH₂)s⁴-C(O)— (wherein the left hand side of this group is attached to T), and —C₆H₄—, wherein s¹ is an integer selected from 2 to 8; s² is an integer selected from 1 to 3; s³ is an integer selected from 1 to 8; s⁴ is an integer selected from 1 to 8; where s¹=5 is preferred;
L² is selected from the group consisting of —NR¹¹(CH₂CH₂O)ₚ¹CH₂CH₂C(O)—, —NR¹¹(CH₂CH₂O)ₚ¹CH₂CH₂—, —NR¹¹(CH₂CH₂O)ₚ¹CH₂C(O)—, —S(CH₂)ₚ¹C(O)—, and a chemical bond, wherein p¹ at each occurrence is an integer selected from 6 to 12; and L² is preferably a chemical bond; wherein the left side of each of the L² groups provided above is attached to L¹;
L³ is a peptide residue composed of 2 to 7 amino acids. wherein the amino acids are selected from Phenylalanine (F), Glycine (G), Valine (V), Lysine (K), Citrulline, Serine (S), Glutamic acid (E), Aspartic acid (N); preferably a peptide residue composed of 1, 2 or more Phenylalanine and Glycine; more preferably is a peptide residue composed of 4 amino acids; the most preferably is a peptide residue composed of GGFG; wherein the left side of each of the L³ groups provided above is attached to L²;
L⁴ is —NR¹²(CR¹³R¹⁴)—, R¹² is H or alkyl, R¹³ and R¹⁴ are each independently selected from H and alkyl, t is 1 or 2; L⁴ is optionally —NR¹²CR¹³R¹⁴—; or L⁴ is preferably —NHCH₂—; wherein the left side of each of the L⁴ groups provided above is attached to the right side of L³ and the right side of each of the L⁴ groups is attached to X²;
R¹⁰, R¹¹ and R¹² are each independently selected from the group consisting of hydrogen, alkyl, haloalkyl, deuterated alkyl and hydroxyalkyl; and
R¹³ and R¹⁴ are each independently selected from the group consisting of hydrogen, halogen, alkyl, haloalkyl, deuterated alkyl and hydroxyalkyl.

Aspect 4: In some embodiments of the present disclosure, provided is a compound or the pharmaceutically acceptable salt or solvate thereof, optionally as a tautomer, a mesomer, a racemate, an enantiomer, a diastereomer, a mixture thereof, where the compound is of formula (IV):

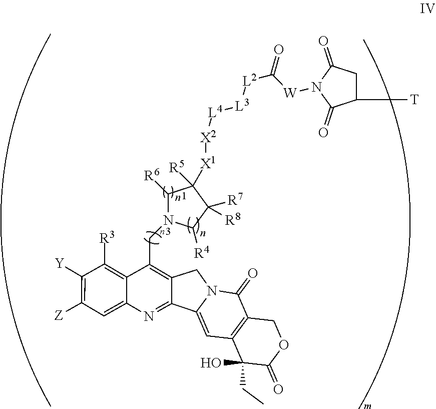

wherein: Y, Z, R³, R⁴, R⁵, R⁶, R⁷, R⁸, X¹, X², L², L³, L⁴, W, n¹, n², T, and m are defined as in formula (III) and any embodiments thereof, including as defined in Embodiment B, and n³ is an integer selected from 0 to 6.

Aspect 4-A: In some embodiments of the present disclosure, provided is a compound or the pharmaceutically acceptable salt or solvate thereof, optionally as a tautomer, a mesomer, a racemate, an enantiomer, a diastereomer, a mixture thereof, where the compound is of formula (IVa):

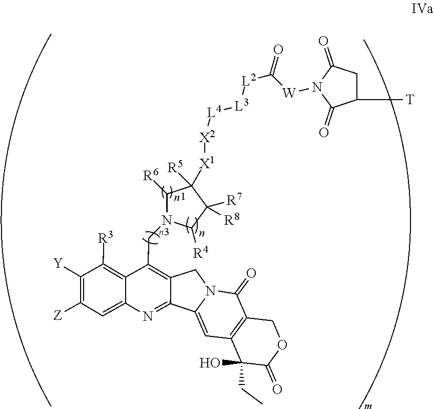

wherein: Y, Z, R³, R⁴, R⁵, R⁶, R⁷, R⁸, X¹, X², L², L³, L⁴, W, n¹, n^{2a}, T, and m are defined as in formula (IIIa) and any embodiments thereof, and n³ is an integer selected from 0 to 6.

In some embodiments of the present disclosure, the provided ligand-drug conjugate, or a pharmaceutically acceptable salt or solvate thereof, of formula (IV) or (IVa), wherein:

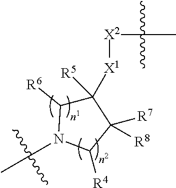

for formula (IV) and

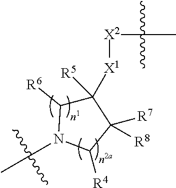

for formula (IVa)
are a structure selected from the following structures below,

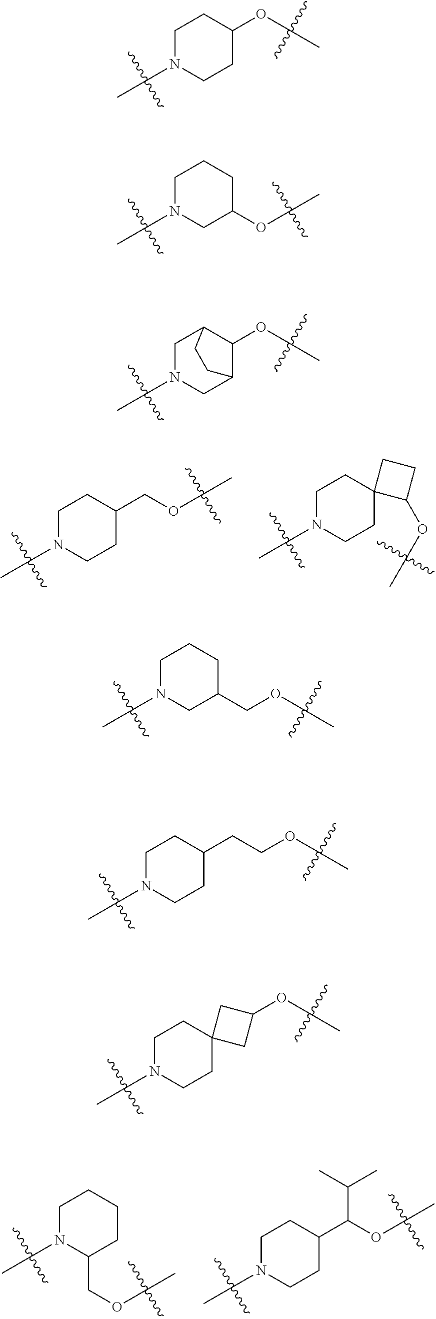

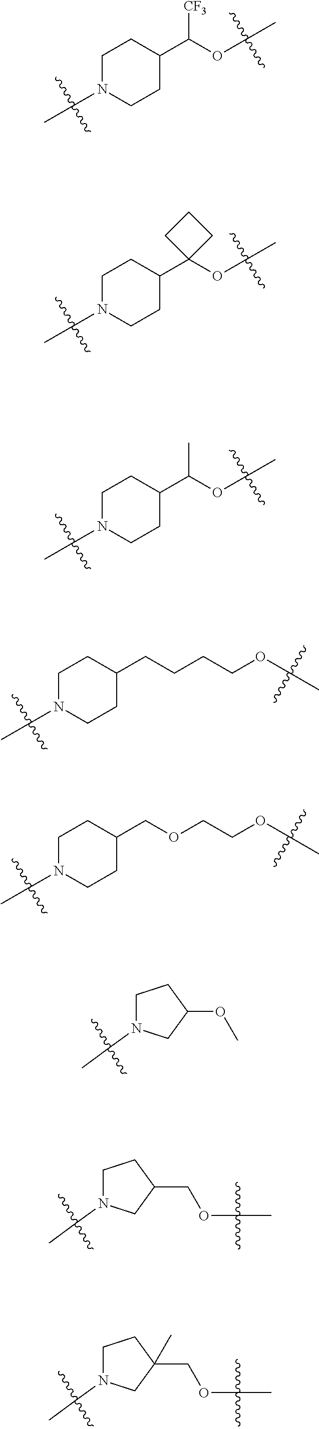

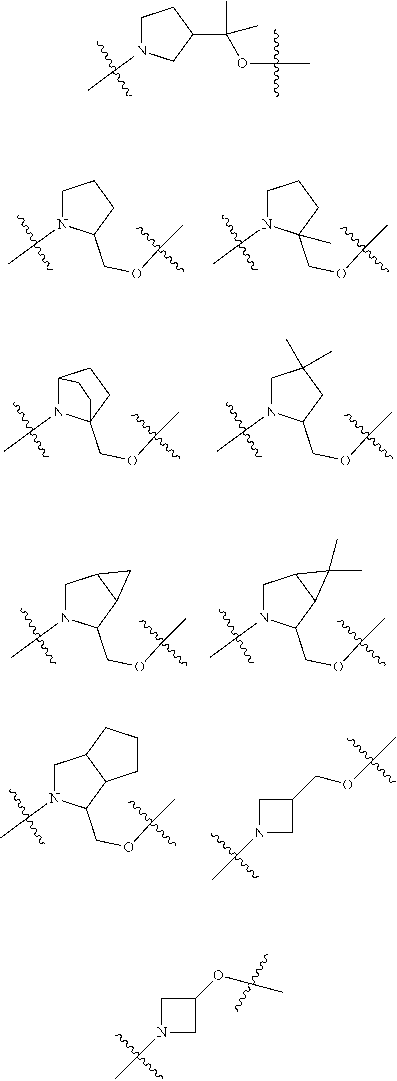

In some embodiments, the structure of any of the formula (IV) or (Ma), or a pharmaceutically acceptable salt or solvate thereof wherein

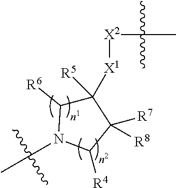

for formula (IV) and

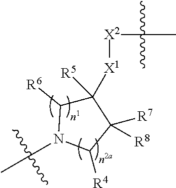

for formula (IVa)
are selected from the following structures below,

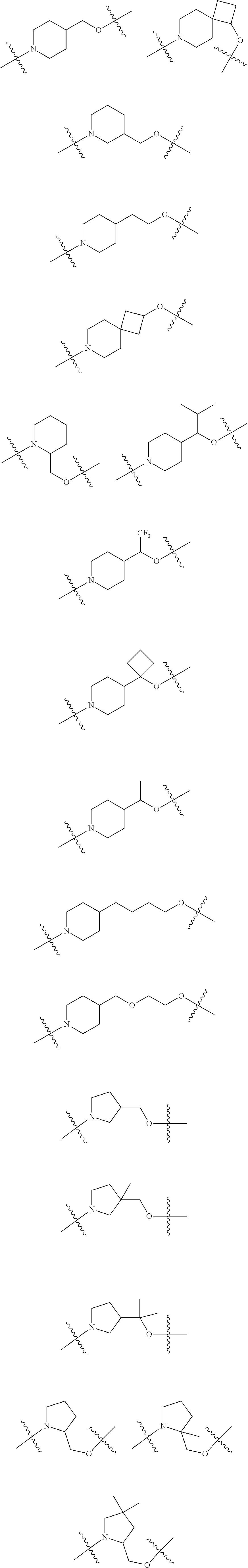

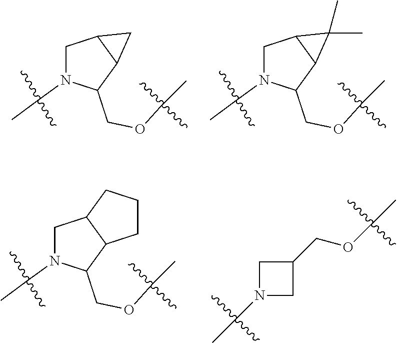 [SMILES: *OCC1C2CC2CN1* ; *OCC1C2C(CN1*)C2(C)C ; *OCC1C2CCCC2CN1* ; *OCC1CN(*)C1]

In some embodiments, the structure of formula (IV) or (IVa), or a pharmaceutically acceptable salt or solvate thereof, wherein

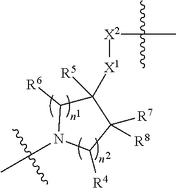

for formula (IV) and

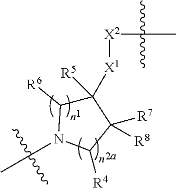

for formula (IVa)
are selected from the following structures below,

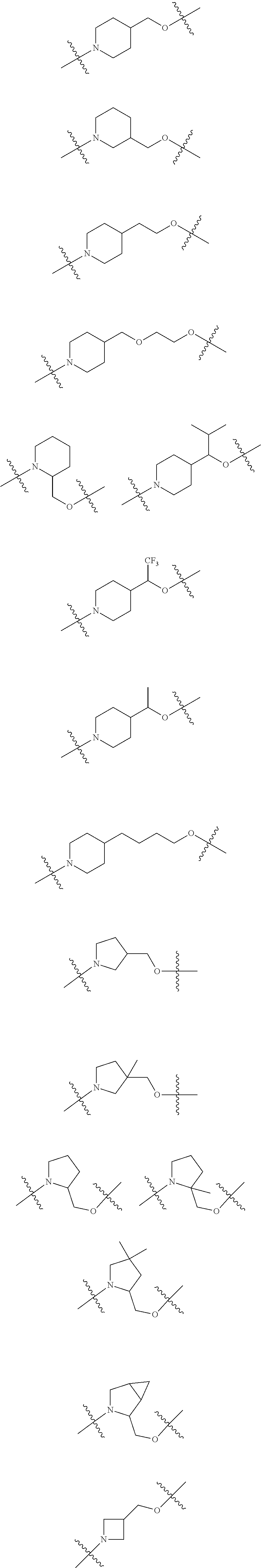

In some embodiments, the structure of formula (IV) or (IVa), or a pharmaceutically acceptable salt or solvate thereof, wherein

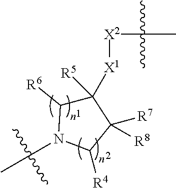

for formula (IV) and

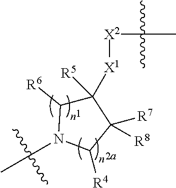

for formula (IVa)
are selected from the following structures below

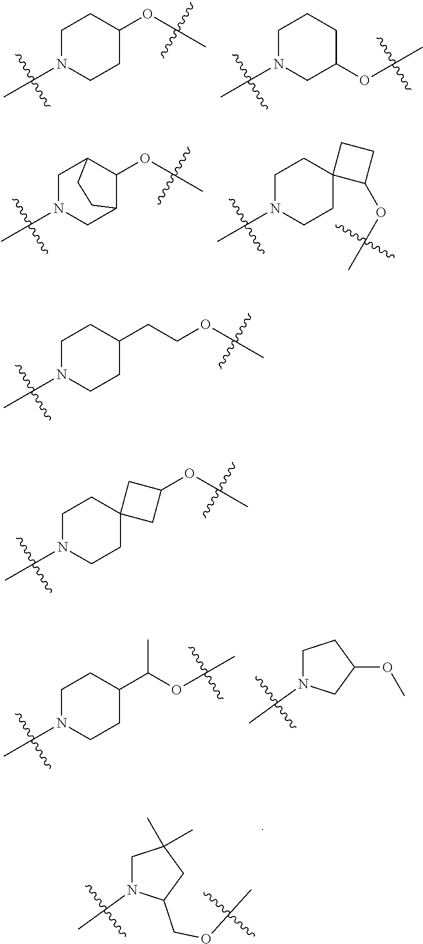

In some embodiments, the structure of formula (IV) or (IVa), or a pharmaceutically acceptable salt or solvate thereof, wherein

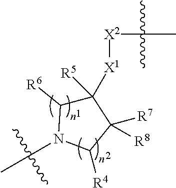

for formula (IV) and

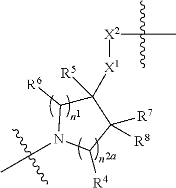

for formula (IVa)
are selected from the following structures below,

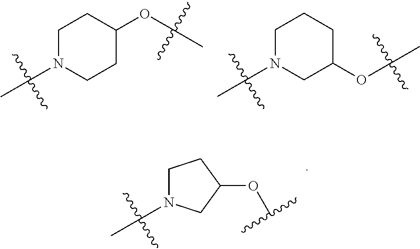 [SMILES: *OC1CCN(*)CC1 ; *OC1CCCN(*)C1 ; *OC1CCN(*)C1]

In some embodiments, the ligand-drug conjugates include, but are not limited to:

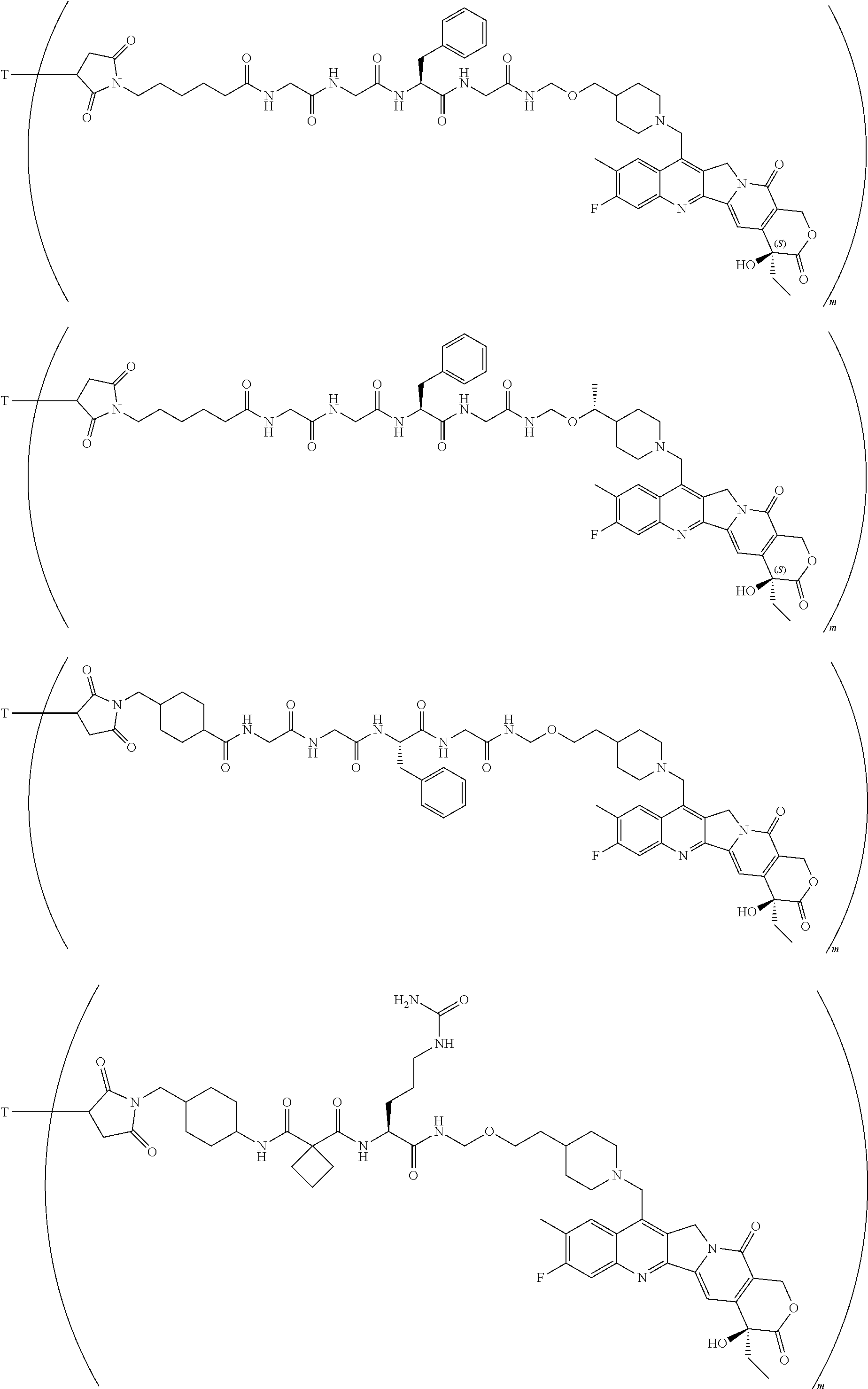

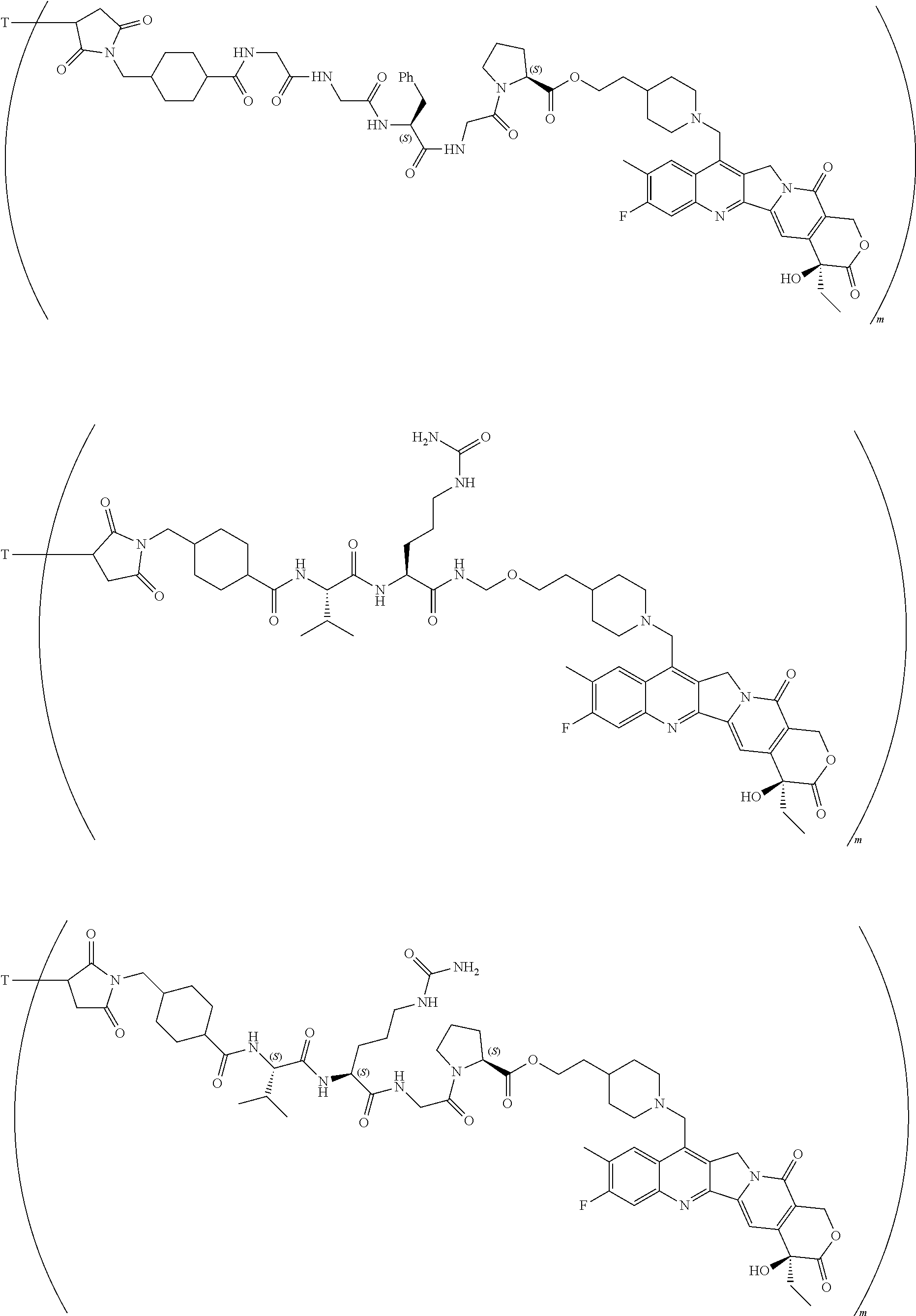

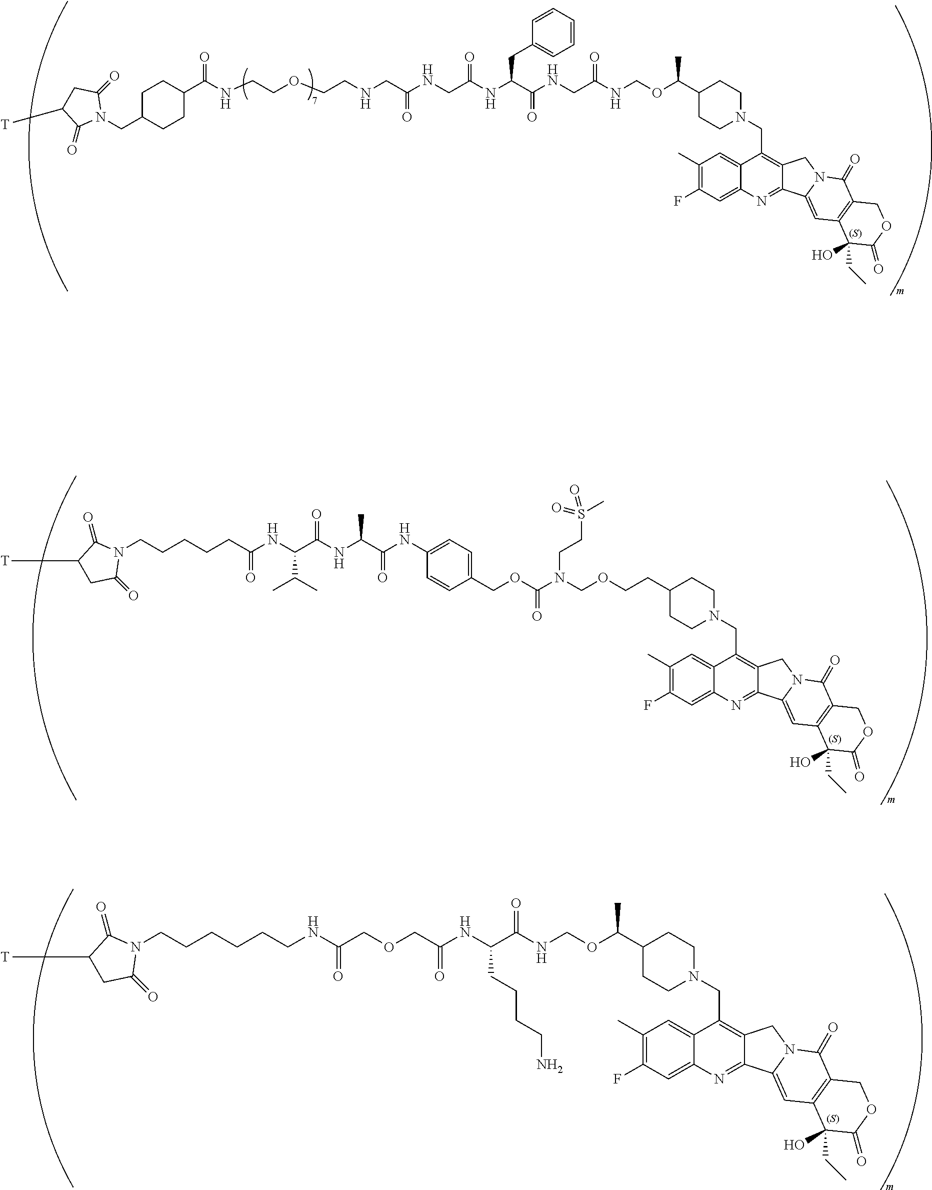

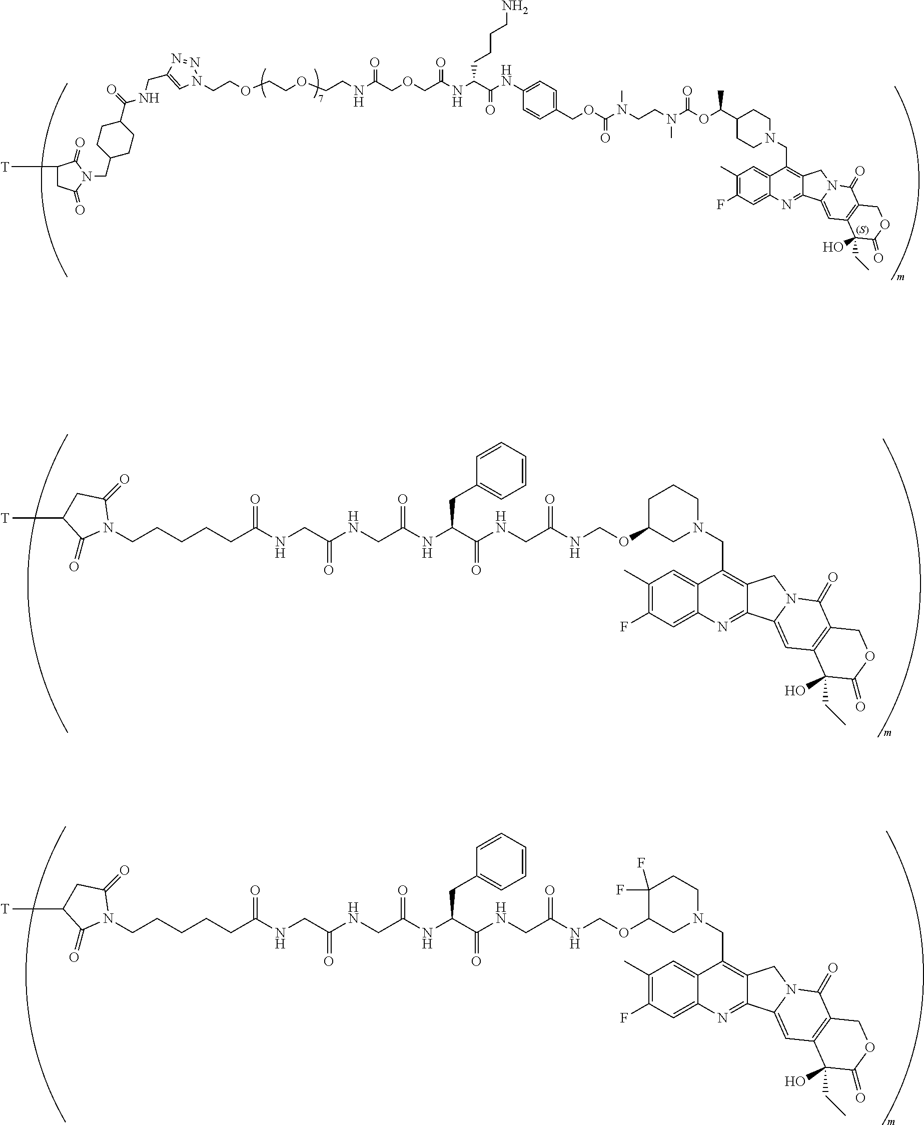

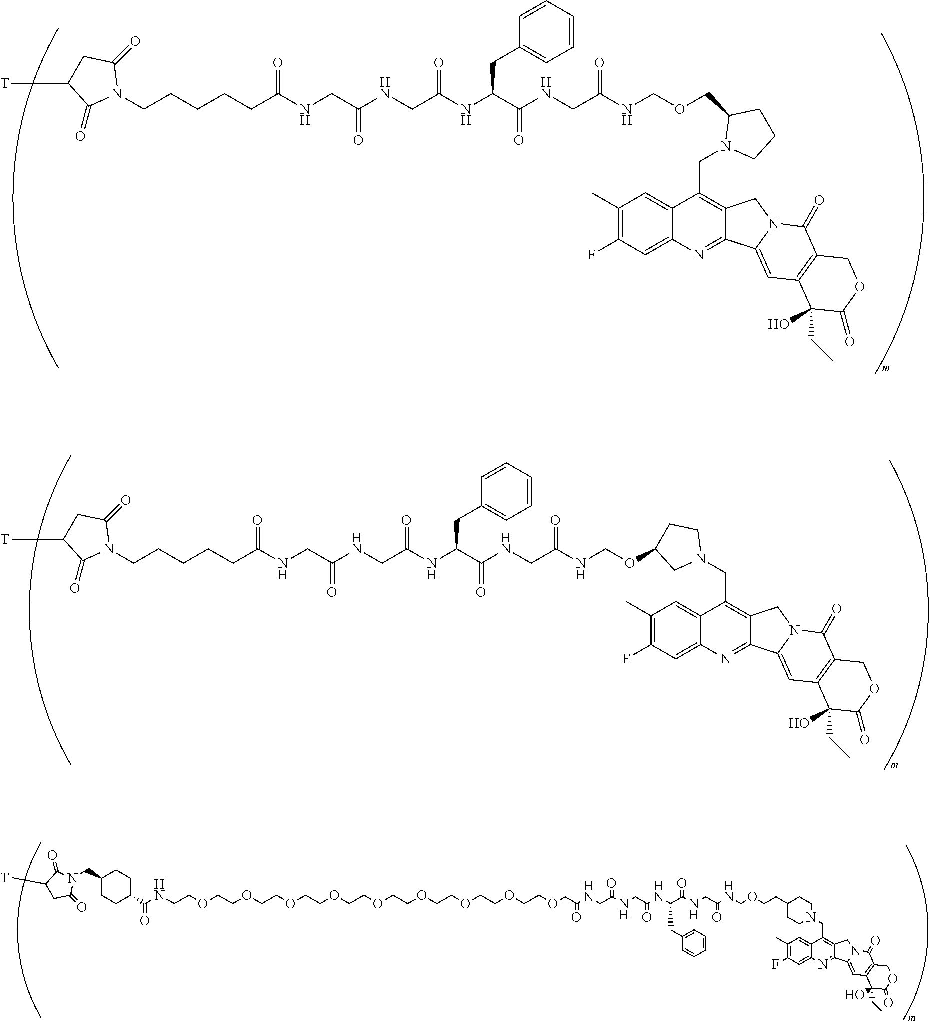

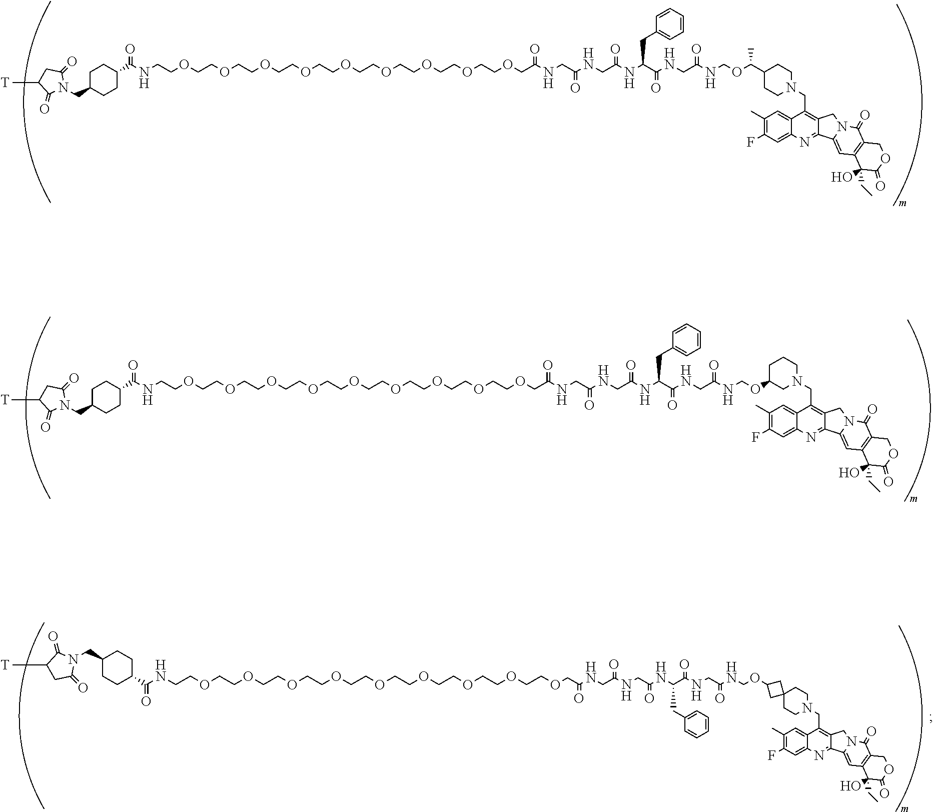

or a pharmaceutically acceptable salt or solvate thereof,
wherein T and m are defined as in formula (III) or (IIIa) and any embodiments thereof, including as defined in Embodiment B; and wherein further embodiments of T are provided below.

In some embodiments of the present disclosure, provided is the ligand-drug conjugate or the pharmaceutically acceptable salt or solvate thereof, wherein:
T is a targeting antibody or ligand binding to antigen; wherein the antibody is selected from chimeric antibody, humanized antibody and human antibody; optionally, wherein T is a monoclonal antibody.

In some embodiments of the present disclosure, provided is the ligand-drug conjugate or the pharmaceutically acceptable salt or solvate thereof, wherein:
T is selected from anti-Her2(ErbB2) antibody, anti-EGFR antibody, anti-B₇H₃ antibody, anti-c-MET antibody, anti-Her3(ErbB3) antibody, anti-Her4(ErbB4) antibody, anti-CD20 antibody, anti-CD22 antibody, anti-CD30 antibody, anti-CD33 antibody, anti-CD44 antibody, anti-CD56 antibody, anti-CD70 antibody, anti-CD73 antibody, anti-CD105 antibody, anti-CEA antibody, anti-A33 antibody, anti-Cripto antibody, anti-EphA2 antibody, anti-G250 antibody, anti-MICI antibody, anti-Lewis Y antibody, anti-VEGFR antibody, anti-GPNMB antibody, anti-Integrin antibody, anti-PSMA antibody, anti-Tenascin-C antibody, anti-SLC44A4 antibody or anti-Mesothelin antibody, anti-ROR1 antibody or the fragment binding to the antigen.

In some embodiments of the present disclosure, provided is the ligand-drug conjugate or the pharmaceutically acceptable salt or solvate thereof, wherein: T is selected from Trastuzumab, Pertuzumab, Nimotuzumab, Enoblituzumab, Emibetuzumab, Inotuzumab, Pinatuzumab, Brentuximab, Gemtuzumab, Bivatuzumab, Lorvotuzumab, cBR96 and Glembatumumab or the fragment binding to the antigen.

In some embodiments, the ligand-drug conjugates include, but are not limited to:

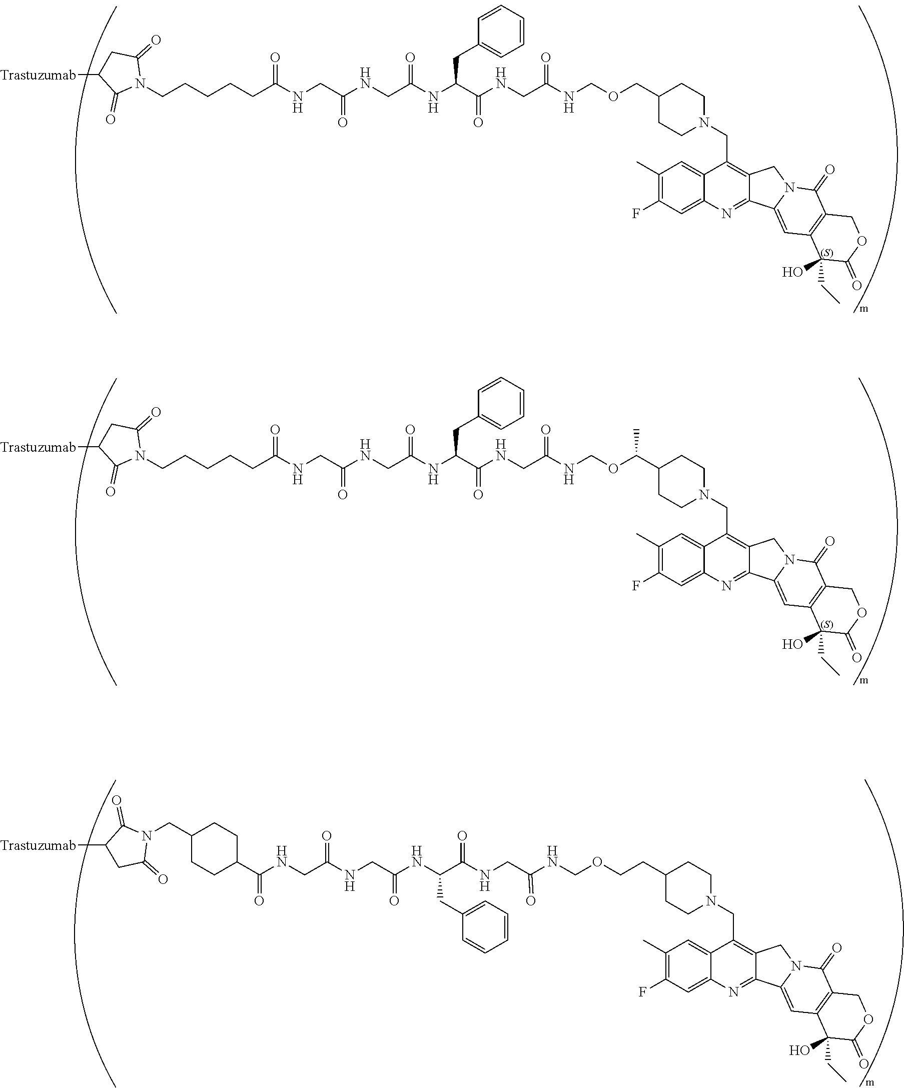

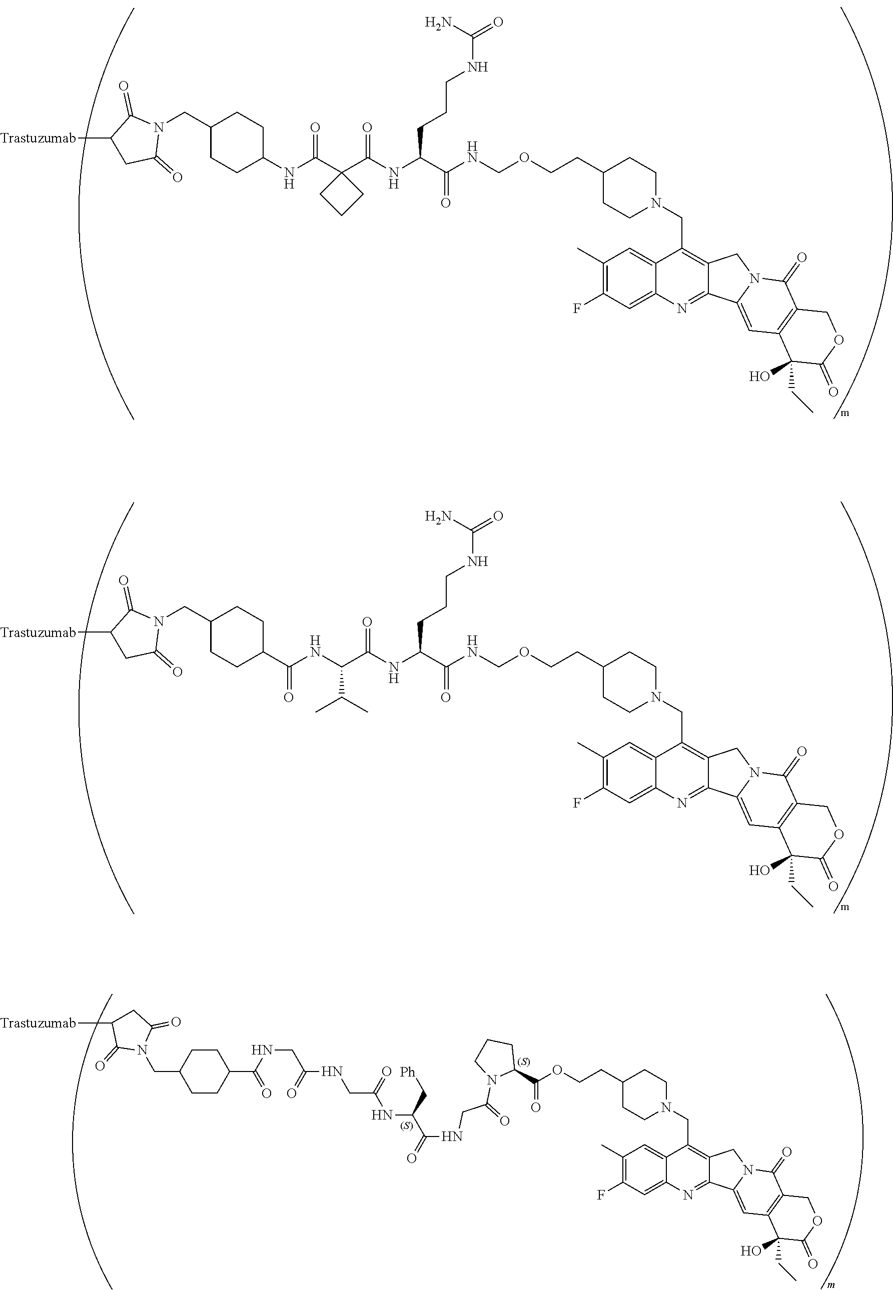

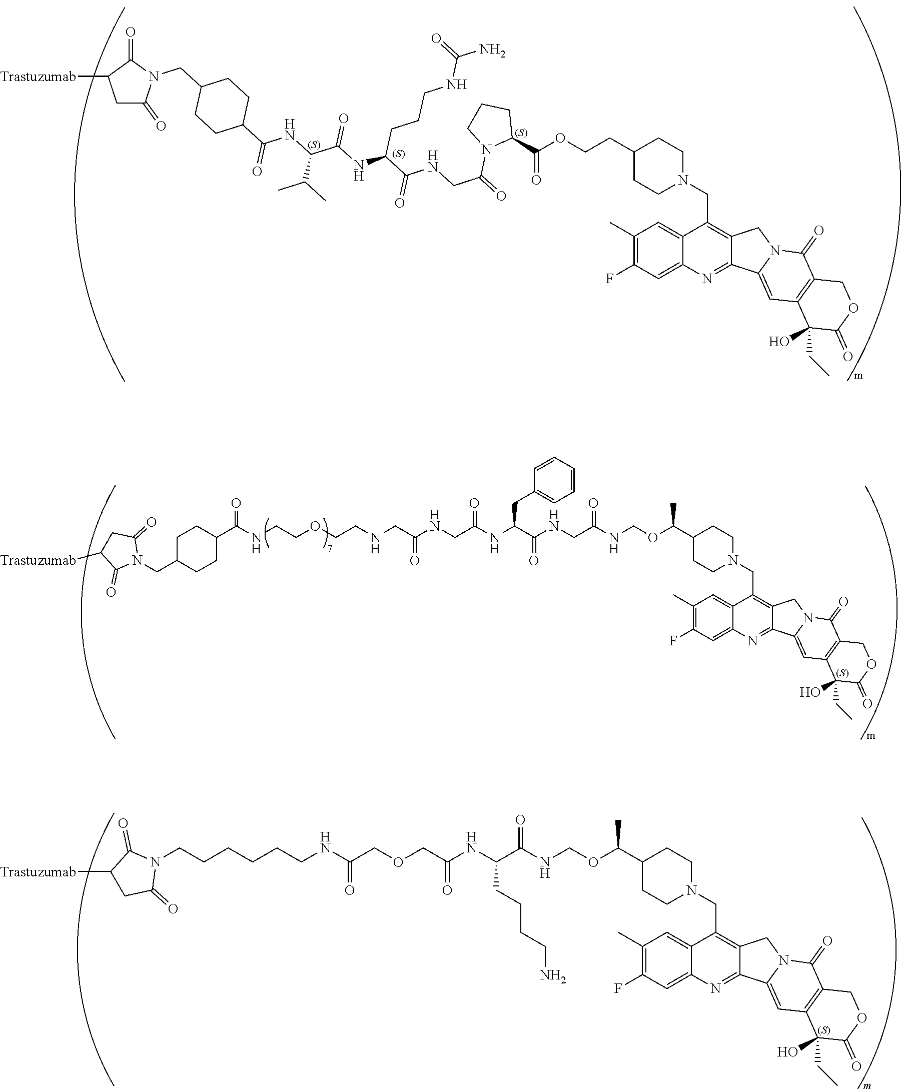

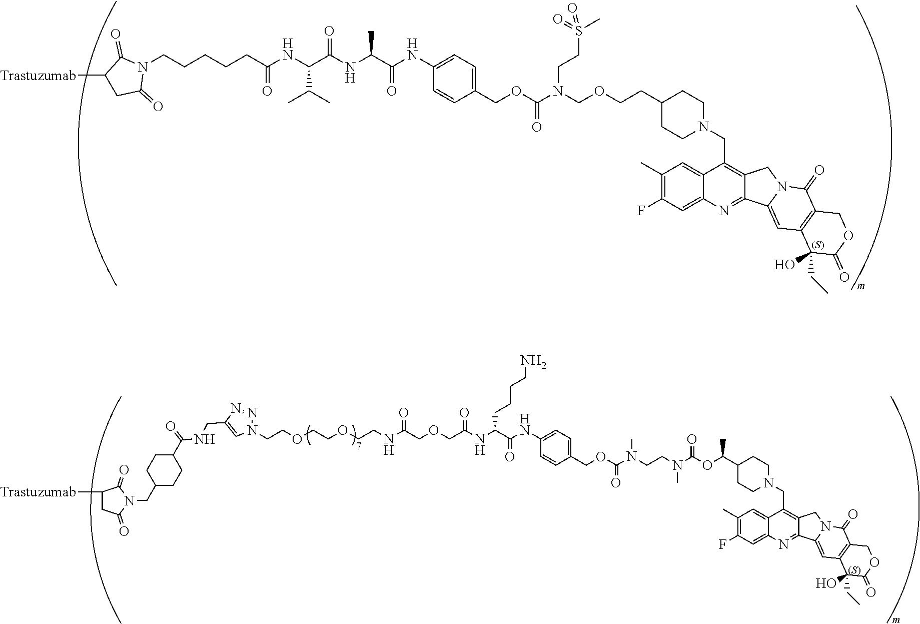

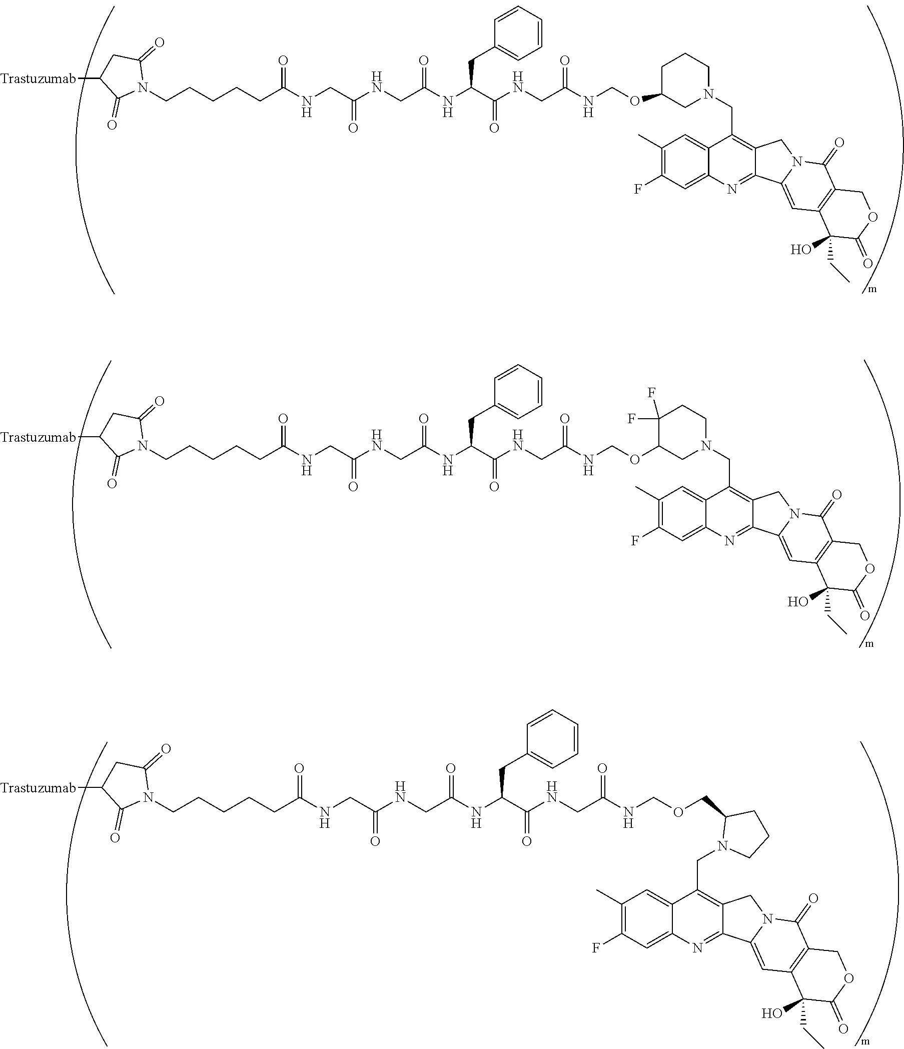

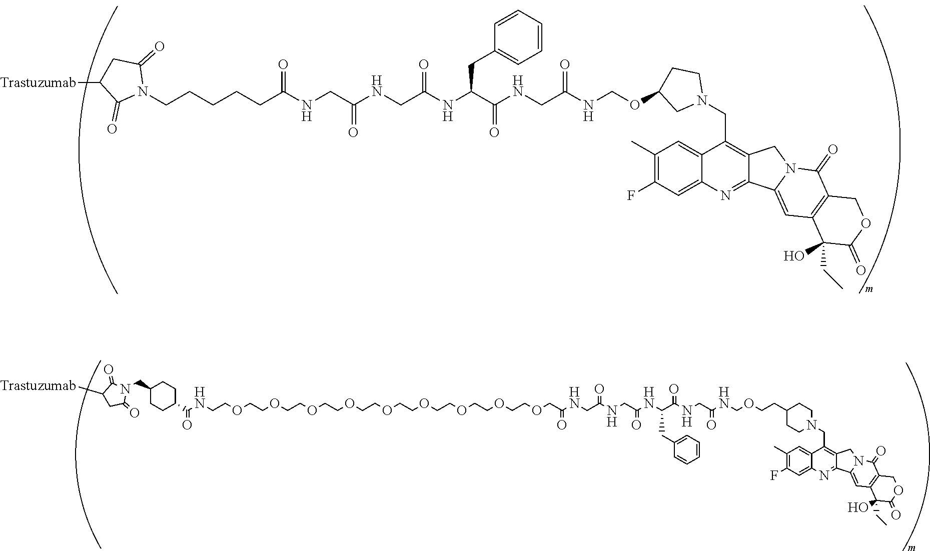

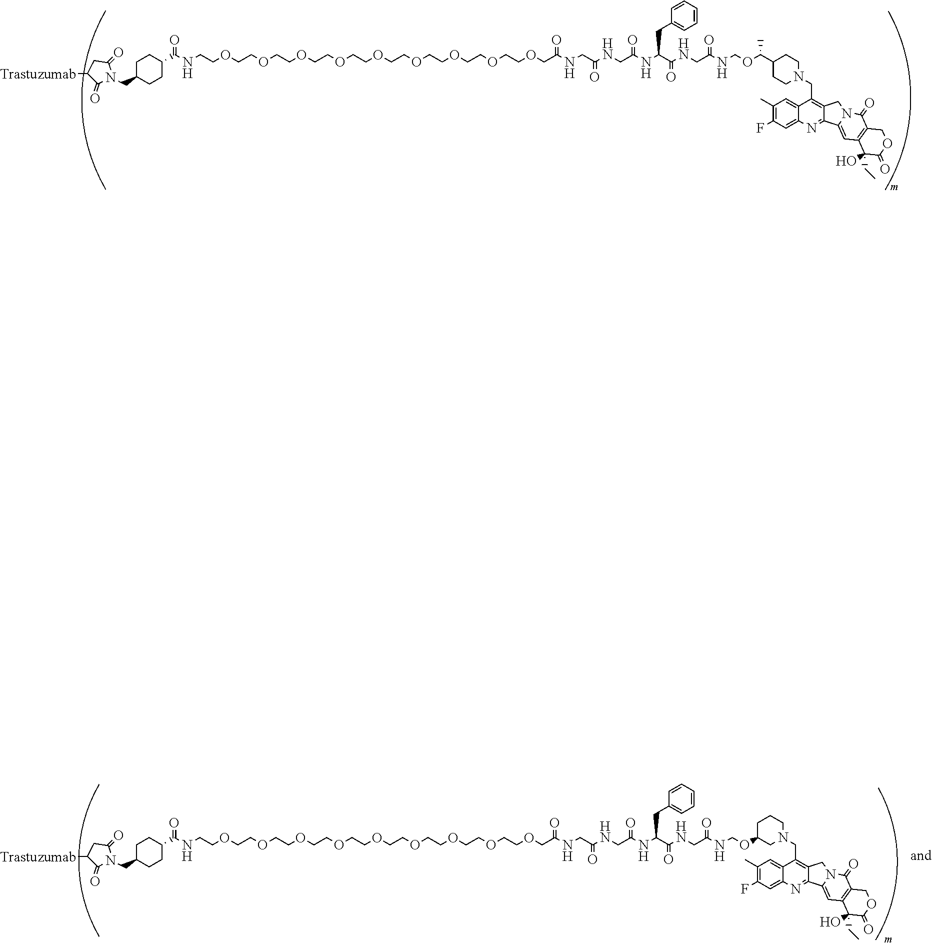

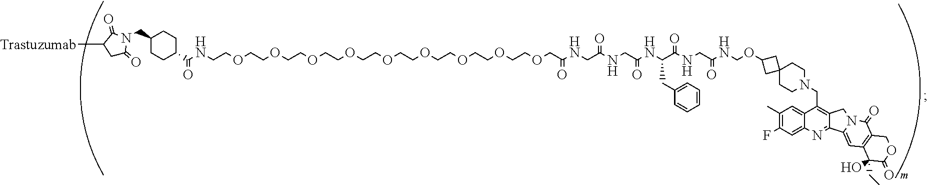

or a pharmaceutically acceptable salt or solvate thereof,
wherein m is defined as in formula (III) or (IIIa) and any embodiments thereof, including as defined in Embodiment B.

Aspect 5: In some other embodiments of the present disclosure, provided is a compound or the pharmaceutically acceptable salt or solvate thereof, optionally as a tautomer, a mesomer, a racemate, an enantiomer, a diastereomer, or combinations thereof, where the compound is of formula (V) thereof:

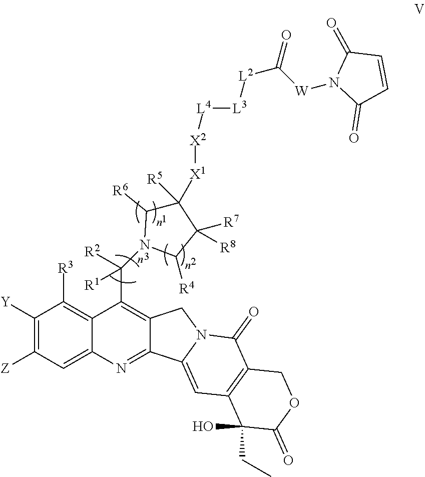

wherein: Y, Z, R¹, R², R³, R⁴, R⁵, R⁶, R⁷, R⁸, X¹, X², n¹, and n² are defined as in formula (III) and any embodiments thereof, including as defined in Embodiment B, and
L² is selected from the group consisting of —NR¹¹(CH₂CH₂O)ₚ¹CH₂CH₂C(O)—, —NR¹¹(CH₂CH₂O)ₚ¹CH₂CH₂—, —NR¹¹(CH₂CH₂O)ₚ¹CH₂C(O)—, —S(CH₂)ₚ¹C(O)— and a chemical bond, wherein p¹ is an integer selected from 1 to 20; and L² is preferably a chemical bond; wherein the left side of each of the L² groups provided above is attached to L¹;
L³ is a peptide residue composed of 2 to 7 amino acids, wherein the amino acids are optionally further substituted by one or more substituent(s) selected from the group consisting of halogen, hydroxy, cyano, amino, alkyl, chloroalkyl, deuterated alkyl, alkoxy and cycloalkyl; wherein the left side of each of the L³ groups provided above is attached to L²;
L⁴ is selected from the group consisting of —NR¹²(CR¹³R¹⁴)t-, —C(O)NR¹²—, —C(O)NR¹²(CH)ₜ—,

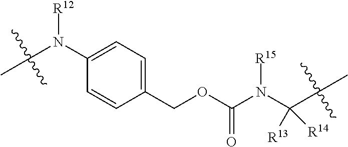

and a chemical bond, wherein t is an integer selected from 1 to 6; and L⁴ is preferably —NR¹²(CR¹³R¹⁴)ₜ⁻; wherein the left side of each of the L⁴ groups provided above is attached to the right side of L³ and the right side of each of the L⁴ groups is attached to X²;
R¹¹ and R¹² are each independently selected from the group consisting of H, alkyl, haloalkyl, deuterated alkyl and hydroxyalkyl;
R¹³ and R¹⁴ are each independently selected from the group consisting of H, halogen. alkyl. haloalkyl, deuterated alkyl, and hydroxyalkyl; and
R¹⁵ is selected from —CH₂CH₂SO₂CH₃, and —CH₂CH₂N(CH₃)₂;
W is selected from the group consisting of C₁-C₈ alkylene, —(C₁-C₈ alkylene)-cycloalkylene-, arylene, heteroarylene, and linear heteroalkylene, wherein the linear heteroalkylene comprise 1 to 8 carbon atom(s), and 1 to 3 heteroatom(s) selected from the group consisting of N, O, S, SO, and SO₂, and wherein the —(C₁-C₈ alkylene)-cycloalkylene-linear heteroalkylene, arylene, and heteroarylene are each independently optionally further substituted by one or more substituent(s) selected from the group consisting of halogen, hydroxy, cyano, amino, alkyl, chloroalkyl, deuterated alkyl, alkoxy and cycloalkyl;
and
n³ is an integer selected from 0 to 6. In some embodiments,
L² is selected from the group consisting of —NR¹¹(CH₂CH₂O)ₚ¹CH₂CH₂C(O)—, —NR¹¹(CH₂CH₂O)ₚ¹CH₂CH₂—, —NR¹¹(CH₂CH₂O)ₚ¹CH₂C(O)—, —S(CH₂)ₚ¹C(O)—, and a chemical bond, wherein p¹ at each occurrence is an integer selected from 6 to 12; and L² is preferably a chemical bond; wherein the left side of each of the L² groups provided above is attached to L¹;
L³ is a peptide residue composed of 2 to 7 amino acids. wherein the amino acids are selected from Phenylalanine (F), Glycine (G), Valine (V), Lysine (K), Citrulline, Serine (S), Glutamic acid (E), Aspartic acid (N); preferably a peptide residue composed of 1, 2 or more Phenylalanine and Glycine; more preferably is a peptide residue composed of 4 amino acids; the most preferably is a peptide residue composed of GGFG; wherein the left side of each of the L³ groups provided above is attached to L²;
L⁴ is —NR¹²(CR¹³R¹⁴)—, R¹² is H or alkyl, R¹³ and R¹⁴ are each independently selected from H and alkyl, t is 1 or 2; L⁴ is optionally —NR¹²CR¹³R¹⁴—; or L⁴ is preferably —NHCH₂—; wherein the left side of each of the L⁴ groups provided above is attached to the right side of L³ and the right side of each of the L⁴ groups is attached to X²;
R¹¹ and R¹² are each independently selected from the group consisting of hydrogen, alkyl, haloalkyl, deuterated alkyl and hydroxyalkyl; and
R¹³ and R¹⁴ are each independently selected from the group consisting of hydrogen, halogen. alkyl. haloalkyl, deuterated alkyl and hydroxyalkyl.

Aspect 5-A: In some other embodiments of the present disclosure, provided is a compound or the pharmaceutically acceptable salt or solvate thereof, optionally as a tautomer, a mesomer, a racemate, an enantiomer, a diastereomer, or combinations thereof, where the compound is of formula (Va) thereof:

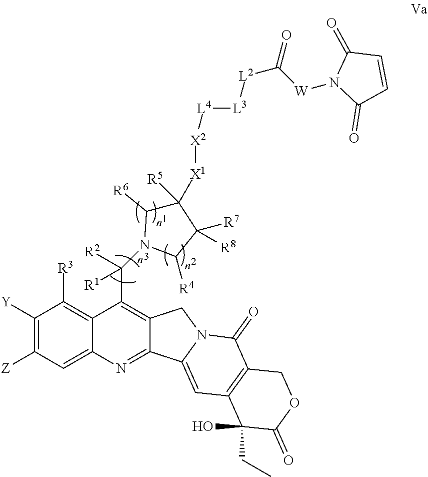

wherein: Y, Z, R¹, R², R³, R⁴, R⁵, R⁶, R⁷, RN, X¹, X², n¹, and n^{2a} are defined as in formula (IIIa) and any embodiments thereof, and L², L³, L⁴, W, and n³ are as defined for formula (V) in Aspect 5 and embodiments thereof.

In some embodiments of the present disclosure, the provided ligand-drug conjugate, or a pharmaceutically acceptable salt or solvate thereof, of formula (V) or (Va), wherein:

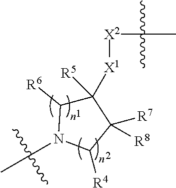

for formula (V) and

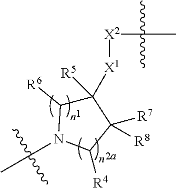

for formula (Va)
are a structure selected from the following structures below,

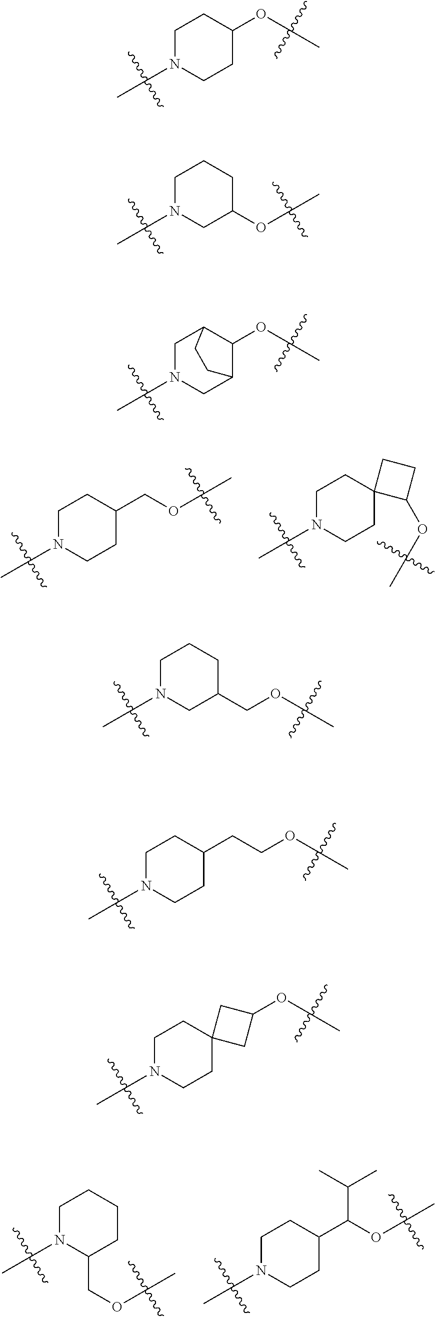

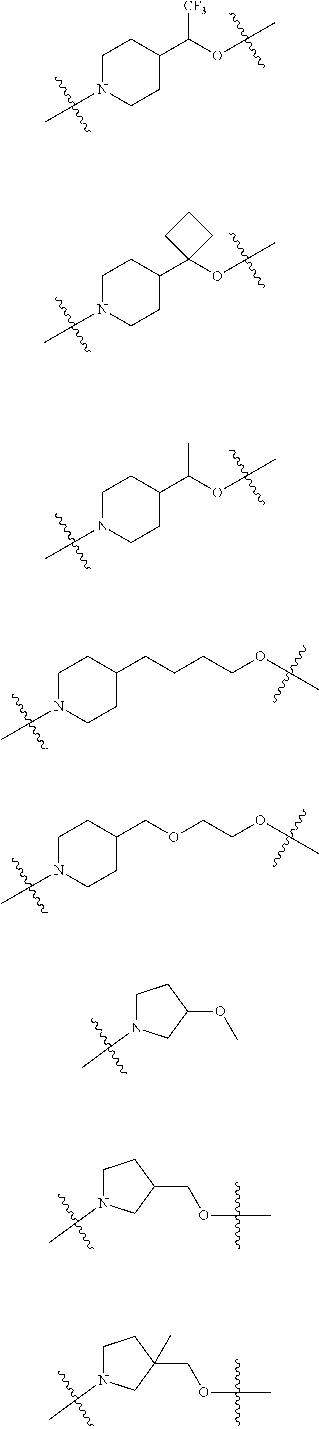

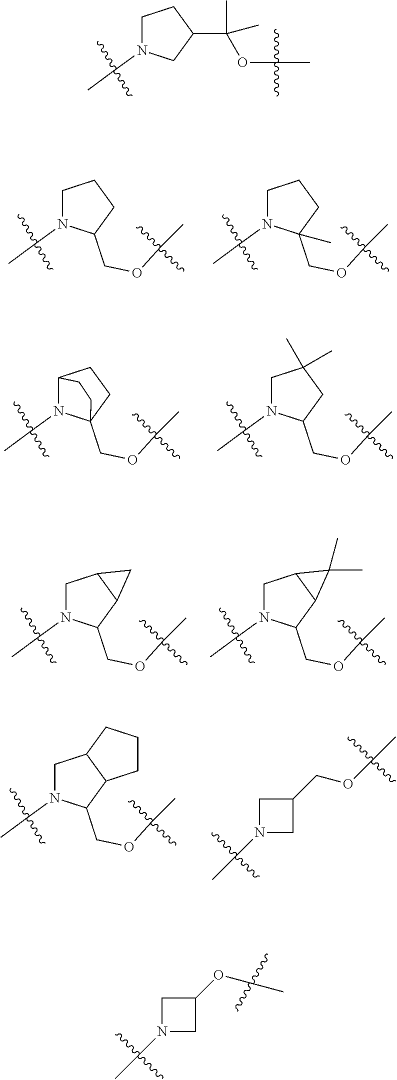

In some embodiments, the structure of any of the formula (V) or (Va), or a pharmaceutically acceptable salt or solvate thereof, wherein

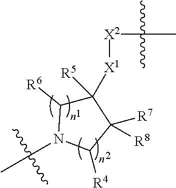

for formula (V) and

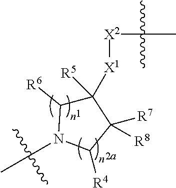

or formula (Va)
are selected from the following structures below,

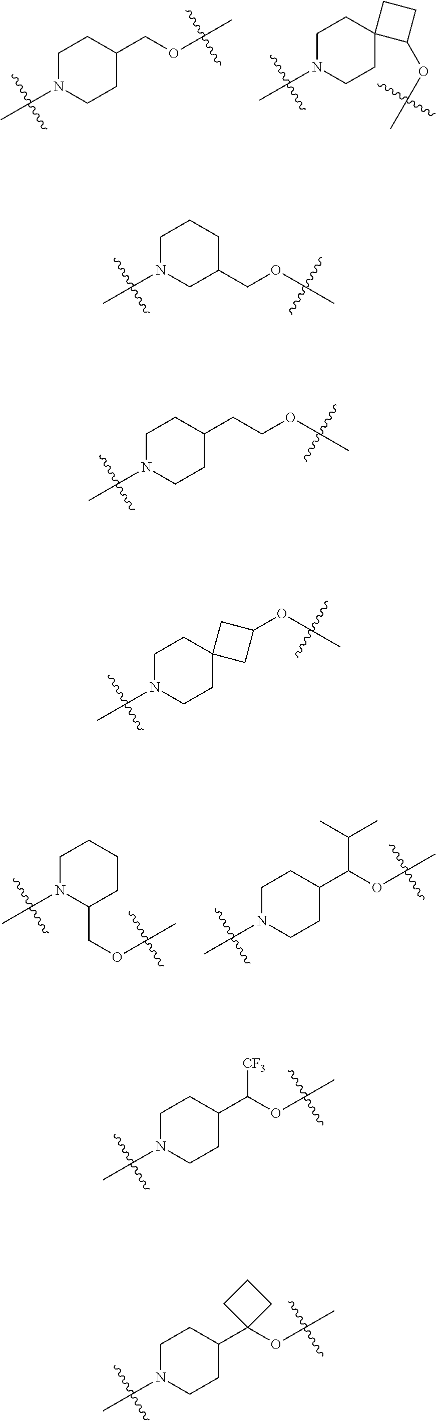

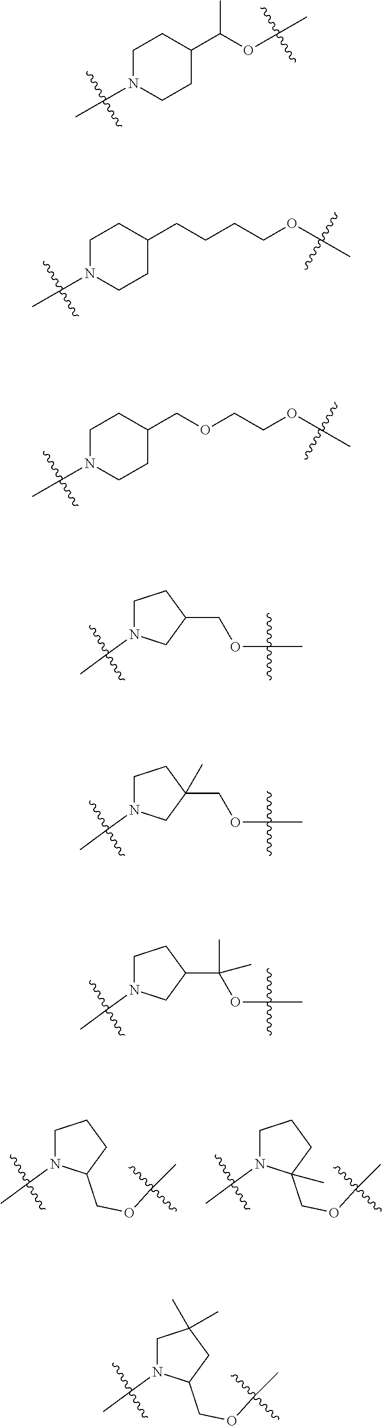

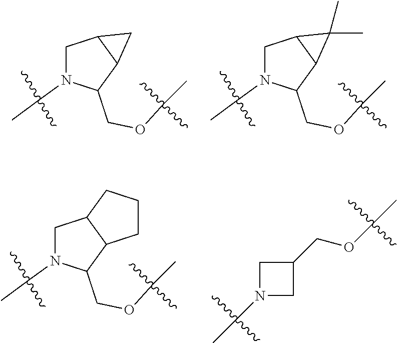 [SMILES: *OCC1C2CC2CN1* ; *OCC1C2C(CN1*)C2(C)C ; *OCC1C2CCCC2CN1* ; *OCC1CN(*)C1]

In some embodiments, the structure of formula (V) or (Va), or a pharmaceutically acceptable salt or solvate thereof, wherein

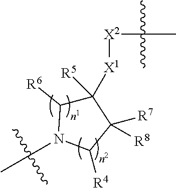

for formula (V) and

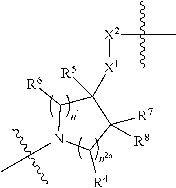

for formula (Va)
are selected from the following structures below,

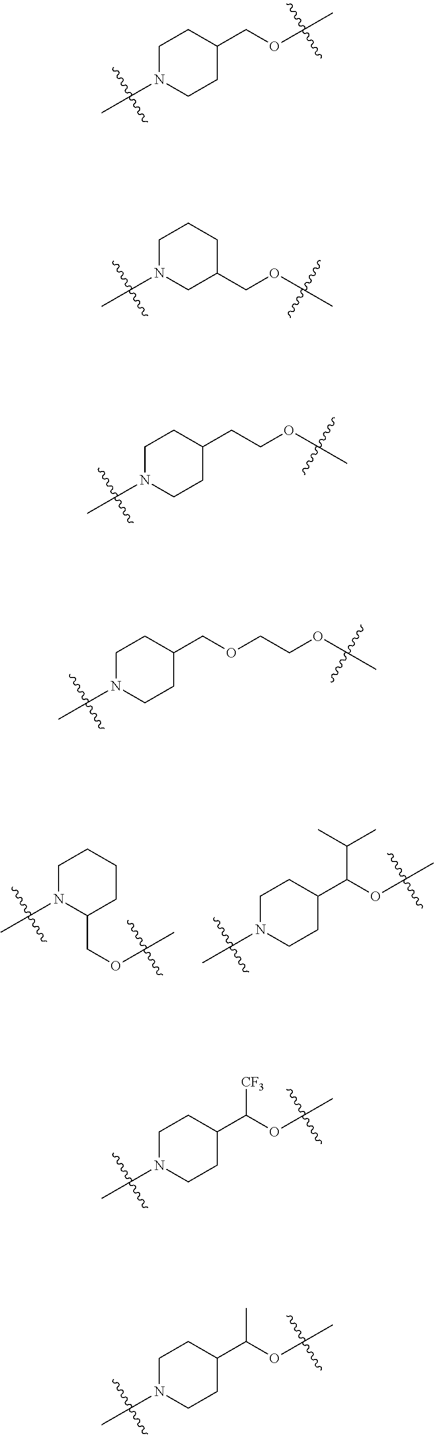

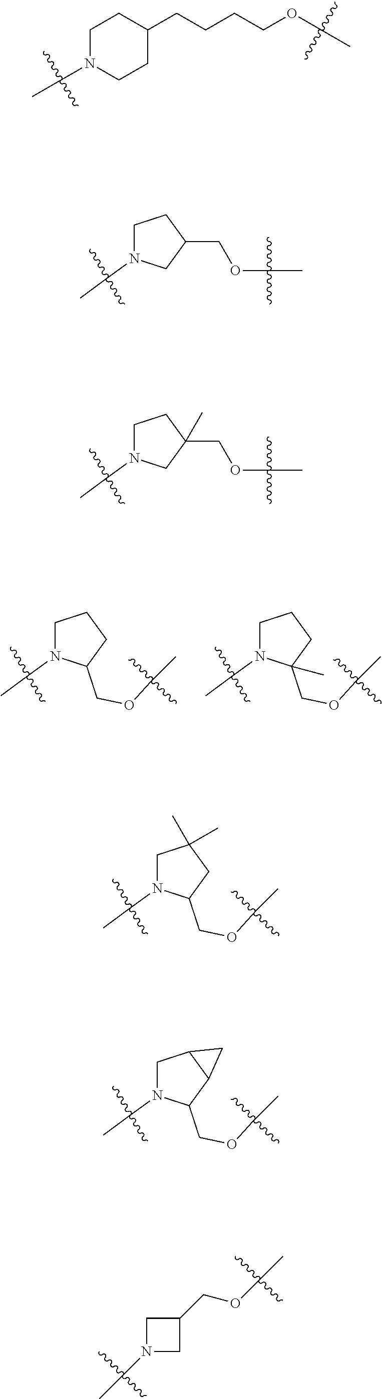

In some embodiments, the structure of formula (V) or (Va), or a pharmaceutically acceptable salt or solvate thereof, wherein

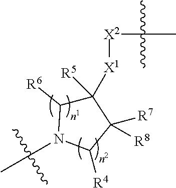

for formula (V) and

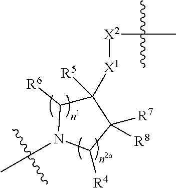

for formula (Va)
are selected from the following structures below,

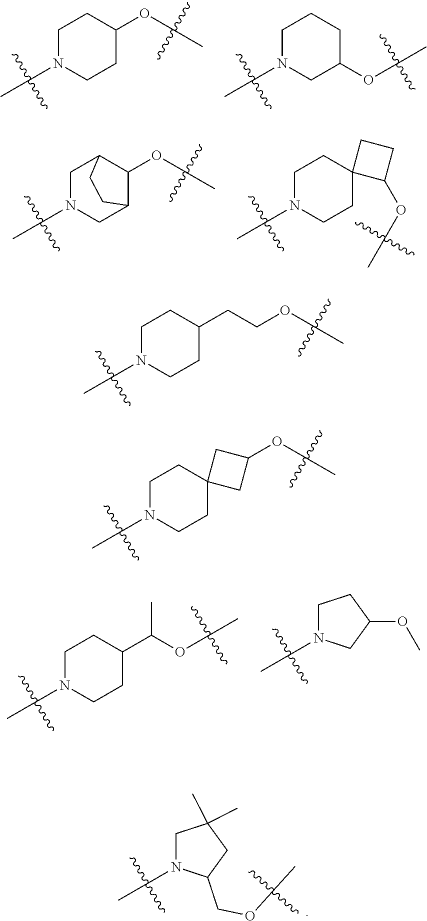

In some embodiments, the structure of formula (V) or (Va), or a pharmaceutically acceptable salt or solvate thereof, wherein

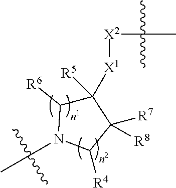

for formula (V) and

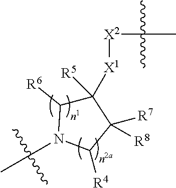

for formula (Va)
are selected from the following structures below,

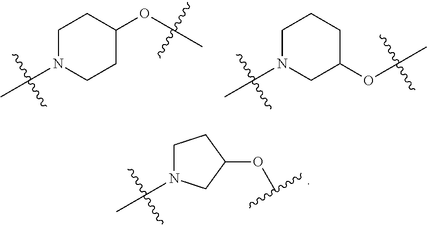 [SMILES: *OC1CCN(*)CC1 ; *OC1CCCN(*)C1 ; *OC1CCN(*)C1]

In some embodiments, the compounds include, but are not limited to:

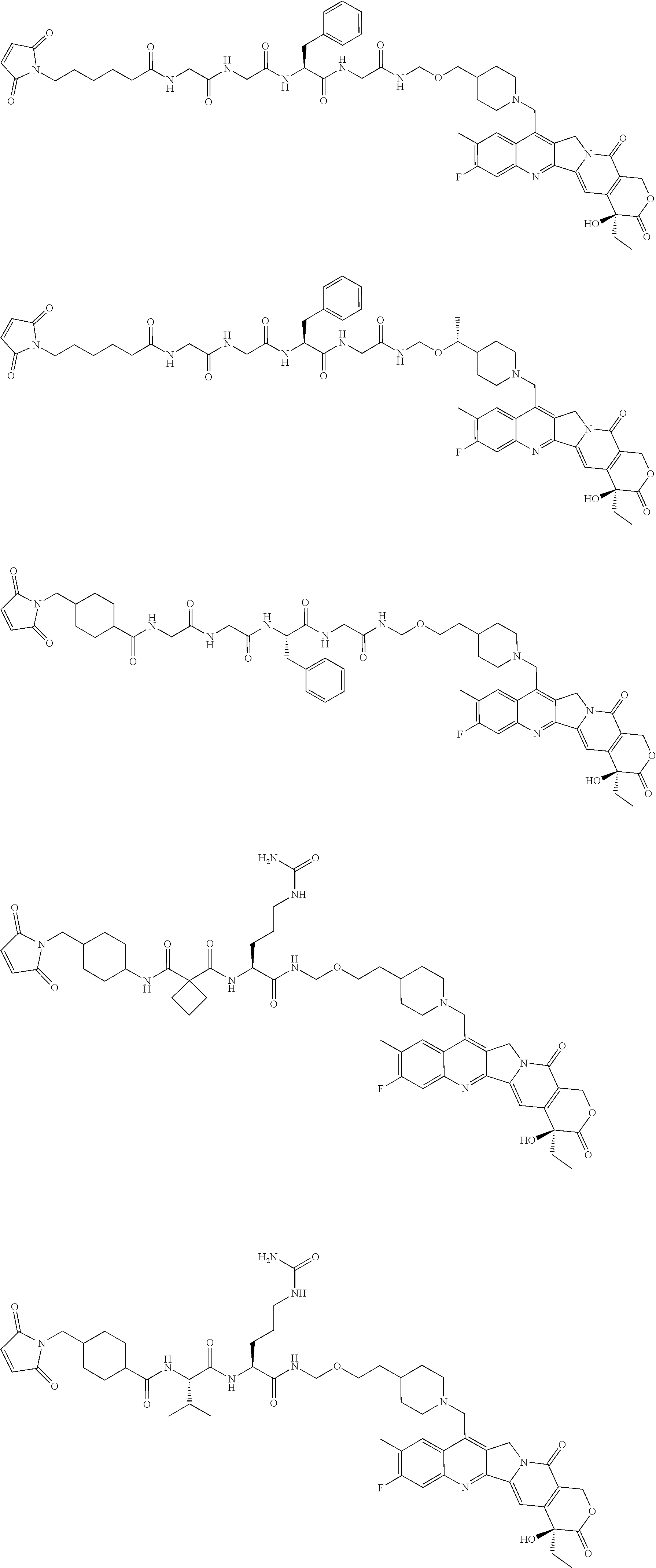

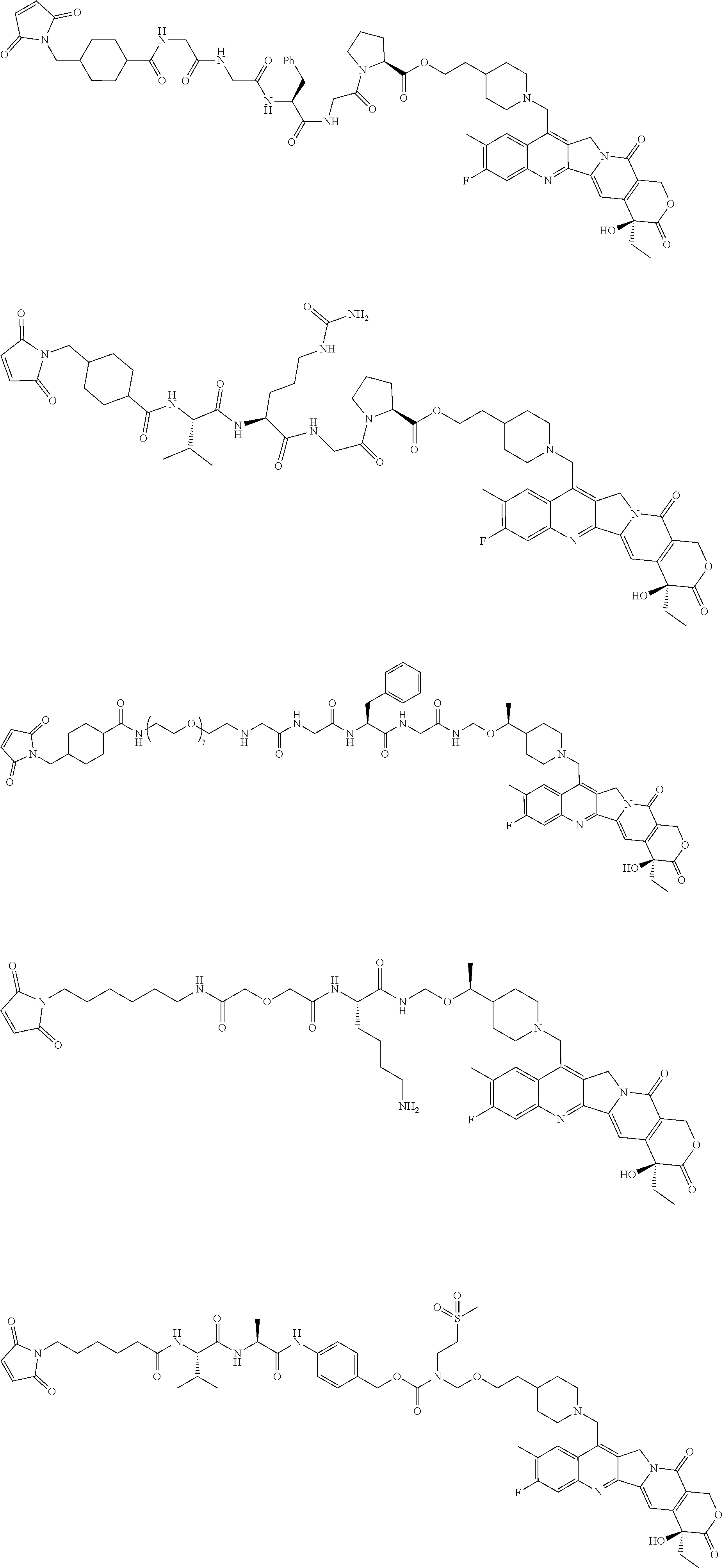

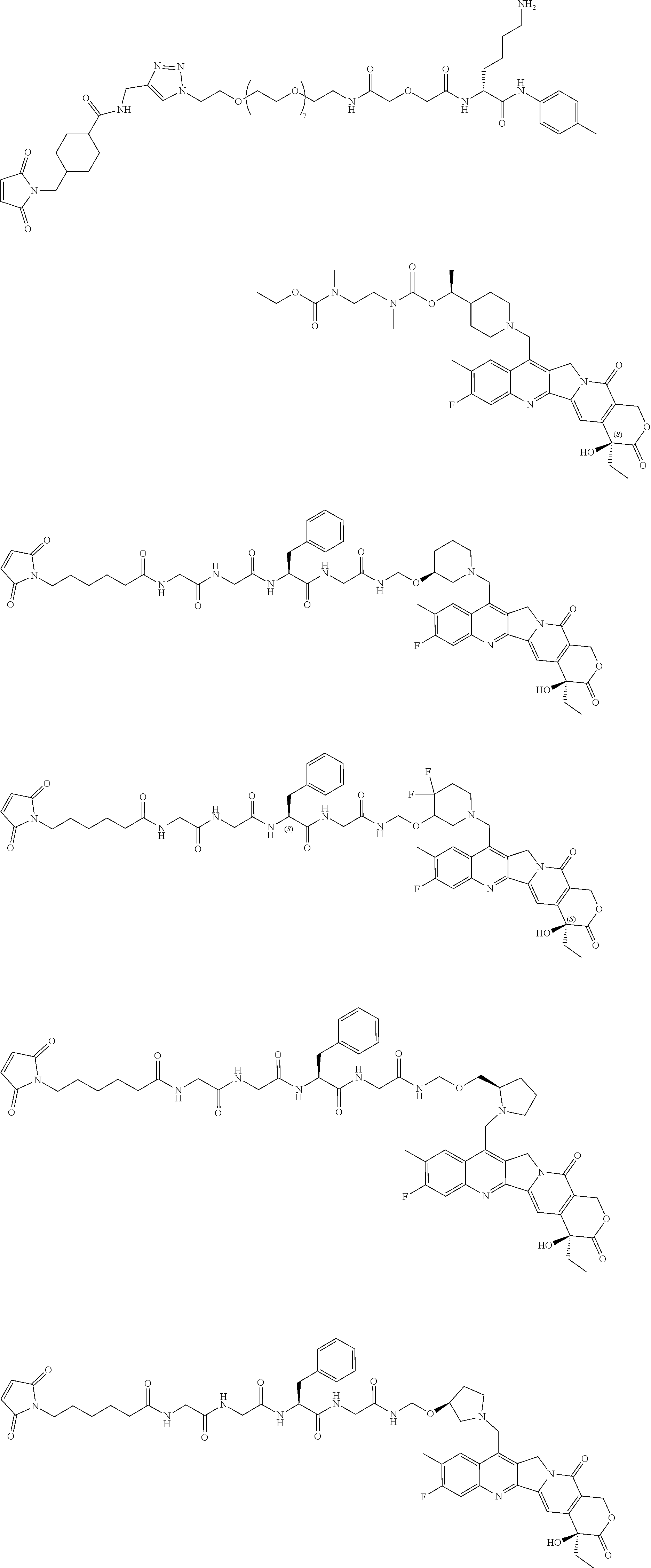

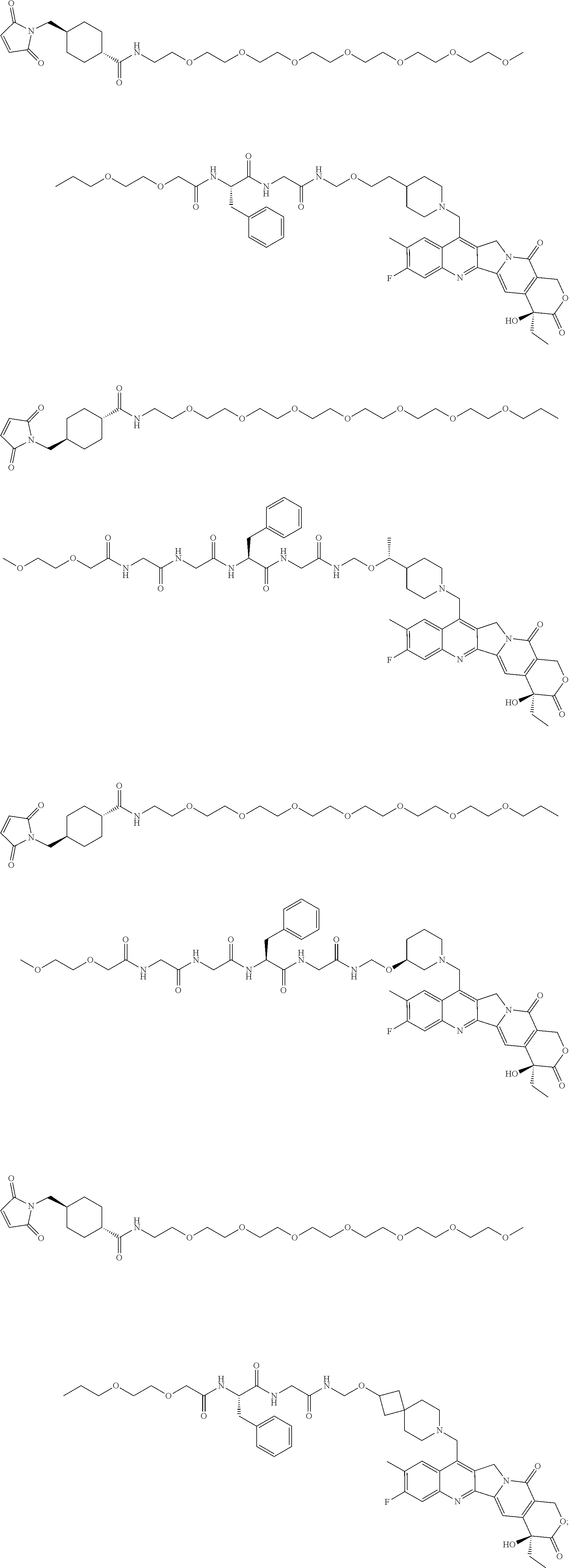

or a pharmaceutically acceptable salt or solvate thereof.

Aspect 6: In some embodiments of the present disclosure, provided is a compound or the pharmaceutically acceptable salt or solvate thereof, optionally as a tautomer, a mesomer, a racemate, an enantiomer, a diastereomers or combinations thereof, where the compound of formula (VI):

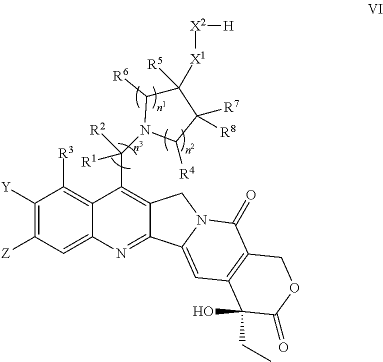

wherein
Y and Z are independently selected from H and a group consisting of halo, hydroxy, cyano, C₁-C₈ alkyl, C₁-C₈ alkoxy, (C₁-C₈ alkoxy)-C₁₋₈ alkyl, amino, (C₁-C₈ alkyl)NHC(O)O—, (C₃-C₆ cycloalkyl)NHC(O)O—, (C₁-C₆ alkyl)NH—, azetidin-1-yl, pyrrolidin-1-yl, piperidin-1-yl, azepane-1-yl, (C₁-C₈ alkyl)C(O)O—, and (C₁-C₈ alkyl)C(O)NH—; or Y and Z taken together with the atom(s) to which they are attached form a 5- to 8-membered heterocycloalkyl or 5- to 8-membered heteroaryl comprise one to three heteroatoms independently selected from N, O, S, S(O), and S(O)₂; wherein azetidin-1-yl, pyrrolidin-1-yl, piperidin-1-yl, azepan-1-yl, 5- to 8-membered heterocycloalkyl, 5- to 8-membered heteroaryl, and each “C₁-C₈ alkyl” are independently optionally substituted with one to three R⁹;
each R¹ and each R² are independently selected from H, C₁-C₈ alkyl, C₃-C₆ cycloalkyl, aryl, heteroaryl; or each pair of R¹ and R² taken together with the atom(s) to which they are attached form 3- to 6-membered cycloalkyl, or 4- to 8-membered heterocycloalkyl; wherein each R¹, R², and R¹/R² ring group is independently optionally substituted with one to four R⁹;
n³ is an integer selected from 0 to 6;
R³ is selected from H and a group consisting of halo, hydroxy, cyano, C₁-C₈ alkyl, C₁-C₈ alkoxy, (C₁-C₈ alkoxy)-C₁-C₈ alkyl, amino, (C₁-C₈ alkyl)amino, (C₁-C₈ alkyl)NHC(O)O—, (C₃-C₆ cycloalkyl)NHC(O)O—, (C₁C₈ alkyl)C(O)O—, (C₁-C₈ alkyl)C(O)NH—, aryl, heteroaryl, and nitro; wherein each “C₁-C₈ alkyl” cycloalkyl, aryl, and heteroaryl are independently optionally substituted with one to three R⁹;
X¹ is absent; or X¹ and R⁵ taken together with the atom(s) to which they are attached form 3- to 6-membered cycloalkyl or 4- to 8-membered heterocycloalkyl; or X¹ and R⁸ taken together with the atom(s) to which they are attached form a 3- to 6-membered cycloalkyl or 4- to 8-membered heterocycloalkyl; each of the 3- to 6-membered cycloalkyl and 4- to 8-membered heterocycloalkyl is optionally substituted with one to three R⁹;
X² comprises one or more groups independently selected from the group consisting of —O—, —S—, —NH—, C₁-C₈ alkylene, C₃-C₆ cycloalkylene, arylene, and heteroarylene; wherein the terminal group in X², connected to the H in —X²—H, is O or S; each of C₁-C₈ alkylene, C₃-C₆ cycloalkylene, arylene, and heteroarylene is independently optionally substituted with one to three R⁹;
R⁴, R⁵, R⁶, R⁷, and R⁸ at each occurrence are independently selected from H and a group consisting of halo, hydroxy, C₁-C₈ alkyl, C₃-C₆ cycloalkyl, aryl, and heteroaryl and wherein each of C₁-C₈ alkyl, C₃-C₆ cycloalkyl, aryl, and heteroaryl is independently optionally substituted with one to four R⁹; or
R⁴ and R⁵ taken together with the atom(s) to which they are attached form 3- to 6-membered cycloalkyl, or 4- to 8-membered heterocycloalkyl; or R⁴ and R⁷ taken together with the atom(s) to which they are attached form 3- to 6-membered cycloalkyl, or 4- to 8-membered heterocycloalkyl; or R⁶ and R⁷ taken together with the atom(s) to which they are attached form 3- to 6-membered cycloalkyl, or 4- to 8-membered heterocycloalkyl; or R⁷ and R⁸ taken together with the atom(s) to which they are attached form oxo, 3- to 6-membered cycloalkyl, or 4- to 8-membered heterocycloalkyl; and wherein each of the 3- to 6-membered cycloalkyl and 4- to 8-membered heterocycloalkyl are independently optionally substituted with one to four R⁹; and the remaining of R⁴, R⁵, R⁶, R⁷, and R⁸ at each occurrence are independently selected from H and the group consisting of halo, hydroxy, C₁-C₈ alkyl, C₃-C₆ cycloalkyl, aryl, and heteroaryl, wherein the C₁-C₈ alkyl, C₃-C₆ cycloalkyl, aryl, and heteroaryl are independently optionally substituted with one to four R⁹;
R⁹ at each occurrence is independently selected from the group consisting of halogen, oxo, hydroxy, cyano, C₁-C₈ alkyl, C₃-C₆ cycloalkyl, C₁-C₈ alkoxy, aryl, and heteroaryl; or two R⁹ groups when attached to adjacent carbons and taken together with the carbons to which they are attached form a fused C₃-C₆ cycloalkyl; or two R⁹ groups when attached to the same carbon and taken together with the carbon to which they are attached form a spiro C₃-C₆ cycloalkyl; wherein each C₁-C₈ alkyl, C₃-C₆ cycloalkyl, C₁-C₈ alkoxy, aryl, heteroaryl, fused C₃-C₆ cycloalkyl, and spiro C₃-C₆ cycloalkyl is optionally independently substituted with one to three fluoro or hydroxy, and C₁-C₃ alkyl;
n¹ and n² are each an integer independently selected from 0, 1, 2, 3, and 4, provided that n¹+n² is 2, 3, or 4; and
n is an integer selected from 0 and 1.

Embodiment C: In some embodiments of the present disclosure, the compound of formula VI, or pharmaceutically acceptable salt or solvate thereof, is wherein n¹ and n² are integer independently selected from 0, 1, and 2 and provided that n¹+n² is ≥2; and all other variables are as defined in Aspect 6.

Aspect 6-A: In some embodiments of the present disclosure, provided is a compound or the pharmaceutically acceptable salt or solvate thereof, optionally as a tautomer, a mesomer, a racemate, an enantiomer, a diastereomers or combinations thereof, where the compound of formula (VIa):

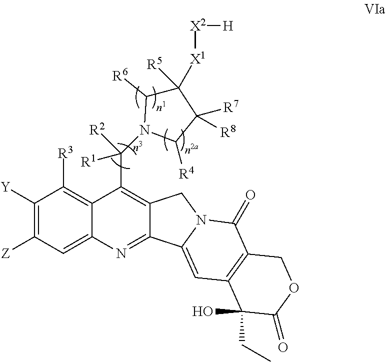

wherein: Y, Z, R¹, R², R³, each R⁴ (independently), each R⁶ (independently), R⁵, R⁷, R⁸, X¹, and X² are as defined as in formula (VI) in Aspect 6; n³ is an integer selected from 0 to 6; and n¹ and n^{2a} are each an integer independently selected from 0, 1, 2, 3, and 4, provided that n¹+n^{2a} is 2, 3, or 4. In some embodiments, Y, Z, R¹, R², R³, each R⁴ (independently), R⁵, each R⁶ (independently), R⁷, and X² are as defined as in formula (VI) in Aspect 6; and wherein X¹ is absent; or X¹ and R⁵ taken together with the atom(s) to which they are attached form a 3- to 6-membered cycloalkyl or 4- to 8-membered heterocycloalkyl, provided that R⁴ and R⁵ do not also form a ring, where the 3- to 6-membered cycloalkyl and 4- to 8-membered heterocycloalkyl is optionally substituted with one to three R⁹; R⁸ is selected from H and a group consisting of halo, hydroxy, C₁-C₆ alkyl, C₃-C₆ cycloalkyl, aryl, and heteroaryl and wherein each of C₁-C₆ alkyl, C₃-C₆ cycloalkyl, aryl, and heteroaryl is independently optionally substituted with one to four R⁹; n³ is an integer selected from 0 to 6; and n¹ and n^{2a} are each an integer independently selected from 0, 1, 2, 3, and 4, provided that n¹+n^{2a} is 2, 3, or 4. In some embodiments, R¹ and R² are each hydrogen.

Aspect 7: In some embodiments of the present disclosure, provided is a compound, or the pharmaceutically acceptable salt or solvate thereof, optionally as a tautomer, a mesomer, racemate, an enantiomer, a diastereomer, or combinations thereof, is a compound with formula (VII) thereof:

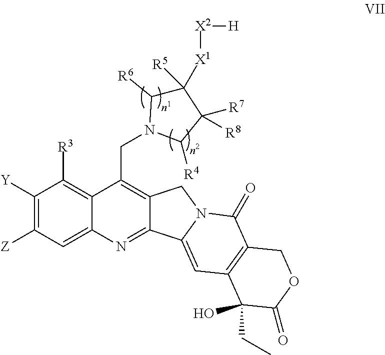

wherein: Y, Z, R³, R⁴, R⁵, R⁶, R⁷, R⁸, X¹, X², n¹, and n² are defined as in formula (VI), including as defined in Embodiment C.

Aspect 7-A: In some embodiments of the present disclosure, provided is a compound, or the pharmaceutically acceptable salt or solvate thereof, optionally as a tautomer, a mesomer, racemate, an enantiomer, a diastereomer, or combinations thereof, is a compound with formula (VIIa) thereof:

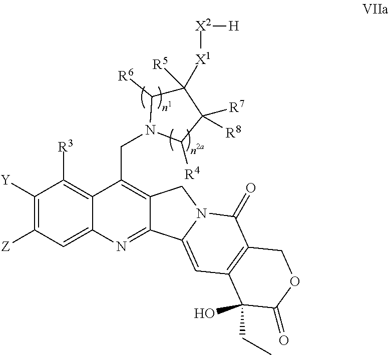

wherein: Y, Z, R³, R⁴, R⁵, R⁶, R⁷, R⁸, X¹, X², n¹, and n^{2a} are defined as in formula (VIa).

In some embodiments, the following compounds are excluded from the compound of formulas (VI), (VIa), (VII), and (VIIa):

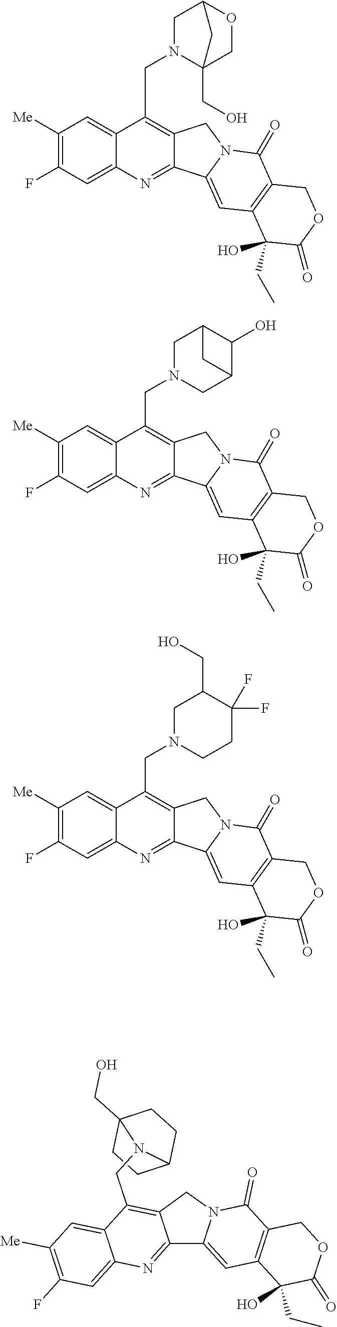

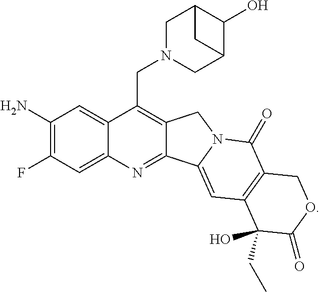 [SMILES: CC[C@@]1(O)C(=O)OCc2c1cc1n(c2=O)Cc2c-1nc1cc(F)c(N)cc1c2CN1CC2CC(C1)C2O]

In some embodiments of the present disclosure, the structure of any of the formula II, IIa, III, IIIa, IV, IVa, V, Va, VI, VIa, VII, and VIIa, or pharmaceutically acceptable salt or solvate thereof, wherein:
Y and Z are independently selected from H, halo, C₁-C₈ alkyl, and C₁-C₈ alkoxy;
R³ is selected from H, halo, C₁-C₈ alkyl, and C₁-C₈ alkoxy;
X¹ is absent;
X² is —O—;
R⁴, R⁵, R⁶, R⁷, and R⁸ at each occurrence are independently selected from H, halo, and C₁-C₈ alkyl;
n¹ and n^{2a} are each an integer independently selected from 0, 1, 2, and 3; provided that n¹+n^{2a} is 2, 3, or 4.

In some embodiments of the present disclosure, provided is the compound, or the pharmaceutically acceptable salt or solvate thereof, of any of the formula (VI), (VIa), (VII), and (VIIa) and any embodiments thereof, including of Embodiment C, wherein:

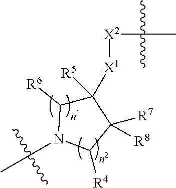

for formulas (VI) and (VII) and

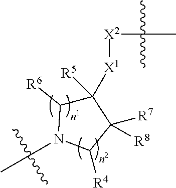

for formulas (VIa) and (VIIa)
are a structure selected from the following structures below,

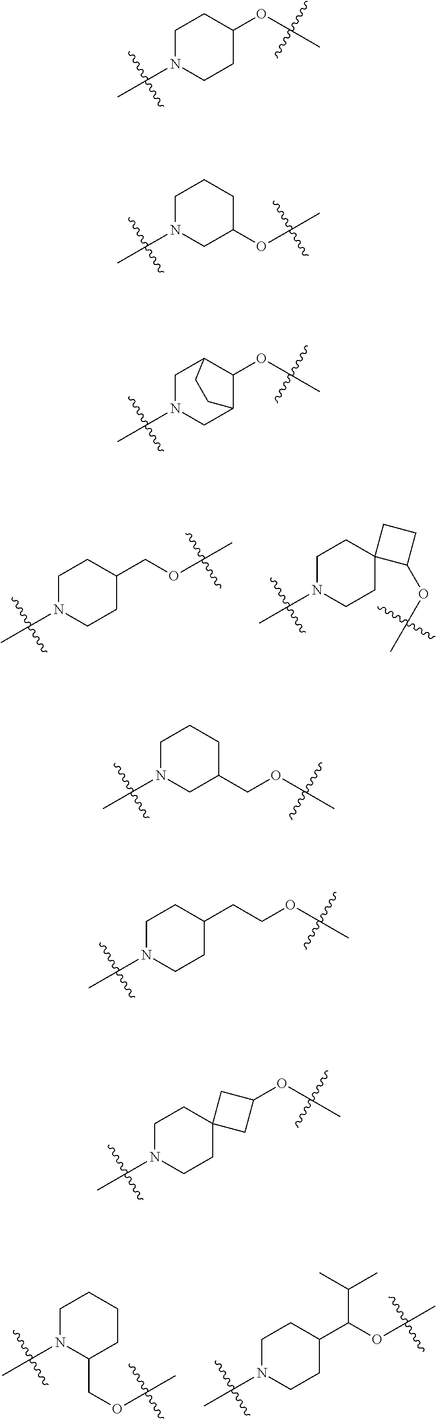

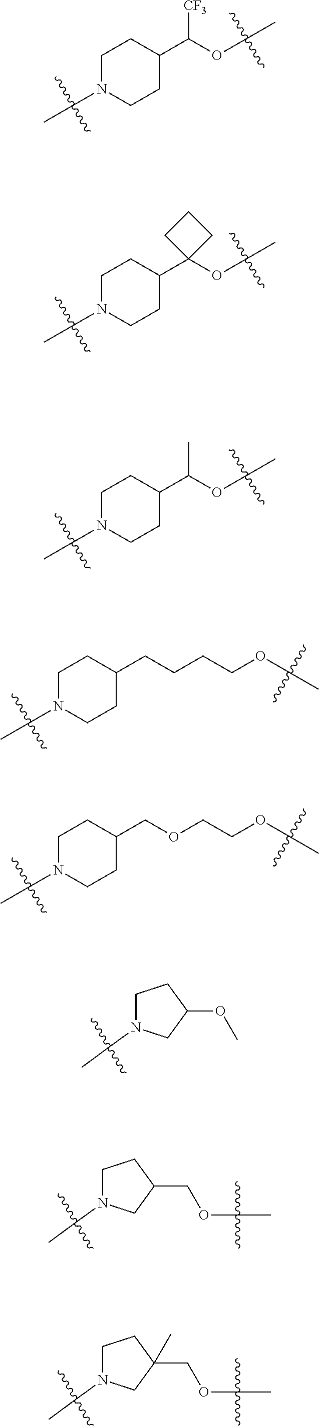

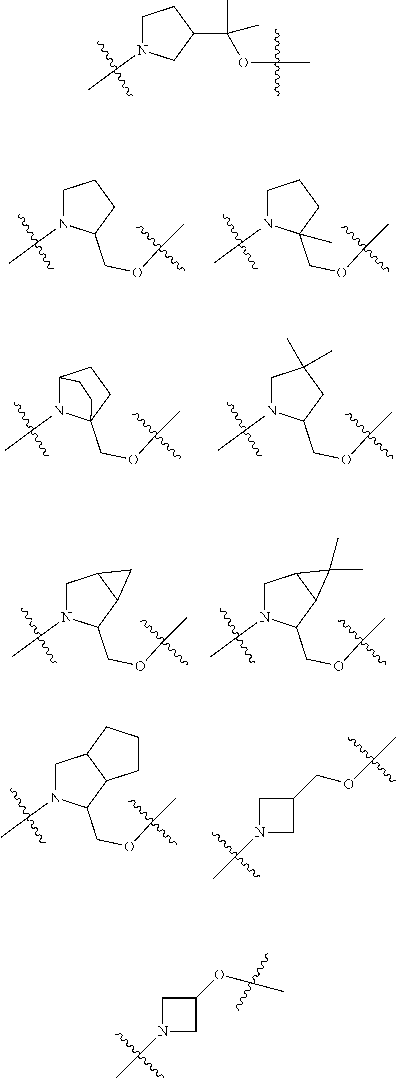

In some embodiments of the present disclosure, provided is the compound, or the pharmaceutically acceptable salt or solvate thereof, of any of the formula (VI), (VIa), (VII), and (VIIa) and any embodiments thereof, including of Embodiment C, wherein:

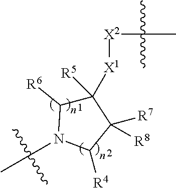

for formulas (VI) and (VII) and

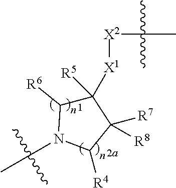

for formulas (VIa) and (VIIa)
are selected from the following structures below,

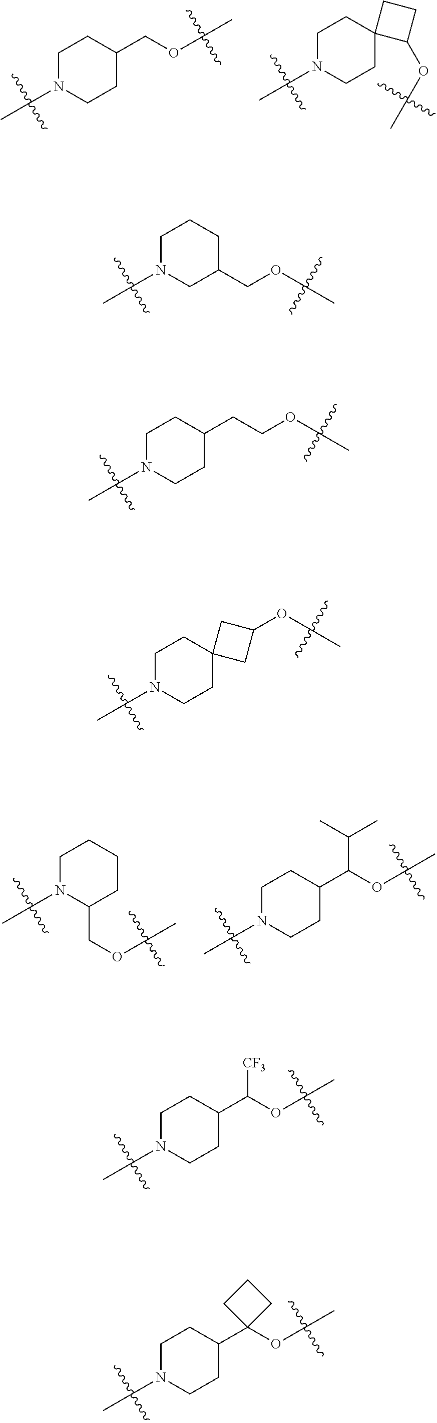

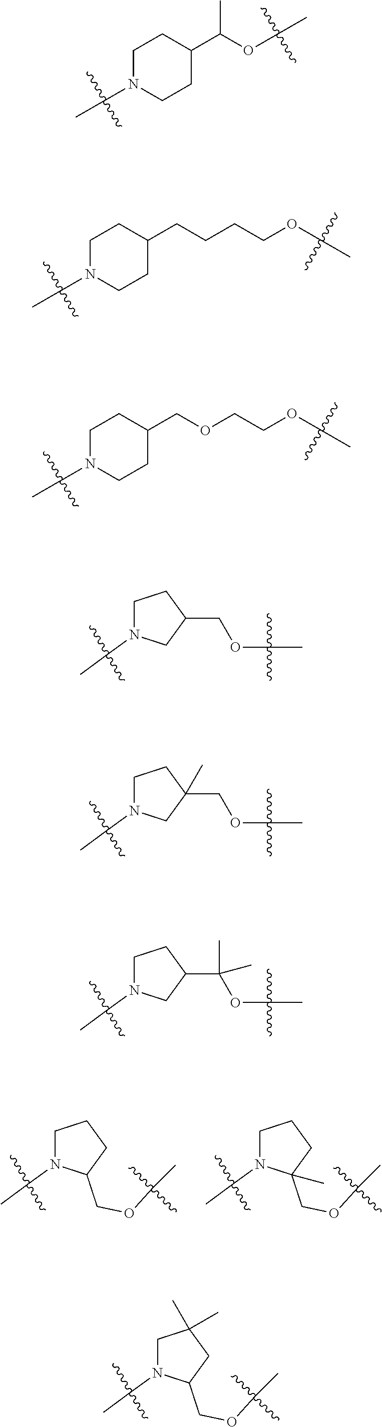

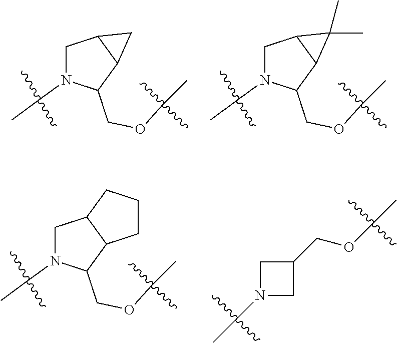 [SMILES: *OCC1C2CC2CN1* ; *OCC1C2C(CN1*)C2(C)C ; *OCC1C2CCCC2CN1* ; *OCC1CN(*)C1]

In some embodiments of the present disclosure, provided is the compound, or the pharmaceutically acceptable salt or solvate thereof, of any of the formula (VI), (VIa), (VII), and (VIIa) and any embodiments thereof, including of Embodiment C, wherein:

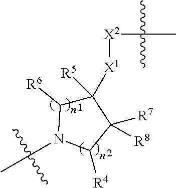

for formulas (VI) and (VII) and

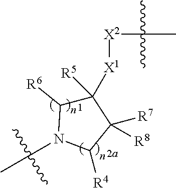

for formulas (VIa) and (VIIa)
are selected from the following structures below,

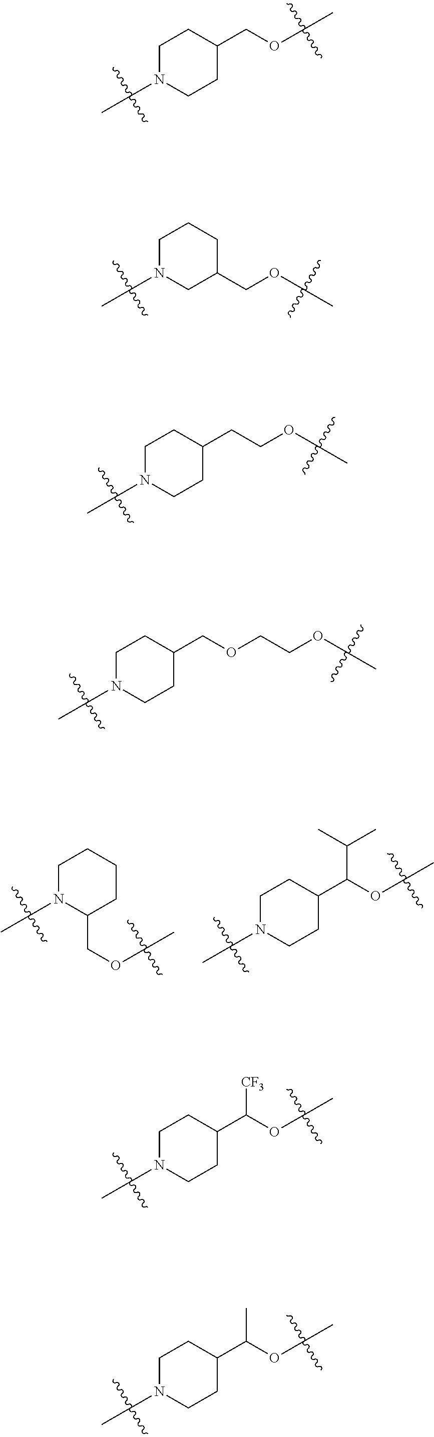

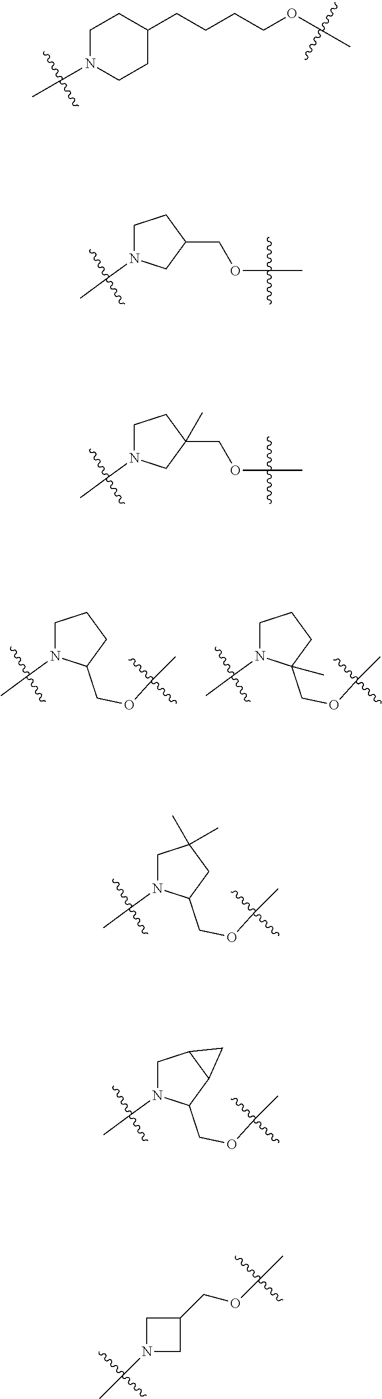

In some embodiments of the present disclosure, provided is the compound, or the pharmaceutically acceptable salt or solvate thereof, of any of the formula (VI), (VIa), (VII), and (VIIa) and any embodiments thereof, including of Embodiment C, wherein:

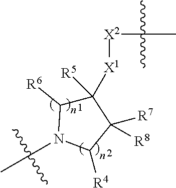

for formulas (VI) and (VII) and

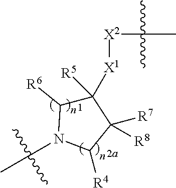

for formulas (VIa) and (VIIa)
are selected from the following structures below,

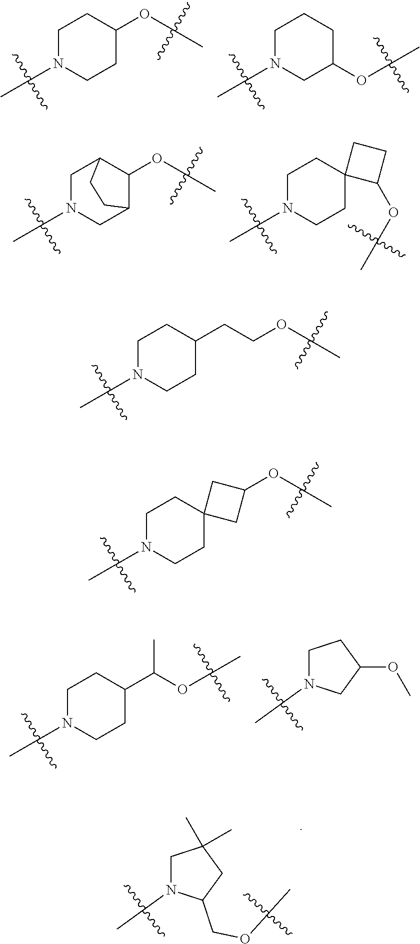

In some embodiments of the present disclosure, provided is the compound, or the pharmaceutically acceptable salt or solvate thereof, of any of the formula (VI), (VIa), (VII), and (VIIa) and any embodiments thereof, including of Embodiment C, wherein:

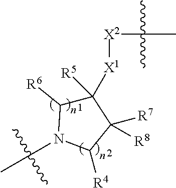

for formulas (VI) and (VII) and

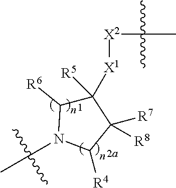

for formulas (VIa) and (VIIa)
are selected from the following structures below,

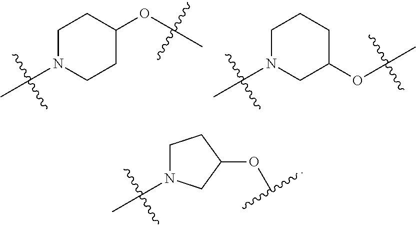 [SMILES: *OC1CCN(*)CC1 ; *OC1CCCN(*)C1 ; *OC1CCN(*)C1]

In some embodiments, provided is the compound, or the pharmaceutically acceptable salt or solvate thereof, of any of the formula (VI), (VIa), (VII), and (VIIa) and any embodiments thereof, including of Embodiment C, selected from:

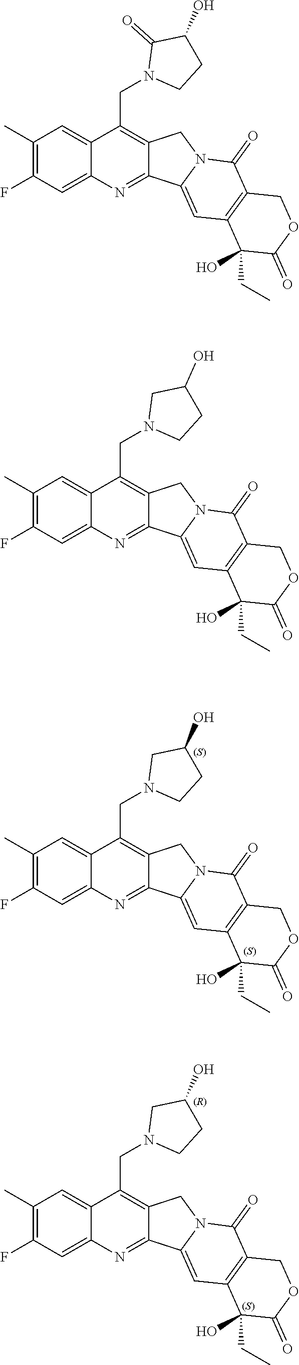

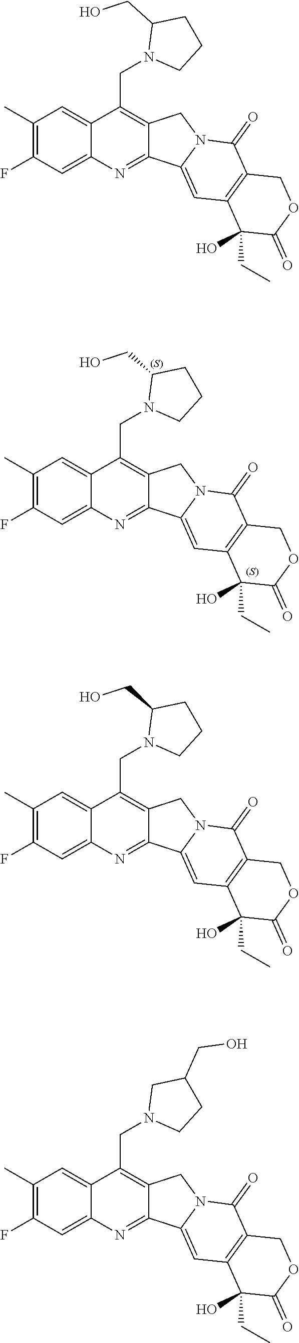

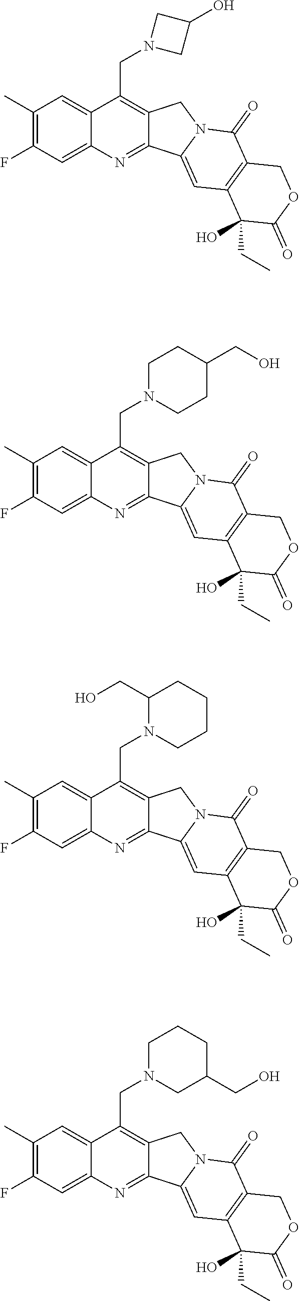 [SMILES: CC[C@@]1(O)C(=O)OCc2c1cc1n(c2=O)Cc2c-1nc1cc(F)c(C)cc1c2CN1CC(O)C1 ; CC[C@@]1(O)C(=O)OCc2c1cc1n(c2=O)Cc2c-1nc1cc(F)c(C)cc1c2CN1CCC(CO)CC1 ; CC[C@@]1(O)C(=O)OCc2c1cc1n(c2=O)Cc2c-1nc1cc(F)c(C)cc1c2CN1CCCCC1CO ; CC[C@@]1(O)C(=O)OCc2c1cc1n(c2=O)Cc2c-1nc1cc(F)c(C)cc1c2CN1CCCC(CO)C1]

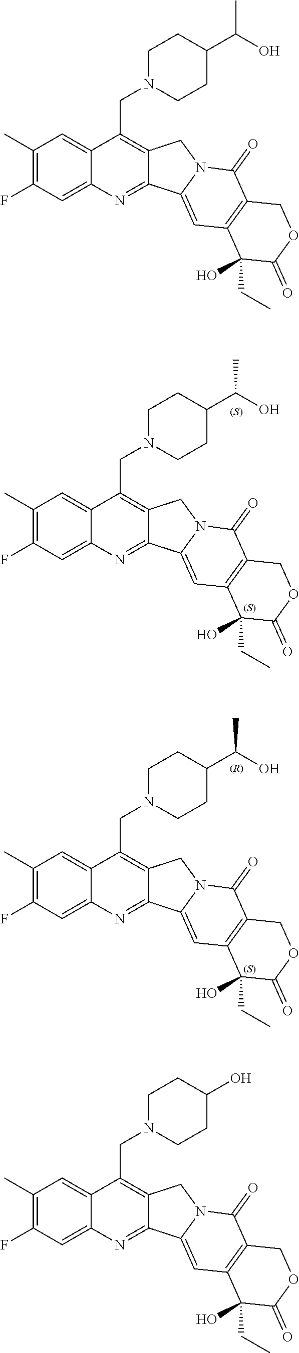

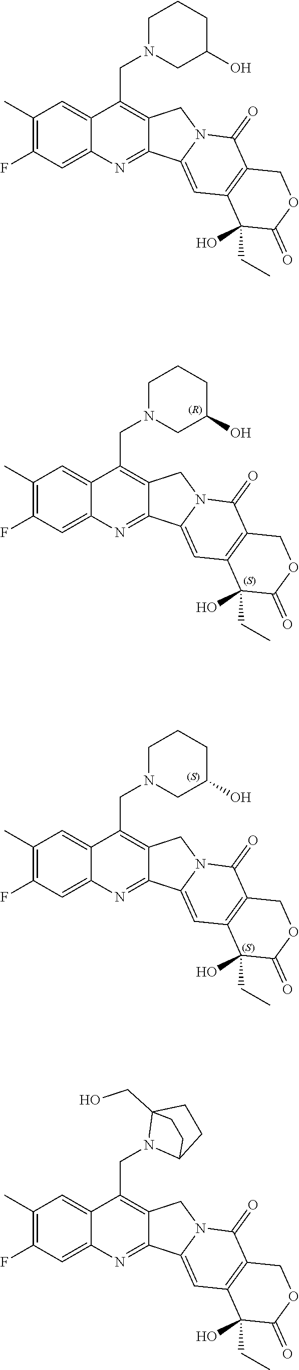

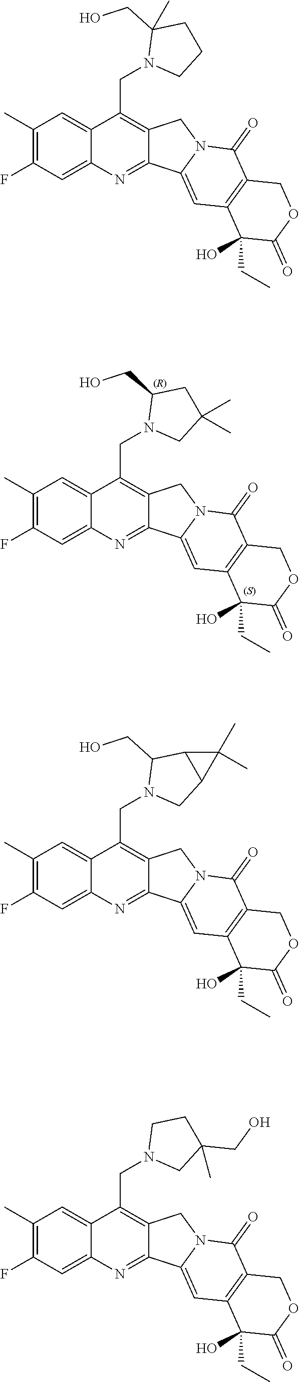

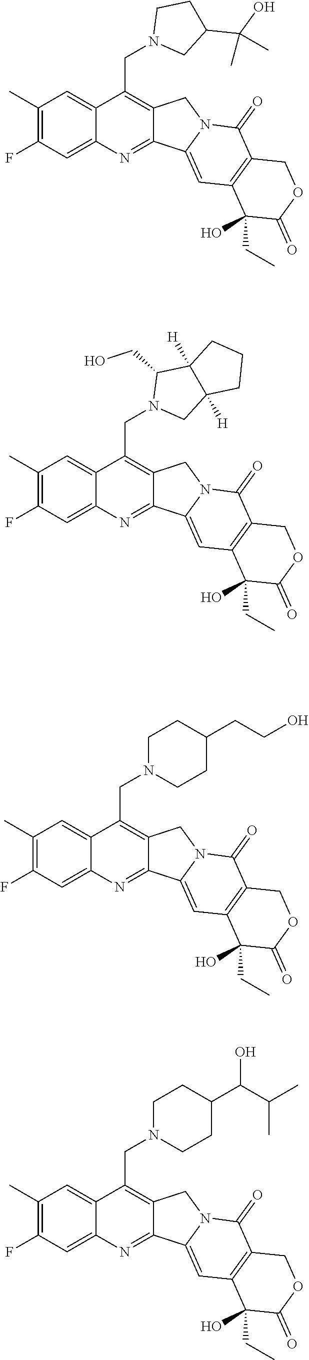 [SMILES: CC[C@@]1(O)C(=O)OCc2c1cc1n(c2=O)Cc2c-1nc1cc(F)c(C)cc1c2CN1CCC(C(C)(C)O)C1 ; CC[C@@]1(O)C(=O)OCc2c1cc1n(c2=O)Cc2c-1nc1cc(F)c(C)cc1c2CN1C[C@@H]2CCC[C@@H]2[C@H]1CO ; CC[C@@]1(O)C(=O)OCc2c1cc1n(c2=O)Cc2c-1nc1cc(F)c(C)cc1c2CN1CCC(CCO)CC1 ; CC[C@@]1(O)C(=O)OCc2c1cc1n(c2=O)Cc2c-1nc1cc(F)c(C)cc1c2CN1CCC(C(O)C(C)C)CC1]

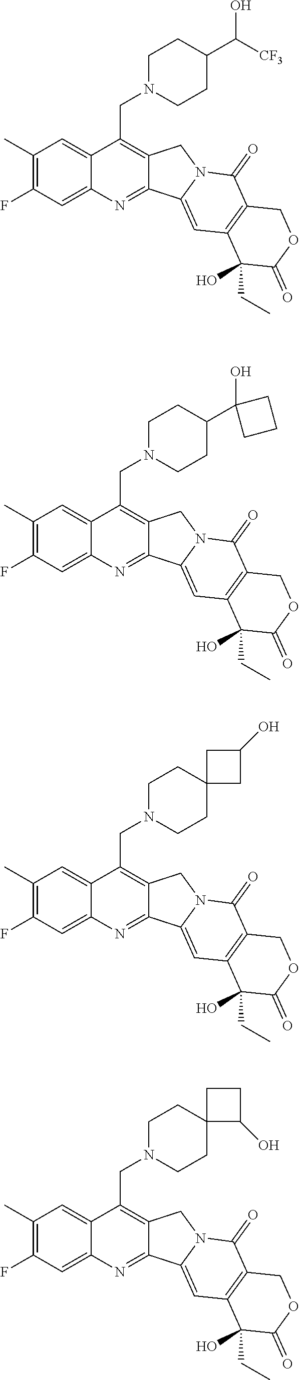 [SMILES: CC[C@@]1(O)C(=O)OCc2c1cc1n(c2=O)Cc2c-1nc1cc(F)c(C)cc1c2CN1CCC(C(O)C(F)(F)F)CC1 ; CC[C@@]1(O)C(=O)OCc2c1cc1n(c2=O)Cc2c-1nc1cc(F)c(C)cc1c2CN1CCC(C2(O)CCC2)CC1 ; CC[C@@]1(O)C(=O)OCc2c1cc1n(c2=O)Cc2c-1nc1cc(F)c(C)cc1c2CN1CCC2(CC1)CC(O)C2 ; CC[C@@]1(O)C(=O)OCc2c1cc1n(c2=O)Cc2c-1nc1cc(F)c(C)cc1c2CN1CCC2(CCC2O)CC1]

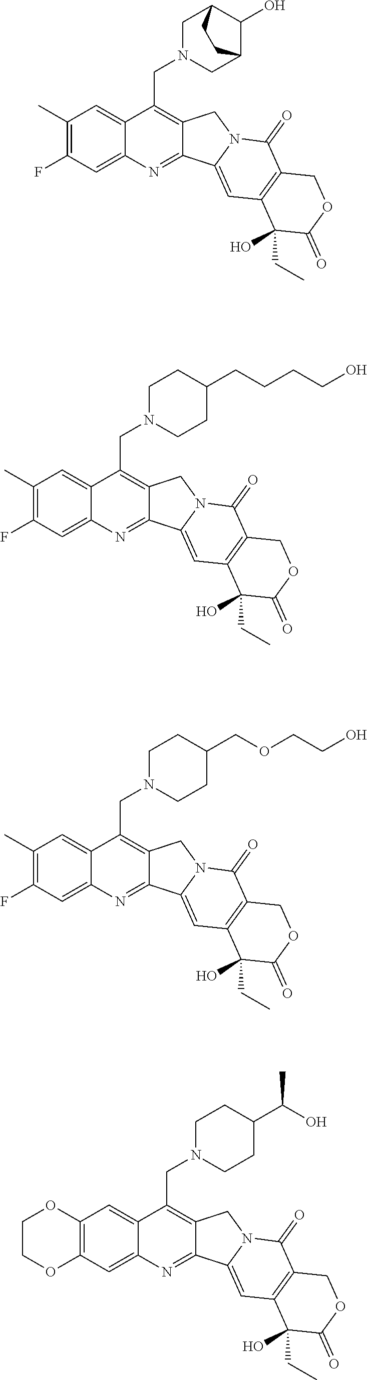 [SMILES: CC[C@@]1(O)C(=O)OCc2c1cc1n(c2=O)Cc2c-1nc1cc(F)c(C)cc1c2CN1C[C@H]2CC[C@@H](C1)C2O ; CC[C@@]1(O)C(=O)OCc2c1cc1n(c2=O)Cc2c-1nc1cc(F)c(C)cc1c2CN1CCC(CCCCO)CC1 ; CC[C@@]1(O)C(=O)OCc2c1cc1n(c2=O)Cc2c-1nc1cc(F)c(C)cc1c2CN1CCC(COCCO)CC1 ; CC[C@@]1(O)C(=O)OCc2c1cc1n(c2=O)Cc2c-1nc1cc3c(cc1c2CN1CCC([C@@H](C)O)CC1)OCCO3]

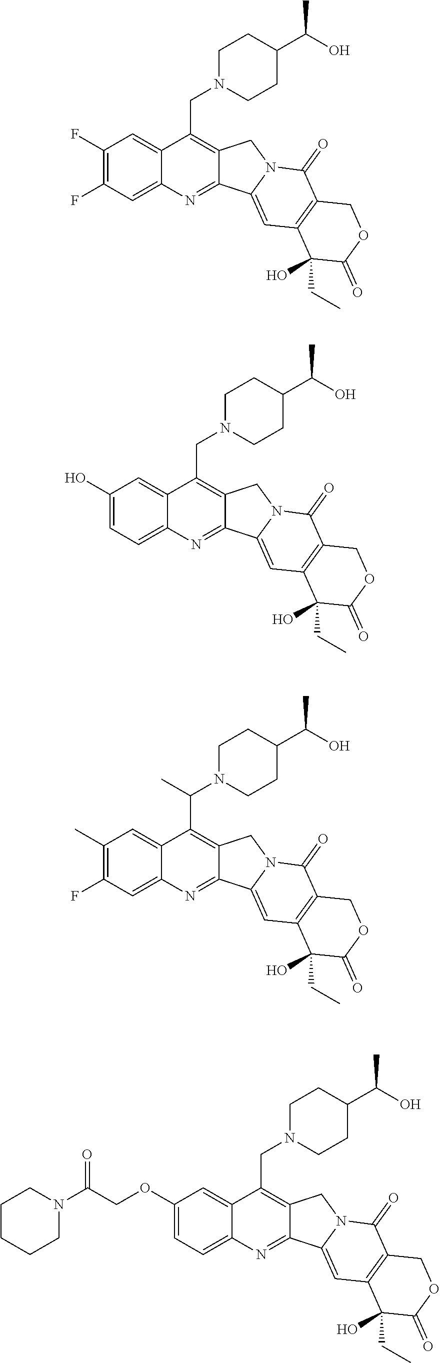

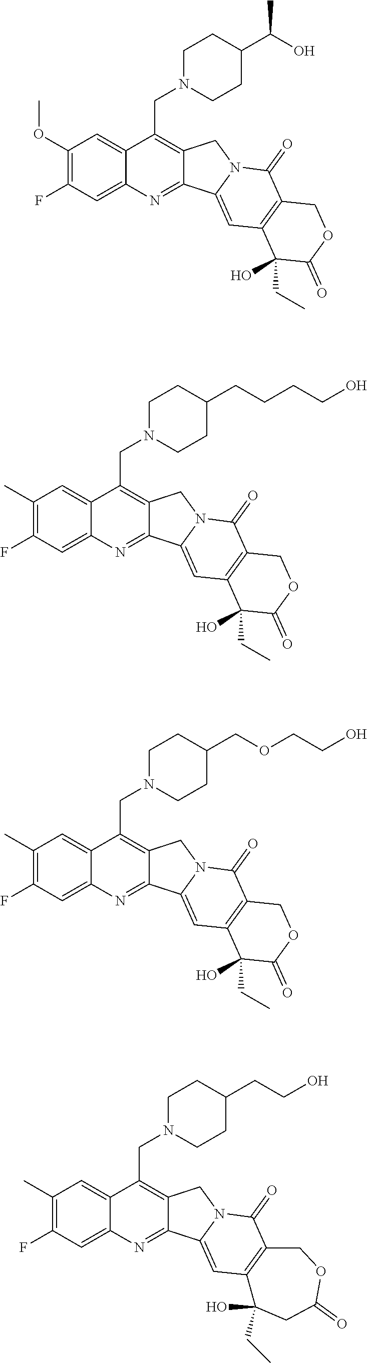 [SMILES: CC[C@@]1(O)C(=O)OCc2c1cc1n(c2=O)Cc2c-1nc1cc(F)c(OC)cc1c2CN1CCC([C@@H](C)O)CC1 ; CC[C@@]1(O)C(=O)OCc2c1cc1n(c2=O)Cc2c-1nc1cc(F)c(C)cc1c2CN1CCC(CCCCO)CC1 ; CC[C@@]1(O)C(=O)OCc2c1cc1n(c2=O)Cc2c-1nc1cc(F)c(C)cc1c2CN1CCC(COCCO)CC1 ; CC[C@@]1(O)CC(=O)OCc2c1cc1n(c2=O)Cc2c-1nc1cc(F)c(C)cc1c2CN1CCC(CCO)CC1]

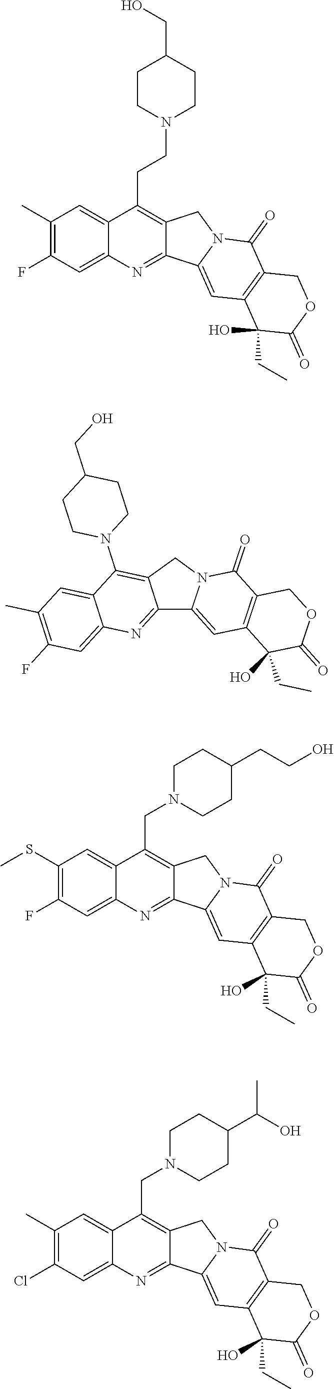 [SMILES: CC[C@@]1(O)C(=O)OCc2c1cc1n(c2=O)Cc2c-1nc1cc(F)c(C)cc1c2CCN1CCC(CO)CC1 ; CC[C@@]1(O)C(=O)OCc2c1cc1n(c2=O)Cc2c-1nc1cc(F)c(C)cc1c2N1CCC(CO)CC1 ; CC[C@@]1(O)C(=O)OCc2c1cc1n(c2=O)Cc2c-1nc1cc(F)c(SC)cc1c2CN1CCC(CCO)CC1 ; CC[C@@]1(O)C(=O)OCc2c1cc1n(c2=O)Cc2c-1nc1cc(Cl)c(C)cc1c2CN1CCC(C(C)O)CC1]

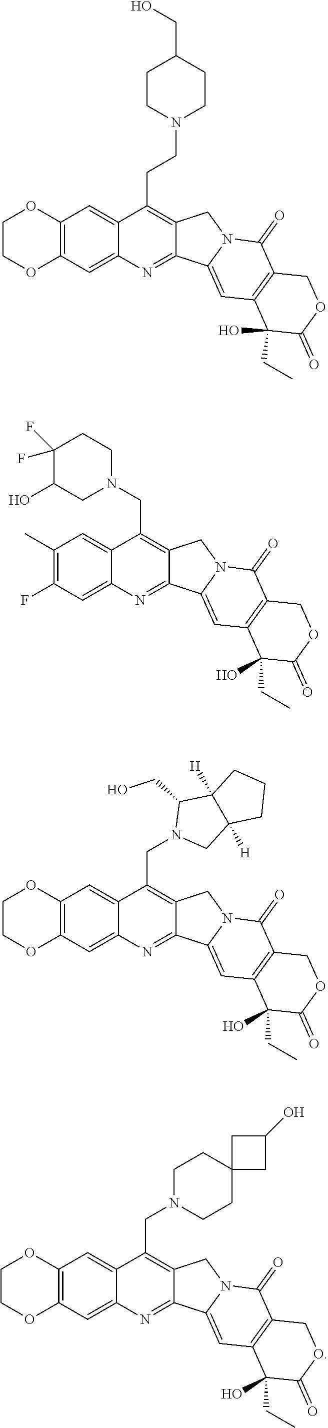 [SMILES: CC[C@@]1(O)C(=O)OCc2c1cc1n(c2=O)Cc2c-1nc1cc3c(cc1c2CCN1CCC(CO)CC1)OCCO3 ; CC[C@@]1(O)C(=O)OCc2c1cc1n(c2=O)Cc2c-1nc1cc(F)c(C)cc1c2CN1CCC(F)(F)C(O)C1 ; CC[C@@]1(O)C(=O)OCc2c1cc1n(c2=O)Cc2c-1nc1cc3c(cc1c2CN1C[C@@H]2CCC[C@@H]2[C@H]1CO)OCCO3 ; CC[C@@]1(O)C(=O)OCc2c1cc1n(c2=O)Cc2c-1nc1cc3c(cc1c2CN1CCC2(CC1)CC(O)C2)OCCO3]

or a tautomer, a mesomer, a racemate, an enantiomer, a diastereomer, or combinations thereof.

In another embodiment, the cell-surface binding molecule T may be of any kind presently known, or which become known cell binding ligands, such as peptides and non-peptides. Generally, the cell-binding molecule T is an antibody; a single chain antibody; an antibody fragment that binds to the target cell; a monoclonal antibody; a single chain monoclonal antibody; or a monoclonal antibody fragment that binds the target cell; a chimeric antibody; a chimeric antibody fragment that binds to the target cell; a domain antibody; a domain antibody fragment that binds to the target cell; adnectins that mimic antibodies, DARPins; a lymphokine; a hormone; a vitamin; a growth factor; a colony stimulating factor; or a nutrient-transport molecule (a transferrin), a binding peptide, or protein, or antibody, or small affinity molecule attached on albumin, polymers, dendrimers, liposomes, nanoparticles, vesicles, (viral)capsids. Preferably the binding molecule T is a monoclonal antibody.

Also provided herein is a compound of the formula (I), (II), (IIa), (III), (IIIa), and (IIIb) and any embodiments thereof, including as defined in the Aspect 1, Embodiment A, Aspect 2, Aspect 2-A, Aspect 3, Aspect 3-A, and Embodiment B, or a pharmaceutically acceptable salt or solvate thereof, wherein the compound is a tautomer, mesomer, racemate, enantiomer, diastereomer or mixture thereof.

Another aspect of the present disclosure provides a method for preparing the ligand-drug conjugate of formula (I), (II), (IIa), (III), (IIIa), and (IIIb), and any embodiments thereof, or the pharmaceutically acceptable salt or solvate thereof, and optionally a tautomer, mesomer, racemate, enantiomer, diastereomer or mixture thereof.

Another aspect of the present disclosure further relates to a pharmaceutical composition comprising a therapeutically effective amount of 1) the ligand-drug conjugate or compound or the pharmaceutically acceptable salt or solvate thereof according to the present disclosure and optionally a tautomer, mesomer, racemate, enantiomer, diastereomer or mixture thereof, and 2) one or more pharmaceutically acceptable carrier(s), diluent(s) or excipient(s).

Another aspect of the present disclosure further relates to a use of 1) the ligand-drug conjugate or compound of the present disclosure, or the pharmaceutically acceptable salt or solvate thereof, and optionally a tautomer, mesomer, racemate, enantiomer, diastereomer or mixture thereof or 2) the pharmaceutical composition comprising the same according to the present disclosure in the preparation of a medicament for treating or preventing a tumor, and preferably the tumor is a cancer related to the expression of HER2, HER3 or EGFR.

Another aspect of the present disclosure further relates to a use of 1) the ligand-drug conjugate or compound, or the pharmaceutically acceptable salt or solvate thereof, and optionally a tautomer, mesomer, racemate, enantiomer, diastereomer or mixture thereof or 2) the pharmaceutical composition comprising the same according to the present disclosure in the preparation of a medicament for treating or preventing a cancer, the cancer is preferably selected from the group consisting of breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer (for example, small cell lung cancer and non-small cell lung cancer), colon cancer, rectal cancer, colorectal cancer, leukemia (for example, acute lymphocytic leukemia, acute myeloid leukemia, acute promyelocytic leukemia, chronic myeloid leukemia, chronic lymphocytic leukemia), bone cancer, skin cancer, thyroid cancer, pancreatic cancer, and lymphoma (for example, Hodgkin's lymphoma, non-Hodgkin's lymphoma, or recurrent anaplastic large cell lymphoma).

The active compound can be formulated into a form suitable for administration by ay appropriate route, and the active compound is preferably in the form of a unit dose, or in a form in which the patient can self-administer in a single dose. The form of the unit dose of the compound or composition of the present disclosure can be tablet, capsule, cachet, bottled portion, powder, granule, lozenge, suppository, regenerating powder or liquid preparation.

The dosage of the compound or composition in the treatment method of the present disclosure will generally vary according to the severity of the disease, the weight of the patient, and the relative efficacy of the compound. However, as a general guide, a suitable unit dose can be 0.1 to 1000 mg.

In addition to the active compound, the pharmaceutical composition of the present disclosure can also comprise one or more auxiliaries including filter (diluent), binder, wetting agent, disintegrant, excipient and the like. Depending on the administration mode, the composition can comprise 0.1 to 99% by weight of the active compound.

The pharmaceutical composition containing the active ingredient can be in a form suitable for oral administration, for example, a tablet, troche, lozenge, aqueous or oily suspension, dispersible powder or granule, emulsion, hard or soft capsule, syrup or elixir. An oral composition can be prepared according to any known method in the art for the preparation of pharmaceutical composition. Such composition can comprise binders, fillers, lubricants, disintegrants or pharmaceutically acceptable wetting agents and the like. Such composition can also comprise one or more components selected from the group consisting of sweeteners, flavoring agents, colorants and preservatives, in order to provide a pleasing and palatable pharmaceutical formulation.

An aqueous suspension comprises an active ingredient in admixture with excipients suitable for the manufacture of an aqueous suspension. The aqueous suspension can also comprise one or more preservative(s), one or more colorant(s), one or more flavoring agent(s), and one or more sweetener(s).

An oil suspension can be formulated by suspending the active ingredient in a vegetable oil. The oil suspension can comprise a thickener. The aforementioned sweeteners and flavoring agents can be added to provide a palatable formulation.

The pharmaceutical composition of the present disclosure can also be in the form of an oil-in-water emulsion.

The pharmaceutical composition can be in the form of a sterile injectable aqueous solution. Acceptable vehicles or solvents that can be used are water, Ringer's solution or isotonic sodium chloride solution. The sterile injectable formulation can be a sterile injectable oil-in-water micro-emulsion in which the active ingredient is dissolved in an oil phase. For example, the active ingredient is dissolved in a mixture of soybean oil and lecithin. The oil solution is then added to a mixture of water and glycerin, and processed to form a micro-emulsion. The injectable solution or micro-emulsion can be introduced into a patient's bloodstream by local bolus injection. Alternatively, the solution and micro-emulsion are preferably administrated in a manner that maintains a constant circulating concentration of the compound of the present disclosure. In order to maintain this constant concentration, a continuous intravenous delivery device can be used. An example of such a device is Deltec CADD-PLUS™ 5400 intravenous injection pump.

The pharmaceutical composition can be in the form of a sterile injectable aqueous or oily suspension for intramuscular and subcutaneous administration. Such a suspension can be formulated with suitable dispersants or wetting agents and suspending agents as described above according to known techniques. The sterile injectable formulation can also be a sterile injectable solution or suspension prepared in a nontoxic parenterally acceptable diluent or solvent. Moreover, sterile fixed oils can easily be used as a solvent or suspending medium.

The compound of the present disclosure can be administrated in the form of a suppository for rectal administration. These pharmaceutical compositions can be prepared by mixing the drug with a suitable non-irritating excipient that is solid at ordinary temperatures, but liquid in the rectum, thereby melting in the rectum to release the drug. Such materials include cocoa butter, glycerin gelatin, hydrogenated vegetable oil; a mixture of polyethylene glycols of various molecular weights and fatty acid esters thereof.

It is well known to those skilled in the art that the dosage of a drug depends on a variety of factors including, but not limited to the following factors: activity of a specific compound, age of the patient, weight of the patient, general health of the patient, behavior of the patient, diet of the patient, administration time, administration route, excretion rate, drug combination and the like. In addition, the optimal treatment, such as treatment mode, daily dose of the compound of formula (I) or the type of pharmaceutically accept-able salt thereof can be verified according to traditional therapeutic regimens.

### BRIEF DESCRIPTION OF THE FIGURES

FIG. 1 shows the results of an in vitro bystander killing test of target cell SK-BR-3 and GFP-labelled tool cell Flip (Example 70).

FIG. 2 shows the results of an in vitro plasma stability study of ADC (Example 71).

FIG. 3 shows the results of an ADC efficacy study on NCI-N87 tumor-bearing nude mice (Example 72).

FIG. 4 shows the change of tumor size produced by ADC treatment after 21 days (Example 72).

### DETAILED DESCRIPTION

Unless otherwise stated, all technical and scientific terms used herein are consistent with the common understanding of those of ordinary skill in the art to which the present disclosure belongs. Although any methods and materials similar or equivalent to those described herein can be used in the practice or testing of the present disclosure, preferred methods and materials are described herein. When describing and protecting the present disclosure, the following terms are used in accordance with the following definitions.

Unless otherwise stated, the terms used in the specification and claims have the meanings described below.

“A” and “an,” as used in herein, mean one or more, unless the context clearly dictates otherwise.

“Ligand” refers to a compound capable of recognizing and binding to an antigen or receptor associated with a target cell. The role of the ligand is to deliver the drug to the target cell population that binds to the ligand. Such ligands include, but are not limited to, protein hormones, lectins, growth factors, antibodies, peptides or other molecules that can bind to cells. In an embodiment of the present disclosure, the ligand is represented by T for trastuzumab. The ligand can form a bond with the Linker via a heteroatom on the ligand. The ligand is preferably an antibody or an antigen binding fragment thereof. The antibody is selected from the group consisting of chimeric antibody, humanized antibody, fully humanized antibody or murine antibody, and preferably a monoclonal antibody.

The term “drug” refers to a cytotoxic drug as provided herein, being a chemical molecule that can strongly disrupt the normal growth of tumor cells. In principle, all cytotoxic drugs can kill tumor cells at a sufficiently high concentration. However, it they may cause the apoptosis of normal cell resulting in serious side effects, even while killing tumor cells due to the lack of specificity.

The term “linker,” “linker unit,” “linking fragment,” or “linking unit” refers to a chemical structural fragment or bond, which is linked to a ligand at one end and linked to a drug at another end. The preferred embodiments of the present disclosure are represented by L and L¹ to L⁴, wherein the L¹ end is linked to the ligand, and the L⁴ end is linked to the drug.

The linker, including extension unit, spacer unit, and amino acid unit, can be synthesized by methods known in the art, such as those described in US 2005-0238649A1. The linker can be a “cleavable linker” or “releasable linker” that facilitates the release of the drug in cell. For example, an acid labile linker (for example, hydrazone), a protease-sensitive (for example, peptidase-sensitive) linker, a light-labile linker, a dimethyl linker or a disulfide-containing linker can be used (Chari et al., Cancer Research 52: 127-131(1992); U.S. Pat. No. 5,208,020).

The term “ligand-drug conjugate” means that a biologically active drug is linked to a ligand as provided herein through a stable linking unit. In the present disclosure, the “ligand-drug conjugate” is preferably an antibody-drug conjugate (ADC), which means that a toxic drug is linked to a monoclonal antibody or antibody fragment with biological activity through a stable linking unit.

The three-letter codes and one-letter codes for amino acids used in the present disclosure are as described in J. Biol. Chem, 243, p 3558 (1968).

The term “antibody” refers to immunoglobulin, a four-peptide chain structure connected together by interchain disulfide bond between two identical heavy chains and two identical light chains. Different immunoglobulin heavy chain constant regions exhibit different amino acid compositions and sequences, hence present different antigenicity. Accordingly, immunoglobulins can be divided into five types, or called immunoglobulin isotypes, namely IgM, IgD, IgG, IgA and IgE, with corresponding heavy chain |i, y, a and e, respectively. According to the amino acid composition of hinge region and the number and location of heavy chain disulfide bonds, the same type of Ig can further be divided into different sub-types, for example, IgG can be divided into IgG1, IgG2, IgG3 and IgG4. Light chain can be divided into K or X chain based on different constant region. Each five types of Ig can have a K or X chain. The antibodies described in the present disclosure are preferably specific antibodies against the cell surface antigens on the target cells, non-limiting examples are one or more of the following antibodies: anti-HER2 (ErbB2) antibody, anti-EGFR antibody, anti-B7-H3 antibody, anti-c-Met antibody, anti-HER3 (ErbB3) antibody, anti-HER4 (ErbB4) antibody, anti-CD20 antibody, anti-CD22 antibody, anti-CD30 antibody, anti-CD33 antibody, anti-CD44 antibody, anti-CD56 antibody, anti-CD70 antibody, anti-CD73 antibody, anti-CD105 antibody, anti-CEA antibody, anti-A33 antibody, anti-Cripto antibody, anti-EphA2 antibody, anti-G250 antibody, anti-MUC1 antibody, anti-Lewis Y antibody, anti-VEGFR antibody, anti-GPNMB antibody, anti-Integrin antibody, anti-PSMA antibody, anti-Tenascin-C antibody, anti-SLC44A4 antibody or anti-Mesothelin antibody, and preferably Trastuzumab (trade name Herceptin), Pertuzumab (also known as 2C4, trade name Peijeta), Nimotuzumab (trade name Taixinsheng), Enoblituzumab, Emibetuzumab, Inotuzumab, Pinatuzumab, Brentuximab, Gemtuzumab, Bivatuzumab, Lorvotuzumab, cBR96 and Glembatumumab.

About 110 amino acid sequence adjacent to the N-terminus of the antibody heavy chains or light chains is highly variable, known as variable region (Fv region); the rest of amino acid sequence adjacent to the C-terminus is relatively stable, known as constant region. The variable region includes three hypervariable regions (HVR) and four relatively conservative framework regions (FR). The three hypervariable regions, which determine the specificity of the antibody, are also known as the complementarity determining regions (CDR). Each light chain variable region (LCVR) or each heavy chain variable region (HCVR) consists of three CDR regions and four FR regions, with sequential order from the amino terminus to carboxyl terminus in the following order: FR1, CDR1, FR2, CDR2, FR3, CDR3, FR4. The three CDR regions of the light chain refer to LCDR1, LCDR2, and LCDR3; and the three CDR regions of the heavy chain refer to HCDR1, HCDR2, and HCDR3.

Antibodies of the present disclosure include murine antibodies, chimeric antibodies, humanized antibodies and fully humanized antibodies, and preferably humanized antibodies and fully humanized antibodies.

The term “murine antibody” in the present disclosure refers to the antibody prepared from murine according to the knowledge and skills of the field. During the preparation, the test subject is injected with specific antigen, and then a hybridoma expressing the antibody which possesses the desired sequence or functional characteristics is isolated.

The term “chimeric antibody,” is an antibody obtained by fusing a variable region of a murine antibody with a constant region of a human antibody, and the chimeric antibody can alleviate the murine antibody-induced immune response. To establish a chimeric antibody, a hybridoma secreting murine specific monoclonal antibody is established, and a variable region gene is cloned from the murine hybridoma cell; then a constant region gene of human antibody is cloned according to requirement; and the constant region gene of human is connected with the variable region gene of murine to form a chimeric gene, which is subsequently inserted into an expression vector; finally, the chimeric antibody molecule is expressed in an eukaryotic or prokaryotic system.

The term “humanized antibody”. which is also known as CDR-grafted antibody, refers to an antibody generated by grafting murine CDR sequences into human antibody variable region framework, i.e., an antibody produced in different types of human germline antibody framework sequences. Humanized antibody can overcome heterologous responses induced by large number of murine protein components carried by chimeric antibody. Such framework sequences can be obtained from public DNA database covering germline antibody gene sequences or published references. For example, germline DNA sequences of human heavy and light chain variable region genes can be found in “VBase” human germline sequence database (available on the world wide web at: www.mrccpe.com.ac.uk/vbase), as well as in Kabat, E A, et al. 1991 Sequences of Proteins of Immunological Interest, 5th Ed. To avoid a decrease in activity caused by the decreased immunogenicity, the framework sequences in the variable region of human antibody can be subjected to minimal reverse mutations or back mutations to maintain the activity. The humanized antibody of the present disclosure also comprises humanized antibody on which CDR affinity maturation is performed by phage display. Documents that further describe methods of using murine antibodies involved in humanization include, for example, Queen et al., Proc., Natl. Acad. Sci. USA, 88, 2869, 1991 and Winter and colleagues' method [Jones et al., Nature, 321, 522(1986), Riechmann et al., Nature, 332, 323-327(1988), Verhoeyen et al., Science, 239, 1534(1988)].

The term “fully humanized antibody,” is also known as “fully humanized monoclonal antibody,” wherein the variable region and constant region of the antibody are both of human origin, eliminating immunogenicity and side effects. The development of monoclonal antibody has gone through four stages, namely: murine monoclonal antibody, chimeric monoclonal antibody, humanized monoclonal antibody and fully humanized monoclonal antibody. The antibody of the present disclosure is a fully humanized monoclonal antibody. The related technologies of fully humanized antibody preparation mainly include human hybridoma technology, EBV transformed B lymphocyte technology, phage display technology, transgenic mouse antibody preparation technology, single B cell antibody preparation technology and the like.

The term “antigen binding fragment” refers to one or more fragments of an antibody retaining the specific binding ability to the antigen. It has been shown that fragments of full-length antibody can be used to achieve the function of binding with an antigen. The examples of binding fragments in the term “antigen binding fragment” include (i) Fab fragment, a monovalent fragment composed of VL, VH, CL and CH1 domain; (ii) F(ab′)2 fragment, a bivalent fragment comprising two Fab fragments connected by a disulphide bond in the hinge region; (iii) Fd fragment, consisting of VH and CH: domains; (iv) Fv fragment, consisting of VH and VL domains of one-arm antibody; (v) single domain or dAb fragment (Ward et al. (1989) Nature 341:544-546) composed of VH domain; and (vi) an isolated complementary determining region (CDR) or (vii) a combination of two or more isolated CDRs optionally connected by a synthetic linker. In addition, although the VL domain and VH domain of the Fv fragment are encoded by two separate genes, they can be connected by a synthetic linker by using recombinant methods, thereby generating a single protein chain of a monovalent molecular formed by pairing the VL and VH domain (referred to as single chain Fv (scFv); see, e.g., Bird et al. (1988) Science: 242:423-426, and Huston et al. (1988) Proc. Natl. Acad. Sci USA 85:5879-5883). This single chain antibody is also intended to be included in the term “antigen binding fragment” of the antibody. Such antibody fragments are obtained using conventional techniques known by those skilled in the art, and screened for functional fragments by using the same method as that for an intact antibody. Antigen binding sites can be produced by recombinant DNA technology or by enzymatic or chemical disruption of an intact immunoglobulin. Antibodies can be antibodies of different isotypes, e.g., IgG (e.g., IgG1, IgG2, IgG3 or IgG4 subtype), IgA1, IgA2, IgD, IgE or IgM antibody.

Fab is an antibody fragment obtained by treating an IgG antibody molecule with a papain (which cleaves the amino acid residue at position 224 of the H chain). The Fab fragment has a molecular weight of about 50,000 and has antigen binding activity, in which about a half of the N-terminal side of H chain and the entire L chain are bound together through a disulfide bond.

The term “CDR” refers to one of the six hypervariable regions within the variable domain of an antibody that primarily contributes to antigen binding. One of the most commonly used definitions for the six CDRs is provided by Kabat E. A. et al. (1991) Sequences of proteins of immunological interest. NIH Publication 91-3242. As used herein, the Kabat definition of CDR only applies to CDR1, CDR2 and CDR3 of the light chain variable domain (CDR L1, CDR L2, CDR L3 or L1, L2, L3), as well as CDR2 and CDR3 of heavy chain variable domain (CDR H2, CDR H3 or H2, H3).

The term “antibody framework” refers to a portion of the variable domain VL or VH, which serves as a scaffold for the antigen binding loop (CDR) of the variable domain. Essentially, it is a variable domain without CDR.

The terms “specific binding,” “selective binding,” “selectively bind” and “specifically bind,” refer to the binding of an antibody to an epitope on a predetermined antigen. Typically, the antibody binds with an affinity (KD) of less than about 10-7M, such as approximately less than about 10-8 M, 10-9M or 10-10 M or less.

Methods for producing and purifying antibodies and antigen binding fragments are well known in the art, such as Cold Spring Harbor Antibody Technical Guide, Chapters 5-8 and 15.

The antigen binding fragment can also be prepared by conventional methods. The antibodies or antigen binding fragments of the disclosure are genetically engineered to add one or more human FR regions in nonhuman CDR regions. The human FR germline sequence(s) can be obtained by aligning IMGT human antibody variable germlines gene databases and MOE software from the ImMunoGeneTics (IMGT) website at http://imgt.cines.fr or from the Journal of Immunoglobulins 20011SBN012441351.

The term “peptide” refers to a compound fragment between amino acid and protein, consisting of two or more amino acid molecules connected to each other through peptide bonds. Peptides are structural and functional fragments of proteins. Hormones, enzymes and the like are essentially peptides.

The term “toxin” refers to any substance that can have a harmful effect on the growth or proliferation of cells. Toxins can be small molecule toxins and their derivatives from bacteria, fungi, plants or animals, including Camptothecin derivatives such as exatecan, maytansinoid and its derivatives (CN101573384) such as DM1, DM3, DM4, auristatin F (AF) and its derivatives such as MMAF, MMAE, 3024 (WO 2016/127790 A1, compound 7), diphtheria toxin, exotoxin, ricin A chain, abrin A chain, modeccin, a-sarcin, Aleurites fordii toxic protein, dianthin toxic protein, Phytolaca americana toxic protein (PAPI, PAPII and PAP-S), Momordica charantia inhibitor, curcin, crotin, Sapaonaria officinalis inhibitor, gelonin, mitogellin, restrictocin, phenomycin, 61datrexa and trichothecenes.

The term “chemotherapeutic drug” refers to a chemical compound that can be used to treat tumors. This definition also includes antihormonal agents that act to modulate, reduce, block, or inhibit the effects of hormones that promote cancer growth, which are often in the form of systemic or holistic therapy. They can be hormones. Examples of chemotherapeutic drugs include alkylating agents, such as thiotepa; cyclosphamide; alkyl sulfonate such as busulfan, improsulfan and piposulfan; aziridine such as benaodopa and uredepa; aziridine and methylamelamine including altretamine, triethylenemelamine, and trimethylolomelamine; nitrogen mustards such as chlorambucil, chlornaphazine; melphalan, novembichin; nitrosoureas such as carmustine, chlorozotocin; antibiotic such as aclacinomycin, actinomycin, authramycin, azaserine, bleomycin, cactinomycin C, calicheamicin, carabicin, chromomycin, carzinophilin, actinomycin D, daunorubicin, detorubicin, doxorubicin, epirubicin, esorubicin, idarubicin, mycophenolic acid, nogalamycin, olivomycin, peplomycin, potfiromycin, puromycin, quelamycin, rodorubicin, streptonigrin; streptozocin, tuberculocidin, ubenimex, zinostatin, zorubicin; antimetabolites such as methotrexate, 5-fluorouracil (5-FU); folic acid analogs such as denopterin, methotrexate, pteropterin, trimetrexate; pterin analogs such as fludarabine, 6-mercaptopterin, thiomethopterin, thioguanopterin; pyrimidine analogs such as ancitabine, datrexate, 6-azuridine, carmofiir, doxitluridine, enocitabine, floxuridine; androgens such as calusterone, dromostanolong propionate, epitiostanol, testolactone; anti-adrenalines such as aminoglutethimide, mitotane, trilostane; folic acid supplements such as frolinic acid; aceglatone; aldophosphamideglycoside; aminolevulinic acid; amsacrine; bestrabucil; biasntrene; defbfamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pintostatin; phenamet; pirarubicin; podophyllinic acid; 2-ethylhydrazide; procarbazine; razoxane; sizofiran; spirogemanium; tenuazonic acid; triaziquone; trichlorrotriethylamine; urethan; vindesine; dacarbazine; mannomustine; mitobronitol; dibromodulcitol; pipobroman; gacytosine; arabinoside; cyclophosphamide; thiotepa; taxanes such as paclitaxel and docetaxel; chlorambucil; gemcitabine; 6-thioguanine; mercaptopurine; methotrexate; platinum analogs such as cisplatin and carboplatin; vinblastine; platinum; etoposide; ifosfamide; mitomycin C; mitoxantrone; vincristine; vinorelbine; navelbine; novantrone; teniposide; daunorubicin; aminopterin; xeloda; ibandronate; topoisomerase inhibitor RFS2000; difluoromethylomithine; retinoic acid esperamicins; capecitabine; and pharmaceutically acceptable salt, acid or derivative of any of the above substances. This definition also includes antihormonal agents.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₆ alkyl refers to alkyl of one to six carbon atoms, inclusive.

The term “alkyl” as used herein refers to a linear or branched-chain saturated hydrocarbyl substituent (i.e., a substituent obtained from a hydrocarbon by removal of a hydrogen); in one embodiment containing from one to eight carbon atoms, in another one to six carbon atoms and in yet another one to three carbon atoms. Non-limiting examples of such substituents include methyl, ethyl, propyl (including n-propyl and isopropyl), butyl (including n-butyl, isobutyl, sec-butyl and tert-butyl, pentyl, isoamyl, hexyl, heptyl, octyl and the like. In another embodiment containing one to three carbons and consisting of methyl, ethyl, n-propyl and isopropyl. The phrase “each ‘C₁-C₈ alkyl” are optionally substituted with one to three R⁹” means that each “C₁-C₈ alkyl,” in a recited list of groups can be substituted with one to three R⁹. For example, in the following list, “C₁-C₈ alkyl, (C₁-C₈ alkyl)NHC(O)O—, (C₁-C₈ alkyl)NH—, (C₁-C₈ alkyl)C(O)O—” each of the C₁-C₈ alkyl can be substituted with one to three R⁹.

The term “alkylene” as used herein refers to a divalent alkyl group, as defined herein.

The term “-alkylene-cycloalkylene-” as used herein refers to an alkylene group, as defined herein, bonded to a cycloalkylene group as defined herein.

The term “alkoxy” refers to an —OR group, wherein R is alkyl, as defined herein, (i.e., a substituent obtained from a hydrocarbon alcohol by removal of the hydrogen from the OH); in one embodiment containing from one to six carbon atoms. Non-limiting examples of such substituents include methoxy, ethoxy, propoxy (including n-propoxy and isopropoxy), butoxy (including n-butoxy, isobutoxy, sec-butoxy and tert-butoxy), pentoxy, hexoxy and the like. In another embodiment having one to three carbons and consisting of methoxy, ethoxy, n-propoxy and isopropoxy. An alkoxy group which is attached to an alkyl group is referred to as an alkoxyalkyl. An example of an alkoxyalkyl group is methoxymethyl.

The term “alkoxyalkyl” as used herein refers to an alkyl group substituted with an alkoxy group, as defined herein.

The term “cycloalkyl” refers to a carbocyclic substituent obtained by removing a hydrogen from a saturated or a partially unsaturated (but does not comprise an aromatic ring) carbocyclic molecule, for example one having three to seven carbon atoms. The term “cycloalkyl” includes monocyclic saturated carbocycles. The term “C₃-C₇, cycloalkyl” means a radical of a three- to seven-membered ring system which includes the groups cyclopropyl, cyclobutyl, cyclopentyl, cyclohexyl, and cycloheptyl. The term “Cₛ-Cₛ cycloalkyl” means a radical of a three- to six-membered ring system which includes the groups cyclopropyl, cyclobutyl, cyclopentenyl, cyclopentyl, cyclohexenyl, and cyclohexyl. The cycloalkyl groups can also be bicyclic or spirocyclic carbocycles. For example, the term “C₃-C₁₂ cycloalkyl” includes monocyclic carbocycles and bicylic and spirocyclic cycloalkyl moieties such as bicydapentyl, bicydohexyl, bicycloheptyl, bicyclooctyl, bicyclononyl, spiropentyl, spirohexyl, spiroheptyl, spirooctyl and spironanyl.

The term “cycloalkylene” refers to a divalent cycloalkyl group, as defined herein.

The term “C₃-C₆ cycloalkoxy” refers to a three- to six-membered cycloalkyl group attached to an oxygen radical. Examples include cyclopropoxy, cyclobutoxy, cyclopentoxy and cyclohexoxy.

In some instances, the number of atoms in a cyclic substituent containing one or more heteroatoms (ie., heteroaryl or heterocycloalkyl is indicated by the prefix “x- to y-membered,” wherein x is the minimum and y is the maximum number of atoms forming the cyclic moiety of the substituent. Thus, for example, “4 to 6-membered heterocycloalkyl” refers to a heterocycloalkyl containing from 4 to 6 atoms, including one to three heteroatoms, in the cyclic moiety of the heterocycloalkyl. Likewise, the phrase “5- to 6-membered heteroaryl” refers to a heteroaryl containing from 5 to 6 atoms, and “5- to 10-membered heteroaryl” refers to a heteroaryl containing from 5 to 10 atoms, each including one or more heteroatoms, in the cyclic moiety of the 30 heteroaryl. Furthermore, the phrases “5-membered heteroaryl” and “6-membered heteroaryl” refer to a five-membered heteroaromatic ring system and a six-membered heteroaromatic ring system, respectively. The heteroatoms present in these ring systems are selected from N, O and S.

The term “hydroxy” or “hydroxyl” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol. The terms cyano and nitrile refer to a —CN group. The term “oxo” means an oxygen which is attached to a carbon by a double bond (i.e., when R⁴ is oxo then R⁴ together with the carbon to which it is attached are a C═O moiety).

The term “hydroxyalkyl” refers to an alkyl group, as defined herein, substituted with 1, 2, or 3 hydroxy groups.

The term “halo” or “halogen” refers to fluorine (which may be depicted as —F), chlorine (which may be depicted as —Cl), bromine (which may be depicted as —Br), or iodine (which may be depicted as —I).

The term “haloalkyl” refers to an alkyl group, as defined herein, substituted with 1, 2, 3, 4, 5, or 6 halo groups. In some embodiments, haloalkyl includes chloroalkyl.

The term “heterocycloalkyl” refers to a substituent obtained by removing a hydrogen from a saturated or partially saturated ring structure containing a total of the specified number of atoms, such as 4 to 6 ring atoms or 4 to 12 atoms, wherein at least one of the ring atoms is a heteroatom (i.e., oxygen, nitrogen, or sulfur), with the remaining ring atoms being independently selected from the group consisting of carbon, oxygen, nitrogen, and sulfur. The sulfur may be oxidized [i.e., S(O) or S(O)₂] or not. In a group that has a heterocycloalkyl substituent, the ring atom of the heterocycloalkyl substituent that is bound to the group may be a nitrogen heteroatom, or it may be a ring carbon atom. Similarly, if the heterocycloalkyl substituent is in turn substituted with a group or substituent, the group or substituent may be bound to a nitrogen heteroatom, or it may be bound to a ring carbon atom. It is to be understood that a heterocyclic group may be monocyclic, bicyclic, polycyclic or spirocyclic.

The term “aryl” refers to a carbocyclic monocyclic or bicyclic ring system, wherein the monocyclic ring is aromatic and the bicyclic ring comprises at least one aromatic ring. The term “C₆-C₁₀ aryl” refers to carbocyclic systems with 6 to 10 atoms and includes phenyl, tetrahydronaphthyl, and naphthyl. In addition to any group specifically recited in any of the embodiments or claims, in some embodiments, the aryl is optionally substituted with 1 to 3 substituents independently selected from halo, —C₁₋₁₂alkyl (unsubstituted or substituted, in one embodiment with 1, 2, or 3 halo), aryl, —OH, —OC₁₋₁₂alkyl, —S(O)ₙC₁₋₄alkyl (wherein n is 0, 1, or 2), —C₁₋₄alkylNH₂, —NHC₁₋₄alkyl, —C(═O)H, C(═O)OR^{a}, —OC(═O)R^{b}, OC(═O)N^{a}R^{c}, OC(═O)heteroaryl, OC(═O)(heterocyclic ring) and C═N—OR^{d} wherein R^{a}, R^{c}, and R^{d} are independently hydrogen or —C₁₋₄alkyl and R^{b} is alkyl.

The term “arylene” as used herein refers to a divalent aryl group, as defined herein.

The term “heteroalkyl” refers to an alkyl group, as defined herein, wherein one or more —CH₂— is replaced by a group independently selected from —O—, —S—, —S(O)—, —S(O)₂, and —NR— where R is hydrogen or alkyl, as defined herein, and/or wherein one or more —CH₃ group is replaced by a group independently selected from —OH, —SH, and —NH₂ where each R is independently hydrogen or alkyl. Heteroalkyl includes 2-thioethyl, 2-amino-prop-1-yl, 2-hydroxy-eth-1-yl, N-methyl-amino-ethyl, and the like. Hydroxyalkyl is a subset of heteroalkyl.

The term “heteroalkylene” refers to a divalent heteroalkyl, as defined herein.

The term “heteroaryl” refers to an aromatic ring structure containing the specified number of ring atoms in which at least one of the ring atoms is a heteroatom (i.e., oxygen, nitrogen, and/or sulfur), with the remaining ring atoms being carbon. Examples of heteroaryl substituents include 6-membered heteroaryl rings such as pyridyl, pyrazyl, pyrimidinyl, and pyridazinyl; and 5-membered heteroaryl rings such as triazolyl, imidazolyl, furanyl, thiophenyl, pyrazolyl, pyrrolyl, oxazolyl, isoxazolyl, thiazolyl, 1,2,3-oxadiazolyl, 1,2,4-oxadiazolyl, 1,2,5-oxadiazolyl, 1,3,4-oxadiazolyl and isothiazolyl. The heteroaryl group can also be a bicyclic heteroaromatic group such as indolyl, benzofuranyl, benzothienyl, benzimidazolyl, benzothiazolyl, benzoxazolyl, benzoisoxazolyl, oxazolopyridinyl, imidazopyridinyl, imidazopyrimidinyl and the like. In a group that has a heteroaryl ring, the ring atom of the heteroaryl ring that is bound to the group may be a nitrogen atom, or it may be a ring carbon atom. Similarly, if the heteroaryl ring is in turn substituted with a group or substituent, the group or substituent may be bound to a nitrogen atom, or it may be bound to a ring carbon atom. The term “heteroaryl” also includes pyridyl N-oxides and groups containing a pyridine N-oxide ring. In addition, the heteroaryl group may contain an oxo group such as the one present in a pyridone group. Further examples include furyl, thienyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, pyridin-2(1H)-onyl, pyridazin-2(1H)-onyl, pyrimidin-2(1H-onyl, pyrazin-2(1H)-onyl, imidazo[1,2-a]pyridinyl, and pyrazolo[1,5-a]pyridinyl. The heteroaryl can be further substituted as defined herein.

Examples of single-ring heteroaryls and heterocycloalkyls include furanyl, dihydrofuranyl, tetrahydrofuranyl, thiophenyl, dihydrothiophenyl, tetrahydrothiophenyl, pyrrolyl, isopyrroyl, pyrrolinyl, pyrolidinyl, imidazolyl, isoimidazolyl, imidazolinyl, imidazolidinyl, pyrazolyl, pyrazolinyl, pyrazalidinyl, tiazolyl, tetrazolyl, dithiolyl, oxathiolyl, oxazalyl, isoxazolyl, thiazolyl, isothiazolyl, thiazolinyl, isothiazolinyl, thiazolidinyl, isothiazolidinyl, thiaoxadiazolyl, oxathiazolyl, Dxadiazolyl (including oxadiazolyl, 1,2,4-oxadiazolyl, 1,2,5-oxadiazolyl, or 1,3,4-oxadiazoly), pyranyl (including 1,2-pyranyl or 1,4-pyranyl),dihydropyranyl, pyridinyl, piperidinyl, diazinyl (including pyridazinyl, pyrimidinyl, piperazinyl, triazinyl (including s-triazinyl, as-triazinyl and v-triazinyl), oxazinyl (including 2H-1,2-oxazinyl, 6H-1,3-oxazinyl, or 2H-1,4-oxazinyl), isoxazinyl (including O-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 2H1,2,4-oxadiazinyl or 2H1,2,5-oxadiazinyl), and morpholinyl.

The term “heteroaryl” can also include, when specified as such, ring systems having two rings wherein such rings may be fused and wherein one ring is aromatic and the other ring is not fully part of the conjugated aromatic system (i.e., the heteroaromatic ring can be fused to a cycloalkyl an heterocycloalkyl ring). Non-limiting examples of such ring systems include 5,6,7,8-tetrahydroisoquinalinyl, 5,6,7,8-tetrahydroquinolinyl, 6,7-dihydro-5H-cyclopenta[b]pyridinyl, 6,7-dihydro-5H-cyclopenta[c]pyridinyl, 1,4,5,6-tetrahydrocyclopenta[c]pyrazolyl, 2,4,5,6-tetrahydrocyclopenta[c]pyrazolyl, 5,6-dihydro-4Hpyrolo[1,2-b]pyrazolyl, 6,7-dihydro-5H-pyrrolo[1,2-b[1,2,4]triazolyl, 5,6,7,8-tetrahydro-[1,2.4]triazolo[1,5-a]pyridinyl, 4.5,6,7-tetrahydropyrazolo[1.5-a]pyridinyl, 4,5,6,7-tetrahydro-1H-indazolyl and 4,5.6,7-tetrahydro-2H-indazolyl.

It is to be understood that if a carbocyclic or heterocyclic moiety may be bonded or otherwise attached to a designated group through differing ring atoms without denoting a specific point of attachment, then all possible points are intended, whether through a carbon atom or, for example, a trivalent nitrogen atom. For example, the term “pyridyl” means 2-, 3- or 4-pyridyl, the term “thienyl” means 2- or 3-thienyl, and so forth.

The term “heteroarylene” as used herein refers to a divalent heteroaryl group, as defined herein.

The term “amino protecting group” refers to a group which prevents an amino group from reaction when other parts of the molecular are subject to a reaction and can be easily removed. Non-limiting examples include 9-fluorenylmethyloxycarbonyl, tert-butoxycarbonyl, acetyl, benzyl, allyl, p-methoxybenzyl and the like. These groups can be optionally substituted by one to three substituent(s) selected from the group consisting of halogen, alkoxy and nitro. The amino protecting group is preferably 9-fluorenylmethyloxycarbonyl.

The term “deuterated alkyl” refers to an alkyl group substituted by one or more deuterium atom(s), wherein the alkyl is as defined above.

The term “unsaturated” in the context of the term cycloalkyl, cycloalkylene, and heterocycle refers to a partially unsaturated, but not aromatic ring.

The term “fused” means bicyclic, tricyclic, or polycyclic structures comprised of at least two carbocyclic or heterocyclic structures sharing at least one chemical bond.

If substituents are described as “independently” having more than one variable, each instance of a substituent is selected independent of the other(s) from the list of variables available. Each substituent therefore may be identical to or different from the other substituent(s).

If substituents are described as being “independently selected” from a group, each instance of a substituent is selected independent of the other(s). Each substituent therefore may be identical to or different from the other substituent(s).

“Optional” or “optionally” means that the subsequently described event or circumstance may, but need not, occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or di-substituted with an alkyl group and situations where the aryl group is not substituted with the alkyl group.

“Substituted” refers to one or more hydrogen atoms in a group, preferably up to 5, and more preferably 1 to 3 hydrogen atoms, independently substituted by a corresponding number of substituents. It goes without saying that the substituents only exist in their possible chemical position. The person skilled in the art is able to determine whether the substitution is possible or impossible by experiments or theory without excessive effort. For example, the combination of amino or hydroxy having free hydrogen and carbon atoms having unsaturated bonds (such as olefinic) may be unstable.

As used herein, the term “a compound of Formula (I)” (or other formula number) is defined to include all forms of the compound of Formula 1, including hydrates, solvates, isomers, crystalline and non-crystalline forms, isomorphs, polymorphs, and metabolites thereof. For example, the compounds disclosed herein, or pharmaceutically acceptable salts thereof, may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm. Compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds provided herein may possess one or more asymmetric centers; such compounds can therefore be produced as individual (R)- or (S)-stereoisomers or as mixtures thereof. Unless indicated otherwise, the description or naming of a particular compound in the specification and Claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

A hydrogen (H) or carbon (C) substitution for compounds of the formula I include a substitution with any isotope of the respective atom. Thus, a hydrogen (H) substitution includes a ¹H, ²H (deuterium), or ³H (tritium) isotope substitution, as may be desired, for example, for a specific therapeutic or diagnostic therapy, or metabolic study application, or metabolic or chemical stability enhancement. Optionally, a compound of this disclosure may incorporate a known in the art radioactive isotope or radioisotope, such as ³H, ¹⁵O, ¹²C, or ¹³N isotope, to afford a respective radiolabeled compound of formula I.

A “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and Claims includes both one and more than one such carrier.

A “pharmaceutically acceptable salt” of a compound means a salt that is pharmaceutically acceptable and that possesses the desired pharmacological activity of the parent compound. Such salts include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2 hydroxyethanesulfonic acid, benzenesulfonic acid, 4-chlorobenzenesulfonic acid, 2-naphthalenesulfonic acid, 4-toluenesulfonic acid, camphorsulfonic acid, 4 methylbicyclo[2.2.2]oct-2-ene-1-carboxylic acid, glucoheptonic acid, 4,4′ methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Treating”, “treatment”, or “therapy” of a disease includes:
(1) preventing the disease, i.e., causing the clinical symptoms of the disease not to develop in a mammal that may be exposed to or predisposed to the disease but does not yet experience or display symptoms of the disease,
(2) inhibiting the disease, i.e., arresting or reducing the development of the disease or its clinical symptoms, or
(3) relieving the disease, i.e., causing regression of the disease or its clinical symptoms.

The term “pharmaceutical composition”, refers to a mixture of one or more of the compounds described herein or physiologically/pharmaceutically acceptable salts or pro drugs thereof with other chemical components, and other components such as physiologically/pharmaceutically acceptable carriers and excipients. The purpose of the pharmaceutical composition is to facilitate administration of a compound to an organism, which is conducive to the absorption of the active ingredient so as to show biological activity.

The term “solvate” refers to a pharmaceutically acceptable solvate formed by a ligand-drug conjugate of the present disclosure with one or more solvent molecule(s). Non-limiting examples of solvent molecules include water, ethanol, acetonitrile, isopropanol, DMSO, ethyl acetate.

The term “carrier” used in the composition of the present disclosure refers to a system that can change the way a drug enters the human body and distribution, control the drug release rate, and deliver the drug to the targeted organ. Drug carrier release and targeting systems can reduce drug degradation and loss, reduce side effects and improve bioavailability.

The term “excipient” is an adjunct in a pharmaceutical formulation other than a main drug, which can also be referred to as an adjuvant, such as adhesives, fillers, disintegrants, lubricants in tablets; matrix parts in the semisolid preparations ointment and cream; preservatives, anti-oxidants, flavoring agents, fragrances, co-solvents, emulsifiers, solubilizers, osmotic pressure regulators, colorants in liquid preparations and the like.

The term “diluent”, also known as filler, is primarily intended to increase the weight and volume of the tablet. The addition of diluent ensures a certain volume, reduces the dose deviation of the main components, and improves the compression profile of the drug. When the tablet contains an oily component, an absorbent is added to absorb the oily substance, thereby keeping the “dry” state to facilitate tablet formation. For example, diluent includes starch, lactose, inorganic salts of calcium, microcrystalline cellulose and the like.

The pharmaceutical composition can be in the form of a sterile injectable aqueous solution. Acceptable vehicles or solvents that can be used are water, Ringer's solution or isotonic sodium chloride solution. The sterile injectable formulation can be a sterile injectable oil-in-water micro-emulsion in which the active ingredient is dissolved in the oil phase. For example, the active ingredient is dissolved in a mixture of soybean oil and lecithin. The oil solution is then added to a mixture of water and glycerin, and processed to form a micro-emulsion. The injectable solution or micro-emulsion can be introduced into a patient's bloodstream by local bolus injection. Alternatively, the solution and micro-emulsion are preferably administrated in a manner that maintains a constant circulating concentration of the compound of the present disclosure. In order to maintain this constant concentration, a continuous intravenous delivery device can be used. An example of such a device is Deltec CADD-PLUS™ 5400 intravenous injection pump.

The pharmaceutical composition can be in the form of a sterile injectable aqueous or oily suspension for intramuscular and subcutaneous administration. Such a suspension can be formulated with suitable dispersants or wetting agents and suspending agents as described above according to known techniques. The sterile injectable formulation can also be a sterile injectable solution or suspension prepared in a nontoxic parenterally acceptable diluent or solvent, for example, a solution prepared in 1,3-butanediol. Moreover, sterile fixed oils can easily be used as a solvent or suspending medium. For this purpose, any blending fixed oils including synthetic mono- or di-glyceride can be employed. Moreover, fatty acids, such as oleic acid, can. also be employed in the preparation of an injection.

The term “drug loading”, refers to the average number of cytotoxic drugs loaded on each ligand in the compound of formula (I), and can also be expressed as the ratio of the number of drug to the number of antibody. The drug loading can range from 0 to 12, preferably from 1 to 10 cytotoxic drugs per ligand. In an embodiment of the present disclosure, the drug loading is expressed as n, and exemplary values can be an average of 1, 2, 3, 4, 5, 6, 7, 8, 9, 10. The average number of drugs per ADC molecule after coupling reaction can be determined by conventional methods such as UV/visible spectroscopy, mass spectrometry, ELISA test and HPLC characterization.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “mammal” refers to all mammals including humans, livestock, and companion animals.

The compounds described herein are generally named according to the IUPAC or CAS nomenclature system. Abbreviations which are well known to one of ordinary skill in the art may be used (e.g. “Ph” for phenyl, “Me” for methyl, “Et” for ethyl, “h” for hour or hours and “r.t.” or “rt” for room temperature).

### Synthesis Method of the Present Disclosure

### Scheme I

A method for preparing the compound of formula (VI) or the pharmaceutically acceptable salt or solvate thereof of the present disclosure, comprises the following step of:

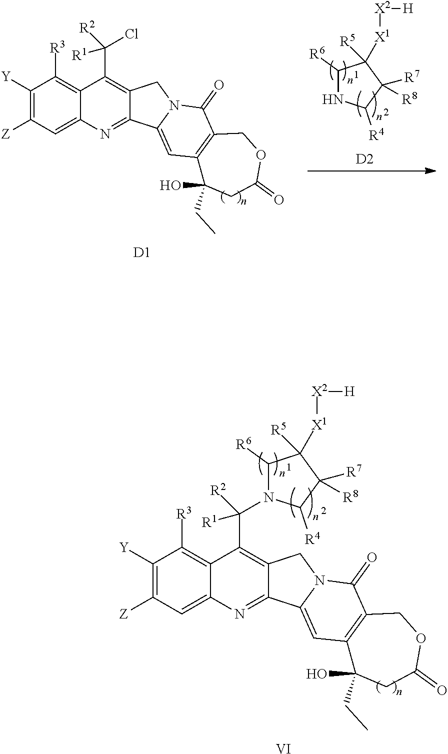

reacting the compound of formula (D1) and compound of formula (D2) optionally under an alkaline condition to obtain the compound of formula (VI);
wherein: Y, Z, R¹, R², R³, R⁴, R⁵, R⁶, R⁷, R⁸, n¹, n², n, are as defined in formula (VI) and any embodiments thereof.

The reagent that provides an alkaline condition includes organic bases and inorganic bases. The organic bases include, but are not limited to, triethylamine, diethylamine, N-methylmorpholine, pyridine, hexahydropyridine, N,N-diisopropylethylamine, n-butyl lithium, lithium diisopropylamide, potassium acetate, sodium tert-butoxide and potassium tert-butoxide. The inorganic bases include, but are not limited to, sodium hydride, potassium phosphate, sodium carbonate, potassium carbonate, cesium carbonate, sodium hydroxide and lithium hydroxide.

### Scheme II

A method for preparing the compound of formula (D7) according to formula (V) or the pharmaceutically acceptable salt or solvate thereof of the present disclosure, comprises the following steps of:

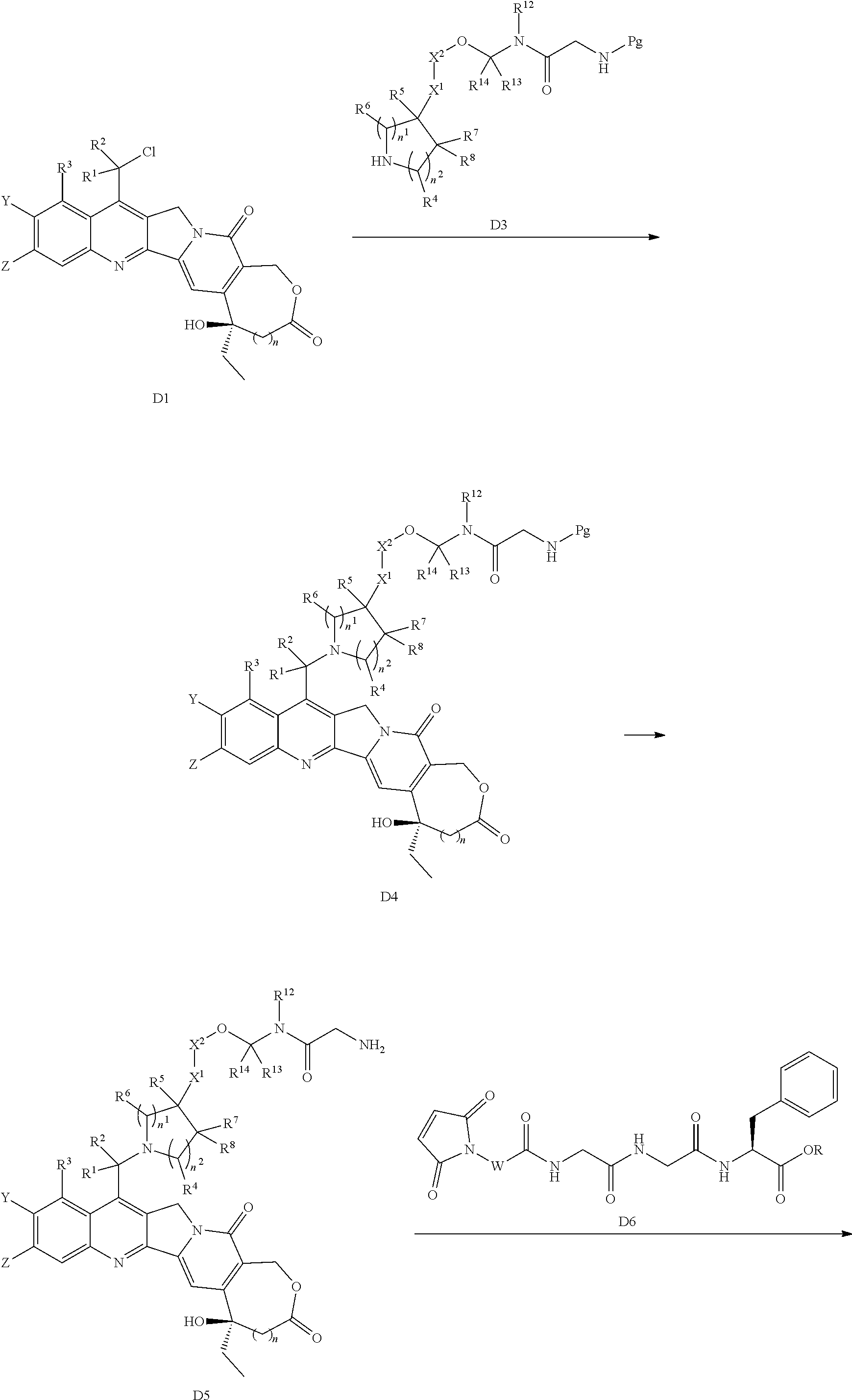

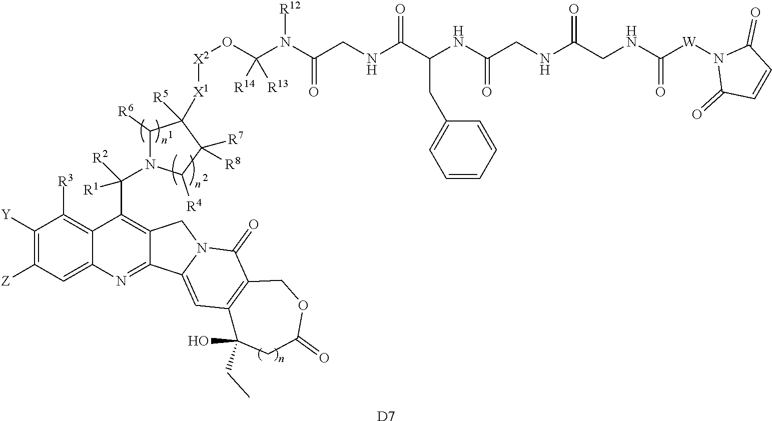

Step 1: reacting the compound of formula (D1) and compound of formula (D3) optionally under an alkaline condition to obtain the compound of formula (D4);
Step 2: the compound of formula (D4) is deprotected to obtain the compound of formula (D5);
Step 3: the compound of formula (D5) and the compound of formula (D6) are reacted in the presence of a condensing agent or under a basic condition and optionally under an alkaline condition to obtain the compound of formula (D7),
Wherein:
PG is an amino protecting group, and preferably benzyloxycarbonyl (Cbz);
Y, Z, R¹, R², R³, R⁴, R⁵, R⁶, R⁷, R⁸, R¹², R¹³, R¹⁴, S¹, n¹, n², n, are as defined in formula (V) and any embodiments thereof.

The reagent that provides an alkaline condition includes organic bases and inorganic bases. The organic bases include, but are not limited to, triethylamine, diethylamine, N-methylmorpholine, pyridine, hexahydropyridine, N,N-diisopropylethylamine, n-butyl lithium, lithium diisopropylamide, potassium acetate, sodium tert-butoxide and potassium tert-butoxide. The inorganic bases include, but are not limited to, sodium hydride, potassium phosphate, sodium carbonate, potassium carbonate, cesium carbonate, sodium hydroxide and lithium hydroxide.

The condensing agent is selected from the group consisting of 4-(4.6-dimethoxy-1.3.5-triazin-2-yl)-4-meth-yl morpholinium chloride, 1-hydroxybenzotriazole, 1-(3-di-methylaminopropyl)-3-ethylcarbodimide hydrochloride, N,N′-dicyclohexylcarbodimide, N,N′-disopropylcarbodimide, O-benzotriazole-N,N,N′,N′-tetramethylurea tetrafluoroborate, 1-hydroxybenzotriazole, 1-hydroxy-7-azobenzotriazole, O-benzotriazole-N,N,N′,N′-tetramethylurea hexafluorophosphate, 2-(7-azobenzotriazole)-N,N,N′,N′-tetramethylurea hexafluorophosphate, benzotriazol-1-yloxytris(dimethyl-amino)phosphonium hexafluorophosphate and benzotriazol-1-yl-oxytripyrrolidinyl phosphorus hexafluorophosphate, and preferably 4-(4.6-dimethoxy-1.3.5-triazin-2-y)-4-methylmorpholinium chloride, 1-hydroxybenzotriazole and 1-(3-dimethylaminopropyl)-3-ethylcarbodimide hydrochloride.

### Scheme III

A method for preparing the compound of formula (D8) according to formula (IV) and any embodiments thereof, or the pharmaceutically acceptable salt or solvate thereof, of the present disclosure, comprises the following step of:

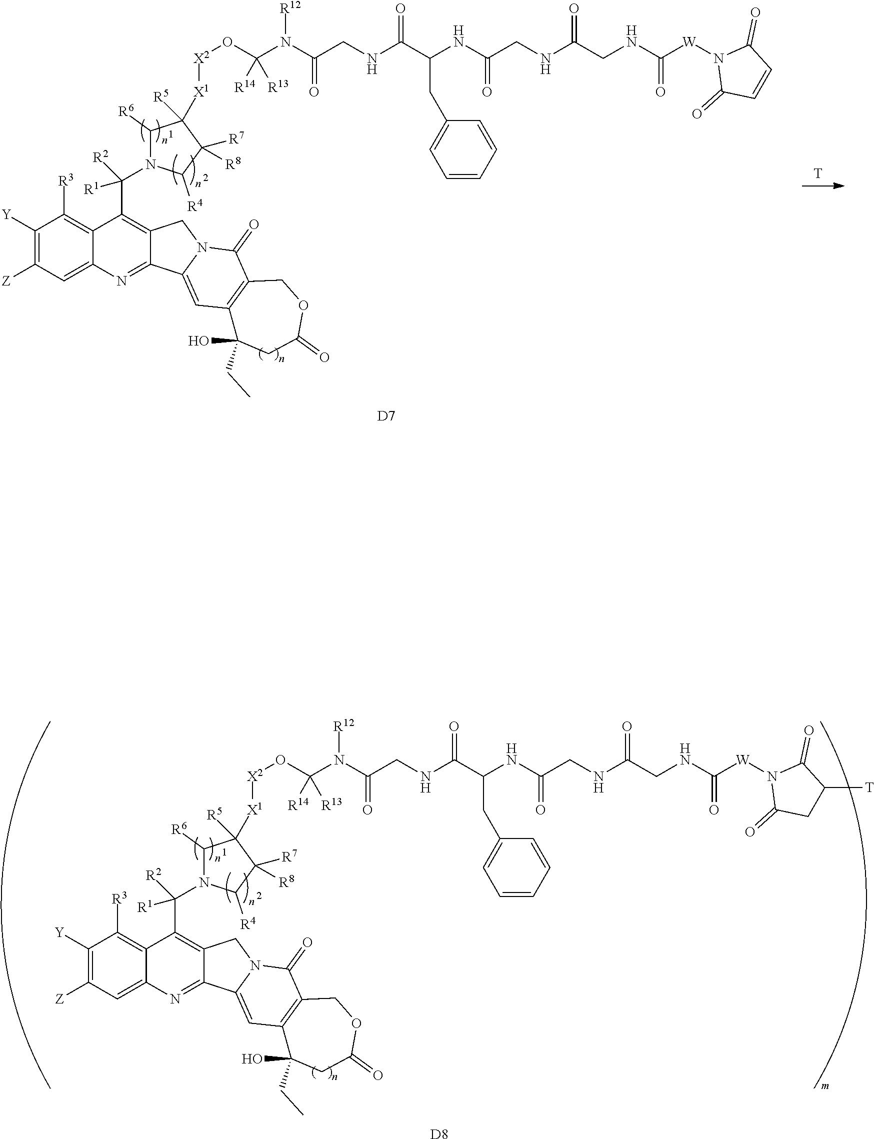

After reduction, T is coupled with the compound of formula D7 to give the ligand drug conjugates of formula (D8); the reducing agent is preferably TCEP;
wherein:
T is a ligand;
Y, Z, R¹, R², R³, R⁴, R⁵, R⁶, R⁷, R⁸, R¹², R¹³, R¹⁴, W, n¹, n², n, m are as defined in formula (IV) or any embodiments thereof.

The present disclosure will be further described with reference to the following examples, but the examples should not be considered as limiting the scope of the present disclosure.

The experimental methods in the examples of the present disclosure for which the specific conditions are not indicated were carried out according to conventional conditions or the conditions recommended by the material or product manufacturers. The reagents for which the specific sources are not indicated are conventional reagents purchased from market.

### EXAMPLES

### Example 1

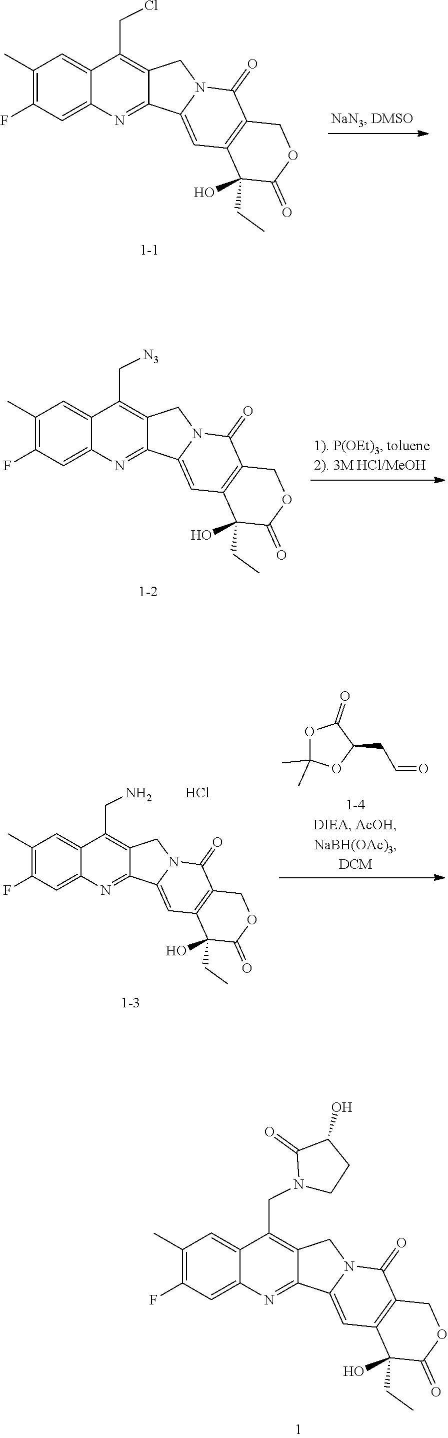 [SMILES: CC[C@@]1(O)C(=O)OCc2c1cc1n(c2=O)Cc2c-1nc1cc(F)c(C)cc1c2CCl ; CC[C@@]1(O)C(=O)OCc2c1cc1n(c2=O)Cc2c-1nc1cc(F)c(C)cc1c2CN=[N+]=[N-] ; CC1(C)OC(=O)[C@@H](CC=O)O1 ; Cl ; CC[C@@]1(O)C(=O)OCc2c1cc1n(c2=O)Cc2c-1nc1cc(F)c(C)cc1c2CN ; CC[C@@]1(O)C(=O)OCc2c1cc1n(c2=O)Cc2c-1nc1cc(F)c(C)cc1c2CN1CC[C@@H](O)C1=O]

To a solution of 1-1 (300 mg, 0.700 mmol) in DMSO (5 mL) was added NaN₃ (120 mg, 1.85 mmol) at 25° C. The reaction mixture was stirred at 25° C. for 12 hrs. LCMS showed 1-1 was consumed completely and desired MS was detected. The reaction mixture was diluted with H₂O (30 mL), filtered. The solid was washed with H₂O (15 mL×3) and dried under reduced pressure to give crude 1-2 (240 mg, 0.551 mmol, 78.8% yield) as a yellow solid. LC-MS: 436.1 [M+H]⁺.

To a solution of 1-2 (190 mg, 0.436 mmol) in toluene (6 mL) was added triethyl phosphite (181 mg, 1.09 mmol) at 25° C. The reaction mixture was heated to 120° C. and stirred for 12 hrs. Then the reaction mixture was cooled to 25° C. and HCl/MeOH (4 mol/L, 3 mL) was added. The reaction mixture was heated to 80° C. and stirred for 12 hrs. TLC (PE/EA=1/1) showed the 1-2 was consumed completely and one new spot was detected by TLC. The reaction mixture was cooled to RT and concentrated under reduced pressure. The residue was triturated with EA (5.0 mL) at 25° C. 1-3 (80.0 mg, 0.179 mmol,

41.2% yield) was obtained as a yellow solid.

To a mixture of 1-3 (50.0 mg, 0.112 mmol), 1-4 (70.9 mg, 0.224 mmol) and DIEA (28 μL, 0.168 mmol) in DCM (2 mL) was added AcOH (0.016 mL, 0.280 mmol) and NaBH(OAc)₃ (94.61 mg, 0.449 mmol) at 25° C. under N₂. The reaction mixture was stirred for 12 hrs under N₂. LCMS showed 1-3 was consumed completely and desired MS was detected. The reaction mixture was washed with brine (5 mL×2), dried over Na₂SO₄, filtered and the filtrate was concentrated under reduced pressure. The residue was purified by prep-HPLC to obtain compound 1 (16.9 mg, 0.034 mmol, 30.5% yield) as a yellow solid.

| | | | | ¹H NMR (400 MHz, |
|---|---|---|---|---|
| Ex | Structure | Name | [M + H]⁺ | DMSO-d₆) |
| 1. | 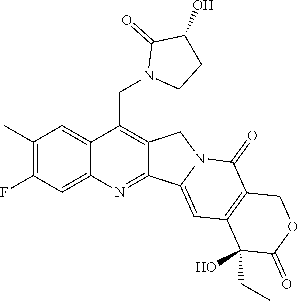 [SMILES: CC[C@@]1(O)C(=O)OCc2c1cc1n(c2=O)Cc2c-1nc1cc(F)c(C)cc1c2CN1CC[C@@H](O)C1=O] | (S)-4-ethyl-8-fluoro- 4-hydroxy-11-(((R)- 3-hydroxy-2- oxopyrrolidin-1- yl)methyl)-9- methyl-1H- pyrano[3′,4′:6,7] indolizino[1,2- b]quinoline- 3,14(4H,12H)-dione | 494.3 | δ 8.35 (br d, J = 8.4 Hz, 1H), 7.93 (d, J = 10.8 Hz, 1H), 7.33 (s, 1H), 6.54 (s, 1H), 5.68 (d, J = 5.6 Hz, 1H), 5.44 (s, 2H), 5.39-5.26 (m, 2H), 5.07-4.91 (m, 2H), 4.16 (dt, J = 8.1, 5.8 Hz, 1H), 3.21 (dt, J = 9.1, 2.3 Hz, 1H), 3.09-2.98 (m, 1H), 2.49-2.48 (s, 3H), 2.26-2.11 (m, 1H), 1.95- 1.79 (m, 2H), 1.62 (br dd, J = 12.4, 8.5 Hz, 1H), 0.88 (br t, J = 7.3 Hz, 3H). |

### Example 2

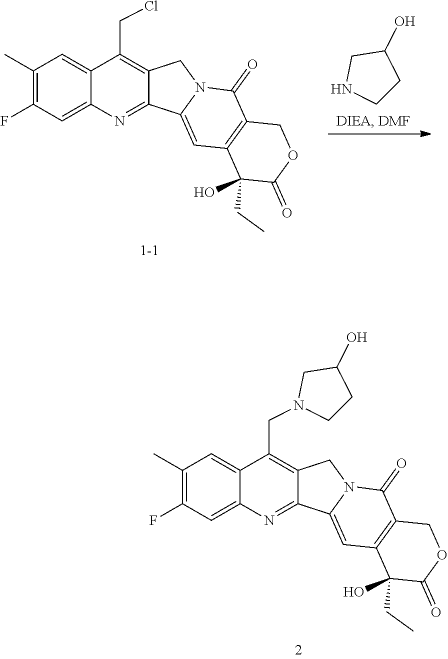 [SMILES: CC[C@@]1(O)C(=O)OCc2c1cc1n(c2=O)Cc2c-1nc1cc(F)c(C)cc1c2CCl ; OC1CCNC1 ; CC[C@@]1(O)C(=O)OCc2c1cc1n(c2=O)Cc2c-1nc1cc(F)c(C)cc1c2CN1CCC(O)C1]

To a solution of 1-1 (30.00 mg, 0.07 mmol) in DMF (3.00 mL) was added pyrrolidin-3-ol (7.31 mg, 0.08 mmol) and DIEA (0.04 mL, 0.21 mmol) and the mixture was stirred at 20° C. for 12 hrs. LCMS showed the reaction was completed. The reaction mixture was purified by prep-HPLC to obtain compound 2 (2.87 mg, 0.01 mmol, 8.94%) as a yellow solid.

The compounds below were synthesized following procedures described for Example 2.

| Ex | Structure | Name | [M + H]⁺ | ¹H NMR |
|---|---|---|---|---|
| 2. | 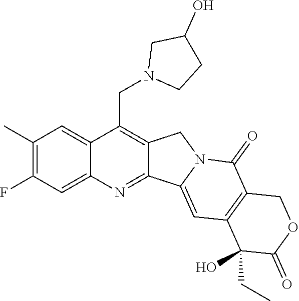 [SMILES: CC[C@@]1(O)C(=O)OCc2c1cc1n(c2=O)Cc2c-1nc1cc(F)c(C)cc1c2CN1CCC(O)C1] | (4S)-4-ethyl-8- fluoro-4-hydroxy- 11-((3- hydroxypyrrolidin-1- yl)methyl)-9-methyl- 1H- pyrano[3′,4′:6,7] indolizino[1,2- b]quinoline- 3,14(4H,12H)-dione | 480.2 | ¹H NMR (400 MHz, MeOD-d₄) δ 8.35 (br d, J = 7.6 Hz, 1H), 7.88 (br d, J = 10.4 Hz, 1H), 7.66 (s, 1H), 5.65-5.37 (m, 4H), 5.10-5.00 (m, 2H), 4.60 (br s, 1H), 3.71-3.37 (m, 4H), 2.61 (s, 3H), 2.41- 2.23 (m, 1H), 2.20-2.05 (m, 1H), 2.01-1.89 (m, 2H), 1.01 (t, J = 7.3 Hz, 3H). |
| 3. | 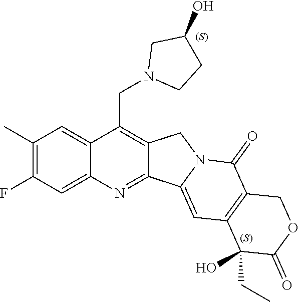 | (S)-4-ethyl-8-fluoro- 4-hydroxy-11-(((S)- 3-hydroxypyrrolidin- 1-yl)methyl)-9- methyl-1H- pyrano[3′,4′:6,7] indolizino[1,2- b]quinoline- 3,14(4H,12H)-dione | 480.2 | ¹H NMR (400 MHz, DMSO-d₆) δ 10.37-9.86 (m, 1H), 8.47 (br d, J = 7.7 Hz, 1H), 8.00 (d, J = 10.0 Hz, 1H),, 7.36 (s, 1H), 6.5 (br s, 1H), , 5.57- 5.31 (m, 4H), 5.20- 4.88 (m, 2H), 4.57-4.33 (m, 1H), 3.79-3.48 (m, 2H), 3.26-3.11 (m, 1H), 2.57 (s, 3H), 2.53 (br s, 1H), 2.11-1.72 (m, 4H), 0.88 (t, J = 7.3 Hz, 3H) |
| 4. | 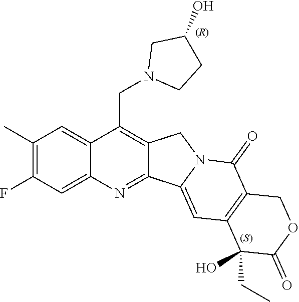 | (S)-4-ethyl-8-fluoro- 4-hydroxy-11-(((R)- 3-hydroxypyrrolidin- 1-yl)methyl)-9- methyl-1H- pyrano[3′,4′:6,7] indolizino[1,2- b]quinoline- 3,14(4H,12H)-dione | 480.2 | ¹H NMR (400 MHz, MeOD-d₄) δ 8.38 (d, J = 7.4 Hz, 1H), 7.94 (d, J = 10.5 Hz, 1H), 7.70 (s, 1H), 5.66-5.54 (m, 2H), 5.53-5.40 (m, 2H), 5.20- 5.01 (m, 2H), 4.68- 4.57 (m, 1H), 3.91-3.39 (m, 6H), 2.64 (s, 3H), 2.07-1.92 (m, 2H), 1.03 (t, J = 7.4 Hz, 3H). |
| 5. | 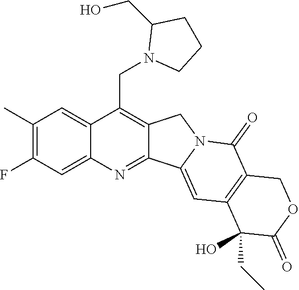 [SMILES: CC[C@@]1(O)C(=O)OCc2c1cc1n(c2=O)Cc2c-1nc1cc(F)c(C)cc1c2CN1CCCC1CO] | (4S)-4-ethyl-8- fluoro-4-hydroxy- 11-((2- (hydroxymethyl) pyrrolidin-1-yl)methyl)- 9-methyl-1H- pyrano[3′,4′:6,7] indolizino[1,2- b]quinoline- 3,14(4H,12H)-dione | 494.3 | ¹H NMR (400 MHz, MeOD-d₄) δ 8.48 (d, J = 7.6 Hz, 1H), 7.98-7.89 (d, J = 8.0 Hz, 1H), 7.69 (s, 1H), 5.70-5.55 (m, 2H), 5.52-5.36 (m, 3H), 4.97-4.92 (m, 1H), 4.21 (br d, J = 9.8 Hz, 1H), 4.10-3.95 (m, 1H), 3.89 (br dd, J = 12.1, 6.4 Hz, 1H), 3.55-3.42 (m, 1H), 2.63 (s, 3H), 2.45-2.32 (m, 1H), 2.29-2.14 (m, 1H), 2.09-1.85 (m, 4H), 1.42-1.29 (m, 1H), 1.03 (t, J = 7.4 Hz, 3H). |
| 6. | 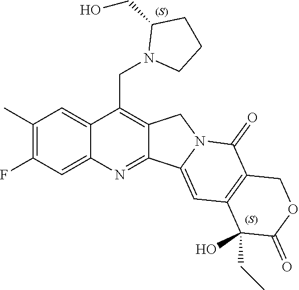 | (S)-4-ethyl-8-fluoro- 4-hydroxy-11-(((S)- 2- (hydroxymethyl) pyrrolidin-1-yl)methyl)- 9-methyl-1H- pyrano[3′,4′:6,7] indolizino[1,2- b]quinoline- 3,14(4H,12H)-dione | 494.3 | ¹H NMR (400 MHz, DMSO-d₆) δ 9.35-9.22 (m, 1H), 8.44 (br d, J = 6.7 Hz, 1H), 8.09-7.98 (m, 1H), 7.37 (s, 1H), 6.63-6.53 (m, 1H), 5.99 (br s, 1H), 5.57-5.28 (m, 5H), 5.02-4.88 (m, 1H), 4.07-3.97 (m, 1H), 3.95- 3.85 (m, 1H), 3.82 (s, 1H), 2.56 (br s, 3H), 2.29- 2.18 (m, 2H), 2.10- 1.98 (m, 1H), 1.94-1.82 (m, 2H), 1.81-1.68 (m, 2H), 0.89 (br t, J = 7.3 Hz, 3H). |
| 7. | 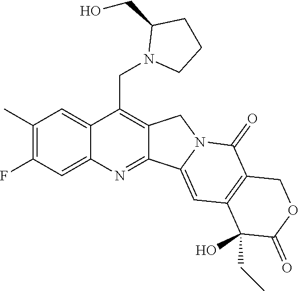 [SMILES: CC[C@@]1(O)C(=O)OCc2c1cc1n(c2=O)Cc2c-1nc1cc(F)c(C)cc1c2CN1CCC[C@@H]1CO] | (S)-4-ethyl-8-fluoro- 4-hydroxy-11-(((R)- 2- (hydroxymethyl) pyrrolidin-1-yl)methyl)- 9-methyl-1H- pyrano[3′,4′:6,7] indolizino[1,2- b]quinoline- 3,14(4H,12H)-dione | 494.3 | ¹H NMR (400 MHz, MeOD-d₄) δ 8.48 (d, J = 7.8 Hz, 1H), 7.92 (d, J = 10.4 Hz, 1H), 7.69 (s, 1H), 5.71-5.54 (m, 2H), 5.46 (t, J = 16.4 Hz, 3H), 4.96-4.92 (m, 1H), 4.28- 4.13 (m, 1H), 4.09- 3.81 (m, 2H), 3.50 (br dd, J = 1.7, 3.2 Hz, 2H), 2.63 (s, 3H), 2.43-2.36 (m, 1H), 2.25-2.16 (m, 1H), 2.06-1.88 (m, 4H), 1.03 (t, J = 7.4 Hz, 3H). |
| 8. | 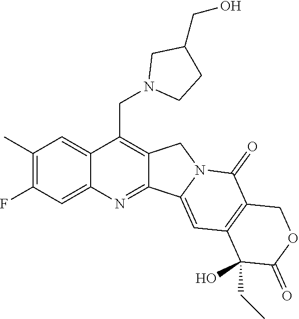 [SMILES: CC[C@@]1(O)C(=O)OCc2c1cc1n(c2=O)Cc2c-1nc1cc(F)c(C)cc1c2CN1CCC(CO)C1] | (4S)-4-ethyl-8- fluoro-4-hydroxy- 11-((3- (hydroxymethyl) pyrrolidin-1-yl)methyl)- 9-methyl-1H- pyrano[3′,4′:6,7] indolizino[1,2- b]quinoline- 3,14(4H,12H)-dione | 494.2 | ¹H NMR (400 MHz, MeOD-d₄) δ 8.35 (d, J = 7.6 Hz, 1H), 7.88 (d, J = 10.4 Hz, 1H), 7.65 (s, 1H), 5.61 (d, J = 16.4 Hz, 1H), 5.52 (s, 2H), 5.42 (d, J = 16.4 Hz, 1H), 5.12 (br s, 2H), 3.76-3.50 (m, 5H), 2.81-2.66 (m, 1H), 2.63 (s, 3H), 2.39-2.15 (m, 1H), 1.98 (br dd, J = 7.4, 4.5 Hz, 3H), 1.02 (t, J = 7.4 Hz, 3H). |
| 9. | 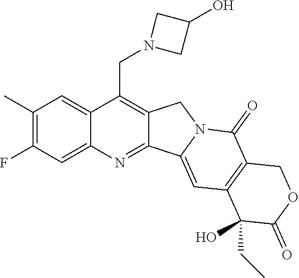 [SMILES: CC[C@@]1(O)C(=O)OCc2c1cc1n(c2=O)Cc2c-1nc1cc(F)c(C)cc1c2CN1CC(O)C1] | (S)-4-ethyl-8-fluoro- 4-hydroxy-11-((3- hydroxyazetidin-1- yl)methyl)-9-methyl- 1H- pyrano[3′,4′:6,7] indolizino[1,2- b]quinoline- 3,14(4H,12H)-dione | 466.2 | δ 10.39-10.32 & 9.81- 9.70 (m, 1H), 8.45 (br d, J = 6.1 Hz, 1H), 8.00 (br d, J = 10.1 Hz, 1H), 7.36 (s, 1H), 6.58 (s, 1H), 6.37-6.02 (m, 1H), 5.58- 5.32 (m, 4H), 5.22- 5.08 (m, 2H), 4.59-3.83 (m, 4H), 2.57 (s, 3H), 1.88 (tt, J = 14.6, 7.1 Hz, 2H), 0.88 (t, J = 7.3 Hz, 3H). |
| 10. | 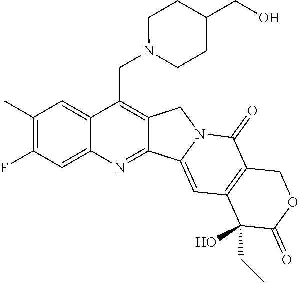 [SMILES: CC[C@@]1(O)C(=O)OCc2c1cc1n(c2=O)Cc2c-1nc1cc(F)c(C)cc1c2CN1CCC(CO)CC1] | (S)-4-ethyl-8-fluoro- 4-hydroxy-11-((4- (hydroxymethyl) piperidin-1-yl)methyl)- 9-methyl-1H- pyrano[3′,4′:6,7] indolizino[1,2- b]quinoline- 3,14(4H,12H)-dione | 508.2 | ¹H NMR (400 MHz, DMSO-d₆) δ 9.19-9.06 (m, 1H), 8.47 (br d, J = 7.7 Hz, 1H), 8.01 (dd, J = 10.0, 5.2 Hz, 1H), 7.37 (s, 1H), 6.58 (s, 1H), 5.53- 5.41 (m, 4H), 5.03- 4.90 (m, 2H), 4.70-4.58 (m, 1H), 3.67-3.53 (m, 2H), 2.59 (s, 3H), 2.53 (br s, 2H), 1.93-1.76 (m, 5H), 1.69-1.62 (m, 1H), 1.49-1.34 (m, 2H), 0.88 (t, J = 7.3 Hz, 3H). |
| 11. | 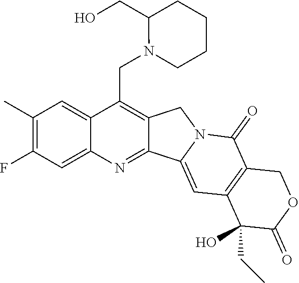 [SMILES: CC[C@@]1(O)C(=O)OCc2c1cc1n(c2=O)Cc2c-1nc1cc(F)c(C)cc1c2CN1CCCCC1CO] | (4S)-4-ethyl-8- fluoro-4-hydroxy- 11-((2- (hydroxymethyl) piperidin-1-yl)methyl)- 9-methyl-1H- pyrano[3′,4′:6,7] indolizino[1,2- b]quinoline- 3,14(4H,12H)-dione | 508.2 | ¹H NMR (400 MHz, MeOD-d₄) δ 8.52 (br s, 1H), 7.90 (d, J = 10.5 Hz, 1H), 7.68 (s, 1H), 5.71- 5.57 (m, 2H), 5.55-5.36 (m, 3H), 4.84-4.61 (m, 2H), 4.60-4.46 (m, 1H), 3.93-3.78 (m, 1H), 3.68- 3.49 (m, 1H), 3.27- 3.16 (m, 1H), 2.61 (s, 3H), 2.23-2.08 (m, 1H), 2.06-1.84 (m, 4H), 1.84- 1.57 (m, 3H), 1.01 (t, J = 7.4 Hz, 3H). |
| 12. | 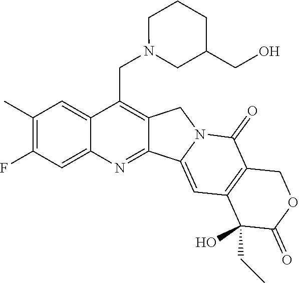 [SMILES: CC[C@@]1(O)C(=O)OCc2c1cc1n(c2=O)Cc2c-1nc1cc(F)c(C)cc1c2CN1CCCC(CO)C1] | (4S)-4-ethyl-8- fluoro-4-hydroxy- 11-((3- (hydroxymethyl) piperidin-1-yl)methyl)- 9-methyl-1H- pyrano[3′,4′:6,7] indolizino[1,2- b]quinoline- 3,14(4H,12H)-dione | 508.3 | ¹H NMR (400 MHz, MeOD-d₄) δ 8.35 (br d, J = 7.3 Hz, 1H), 7.92 (d, J = 10.3 Hz, 1H), 7.69 (s, 1H), 5.67-5.57 (m, 1H), 5.51 (s, 2H), 5.43 (d, J = 16.5 Hz, 1H), 5.09-4.89 (m, 2H), 3.73-3.52 (m, 3H), 3.50-3.37 (m, 1H), 3.28-3.16 (m, 1H), 3.13- 2.93 (m, 1H), 2.64 (s, 3H), 2.10-1.93 (m, 4H), 1.90-1.80 (m, 2H), 1.42- 1.33 (m, 1H), 1.03 (t, J = 7.4 Hz, 3H). |
| 13. | 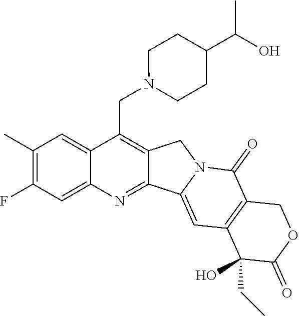 [SMILES: CC[C@@]1(O)C(=O)OCc2c1cc1n(c2=O)Cc2c-1nc1cc(F)c(C)cc1c2CN1CCC(C(C)O)CC1] | (4S)-4-ethyl-8- fluoro-4-hydroxy- 11-((4-(1- hydroxyethyl) piperidin-1-yl)methyl)- 9- methyl-1H- pyrano[3′,4′:6,7] indolizino[1,2- b]quinoline- 3,14(4H,12H)-dione | 522.2 | ¹H NMR (400 MHz, MeOD-d₄) δ 8.35 (br d, J = 7.6 Hz, 1H), 7.94 (d, J = 10.5 Hz, 1H), 7.70 (s, 1H), 5.63 (d, J = 16.4 Hz, 1H), 5.52 (s, 2H), 5.44 (d, J = 16.4 Hz, 1H), 5.03- 4.95 (m, 2H), 3.84- 3.39 (m, 5H), 2.64 (s, 3H), 2.17-2.07 (m, 1H), 2.06-1.95 (m, 2H), 1.94- 1.84 (m, 1H), 1.72-1.50 (m, 3H), 1.19 (br d, J = 6.1 Hz, 3H), 1.03 (t, J = 7.4 Hz, 3H). |
| 14. | 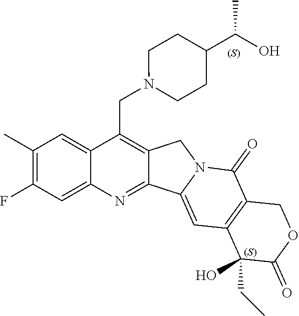 | (S)-4-ethyl-8-fluoro- 4-hydroxy-11-((4- ((S)-1- hydroxyethyl) piperidin-1-yl)methyl)- 9-methyl-1H- pyrano[3′,4′:6,7] indolizino[1,2- b]quinoline- 3,14(4H,12H)-dione | 522.3 | ¹H NMR (400 MHz, MeOH-d₄) δ 8.34 (br d, J = 7.8 Hz, 1H), 7.91 (d, J = 10.4 Hz, 1H), 7.69 (s, 1H), 5.63 (d, J = 16.4 Hz, 1H), 5.52 (s, 2H), 5.43 (d, J = 16.4 Hz, 1H), 4.93 (br d, J = 11.6 Hz, 2H), 3.77-3.52 (m, 3H), 3.31- 3.20 (m, 2H), 2.63 (s, 3H), 2.17-2.06 (m, 1H), 2.05-1.94 (m, 2H), 1.90 (br d, J = 8.0 Hz, 1H), 1.71-1.56 (m, 3H), 1.19 (br d, J = 6.1 Hz, 3H), 1.03 (t, J = 7.4 Hz, 3H). |
| 15. | 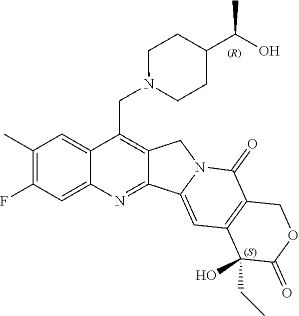 | (S)-4-ethyl-8-fluoro- 4-hydroxy-11-((4- ((R)-1- hydroxyethyl) piperidin-1-yl)methyl)- 9-methyl-1H- pyrano[3′,4′:6,7] indolizino[1,2- b]quinoline- 3,14(4H,12H)-dione | 522.3 | ¹H NMR (400 MHz, MeOH-d₄) δ 8.34 (br d, J = 7.7 Hz, 1H), 7.93 (d, J = 10.5 Hz, 1H), 7.70 (s, 1H), 5.63 (d, J = 16.3 Hz, 1H), 5.52 (s, 2H), 5.44 (d, J = 16.5 Hz, 1H), 5.09- 4.88 (m, 2H), 3.82- 3.54 (m, 3H), 3.46-3.34 (m, 2H), 2.64 (s, 3H), 2.14-2.04 (m, 1H), 2.03- 1.93 (m, 2H), 1.91- 1.79 (m, 1H), 1.78-1.52 (m, 3H), 1.19 (d, J = 6.0 Hz, 3H), 1.03 (t, J = 7.2 Hz, 3H). |
| 16. | 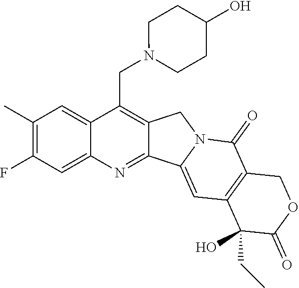 [SMILES: CC[C@@]1(O)C(=O)OCc2c1cc1n(c2=O)Cc2c-1nc1cc(F)c(C)cc1c2CN1CCC(O)CC1] | (S)-4-ethyl-8-fluoro- 4-hydroxy-11-((4- hydroxypiperidin-1- yl)methyl)-9-methyl- 1H- pyrano[3′,4′:6,7] indolizino[1,2- b]quinoline- 3,14(4H,12H)-dione | 494.2 | ¹H NMR (400 MHz, MeOD-d₄) δ 8.36 (br d, J = 7.6 Hz, 1H), 7.93 (br d, J = 9.9 Hz, 1H), 7.70 (s, 1H), 5.68-5.56 (m, 1H), 5.54-5.40 (m, 3H), 5.10- 4.95 (m, 2H), 3.72- 3.51 (m, 2H), 2.64 (s, 3H), 2.19-1.87 (m, 1H), 1.85-1.63 (m, 1H), 1.03 (t, J = 7.3 Hz, 3H). |
| 17. | 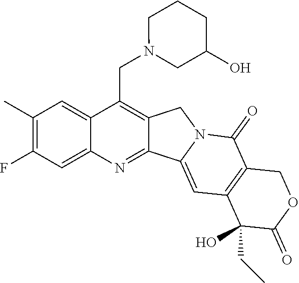 [SMILES: CC[C@@]1(O)C(=O)OCc2c1cc1n(c2=O)Cc2c-1nc1cc(F)c(C)cc1c2CN1CCCC(O)C1] | (4S)-4-ethyl-8- fluoro-4-hydroxy- 11-((3- hydroxypiperidin-1- yl)methyl)-9-methyl- 1H- pyrano[3′,4′:6,7] indolizino[1,2- b]quinoline- 3,14(4H,12H)-dione | 494.2 | ¹H NMR (400 MHz, MeOD-d₄) δ 8.37 (d, J = 7.6 Hz, 1H), 7.91 (br d, J = 10.0 Hz, 1H), 7.70 (s, 1H), 5.68-5.59 (m, 1H), 5.52 (s, 2H), 5.44 (d, J = 16.3 Hz, 1H), 4.84-4.67 (m, 3H), 4.35-3.63 (m, 2H), 2.63 (s, 3H), 2.25- 1.88 (m, 4H), 1.86-1.67 (m, 2H), 1.03 (t, J = 7.4 Hz, 3H) |
| 18. | 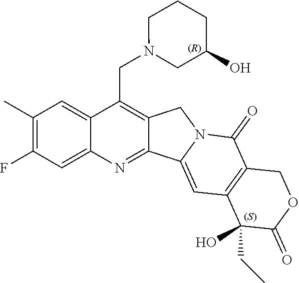 | (S)-4-ethyl-8-fluoro- 4-hydroxy-11-(((R)- 3-hydroxypiperidin- 1-yl)methyl)-9- methyl-1H- pyrano[3′,4′:6,7] indolizino[1,2- b]quinoline- 3,14(4H,12H)-dione | 494.2 | ¹H NMR (400 MHz, MeOH-d₄) δ 8.37 (d, J = 7.6 Hz, 1H), 7.90 (br d, J = 10.4 Hz, 1H), 7.68 (s, 1H), 5.62 (d, J = 16.4 Hz, 1H), 5.55-5.49 (m, 2H), 5.43 (d, J = 16.4 Hz, 1H), 5.00-4.85 (m, 2H), 4.04- 4.00 (m, 1H), 3.44- 3.34 (m, 4H), 2.63 (s, 3H), 2.18-2.10 (m, 1H), 2.05-1.94 (m, 3H), 1.80- 1.70 (m, 2H), 1.03 (t, J = 7.4 Hz, 3H). |
| 19. | 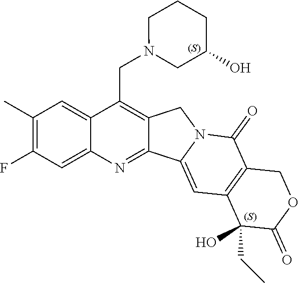 | (S)-4-ethyl-8-fluoro- 4-hydroxy-11-(((S)- 3-hydroxypiperidin- 1-yl)methyl)-9- methyl-1H- pyrano[3′,4′:6,7] indolizino[1,2- b]quinoline- 3,14(4H,12H)-dione | 494.1 | ¹H NMR (400 MHz, METHANOL-d₄) δ 8.37 (d, J = 7.9 Hz, 1H), 7.91 (br d, J = 10.3 Hz, 1H), 7.69 (s, 1H), 5.63 (d, J = 16.3 Hz, 1H), 5.55-5.49 (m, 2H), 5.43 (d, J = 16.4 Hz, 1H), 4.85-4.60 (m, 2H), 4.23-3.75 (m, 1H), 3.47-3.34 (m, 4H), 2.63 (s, 3H), 2.24-1.94 (m, 3H), 1.92-1.50 (m, 3H), 1.03 (t, J = 7.4 Hz, 3H). |
| 20. | 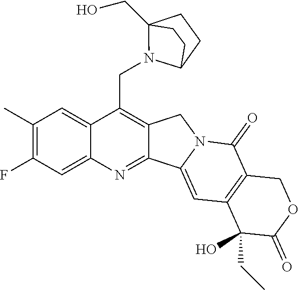 [SMILES: CC[C@@]1(O)C(=O)OCc2c1cc1n(c2=O)Cc2c-1nc1cc(F)c(C)cc1c2CN1C2CCC1(CO)CC2] | (S)-4-ethyl-8-fluoro- 4-hydroxy-11-((1- (hydroxymethyl)-7- azabicyclo[2.2.1] heptan-7-yl)methyl)- 9-methyl-1H- pyrano[3′,4′:6,7] indolizino[1,2- b]quinoline- 3,14(4H,12H)-dione | 520.1 | ¹H NMR (400 MHz, MeOD-d₄) δ 8.45 (br d, J = 7.5 Hz, 1H), 7.93 (d, J = 10.4 Hz, 1H), 7.70 (s, 1H), 5.78-5.58 (m, 2H), 5.53-5.32 (m, 2H), 5.14- 5.00 (m, 1H), 4.80- 4.61 (m, 1H), 4.38-4.23 (m, 1H), 4.19-4.02 (m, 2H), 2.95-2.76 (m, 1H), 2.62 (s, 3H), 2.45-2.12 (m, 3H), 2.09-1.79 (m, 6H), 1.03 (t, J = 7.3 Hz, 3H). |
| 21. | 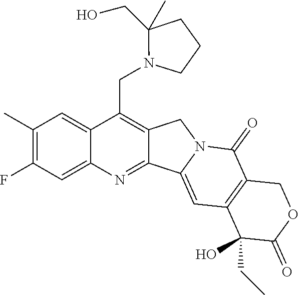 [SMILES: CC[C@@]1(O)C(=O)OCc2c1cc1n(c2=O)Cc2c-1nc1cc(F)c(C)cc1c2CN1CCCC1(C)CO] | (4S)-4-ethyl-8- fluoro-4-hydroxy- 11-((2- (hydroxymethyl)-2- methylpyrrolidin-1- yl)methyl)-9-methyl- 1H- pyrano[3′,4′:6,7] indolizino[1,2- b]quinoline- 3,14(4H,12H)-dione | 508.2 | ¹H NMR (400 MHz, MeOD-d₄) δ 8.48 (br d, J = 7.8 Hz, 1H), 7.94 (br d, J = 10.3 Hz, 1H), 7.70 (s, 1H), 5.75-5.59 (m, 2H), 5.55-5.37 (m, 2H), 5.31- 5.15 (m, 1H), 4.86- 4.63 (m, 2H), 4.23-4.07 (m, 1H), 4.02-3.87 (m, 1H), 3.81-3.58 (m, 1H), 2.63 (s, 3H), 2.36-2.14 (m, 2H), 2.11-1.96 (m, 3H), 1.95-1.81 (m, 1H), 1.64 (br s, 3H), 1.03 (t, J = 7.3 Hz, 3H) |
| 22. | 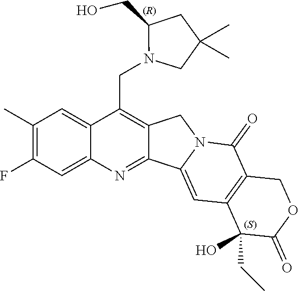 | (S)-4-ethyl-8-fluoro- 4-hydroxy-11-(((R)- 2-(hydroxymethyl)- 4,4- dimethylpyrrolidin- 1-yl)methyl)-9- methyl-1H- pyrano[3′,4′:6,7] indolizino[1,2- b]quinoline- 3,14(4H,12H)-dione | 522.1 | ¹H NMR (400 MHz, MeOD-d₄) δ 8.48 (br d, J = 7.6 Hz, 1H), 7.90 (br d, J = 10.6 Hz, 1H), 7.69 (s, 1H), 5.69-5.56 (m, 2H), 5.46 (t, J = 16.8 Hz, 2H), 4.44-4.06 (m, 2H), 3.95 (br dd, J = 4.3, 11.5 Hz, 1H), 3.48-3.34 (m, 2H), 3.31-2.99 (m, 2H), 2.63 (s, 3H), 2.18-2.07 (m, 1H), 2.05-1.94 (m, 2H), 1.93-1.77 (m, 1H), 1.41- 1.27 (m, 3H), 1.11 (s, 3H), 1.03 (t, J = 7.4 Hz, 3H). |
| 23. | 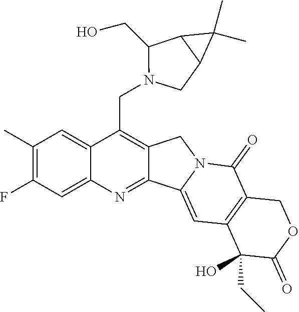 [SMILES: CC[C@@]1(O)C(=O)OCc2c1cc1n(c2=O)Cc2c-1nc1cc(F)c(C)cc1c2CN1CC2C(C1CO)C2(C)C] | (4S)-4-ethyl-8- fluoro-4-hydroxy- 11-((2- (hydroxymethyl)- 6,6-dimethyl-3- azabicyclo[3.1.0] hexan-3-yl)methyl)-9- methyl-1H- pyrano[3′,4′:6,7] indolizino[1,2- b]quinoline- 3,14(4H,12H)-dione | 534.3 | ¹H NMR (400 MHz, MeOD-d₄) δ 8.44 (d, J = 7.9 Hz, 1H), 7.91 (br d, J = 10.5 Hz, 1H), 7.70 (s, 1H), 5.69-5.54 (m, 2H), 5.50-5.41 (m, 2H), 4.96 (br d, J = 2.0 Hz, 2H), 4.56-3.99 (m, 2H), 3.88- 3.42 (m, 3H), 2.62 (s, 3H), 2.07-1.93 (m, 2H), 1.75-1.56 (m, 2H), 1.50- 1.31 (m, 3H), 1.14 (s, 3H), 1.03 (t, J = 7.4 Hz, 3H) |
| 24 | 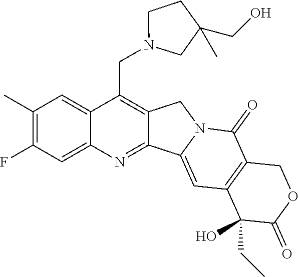 [SMILES: CC[C@@]1(O)C(=O)OCc2c1cc1n(c2=O)Cc2c-1nc1cc(F)c(C)cc1c2CN1CCC(C)(CO)C1] | (4S)-4-ethyl-8- fluoro-4-hydroxy- 11-((3- (hydroxymethyl)-3- methylpyrrolidin-1- yl)methyl)-9-methyl- 1H- pyrano[3′,4′:6,7] indolizino[1,2- b]quinoline- 3,14(4H,12H)-dione | 508.3 | ¹H NMR (400 MHz, MeOD-d₄) δ 8.36 (br d, J = 7.6 Hz, 1H), 7.93 (dd, J = 10.3, 3.3 Hz, 1H), 7.69 (s, 1H), 5.63 (d, J = 16.3 Hz, 1H), 5.51 (br s, 2H), 5.43 (d, J = 16.5 Hz, 1H), 5.21-4.96 (m, 2H), 3.75-3.37 (m, 6H), 2.64 (s, 3H), 2.29-2.10 (m, 1H), 2.06-1.91 (m, 3H), 1.13 (s, 3H), 1.03 (t, J = 7.4 Hz, 3H). |
| 25. | 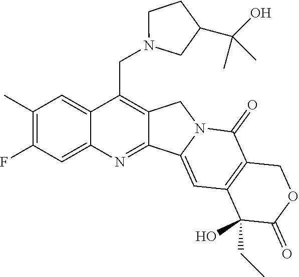 [SMILES: CC[C@@]1(O)C(=O)OCc2c1cc1n(c2=O)Cc2c-1nc1cc(F)c(C)cc1c2CN1CCC(C(C)(C)O)C1] | (4S)-4-ethyl-8- fluoro-4-hydroxy- 11-((3-(2- hydroxypropan-2- yl)pyrrolidin-1- yl)methyl)-9-methyl- 1,12-dihydro-14H- pyrano[3′,4′:6,7] indolizino[1,2- b]quinoline- 3,14(4H)-dione | 522.3 | ¹H NMR (400 MHz, MeOD-d₄) δ 8.36 (d, J = 7.7 Hz, 1H), 7.93 (d, J = 9.2 Hz, 1H), 7.69 (s, 1H), 5.63 (d, J = 16.5 Hz, 1H), 5.52 (s, 2H), 5.43 (d, J = 16.5 Hz, 1H), 5.22-5.01 (m, 2H), 3.79-3.39 (m, 4H), 2.64 (s, 3H), 2.58- 2.40 (m, 1H), 2.32-2.06 (m, 2H), 2.04-1.90 (m, 2H), 1.29-1.21 (m, 6H), 1.03 (t, J = 7.3 Hz, 3H). |
| 26. | 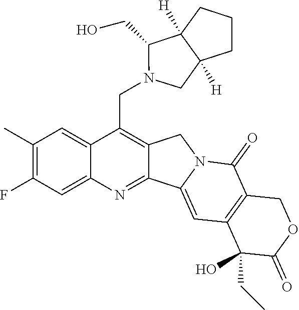 [SMILES: CC[C@@]1(O)C(=O)OCc2c1cc1n(c2=O)Cc2c-1nc1cc(F)c(C)cc1c2CN1C[C@@H]2CCC[C@@H]2[C@H]1CO] | (S)-4-ethyl-8-fluoro- 4-hydroxy-11- (((1S,3aR,6aS)-1- (hydroxymethyl) hexahydrocyclopenta [c]pyrrol-2(1H)- yl)methyl)-9-methyl- 1,12-dihydro-14H- pyrano[3′,4′:6,7] indolizino[1,2- b]quinoline- 3,14(4H)-dione | 534.1 | ¹H NMR (400 MHz, MeOD-d₄) δ 8.40 (d, J = 7.6 Hz, 1H), 7.94 (d, J = 10.4 Hz, 1H), 7.71 (s, 1H), 5.69-5.55 (m, 2H), 5.54-5.38 (m, 3H), 4.44- 4.35 (m, 1H), 4.05 (dd, J = 12.8, 2.7 Hz, 1H), 3.58-3.52 (m, 1H), 3.46- 3.39 (m, 1H), 3.13- 3.02 (m, 1H), 2.93-2.81 (m, 1H), 2.75-2.66 (m, 1H), 2.63 (s, 3H), 2.06- 1.95 (m, 2H), 1.94-1.74 (m, 5H), 1.72-1.63 (m, 2H), 1.03 (t, J = 7.4 Hz, 3H). |
| 27. | 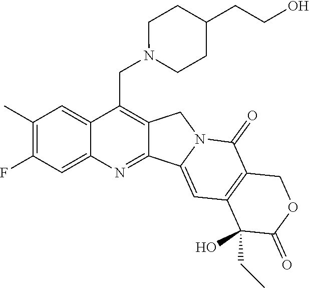 [SMILES: CC[C@@]1(O)C(=O)OCc2c1cc1n(c2=O)Cc2c-1nc1cc(F)c(C)cc1c2CN1CCC(CCO)CC1] | (S)-4-ethyl-8-fluoro- 4-hydroxy-11-((4-(2- hydroxyethyl)piperidin- 1-yl)methyl)-9- methyl-1H- pyrano[3′,4′:6,7] indolizino[1,2- b]quinoline- 3,14(4H,12H)-dione | 522.2 | ¹H NMR (400 MHz, METHANOL-d₄) δ 8.34 (br d, J = 7.2 Hz, 1H), 7.93 (d, J = 10.3 Hz, 1H), 7.70 (s, 1H), 5.63 (d, J = 16.8 Hz, 1H), 5.52 (s, 2H), 5.44 (d, J = 16.3 Hz, 1H), 5.05-4.90 (m, 2H), 3.83-3.53 (m, 4H), 3.47- 3.35 (m, 2H), 2.64 (s, 3H), 2.10-1.94 (m, 4H), 1.93-1.78 (m, 1H), 1.65- 1.39 (m, 4H), 1.03 (t, J = 7.3 Hz, 3H). |
| 28. | 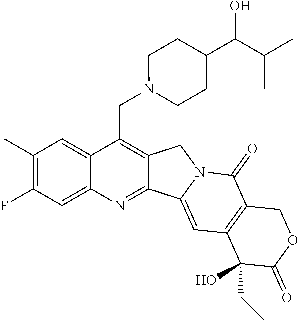 [SMILES: CC[C@@]1(O)C(=O)OCc2c1cc1n(c2=O)Cc2c-1nc1cc(F)c(C)cc1c2CN1CCC(C(O)C(C)C)CC1] | (4S)-4-ethyl-8- fluoro-4-hydroxy- 11-((4-(1-hydroxy-2- methylpropyl)piperidin- 1-yl)methyl)-9- methyl-1H- pyrano[3′,4′:6,7] indolizino[1,2- b]quinoline- 3,14(4H,12H)-dione | 550.1 | ¹H NMR (400 MHz, METHANOL-d₄) δ 8.34 (br d, J = 7.7 Hz, 1H), 7.93 (d, J = 10.5 Hz, 1H), 7.70 (s, 1H), 5.63 (d, J = 16.3 Hz, 1H), 5.52 (s, 2H), 5.44 (d, J = 16.5 Hz, 1H), 5.09-4.88 (m, 2H), 3.82-3.54 (m, 2H), 3.46- 3.34 (m, 2H), 3.10- 3.00 (m, 1H), 2.64 (s, 3H), 2.14-2.04 (m, 1H), 2.03-1.93 (m, 2H), 1.91- 1.52 (m, 5H), 1.03 (t, J = 7.2 Hz, 3H), 1.00-0.91 (m, 6H). |
| 29. | 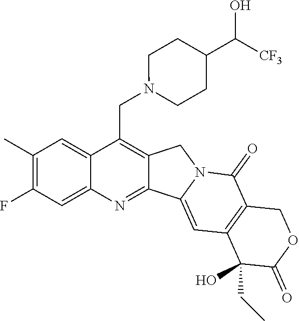 [SMILES: CC[C@@]1(O)C(=O)OCc2c1cc1n(c2=O)Cc2c-1nc1cc(F)c(C)cc1c2CN1CCC(C(O)C(F)(F)F)CC1] | (4S)-4-ethyl-8- fluoro-4-hydroxy-9- methyl-11-((4- (2,2,2-trifluoro-1- hydroxyethyl)piperidin- 1-yl)methyl)-1H- pyrano[3′,4′:6,7] indolizino[1,2- b]quinoline- 3,14(4H,12H)-dione | 576.1 | ¹H NMR (400 MHz, METHANOL-d₄) δ 8.34 (br d, J = 7.6 Hz, 1H), 7.91 (d, J = 10.4 Hz, 1H), 7.68 (s, 1H), 5.62 (d, J = 16.5 Hz, 1H), 5.51 (s, 2H), 5.43 (d, J = 16.3 Hz, 1H), 5.01-4.89 (m, 2H), 3.87-3.82 (m, 1H), 3.76- 3.57 (m, 2H), 3.48- 3.34 (m, 2H), 2.63 (s, 3H), 2.20-2.04 (m, 2H), 2.03-1.76 (m, 5H), 1.03 (t, J = 7.4 Hz, 3H). |
| 30. | 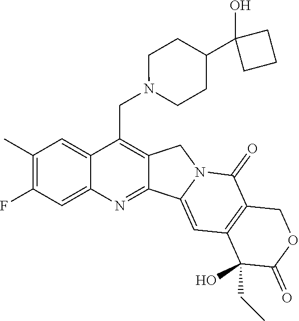 [SMILES: CC[C@@]1(O)C(=O)OCc2c1cc1n(c2=O)Cc2c-1nc1cc(F)c(C)cc1c2CN1CCC(C2(O)CCC2)CC1] | (S)-4-ethyl-8-fluoro- 4-hydroxy-11-((4-(1- hydroxycyclobutyl) piperidin-1- yl)methyl)-9-methyl- 1H- pyrano[3′,4′:6,7] indolizino[1,2- b]quinoline- 3,14(4H,12H)-dione | 548.3 | ¹H NMR (400 MHz, MeOD-d₄) δ 8.35 (br d, J = 7.3 Hz, 1H), 7.93 (d, J = 10.4 Hz, 1H), 7.70 (s, 1H), 5.63 (d, J = 16.5 Hz, 1H), 5.53 (s, 2H), 5.44 (d, J = 16.3 Hz, 1H), 5.00 (br s, 2H), 3.79-3.64 (m, 2H), 3.44-3.34 (m, 2H), 2.64 (s, 3H), 2.27-2.12 (m, 2H), 2.07-1.93 (m, 6H), 1.91-1.80 (m, 2H), 1.80-1.66 (m, 2H), 1.65- 1.53 (m, 1H), 1.03 (t, J = 7.4 Hz, 3H). |
| 31. | 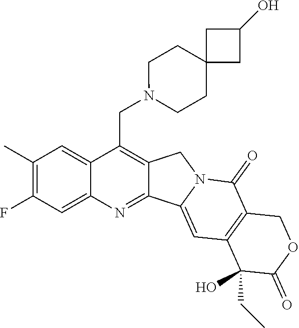 [SMILES: CC[C@@]1(O)C(=O)OCc2c1cc1n(c2=O)Cc2c-1nc1cc(F)c(C)cc1c2CN1CCC2(CC1)CC(O)C2] | (S)-4-ethyl-8-fluoro- 4-hydroxy-11-((2- hydroxy-7- azaspiro[3.5]nonan- 7-yl)methyl)-9- methyl-1H- pyrano[3′,4′:6,7] indolizino[1,2- b]quinoline- 3,14(4H,12H)-dione | 534.3 | ¹H NMR (400 MHz, MeOD-d₄) δ 8.33 (d, J = 7.5 Hz, 1H), 7.91 (d, J = 10.3 Hz, 1H), 7.68 (s, 1H), 5.63 (d, J = 16.3 Hz, 1H), 5.51 (s, 2H), 5.43 (d, J = 16.3 Hz, 1H), 5.05- 4.93 (m, 2H), 4.35- 4.25 (m, 1H), 3.68-3.34 (m, 4H), 2.63 (s, 3H), 2.30-2.10 (m, 2H), 2.09- 1.95 (m, 2H), 1.95- 1.75 (m, 6H), 1.03 (t, J = 7.4 Hz, 3H). |
| 32. | 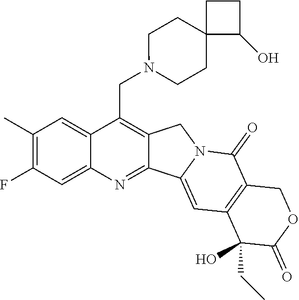 [SMILES: CC[C@@]1(O)C(=O)OCc2c1cc1n(c2=O)Cc2c-1nc1cc(F)c(C)cc1c2CN1CCC2(CCC2O)CC1] | (4S)-4-ethyl-8- fluoro-4-hydroxy- 11-((1-hydroxy-7- azaspiro[3.5]nonan- 7-yl)methyl)-9- methyl-1H- pyrano[3′,4′:6,7] indolizino[1,2- b]quinoline- 3,14(4H,12H)-dione | 534.3 | ¹H NMR (400 MHz, MeOD-d₄) δ 8.35 (d, J = 7.6 Hz, 1H), 7.92 (br d, J = 10.4 Hz, 1H), 7.69 (s, 1H), 5.63 (d, J = 16.5 Hz, 1H), 5.51 (s, 2H), 5.44 (d, J = 16.3 Hz, 1H), 5.04- 4.90 (m, 2H), 4.14- 3.86 (m, 1H), 3.79-3.53 (m, 2H), 3.48-3.35 (m, 3H), 2.63 (s, 3H), 2.35- 2.22 (m, 1H), 2.06-1.65 (m, 8H), 1.54-1.42 (m, 1H), 1.03 (t, J = 7.4 Hz, 3H). |
| 33 | 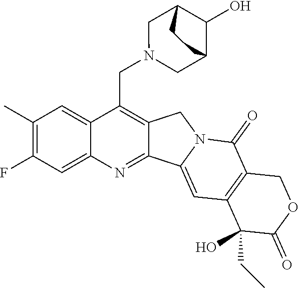 [SMILES: CC[C@@]1(O)C(=O)OCc2c1cc1n(c2=O)Cc2c-1nc1cc(F)c(C)cc1c2CN1C[C@H]2CC[C@@H](C1)C2O] | (4S)-4-ethyl-8- fluoro-4-hydroxy- 11-(((1R,5S)-8- hydroxy-3- azabicyclo[3.2.1] octan-3-yl)methyl)-9- methyl-1H- pyrano[3′,4′:6,7] indolizino[1,2- b]quinoline- 3,14(4H,12H)-dione | 520.2 | ¹H NMR (400 MHz, DMSO-d₆) δ 8.50 (br d, J = 7.6 Hz, 1H), 7.87 (br d, J = 9.6 Hz, 1H), 7.42- 7.24 (m, 1H), 6.88-6.13 (m, 1H), 5.60-5.19 (m, 3H), 5.11-4.63 (m, 1H), 4.20-3.88 (m, 2H), 3.82- 3.58 (m, 3H), 3.28- 2.92 (m, 2H), 2.65-2.54 (m, 2H), 2.38-2.09 (m, 2H), 1.98-1.79 (m, 4H), 1.80-1.55 (m, 2H), 1.60- 1.30 (m, 2H), 0.88 (br t, J = 7.3 Hz, 3H). |
| 34. | 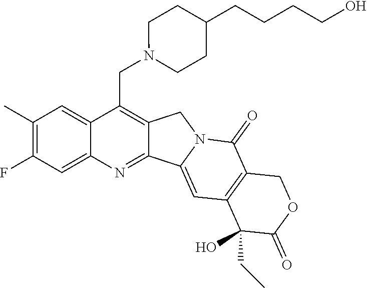 [SMILES: CC[C@@]1(O)C(=O)OCc2c1cc1n(c2=O)Cc2c-1nc1cc(F)c(C)cc1c2CN1CCC(CCCCO)CC1] | (S)-4-ethyl-8-fluoro- 4-hydroxy-11-((4-(4- hydroxybutyl)piperidin- 1-yl)methyl)-9- methyl-1,12- dihydro-14H- pyrano[3′,4′:6,7] indolizino[1,2- b]quinoline- 3,14(4H)-dione | 550.4 | ¹H NMR (400 MHz, MeOD-d₄) δ 8.34 (d, J = 7.6 Hz, 1H), 7.92 (br d, J = 10.4 Hz, 1H), 7.69 (s, 1H), 5.63 (d, J = 16.4 Hz, 1H), 5.52 (s, 2H), 5.44 (d, J = 16.4 Hz, 1H), 4.97- 4.90 (m, 2H), 3.65- 3.55 (m, 2H), 2.64 (s, 3H), 2.12-1.95 (m, 4H), 1.70-1.30 (m, 13H), 1.03 (t, J = 7.4 Hz, 3H). |
| 35. | 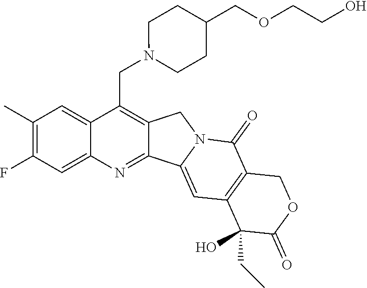 [SMILES: CC[C@@]1(O)C(=O)OCc2c1cc1n(c2=O)Cc2c-1nc1cc(F)c(C)cc1c2CN1CCC(COCCO)CC1] | (S)-4-ethyl-8-fluoro- 4-hydroxy-11-((4- ((2- hydroxyethoxy) methyl)piperidin-1- yl)methyl)-9-methyl- 1,12-dihydro-14H- pyrano[3′,4′:6,7] indolizino[1,2- b]quinoline- 3,14(4H)-dione | 552.2 | ¹H NMR (400 MHz, MeOD-d₄) δ 8.35 (d, J = 7.6 Hz, 1H), 7.91 (br d, J = 10.4 Hz, 1H), 7.69 (s, 1H), 5.63 (d, J = 16.4 Hz, 1H), 5.51 (s, 2H), 5.44 (d, J = 16.4 Hz, 1H), 5.04- 4.90 (m, 2H), 3.75- 3.60 (m, 4H), 3.55-3.45 (m, 2H), 3.45-3.35 (m, 4H), 2.64 (s, 3H), 2.10- 1.95 (m, 5H), 1.67 (d, J = 6.8 Hz, 1H), 1.65-1.50 (m, 1H), 1.03 (t, J = 7.4 Hz, 3H). |
| 46 | 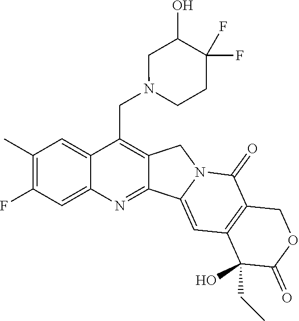 [SMILES: CC[C@@]1(O)C(=O)OCc2c1cc1n(c2=O)Cc2c-1nc1cc(F)c(C)cc1c2CN1CCC(F)(F)C(O)C1] | (4S)-11-((4,4- difluoro-3- hydroxypiperidin-1- yl)methyl)-4-ethyl- 8-fluoro-4-hydroxy- 9-methyl-1,12- dihydro-14H- pyrano[3′,4′:6,7] indolizino[1,2- b]quinoline- 3,14(4H)-dione | 530.15 | ¹H NMR (400 MHz, MeOD-d₄) δ 8.39 (d, J = 8.3 Hz, 1H), 7.82 (d, J = 10.5 Hz, 1H), 7.68 (s, 1H), 5.63 (d, J = 16.3 Hz, 1H), 5.59-5.52 (m, 1H), 5.49-5.40 (m, 2H), 4.50- 4.30 (m, 2H), 3.96- 3.85 (m, 1H), 3.12-2.89 (m, 2H), 2.89-2.69 (m, 2H), 2.58 (s, 3H), 2.39- 2.19 (m, 1H), 2.12-1.92 (m, 3H), 1.03 (t, J = 7.4 Hz, 3H). |
| 47 | 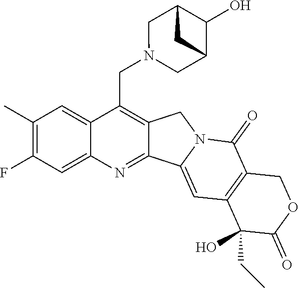 [SMILES: CC[C@@]1(O)C(=O)OCc2c1cc1n(c2=O)Cc2c-1nc1cc(F)c(C)cc1c2CN1C[C@H]2C[C@@H](C1)C2O] | (4S)-4-ethyl-8- fluoro-4-hydroxy- 11-(((1R,5S)-6- hydroxy-3- azabicyclo[3.1.1] heptan-3-yl)methyl)-9- methyl-1,12- dihydro-14H- pyrano[3',4':6,7] indolizino[1,2- b]quinoline- 3,14(4H)-dione | 506.2 | ¹H NMR (400 MHz, MeOD) δ 8.37 (br d, J = 7.2 Hz, 1H), 7.93 (br d, J = 10.8 Hz, 1H), 7.69 (s, 1H), 5.65-5.40 (m, 4H), 5.25-5.10 (m, 2H), 4.35- 4.20 (m, 1H), 3.90- 3.60 (m, 4H), 2.63 (s, 3H), 2.59 (br s, 2H), 2.03- 1.90 (m, 2H), 1.80- 1.70 (m, 2H), 1.03 (t, J = 6.8 Hz, 3H). |

### Example 36

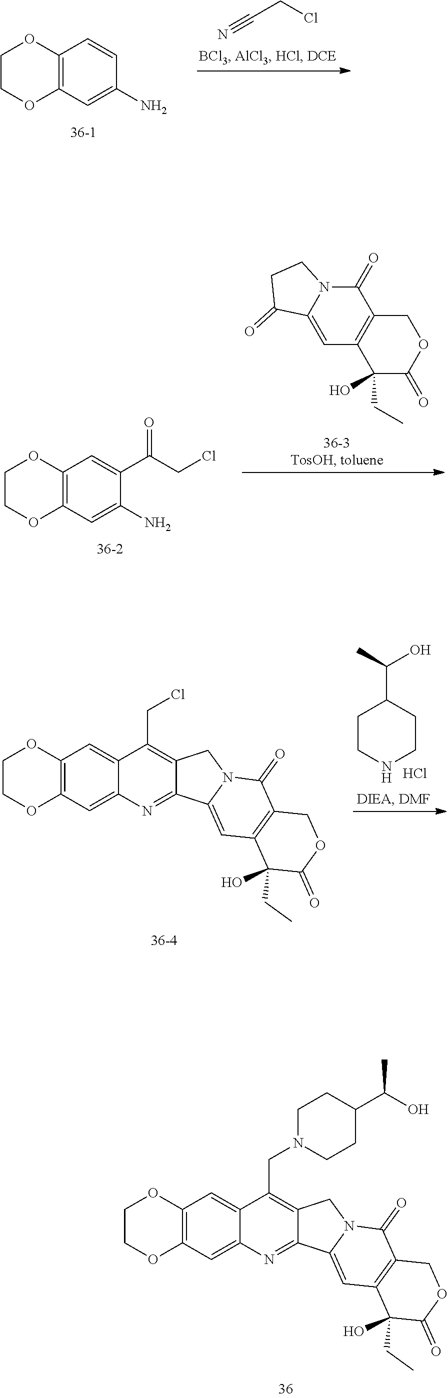

To a solution of trichloroborane (18.6 mL, 18.6 mmol) in DCE (5.00 mL) at 0° C. was added 36-1 (3.00 g, 19.85 mmol) in portion-wise and the mixture was stirred for 10 mins before 2-chloroacetonitrile (1.51 mL, 23.8 mmol) and AlCl₃ (3.47 g, 26.0 mmol) was added. The mixture was stirred at 20° C. for 20 mins before being heated to 75° C. and stirred for 11 hours. LC-MS showed 36-1 was consumed completely and one main peak with desired mass was detected. The reaction mixture was cooled to 0° C., adjusted to pH=1 with 2N HCl and stirred for 1 hr. The aqueous phase was extracted with DCM (50.0 mL×3). The combined organic phase was washed with brine (30.0 mL), dried with anhydrous Na₂SO₄, filtered and the filtrate was concentrated. The residue was purified by prep-HPLC to obtain 36-2 (1.30 g, 5.71 mmol, 28.8% yield) as a yellow solid. LC-MS: 228.0 [M+H]⁺, rt=0.587 min.

To a solution of 36-2 (200 mg, 1.12 mmol) in Toluene (5.00 mL) was added 36-3 (231 mg, 0.879 mmol) and 4-methylbenzene-1-sulfonic acid (7.56 mg, 0.0440 mmol) at 25° C. The mixture was stirred at 110° C. for 12 hrs. LC-MS showed 36-2 was consumed completely and one main peak with desired mass was detected. The reaction mixture was cooled to RT, filtered and the solid was dried under reduced pressure. The crude product was triturated with ethyl acetate (10 mL) at 25° C. for 3 hrs. 36-4 (380 mg, 0.835 mmol, 95.1% yield) was obtained as a brown solid. LC-MS: 455.1 [M+H]⁺, rt=0.844 min.

To a solution of 36-4 (50.0 mg, 0.110 mmol) in DMF (2.00 mL) was added (R)-1-(piperidin-4-yl)ethan-1-ol hydrochloride (21.5 mg, 0.13 mmol) and DIEA (77 μL, 0.440 mmol) at 0° C. The mixture was stirred at 25° C. for 15 mins. LC-MS showed 36-4 was consumed completely and one main peak with desired mass was detected. The mixture was purified by prep-HPLC. Compound 36 (13.7 mg, 0.0250 mmol, 22.8% yield) was obtained as a yellow solid.

The compounds below were synthesized following procedures described for Example 36.

| Ex | Structure | Name | [M + H]⁺ | ¹H NMR |
|---|---|---|---|---|
| 36. | 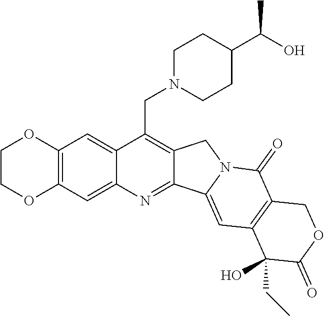 [SMILES: CC[C@@]1(O)C(=O)OCc2c1cc1n(c2=O)Cc2c-1nc1cc3c(cc1c2CN1CCC([C@@H](C)O)CC1)OCCO3] | (S)-8-ethyl-8- hydroxy-15-((4- ((R)-1- hydroxyethyl) piperidin-1-yl)methyl)- 2,3,11,14- tetrahydro-12H- [1,4]dioxino[2,3- glpyrano[3′,4′:6,7] indolizino[1,2- b]quinoline- 9,12(8H)-dione | 548.4 | ¹H NMR (400 MHz, METHANOL-d₄) δ 7.84 (s, 1H), 7.71 (s, 1H), 7.66 (s, 1H), 5.62 (d, J = 16.2 Hz, 1H), 5.47 (s, 2H), 5.45-5.39 (m, 1H), 4.83-4.76 (m, 2H), 4.50 (s, 4H), 3.80-3.62 (m, 2H), 3.62-3.51 (m, 2H), 2.17-2.06 (m, 1H), 2.03- 1.93 (m, 2H), 1.95- 1.88 (m, 1H), 1.72-1.48 (m, 3H), 1.22-1.14 (m, 4H), 1.03 (t, J = 7.5 Hz, 3H). |
| 37. | 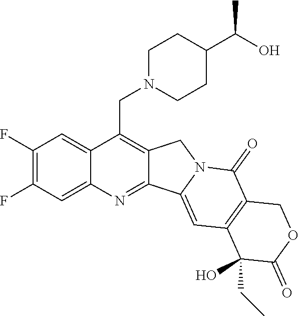 [SMILES: CC[C@@]1(O)C(=O)OCc2c1cc1n(c2=O)Cc2c-1nc1cc(F)c(F)cc1c2CN1CCC([C@@H](C)O)CC1] | (S)-4-ethyl-8,9- difluoro-4- hydroxy-11-((4- ((R)-1- hydroxyethyl) piperidin-1-yl)methyl)- 1,12-dihydro-14H- pyrano[3′,4′:6,7] indolizino[1,2- b]quinoline- 3,14(4H)-dione | 526.3 | ¹H NMR (400 MHz, METHANOL-d₄) δ 8.48- 8.37 (m, 1H), 8.25- 8.11 (m, 1H), 7.68 (s, 1H), 5.68-5.59 (m, 1H), 5.53 (s, 2H), 5.48-5.39 (m, 1H), 4.81-4.55 (m, 2H), 3.75-3.44 (m, 4H), 2.18-2.05 (m, 1H), 2.04- 1.94 (m, 2H), 1.93- 1.81 (m, 1H), 1.71-1.43 (m, 3H), 1.24-1.15 (m, 4H), 1.03 (t, J = 7.4 Hz, 3H). |
| 38. | 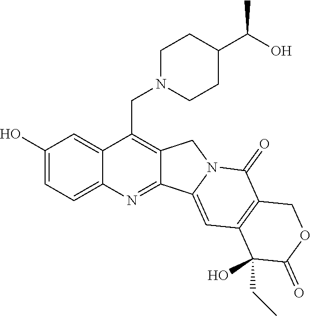 [SMILES: CC[C@@]1(O)C(=O)OCc2c1cc1n(c2=O)Cc2c-1nc1ccc(O)cc1c2CN1CCC([C@@H](C)O)CC1] | (S)-4-ethyl-4,9- dihydroxy-11-((4- ((R)-1- hydroxyethyl) piperidin-1-yl)methyl)- 1,12-dihydro-14H- pyrano[3′,4′:6,7] indolizino[1,2- b]quinoline- 3,14(4H)-dione | 506.3 | ¹H NMR (400 MHz, METHANOL-d₄) δ 8.17 (d, J = 9.3 Hz, 1H), 7.64 (s, 1H), 7.59-7.52 (m, 2H), 5.61 (d, J = 16.2 Hz, 1H), 5.48 (s, 2H), 5.41 (d, J = 16.3 Hz, 1H), 3.76- 3.62 (m, 2H), 2.20- 2.05 (m, 1H), 1.98 (td, J = 11.9, 7.1 Hz, 2H), 1.92- 1.80 (m, 1H), 1.66- 1.54 (m, 3H), 1.40-1.34 (m, 2H), 1.32-1.28 (m, 3H), 1.17 (br d, J = 6.2 Hz, 3H), 1.02 (t, J = 7.4 Hz, 3H). |
| 39. | 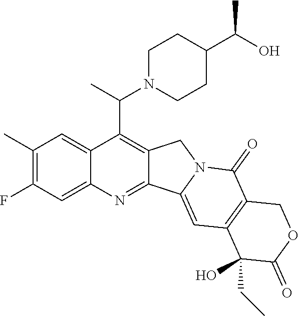 [SMILES: CC[C@@]1(O)C(=O)OCc2c1cc1n(c2=O)Cc2c-1nc1cc(F)c(C)cc1c2C(C)N1CCC([C@@H](C)O)CC1] | (4S)-4-ethyl-8- fluoro-4-hydroxy- 11-(1-(4-((R)-1- hydroxyethyl) piperidin-1-yl)ethyl)-9- methyl-1,12- dihydro-14H- pyrano[3′,4′:6,7] indolizino[1,2- b]quinoline- 3,14(4H)-dione | 536.4 | ¹H NMR (400 MHz, METHANOL-d₄) δ 8.55- 8.37 (m, 1H), 7.90- 7.85 (m, 1H), 7.68 (s, 1H), 5.65 (br d, J = 16.4 Hz, 1H), 5.59 (br s, 2H), 5.43 (br d, J = 16.4 Hz, 1H), 4.95-4.90 (m, 1H), 4.11-3.85 (m, 1H), 3.63- 3.51 (m, 1H), 3.11- 2.85 (m, 1H), 2.63 (s, 3H), 2.31-1.86 (m, 7H), 1.82-1.41 (m, 4H), 1.16 (t, J = 6.8 Hz, 4H), 1.03 (t, J = 7.3 Hz, 3H). |
| 40. | 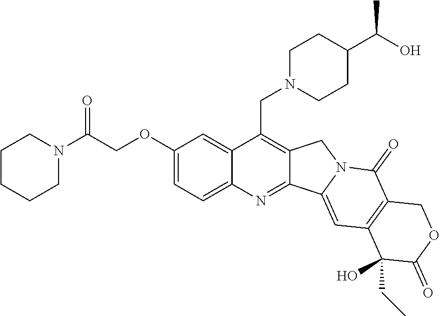 [SMILES: CC[C@@]1(O)C(=O)OCc2c1cc1n(c2=O)Cc2c-1nc1ccc(OCC(=O)N3CCCCC3)cc1c2CN1CCC([C@@H](C)O)CC1] | (S)-4-ethyl-4- hydroxy-11-((4- ((R)-1- hydroxyethyl) piperidin-1-yl)methyl)- 9-(2-oxo-2- (piperidin-1- yl)ethoxy)-1,12- dihydro-14H- pyrano[3′,4′:6,7] indolizino[1,2- b]quinoline- 3,14(4H)-dione | 631.4 | ¹H NMR (400 MHz, METHANOL-d₄) δ8.25 (d, J = 9.1 Hz, 1H), 7.78- 7.66 (m, 2H), 7.65 (s, 1H), 5.60 (d, J = 16.4 Hz, 1H), 5.51 (s, 2H), 5.41 (d, J = 16.3 Hz, 1H), 5.15 (s, 2H), 4.99 (br s, 2H), 3.74-3.50 (m, 7H), \3.40- 3.30 (m, 2H), 2.24- 2.03 (m, 1H), 2.00-1.95 (m, 2H), 1.93-1.86 (m, 1H), 1.76-1.63 (m, 6H), 1.56-1.50 (m, 2H), 1.22- 1.13 (m, 3H), 1.01 (t, J = 7.3 Hz, 3H). |
| 41. | 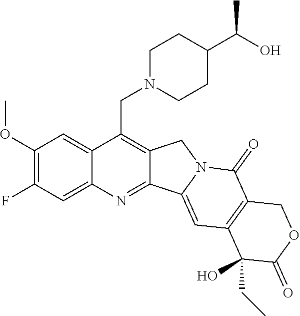 [SMILES: CC[C@@]1(O)C(=O)OCc2c1cc1n(c2=O)Cc2c-1nc1cc(F)c(OC)cc1c2CN1CCC([C@@H](C)O)CC1] | (S)-4-ethyl-8- fluoro-4-hydroxy- 11-((4-((R)-1- hydroxyethyl) piperidin-1-yl)methyl)- 9-methoxy-1,12- dihydro-14H- pyrano[3′,4′:6,7] indolizino[1,2- b]quinoline- 3,14(4H)-dione | 538.4 | ¹H NMR (400 MHz, METHANOL-d₄) δ 8.06- 7.91 (m, 1H), 7.89- 7.72 (m, 1H), 7.66 (s, 1H), 5.63 (d, J = 16.3 Hz, 1H), 5.52 (br s, 2H), 5.43 (d, J = 16.3 Hz, 1H), 5.14- 4.97 (m, 2H), 4.20 (s, 3H), 3.83-3.63 (m, 2H), 3.59-3.54 (m, 1H), 2.25- 2.08 (m, 1H), 2.05- 1.86 (m, 3H), 1.73-1.48 (m, 3H), 1.37-1.27 (m, 1H), 1.22-1.16 (m, 4H), 1.03 (t, J = 7.4 Hz, 3H). |
| 42. | 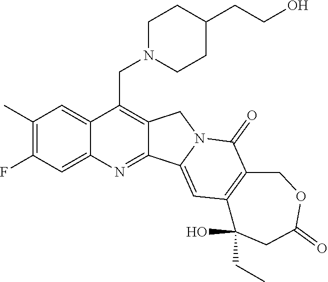 [SMILES: CC[C@@]1(O)CC(=O)OCc2c1cc1n(c2=O)Cc2c-1nc1cc(F)c(C)cc1c2CN1CCC(CCO)CC1] | ((R)-5-ethyl-9- fluoro-5-hydroxy- 12-((4-(2- hydroxyethyl) piperidin-1-yl)methyl)- 10-methyl- 1,4,5,13- tetrahydro- 3H,15H- oxepino[3′,4′:6,7] indolizino[1,2- b]quinoline-3,15- dione | 536.3 | / |
| 44. | 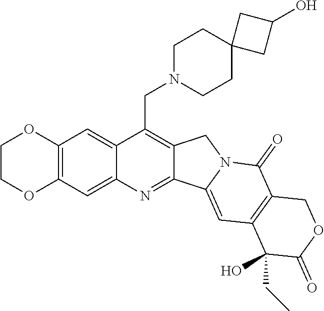 [SMILES: CC[C@@]1(O)C(=O)OCc2c1cc1n(c2=O)Cc2c-1nc1cc3c(cc1c2CN1CCC2(CC1)CC(O)C2)OCCO3] | (S)-8-ethyl-8- hydroxy-15-((2- hydroxy-7- azaspiro[3.5]nonan- 7-yl)methyl)- 2,3,11,14- tetrahydro-12H- [1,4]dioxino[2,3- glpyrano[3′,4′:6,7] indolizino[1,2- b]quinoline- 9,12(8H)-dione | 560.3 | ¹H NMR (400 MHz, METHANOL-d₄) δ 7.80 (s, 1H), 7.66 (s, 1H), 7.62 (s, 1H), 5.59 (d, J = 16.0 Hz, 1H), 5.43 (s, 2H), 5.40-5.35 (m, 1H), 4.90-4.85 (m, 2H), 4.48 (s, 4H), 4.27 (t, J = 7.2 Hz, 1H), 3.70-3.55 (m, 2H), 3.40-3.35 (m, 2H), 2.60-2.50 (m, 1H), 2.28- 2.20 (m, 1H), 1.97 (dt, J = 4.0, 1.7 Hz, 1H), 1.90- 1.80 (m, 5H), 1.01 (t, J = 8.4 Hz, 3H). |
| 45. | 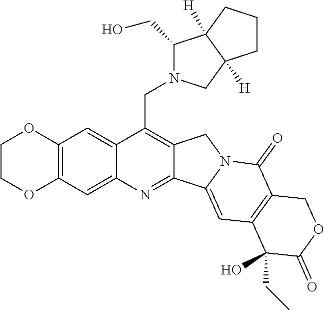 [SMILES: CC[C@@]1(O)C(=O)OCc2c1cc1n(c2=O)Cc2c-1nc1cc3c(cc1c2CN1C[C@@H]2CCC[C@@H]2[C@H]1CO)OCCO3] | (S)-8-ethyl-8- hydroxy-15- (((1S,3aR,6aS)-1- (hydroxymethyl) hexahydrocyclopenta [c]pyrrol-2(1H)- yl)methyl)- 2,3,11,14- tetrahydro-12H- [1,4]dioxino[2,3- glpyrano[3′,4′:6,7] indolizino[1,2- b]quinoline- 9,12(8H)-dione | 560.1 | ¹H NMR (400 MHz, METHANOL-d₄) δ 7.90 (s, 1H), 7.71 (s, 1H), 7.67 (s, 1H), 5.60 (t, J = 16.4 Hz, 2H), 5.50 (d, J = 16.4 Hz, 2H), 4.90- 4.85 (m, 2H), 4.50 (s, 4H), 4.38-4.33 (m, 1H), 4.02 (d, J = 12.0 Hz, 1H), 3.48-3.40 (m, 2H), 3.15- 3.05 (m, 1H), 2.85- 2.65 (m, 2H), 2.05-1.95 (m, 2H), 1.90-1.85 (m, 2H), 1.80-1.60 (m, 4H), 1.03 (t, J = 87.2 Hz, 3H). |

### Example 43

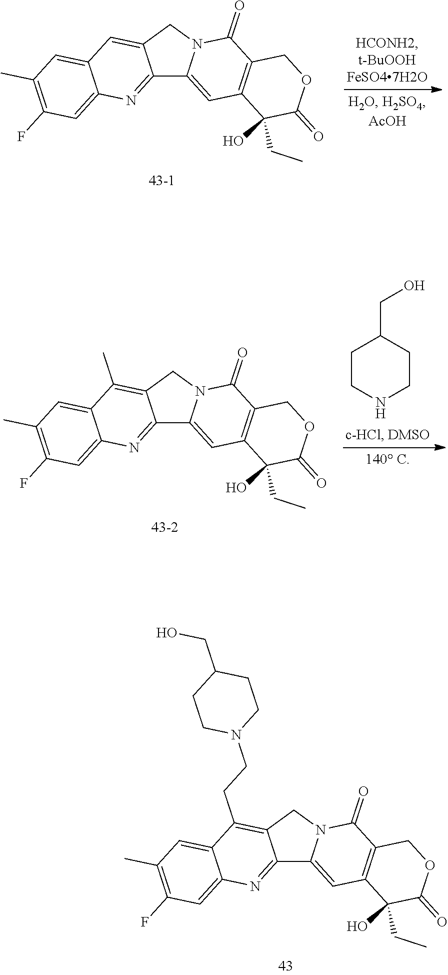 [SMILES: CC[C@@]1(O)C(=O)OCc2c1cc1n(c2=O)Cc2cc3cc(C)c(F)cc3nc2-1 ; CC[C@@]1(O)C(=O)OCc2c1cc1n(c2=O)Cc2c-1nc1cc(F)c(C)cc1c2C ; OCC1CCNCC1 ; CC[C@@]1(O)C(=O)OCc2c1cc1n(c2=O)Cc2c-1nc1cc(F)c(C)cc1c2CCN1CCC(CO)CC1]

43-1 (110 mg, 0.29 mmol) was suspended in a mixture of CF₃COOH (3.5 ml) and H₂SO₄ (1.2 ml). FeSO₄. 7H₂O (0.21 g, 0.76 mmol) and H₂O (6 ml) were added successively. After cooling to 5° C., HCONH₂ (0.2 ml) was added. To the mixture, 65% (CH₃)₃COOH (0.1 ml, 0.68 mmol) was added dropwise at 2° C. The mixture was stirred for 1 h at 0° C. and then poured into ice water. The precipitated material in the solution was collected by suction, washed with water, and dried in vacuo. 43-2 was obtained as yellow powder (0.100 g, Yield: 96.1%).

To a solution of 43-2 (43 mg, 0.11 mmol) and piperidin-4-ylmethanol (75 mg, 0.65 mmol, 0.328 mol) in 2 mL DMSO was added concentrated hydrochloric acid (0.1) and the mixture was heated at 140° C. for 1 h. H₂O (4 mL) was added to the reaction mixture and then filtered. After the filtrate was loaded on to 4 mL of activated HP-20 resin, it was washed with H₂O until the pH of the eluent reached 6. It was then eluted with 20% methanol-dichloromethane solution to obtain the product. The solvent was evaporated and the residue was purified by SGC (DCM/MeOH=10:1) to give 11 mg of compound 43 as yellow solid (21% yield).

| Ex | Structure | Name | [M + H]⁺ | ¹H NMR |
|---|---|---|---|---|
| 43. | 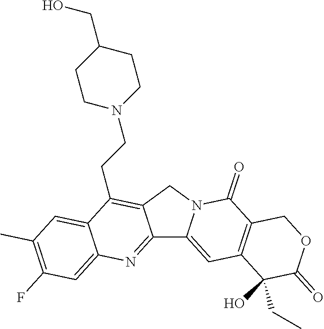 [SMILES: CC[C@@]1(O)C(=O)OCc2c1cc1n(c2=O)Cc2c-1nc1cc(F)c(C)cc1c2CCN1CCC(CO)CC1] | (S)-4-ethyl-8- fluoro-4-hydroxy- 11-(2-(4- (hydroxymethyl) piperidin-1-yl)ethyl)- 9-methyl-1,12- dihydro-14H- pyrano[3′,4′:6,7] indolizino[1,2- b]quinoline- 3,14(4H)-dione | 522.6 | / |
| 48. | 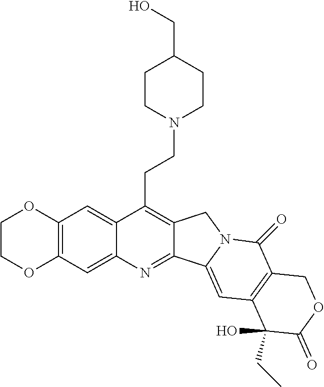 [SMILES: CC[C@@]1(O)C(=O)OCc2c1cc1n(c2=O)Cc2c-1nc1cc3c(cc1c2CCN1CCC(CO)CC1)OCCO3] | (S)-8-ethyl-8- hydroxy-15-(2-(4- (hydroxymethyl) piperidin-1-yl)ethyl)- 2,3,11,14- tetrahydro-12H- [1,4]dioxino[2,3- g]pyrano[3′,4′:6,7] indolizino[1,2- b]quinoline- 9,12(8H)-dione | 548.2 | ¹H NMR (400 MHz, MeOH-d₄) δ 7.62-7.59 (m, 2H), 7.58 (s, 1H), 5.58 (d, J = 16.3 Hz, 1H), 5.39 (d, J = 16.1 Hz, 1H), 5.30 (s, 2H), 4.45 (s, 4H), 3.86 (br d, J = 11.8 Hz, 2H), 3.64-3.57 (m, 2H), 3.56-3.50 (m, 2H), 3.45 (br d, J = 4.0 Hz, 1H), 3.15 (br s, 1H), 2.12 (br d, J = 13.9 Hz, 2H), 2.00- 1.90 (m, 2H), 1.84 (ddd, J = 8.6, 5.9, 2.3 Hz, 1H), 1.65 (d, J = 7.0 Hz, 1H), 1.60 (br s, 1H), 1.36-1.25 (m, 4H), 1.01 (t, J = 7.4 Hz, 3H). |

### Example 49

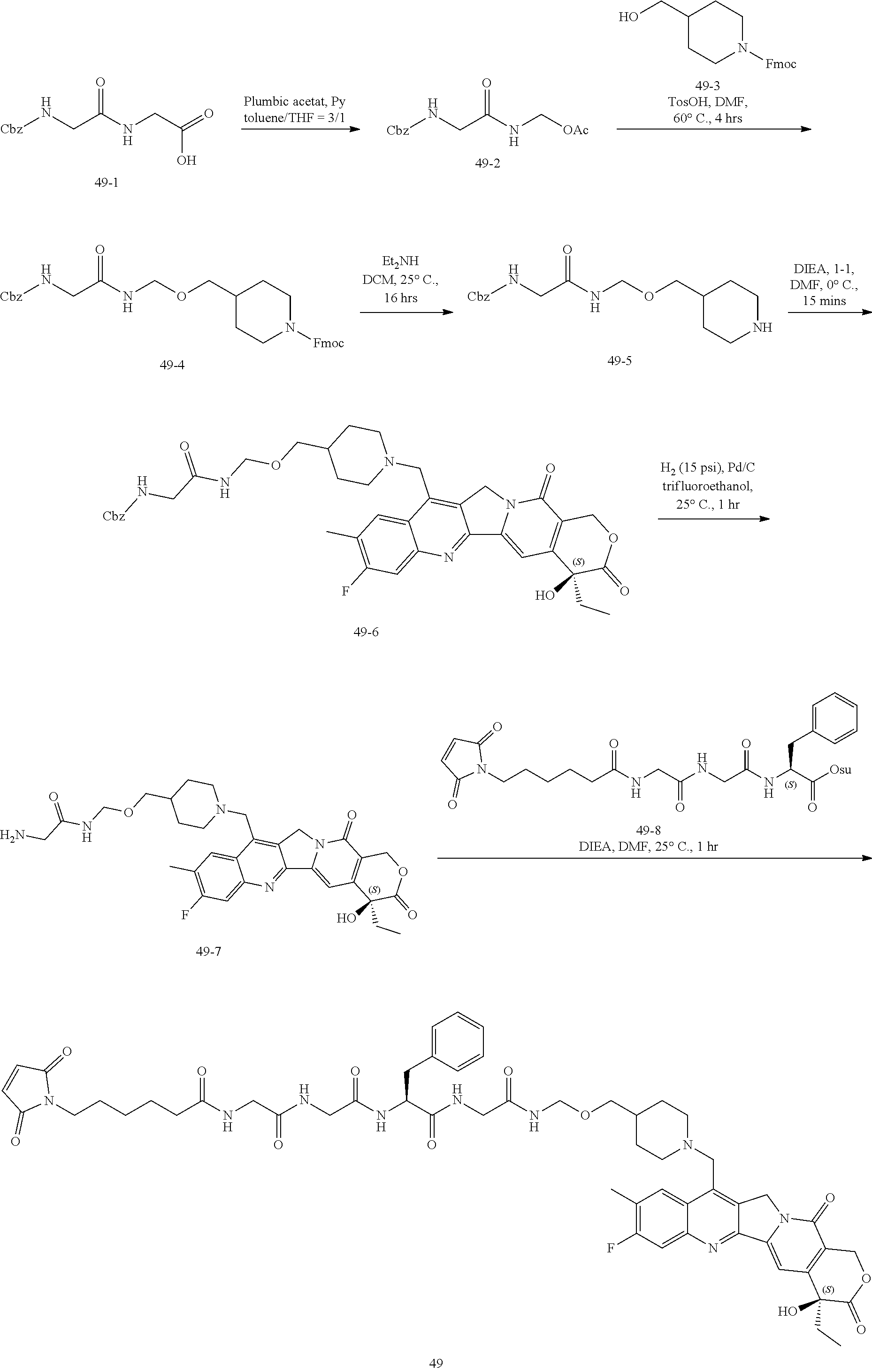

To a solution of 49-1 (15 g, 56.3 mmol) and Py (5.70 mL, 70.4 mmol) in THF (60 mL) and toluene (180 mL) was added Pb(OAc)₄ (35.0 g, 78.9 mmol) at 25° C. and the mixture was stirred at 80° C. for 12 hrs. TLC (P/E=0/1) showed 49-1 was consumed completely. The reaction mixture was cooled to RT, filtered and the filtrate was concentrated under reduced pressure. The residue was purified by SGC (Petroleum ether/Ethyl acetate=10/1 to 1/1). 49-2 (6.80 g, 24.3 mmol, 43.1% yield) was obtained as a light yellow solid.

To a solution of 49-3 (1.00 g, 2.96 mmol) and 49-2 (1.08 g, 3.85 mmol) in DMF (10 mL) was added TosOH (0.03 g, 0.148 mmol) at 25° C. The reaction mixture was heated to 60° C. and stirred for 12 hrs. TLC (PE/EA=0/1) showed most of 49-3 was consumed and one main spot was detected. The reaction mixture was cooled to RT, concentrated under reduced pressure. The residue was purified by SGC (Petroleum ether/Ethyl acetate=50/1 to 0/1). 49-4 (1.00 g, 1.79 mmol, 60.5% yield) was obtained as a yellow solid.

To a solution of 49-4 (1.00 g, 1.79 mmol) in DCM (10 mL) was added diethylamine (1.86 mL, 17.9 mmol) at 25° C. and the mixture was stirred for 12 hrs. LCMS showed 49-4 was consumed completely and desired MS was detected. The reaction mixture was concentrated under reduced pressure. The residue was purified by prep-HPLC. 49-5 (270 mg, 0.805 mmol, 44.9% yield) was obtained as yellow oil.

To a mixture of 49-5 (340 mg, 0.793 mmol) and 1-1 (265.91 mg, 0.793 mmol) in DMF (3 mL) was added DIEA (307 mg, 2.38 mmol) at 0° C. The reaction mixture was warmed to 25° C. and stirred for 15 mins. HPLC showed 49-5 was consumed completely. The reaction mixture was purified by prep-HPLC. 49-6 (110 mg, 0.151 mmol, 19.1% yield) was obtained as a yellow solid.

To a solution of 49-6 (110 mg, 0.151 mmol) in 2,2,2-trifluoroethan-1-ol (5.0 mL) was added Pd/C (30.0 mg, 0.282 mmol) at 25° C. under N₂. The suspension was degassed and purged with H₂ (15 psi) for 3 times before being stirred under H₂ (15 Psi) at 25° C. for 2 hrs. LCMS showed 49-6 was consumed completely and desired MS was detected. The reaction mixture was filtered and concentrated under reduced pressure. The residue was purified by prep-HPLC. 49-7 (30.0 mg, 0.042 mmol, 28.1% yield) was obtained as a yellow solid. LC-MS: 594.3 [M+H]⁺.

To a mixture of 49-7 (30.0 mg, 0.042 mmol) and compound 49-8 (96.6 mg, 0.085 mmol) in DMF (2.0 mL) was added DIEA (16.3 mg, 0.126 mmol) at 25° C. and the mixture was stirred for 12 hrs. LCMS showed 49-7 was consumed completely and desired MS was detected. The mixture was purified by prep-HPLC. Compound 49 (4.39 mg, 0.004 mmol, 9.88%) was obtained as a yellow solid.

The compounds below were synthesized following procedures described for Example 49.

| Ex. | Structure | Name | [M + H]⁺ | ¹H NMR |
|---|---|---|---|---|
| 49. | 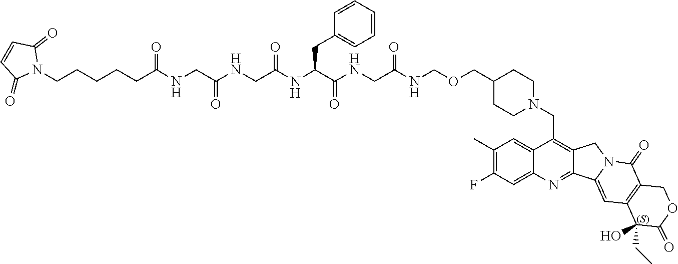 | N-((S)-9-benzyl-1-(1-(((S)-4- ethyl-8-fluoro-4-hydroxy-9- methyl-3,14-dioxo-3,4,12,14- tetrahydro-1H- pyrano[3′,4′:6,7]indolizino[1,2- b]quinolin-11- yl)methyl)piperidin-4-yl)- 5,8,11,14-tetraoxo-2-oxa- 4,7,10,13-tetraazapentadecan- 15-yl)-6-(2,5-dioxo-2,5-dihydro- 1H-pyrrol-1-yl)hexanamide | 1048.5 | ¹H NMR (400 MHz, DMSO-d₆) δ 8.53-8.41 (m, 1H), 8.40-8.33 (m, 1H), 8.30-8.22 (m, 1H), 8.18-7.96 (m, 3H), 7.94-7.77 (m, 1H), 7.34-7.29 (m, 1H), 7.25-7.14 (m, 5H), 7.08-6.96 (m, 1H), 6.63-6.44 (m, 1H), 5.51-5.38 (m, 2H), 5.38-5.21 (m, 2H), 4.59-4.43 (m, 3H), 4.21-4.00 (m, 4H), 3.64 (br s, 6H), 3.23 (br d, J = 5.9 Hz, 3H), 3.08-3.01 (m, 2H), 2.90-2.80 (m, 4H), 2.15-2.05 (m, 4H), 1.87 (td, J = 14.8, 7.3 Hz, 2H), 1.64-1.57 (m, 2H), 1.55-1.43 (m, 5H), 1.22-1.08 (m, 5H), 0.88 (br t, J = 7.1 Hz, 3H). |
| 50. | 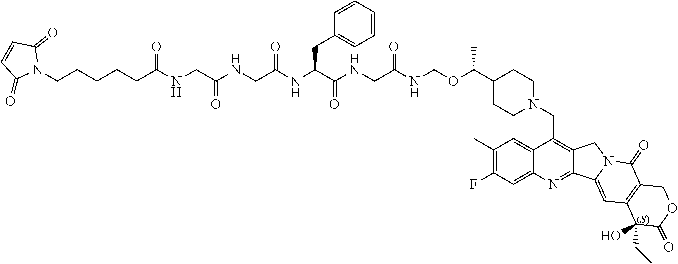 | N-((2R,10S)-10-benzyl-2-(1- (((S)-4-ethyl-8-fluoro-4- hydroxy-9-methyl-3,14-dioxo- 3,4,12,14-tetrahydro-1H- pyrano[3′,4′:6,7]indolizino[1,2- b]quinolin-11- yl)methyl)piperidin-4-yl)- 6,9,12,15-tetraoxo-3-oxa- 5,8,11,14-tetraazahexadecan-16- yl)-6-(2,5-dioxo-2,5-dihydro- 1H-pyrrol-1-yl)hexanamide | 1062.4 | ¹H NMR (400 MHz, DMSO-d₆) δ 8.53-8.32 (m, 2H), 8.32-8.22 (m, 1H), 8.17-8.05 (m, 2H), 8.01 (br t, J = 4.8 Hz, 1H), 7.93-7.79 (m, 1H), 7.33 (s, 1H), 7.27-7.16 (m, 5H), 7.00 (s, 1H), 6.54 (s, 1H), 5.44 (br s, 2H), 5.34 (br s, 2H), 4.61-4.45 (m, 3H), 4.21-3.97 (m, 3H), 3.79-3.65 (m, 5H), 3.63- 3.54 (m, 2H), 3.17-2.99 (m, 3H), 2.90 (br d, J = 5.6 Hz, 2H), 2.79 (br dd, J = 13.5, 9.9 Hz, 1H), 2.32-2.21 (m, 1H), 2.11 (br t, J = 7.3 Hz, 4H), 1.95-1.80 (m, 2H), 1.69 (br d, J = 10.1 Hz, 1H), 1.48 (qd, J = 14.5, 7.1 Hz, 6H), 1.34- |
| | | | | 1.27 (m, 1H), 1.27-1.13 (m, 4H), |
| | | | | 1.04 (br d, J = 6.0 Hz, 3H), 0.89 |
| | | | | (br t, J = 7.2 Hz, 3H). |
| 51. | 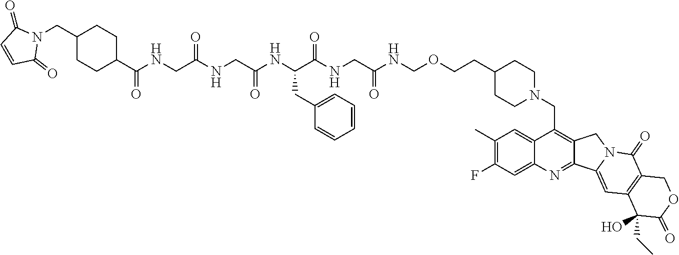 [SMILES: CC[C@@]1(O)C(=O)OCc2c1cc1n(c2=O)Cc2c-1nc1cc(F)c(C)cc1c2CN1CCC(CCOCNC(=O)CNC(=O)[C@H](Cc2ccccc2)NC(=O)CNC(=O)CNC(=O)C2CCC(CN3C(=O)C=CC3=O)CC2)CC1] | N-((10S)-10-benzyl-1-(1-((4- ethyl-8-fluoro-4-hydroxy-9- methyl-3,14-dioxo-3,4,12,14- tetrahydro-1H- pyrano[3′,4′:6,7]indolizino[1,2- b]quinolin-11- yl)methyl)piperidin-4-yl)- 6,9,12,15-tetraoxo-3-oxa- 5,8,11,14-tetraazahexadecan-16- yl)-4-((2,5-dioxo-2,5-dihydro- 1H-pyrrol-1- yl)methyl)cyclohexane-1- carboxamide | 1088.6 | ¹H NMR (400MHz, DMSO-d₆) δ 9.30 (br s, 1H), 8.47-8.35 (m, 2H), 8.23 (t, J = 6.0 Hz, 1H), 8.09 (d, J = 8.0 Hz, 3H), 7.94-7.77 (m, 3H), 7.32 (s, 1H), 7.25-7.11 (m, 6H), 7.10-7.04 (m, 1H), 6.98- 6.92 (m, 3H), 6.53 (br s, 1H), 5.46- 5.37 (m, 4H), 5.00-4.84 (m, 2H), 4.54-4.36 (m, 3H), 3.74- 3.66 (m, 1H), 3.60-3.63 (m, 2H), 3.62-3.58 (m, 2H), 3.57-3.55 (m, 1H), 3.54-3.51 (m, 1H), 3.20- 3.16 (m, 3H), 3.02-2.96 (m, 1H), 2.65-2.55 (m, 1H), 2.53 (s, 3H), 2.10-2.00 (m, 1H), 1.88- 1.75 (m, 4H), 1.73-1.66 (m, 3H), 1.61-1.52 (m, 3H), 1.50- |
| | | | | 1.41 (m, 1H), 1.39-1.29 (m, 3H), |
| | | | | 1.25-1.13(m, 3H), 0.92-0.79 (m, |
| | | | | 5H). |
| | 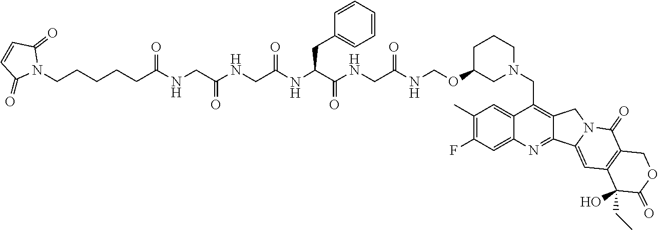 [SMILES: CC[C@@]1(O)C(=O)OCc2c1cc1n(c2=O)Cc2c-1nc1cc(F)c(C)cc1c2CN1CCC[C@H](OCNC(=O)CNC(=O)[C@H](Cc2ccccc2)NC(=O)CNC(=O)CNC(=O)CCCCCN2C(=O)C=CC2=O)C1] | N-((S)-7-benzyl-1-(((S)-1-(((S)- 4-ethyl-8-fluoro-4-hydroxy-9- methyl-3,14-dioxo-3,4,12,14- tetrahydro-1H- pyrano[3′,4′:6,7]indolizino[1,2- b]quinolin-11- yl)methyl)piperidin-3-yl)oxy)- 3,6,9,12-tetraoxo-2,5,8,11- tetraazatridecan-13-yl)-6-(2,5- dioxo-2,5-dihydro-1H-pyrrol-1- yl)hexanamide | 1034.2 | ¹H NMR (400 MHz, MeOD-d₄) δ 8.42 (br t, J = 6.9 Hz, 1H), 8.38 (br d, J = 8.1 Hz, 1H), 8.25 (br t, J = 5.7 Hz, 1H), 8.14-8.04 (m, 2H), 7.99 (br t, J = 5.8 Hz, 1H), 7.87 (d, J = 10.7 Hz, 1H), 7.31 (s, 1H), 7.26-7.15 (m, 5H), 6.99 (s, 2H), 6.52 (s, 1H), 5.42 (s, 2H), 5.34 (s, 2H), 4.60-4.52 (m, 2H), 4.50- 4.42 (m, 1H), 4.18-3.99 (m, 2H), 3.70-3.47 (m, 7H), 3.39-3.34 (m, 2H), 3.12-2.98 (m, 1H), 2.96- 2.89 (m, 1H), 2.83-2.71 (m, 1H), 2.68-2.55 (m, 1H), 2.26- 2.03 (m, 5H), 1.95-1.79 (m, 3H), |
| | | | | 1.70-1.56 (m, 1H), 1.55-1.39 |
| | | | | (m, 5H), 1.38-1.28 (m, 1H), 1.27- |
| | | | | 1.09 (m, 4H), 0.87 (t, J = 7.3 Hz, |
| | | | | 3H). |
| 53. | 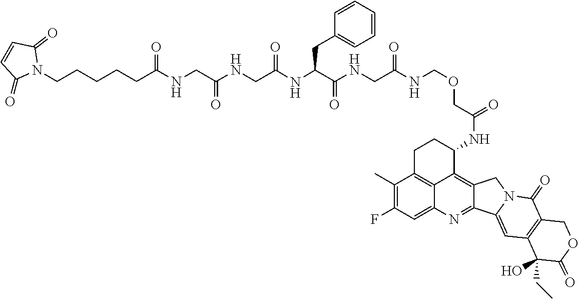 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3] | N-((S)-10-benzyl-1-(((1S,9S)-9- ethyl-5-fluoro-9-hydroxy-4- methyl-10,13-dioxo- 2,3,9,10,13,15-hexahydro- 1H,12H- benzo[de]pyrano[3′,4′:6,7] indolizino[1,2-b]quinolin-1- yl)amino)-1,6,9,12,15-pentaoxo- 3-oxa-5,8,11,14- tetraazahexadecan-16-yl)-6-(2,5- dioxo-2,5-dihydro-1H-pyrrol-1- yl)hexanamide | 1034.5 | ¹H NMR (400 MHz, DMSO-d₆) δ 8.62 (t, J = 6.7 Hz, 1H), 8.50 (d, J = 8.8 Hz, 1H), 8.29 (t, J = 5.8 Hz, 1H), 8.11 (d, J = 8.0 Hz, 1H), 8.05 (t, J = 5.8 Hz, 1H), 7.99 (t, J = 6.0 Hz, 1H), 7.78 (d, J = 10.8 Hz, 1H), 7.31 (s, 1H), 7.26-7.15 (m, 5H), 6.99 (s, 2H), 6.51 (s, 1H), 5.60 (q, J = 7.6 Hz, 1H), 5.42 (s, 2H), 5.20 (s, 2H), 4.63 (d, J = 6.6 Hz, 2H), 4.51-4.39 (m, 1H), 4.01 (s, 2H), 3.78-3.68 (m, 3H), 3.65 (d, J = 5.7 Hz, 2H), 3.62-3.51 (m, 1H), 3.40-3.33 (m, 2H), 3.24-3.08 (m, 2H), 3.02 (dd, J = 13.8, 4.4 Hz, 1H), 2.77 (dd, J = 13.8, 9.5 Hz, 1H), 2.38 (s, 3H), 2.25-2.14 (m, 2H), 2.08 (t, J = 7.5 Hz, 2H), 1.91- |
| | | | | 1.80 (m, 2H), 1.50-1.38 (m, |
| | | | | 4H), 1.23-1.11 (m, 2H), 0.86 (t, |
| | | | | J = 7.3 Hz, 3H). |
| 54. | 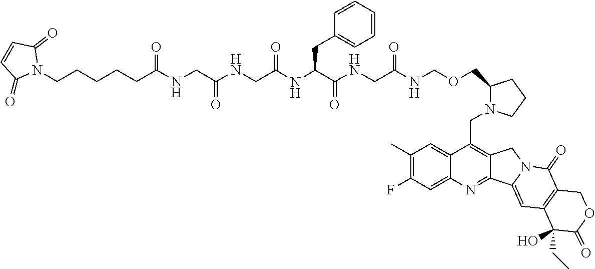 [SMILES: CC[C@@]1(O)C(=O)OCc2c1cc1n(c2=O)Cc2c-1nc1cc(F)c(C)cc1c2CN1CCC[C@@H]1COCNC(=O)CNC(=O)[C@H](Cc1ccccc1)NC(=O)CNC(=O)CNC(=O)CCCCCN1C(=O)C=CC1=O] | N-((S)-9-benzyl-1-((R)-1-(((S)- 4-ethyl-8-fluoro-4-hydroxy-9- methyl-3,14-dioxo-3,4,12,14- tetrahydro-1H- pyrano[3′,4′:6,7]indolizino[1,2- b]quinolin-11- yl)methyl)pyrrolidin-2-yl)- 5,8,11,14-tetraoxo-2-oxa- 4,7,10,13-tetraazapentadecan- 15-yl)-6-(2,5-dioxo-2,5-dihydro- 1H-pyrrol-1-yl)hexanamide | 1034.2 | ¹H NMR (400 MHz, DMSO-d₄) δ 8.61 (br t, J = 6.3 Hz, 1H), 8.44 (br d, J = 8.9 Hz, 1H), 8.31-8.24 (m, 1H), 8.12-8.04 (m, 2H), 8.00 (br t, J = 5.5 Hz, 1H), 7.87 (d, J = 10.8 Hz, 1H), 7.33 (s, 1H), 7.26-7.07 (m, 6H), 6.99 (s, 1H), 6.61-6.48 (m, 1H), 5.44 (s, 2H), 5.35 (br d, J = 7.1 Hz, 2H), 4.74- 4.60 (m, 3H), 4.55-4.41 (m, 1H), 4.05 (br d, J = 13.8 Hz, 1H), 3.77-3.70 (m, 3H), 3.66 (br d, J = 5.6 Hz, 2H), 3.63-3.53 (m, 4H), 2.98 (br d, J = 4.4 Hz, 1H), 2.95-2.83 (m, 2H), 2.79-2.70 (m, 3H), 2.31-2.24 (m, 1H), 2.10 (br t, J = 7.7 Hz, 3H), 1.97- |
| | | | | 1.81 (m, 3H), 1.68-1.60 (m, |
| | | | | 1H), 1.58-1.51 (m, 2H), 1.50- |
| | | | | 1.43 (m, 4H), 1.26-1.11 (m, 3H), |
| | | | | 0.88 (br t, J = 7.3 Hz, 3H). |
| 55. | 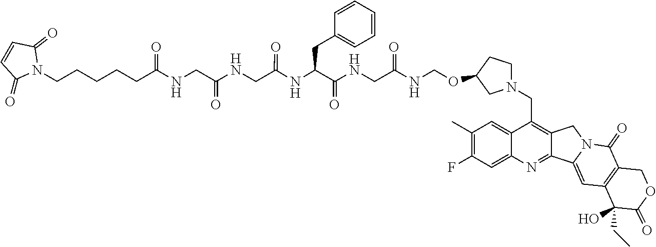 [SMILES: CC[C@@]1(O)C(=O)OCc2c1cc1n(c2=O)Cc2c-1nc1cc(F)c(C)cc1c2CN1CC[C@H](OCNC(=O)CNC(=O)[C@H](Cc2ccccc2)NC(=O)CNC(=O)CNC(=O)CCCCCN2C(=O)C=CC2=O)C1] | N-((S)-7-benzyl-1-(((S)-1-(((S)- 4-ethyl-8-fluoro-4-hydroxy-9- methyl-3,14-dioxo-3,4,12,14- tetrahydro-1H- pyrano[3′,4′:6,7]indolizino[1,2- b]quinolin-11- yl)methyl)pyrrolidin-3-yl)oxy)- 3,6,9,12-tetraoxo-2,5,8,11- tetraazatridecan-13-yl)-6-(2,5- dioxo-2,5-dihydro-1H-pyrrol-1- yl)hexanamide | 1020.3 | ¹H NMR (400 MHz, DMSO-d₆) δ 8.43 (br t, J = 6.8 Hz, 1H), 8.37 (br d, J = 8.4 Hz, 1H), 8.25 (br t, J = 5.8 Hz, 1H), 8.12-8.02 (m, 2H), 7.99 (br t, J = 5.6 Hz, 1H), 7.88 (d, J = 11.0 Hz, 1H), 7.32 (s, 2H), 7.26-7.11 (m, 5H), 6.98 (s, 1H), 6.51 (s, 1H), 5.42 (s, 2H), 5.33 (s, 2H), 4.49 (br d, J = 6.6 Hz, 3H), 4.25-4.10 (m, 3H), 3.72-3.64 (m, 5H), 3.58 (br dd, J = 16.5, 5.5 Hz, 3H), 3.02 (br dd, J = 13.5, 4.3 Hz, 1H), 2.87-2.75 (m, 3H), 2.68 (br d, J = 7.5 Hz, 2H), 2.61-2.56 (m, 2H), 2.14- 2.00 (m, 3H), 1.94-1.80 (m, |
| | | | | 2H), 1.71-1.62 (m, 1H), 1.46 (br |
| | | | | dd, J = 14.7, 7.3 Hz, 4H), 1.29- |
| | | | | 1.11 (m, 3H), 0.87 (br t, J = 7.3 |
| | | | | Hz, 3H). |
| 56. | 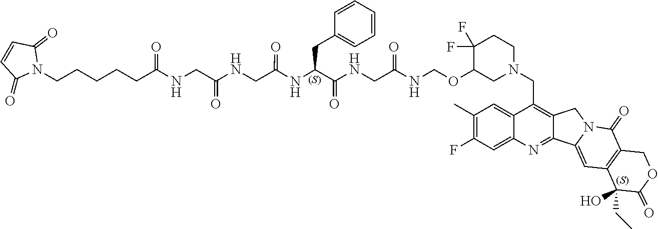 | N-((S)-7-benzyl-1-(((R)-1-(((S)- 4-ethyl-8-fluoro-4-hydroxy-9- methyl-3,14-dioxo-3,4,12,14- tetrahydro-1H- pyrano[3′,4′:6,7]indolizino[1,2- b]quinolin-11-yl)methyl)-4,4- difluoropiperidin-3-yl)oxy)- 3,6,9,12-tetraoxo-2,5,8,11- tetraazatridecan-13-yl)-6-(2,5- dioxo-2,5-dihydro-1H-pyrrol-1- yl)hexanamide | 1070.2 | ¹H NMR (400 MHz, DMSO-d₆) δ 8.62 (br t, J = 6.4 Hz, 1H), 8.41 (br d, J = 8.4 Hz, 1H), 8.28 (br t, J = 4.2 Hz, 1H), 8.12-8.04 (m, 2H), 7.99 (br t, J = 5.8 Hz, 1H), 7.87 (d, J = 10.8 Hz, 1H), 7.32 (s, 1H), 7.23-7.10 (m, 5H), 6.98 (s, 2H), 6.52 (s, 1H), 5.43 (s, 2H), 5.35 (s, 2H), 4.70-4.65 (m, 2H), 4.48-4.42 (m, 1H), 4.20 (q, J = 11.2 Hz, 2H), 3.73-3.47 (m, 7H), 3.45-3.40 (m, 2H), 3.12- 2.98 (m, 1H), 3.00-2.89 (m, 2H), 2.83-2.71 (m, 2H), 2.68- 2.55 (m, 2H), 2.12-2.05 (m, |
| | | | | 3H), 1.90-1.79 (m, 3H), 1.50- |
| | | | | 1.40 (m, 4H), 1.20-1.15 (m, |
| | | | | 2H), 1.05 (t, J = 7.3 Hz, 1H), 0.87 |
| | | | | (t, J = 7.2 Hz, 3H). |

### Example 57

| Ex | Structure | Name |
|---|---|---|
| 57. | 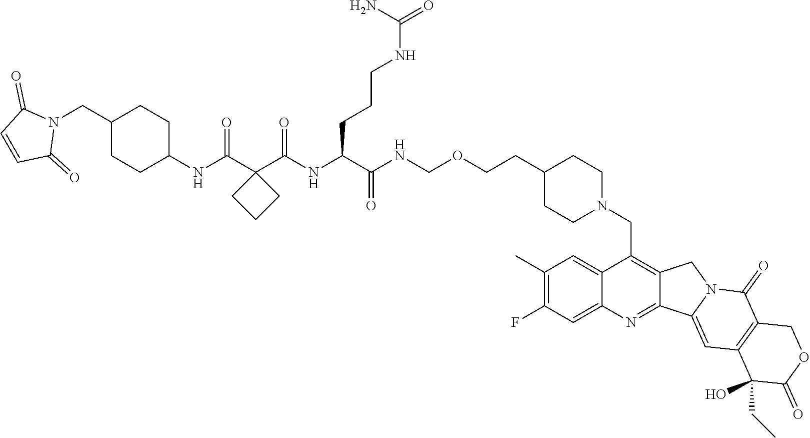 [SMILES: CC[C@@]1(O)C(=O)OCc2c1cc1n(c2=O)Cc2c-1nc1cc(F)c(C)cc1c2CN1CCC(CCOCNC(=O)[C@H](CCCNC(N)=O)NC(=O)C2(C(=O)NC3CCC(CN4C(=O)C=CC4=O)CC3)CCC2)CC1] | N-(4-((2,5-dioxo-2,5-dihydro-1H- pyrrol-1-yl)methyl)cyclohexyl)-N- ((2S)-1-(((2-(1-((4-ethyl-8-fluoro-4- hydroxy-9-methyl-3,14-dioxo- 3,4,12,14-tetrahydro-1H- pyrano[3′,4′:6,7]indolizino[1,2- b]quinolin-11-yl)methyl)piperidin-4- yl)ethoxy)methyl)amino)-1-oxo-5- ureidopentan-2-yl)cyclobutane-1,1- dicarboxamide |
| 58. | 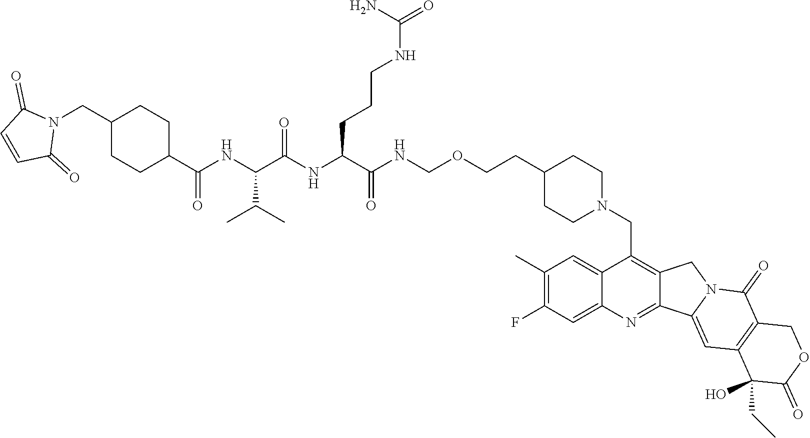 [SMILES: CC[C@@]1(O)C(=O)OCc2c1cc1n(c2=O)Cc2c-1nc1cc(F)c(C)cc1c2CN1CCC(CCOCNC(=O)[C@H](CCCNC(N)=O)NC(=O)[C@@H](NC(=O)C2CCC(CN3C(=O)C=CC3=O)CC2)C(C)C)CC1] | 4-((2,5-dioxo-2,5-dihydro-1H-pyrrol- 1-yl)methyl)-N-((2S)-1-(((2S)-1-(((2- (1-((4-ethyl-8-fluoro-4-hydroxy-9- methyl-3,14-dioxo-3,4,12,14- tetrahydro-1H- pyrano[3′,4′:6,7]indolizino[1,2- b]quinolin-11-yl)methyl)piperidin-4- yl)ethoxy)methyl)amino)-1-oxo-5- ureidopentan-2-yl)amino)-3-methyl- 1-oxobutan-2-yl)cyclohexane-1- carboxamide |
| 59. | 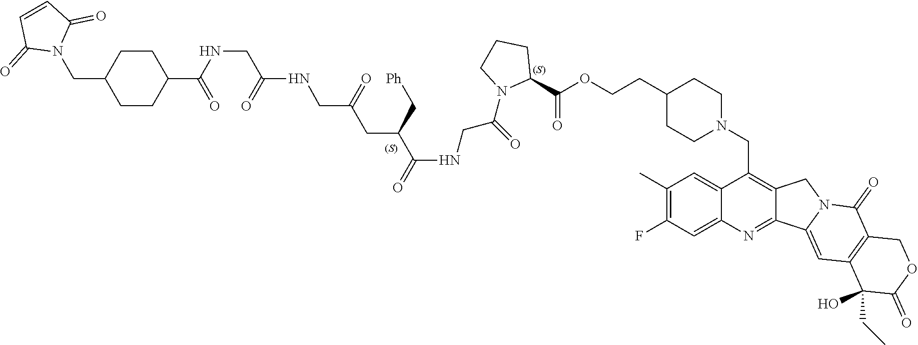 | 2-(1-((4-ethyl-8-fluoro-4-hydroxy-9- methyl-3,14-dioxo-3,4,12,14- tetrahydro-1H- pyrano[3′,4′:6,7]indolizino[1,2- b]quinolin-11-yl)methyl)piperidin-4- yl)ethyl (4-((2,5-dioxo-2,5-dihydro- 1H-pyrrol-1-yl)methyl)cyclohexane- 1-carbonyl)glycylglycyl-L- phenylalanylglycyl-L-prolinate |
| 60. | 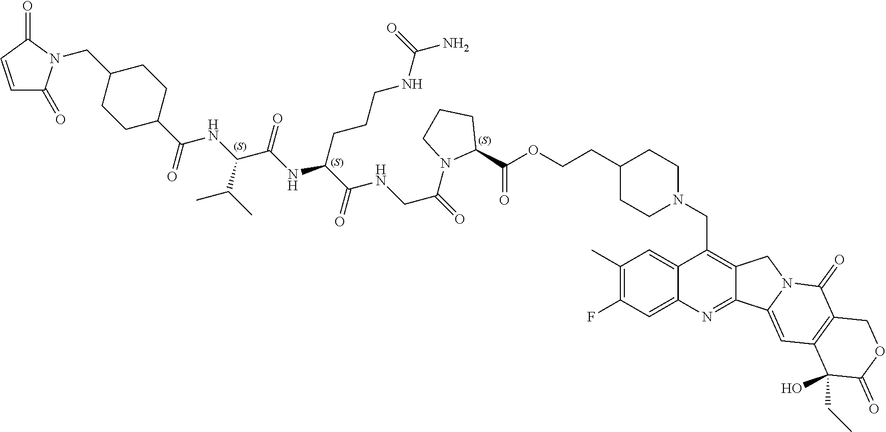 | 2-(1-((4-ethyl-8-fluoro-4-hydroxy-9- methyl-3,14-dioxo-3,4,12,14- tetrahydro-1H- pyrano[3′,4′:6,7]indolizino[1,2- b]quinolin-11-yl)methyl)piperidin-4- yl)ethyl ((S)-2-((S)-2-(4-((2,5-dioxo- 2,5-dihydro-1H-pyrrol-1- yl)methyl)cyclohexane-1- carboxamido)-3-methylbutanamido)- 5-ureidopentanoyl)glycyl-L-prolinate |
| 61. | 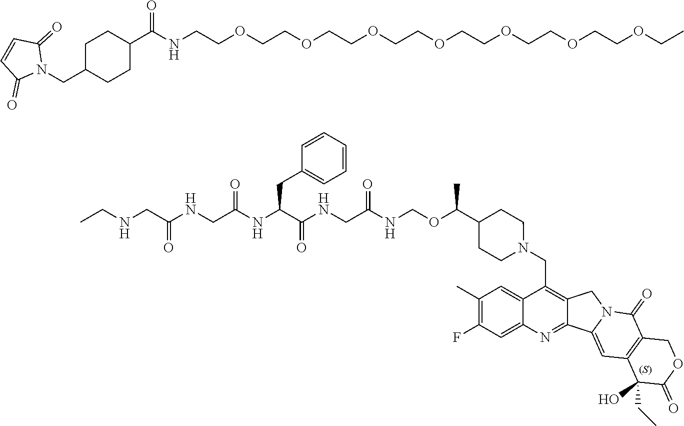 | N-((2S,10S)-10-benzyl-2-(1-(((S)-4- ethyl-8-fluoro-4-hydroxy-9-methyl- 3,14-dioxo-3,4,12,14-tetrahydro-1H- pyrano[3′,4′:6,7]indolizino[1,2- b]quinolin-11-yl)methyl)piperidin-4- yl)-6,9, 12,15-tetraoxo- 3,20,23,26,29,32,35,38-octaoxa- 5,8,11,14,17-pentaazatetracontan-40- yl)-4-((2,5-dioxo-2,5-dihydro-1H- pyrrol-1-yl)methyl)cyclohexane-1- carboxamide |
| 62. | 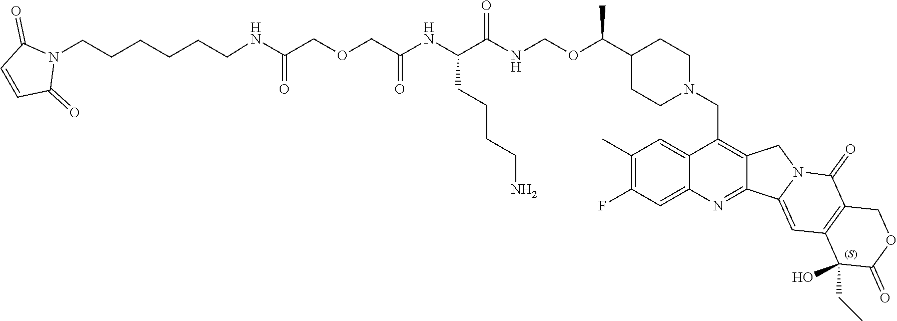 | (S)-6-amino-2-(2-(2-((6-(2,5-dioxo- 2,5-dihydro-1H-pyrrol-1- yl)hexyl)amino)-2- oxoethoxy)acetamido)-N-(((S)-1-(1- (((S)-4-ethyl-8-fluoro-4-hydroxy-9- methyl-3,14-dioxo-3,4,12,14- tetrahydro-1H- pyrano[3′,4′:6,7]indolizino[1,2- b]quinolin-11-yl)methyl)piperidin-4- yl)ethoxy)methyl)hexanamide |
| 63. | 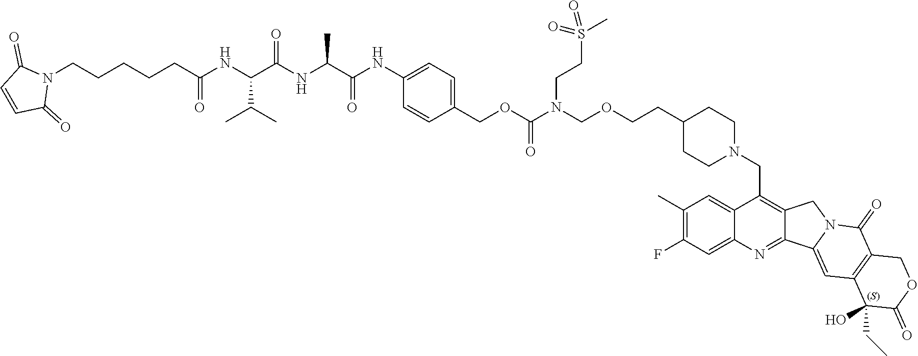 | 4-((S)-2-((S)-2-(6-(2,5-dioxo-2,5- dihydro-1H-pyrrol-1- yl)hexanamido)-3- methylbutanamido)propanamido) benzyl ((2-(1-(((S)-4-ethyl-8-fluoro-4- hydroxy-9-methyl-3,14-dioxo- 3,4,12,14-tetrahydro-1H- pyrano[3′,4′:6,7]indolizino[1,2- b]quinolin-11-yl)methyl)piperidin-4- yl)ethoxy)methyl)(2- (methylsulfonyl)ethyl)carbamate |

### Example 61

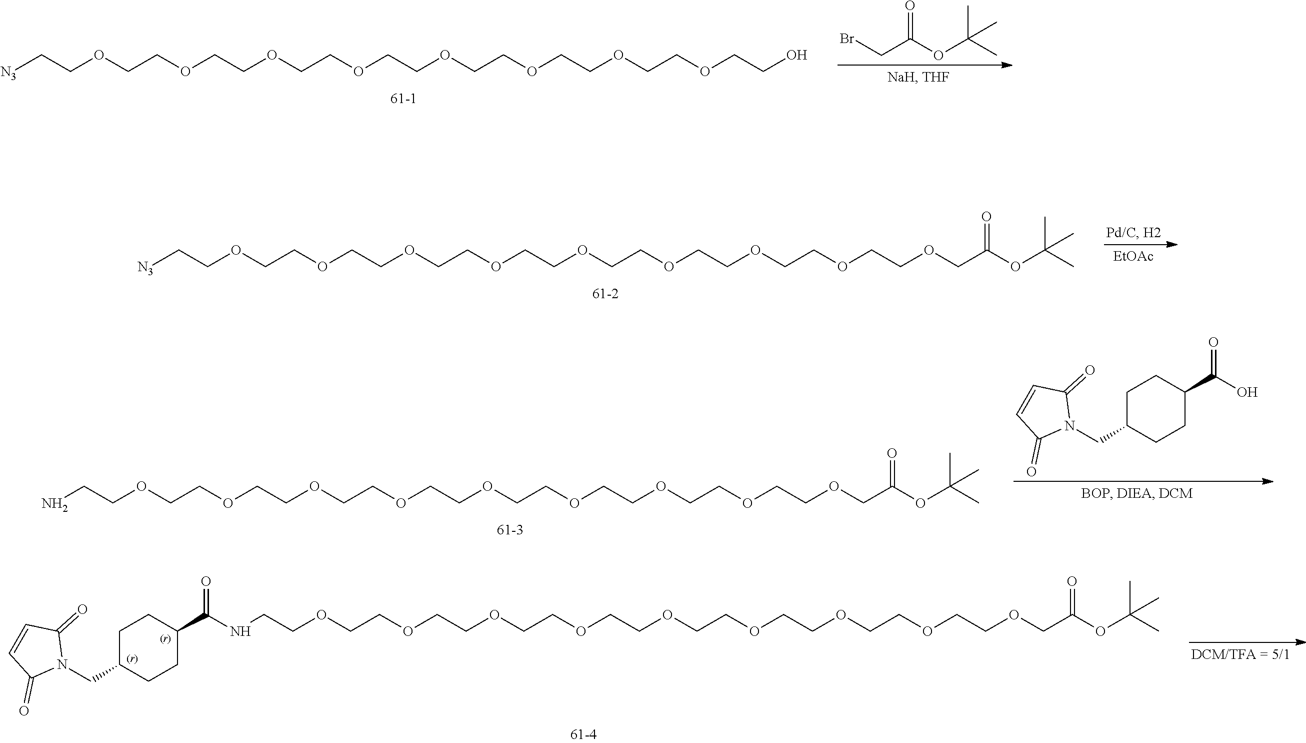

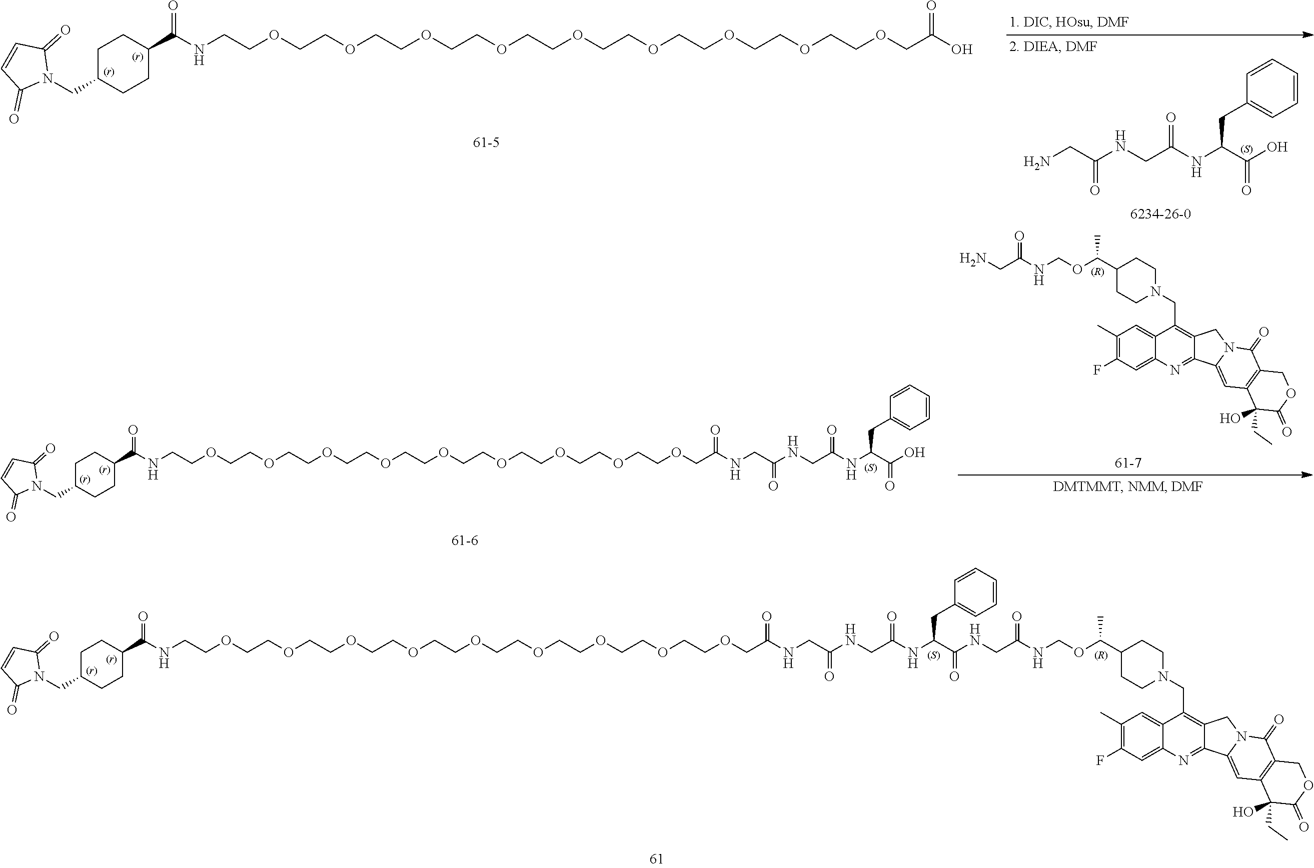

61-7 was prepared by the same method as 49-7

To a solution of 61-1 (1.90 g, 4.32 mmol) in THF (30.0 mL) was added NaH (0.260 g, 6.48 mmol) at 0° C. and the mixture was stirred for 1 hr before tert-butyl 2-bromoacetate (1.01 g, 5.19 mmol) was added. The reaction mixture was warmed to 25° C. slowly and stirred for 2 hrs. TLC (EA) showed the SM was consumed completely and one main spot was detected. The reaction mixture was quenched by saturated solution of NH₄Cl (50 mL) at 25° C., diluted with H₂O (20 mL) and extracted with EtOAc (30 mL×3). The combined organic layers were washed with brine (20 mL×2), dried over Na₂SO₄, filtered and the filtrate was concentrated under reduced pressure. The residue was purified by SGC (Petroleum ether/Ethyl acetate=100/1 to 0/1) to obtain 61-2 (1.30 g, 2.35 mmol, 54.3%) as yellow oil.

To a solution of 61-2 (1.00 g, 1.81 mmol) in EtOAc (10.0 mL) was added Pd/C (10%, 0.100 g, 0.903 mmol) under N₂. The suspension was degassed under vacuum and purged with H₂ for several times. The reaction mixture was stirred under H₂ (0.0400 g, 18.1 mmol) (15 psi) at 25° C. for 4 hours. LCMS showed the SM was consumed completely and desired MS was detected. The reaction mixture was filtered and the filtrate was concentrated under reduced pressure to give crude 61-3 (900 mg, 1.71 mmol, 94.4%) as yellow oil. LC-MS: 528.4 [M+H]⁺.

To a mixture of 61-3 (900 mg, 1.706 mmol) and (1r,4r)-4-[(2,5-dioxo-2,5-dihydro-1H-pyrrol-1-yl)methyl]cyclohexane-1-carboxylic acid (486 mg, 2.05 mmol) in DCM (10.0 mL) was added DIEA (0.9 mL, 661 mg, 5.12 mmol) and BOP (1.13 g, 2.56 mmol) at 25° C. and the reaction mixture was stirred for 12 hrs. LCMS showed the SM was consumed completely and desired MS was detected. The reaction mixture was concentrated under reduced pressure. The residue was purified by prep-HPLC (water(FA)-ACN, Phenomenex luna C18 (250*70 mm, 15 um)) to obtain 61-4 (400 mg, 0.536 mmol, 31.4%) as a yellow oil. LC-MS: 747.35 [M+H]⁺.

A solution of 61-4 (400 mg, 0.536 mmol) in DCM (5.00 mL) and TFA (1.00 mL) was stirred at 25° C. for 12 hrs. LCMS showed the SM was consumed completely and desired MS was detected. The reaction mixture was concentrated under reduced pressure to obtain 61-5 (370 mg, 0.536 mmol, crude) as a yellow oil. LC-MS: 691.35 [M+H]⁺.

To a solution of 61-5 (135 mg, 0.195 mmol) and HOSu (22.5 mg, 0.195 mmol) in DMF (5.00 mL) was added DIC (24.7 mg, 0.195 mmol) at 25° C. The mixture was stirred for 12 hrs before being filtered. To the filtrate was added DIEA (50.5 mg, 0.391 mmol) and 61-6 (54.6 mg, 0.195 mmol) at 25° C. and the reaction mixture was stirred for 12 hrs. LCMS showed the SM was consumed completely and desired MS was detected. The reaction mixture was purified by prep-HPLC (water(FA)-ACN, Phenomenex Luna C18 75*30 mm*3 um) to obtain 61-7 (100 mg, 0.105 mmol, 53.7%) as a yellow oil. LC-MS: 952.4 [M+H]⁺.

To a mixture of 61-7 (35.0 mg, 0.037 mmol) and 61-6 (22.3 mg, 0.037 mmol) in DMF (3.00 mL) was added NMM (0.012 mL, 0.110 mmol) and DMTMMT (12.7 mg, 0.040 mmol) at 25° C. and the reaction mixture was stirred for 12 hrs. LCMS showed the SM was consumed completely and desired MS was detected. The reaction mixture was purified by prep-HPLC (water(FA)-ACN, Phenomenex Luna C18 75*30 mm*3 um). 61(20.0 mg, 0.012 mmol, 32.6%) was obtained as an off-white solid.

The compounds below were synthesized following procedures described for Example 61.

| Ex. | Structure | Name | [M + H]⁺ | ¹H NMR |
|---|---|---|---|---|
| 61. | 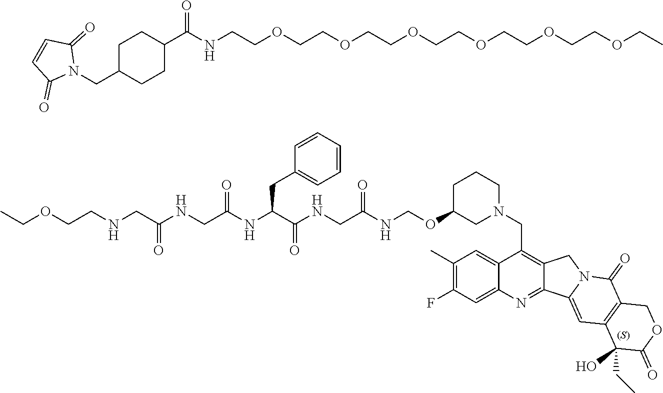 | N-((2S,10S)-10-benzyl-2-(1- (((S)-4-ethyl-8-fluoro-4- hydroxy-9-methyl-3,14-dioxo- 3,4,12,14-tetrahydro-1H- pyrano[3′,4′:6,7]indolizino[1,2- b]quinolin-11- yl)methyl)piperidin-4-yl)- 6,9,12,15-tetraoxo- 3,20,23,26,29,32,35,38-octaoxa- 5,8,11,14,17- pentaazatetracontan-40-yl)-4- ((2,5-dioxo-2,5-dihydro-1H- pyrrol-1-yl)methyl)cyclohexane- 1-carboxamide | 1542.1 | ¹H NMR (400 MHz, DMSO-d₆) δ 8.49-8.33 (m, 2H), 8.26 (br t, J = 5.7 Hz, 1H), 8.13-8.04 (m, 2H), 7.92-7.85 (m, 2H), 7.74-7.68 (m, 1H), 7.32 (s, 1H), 7.26-7.13 (m, 5H), 7.01 (s, 1H), 6.52 (s, 1H), 5.43 (s, 2H), 5.33 (s, 2H), 4.56- 4.45 (m, 3H), 4.05 (br s, 2H), 3.92 (s, 2H), 3.79-3.66 (m, 6H), 3.64- 3.54 (m, 7H), 3.50 (s, 28H), 3.23 (br d, J = 7.1 Hz, 2H), 3.15 (q, J = 5.7 Hz, 2H), 3.03 (br dd, J = 13.7, 4.2 Hz, 1H), 2.93-2.85 (m, 2H), 2.77 (br dd, J = 13.8, 9.7 Hz, 1H), 2.16-1.97 (m, 4H), 1.92-1.81 (m, 2H), 1.73-1.57 (m, 6H), 1.50 (br d, J = 8.0 Hz, 2H), 1.32-1.15 (m, 6H), 1.03 (br d, J = 6.1 Hz, 3H), 0.88 (br t, J = 7.3 Hz, 6H). |
| 64. | 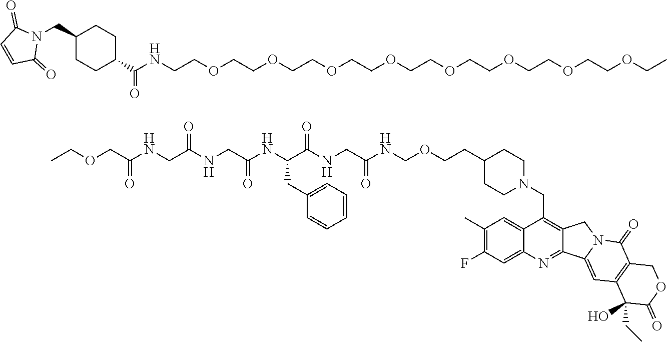 | N-((10S)-10-benzyl-1-(1-((4- ethyl-8-fluoro-4-hydroxy-9- methyl-3,14-dioxo-3,4,12,14- tetrahydro-1H- pyrano[3′,4′:6,7]indolizino[1,2- b]quinolin-11- yl)methyl)piperidin-4-yl)- 6,9,12,15,18-pentaoxo- 3,20,23,26,29,32,35,38,41,44- decaoxa-5,8,11,14,17- pentaazahexatetracontan-46-yl)- 4-((2,5-dioxo-2,5-dihydro-1H- pyrrol-1-yl)methyl)cyclohexane- 1-carboxamide | 1541.6 | ¹H NMR (400 MHz, DMSO-d₆) δ 8.44 (br t, J = 6.6 Hz, 1H), 8.36 (br d, J = 8.4 Hz, 1H), 8.27 (br t, J = 5.8 Hz, 1H), 8.11 (br d, J = 8.1 Hz, 1H), 8.06 (br t, J = 5.6 Hz, 1H), 7.92-7.84 (m, 2H), 7.71 (br t, J = 5.5 Hz, 1H), 7.34-7.29 (m, 1H), 7.27-7.20 (m, 4H), 7.19- 7.14 (m, 1H), 7.00 (s, 1H), 6.51 (s, 1H), 5.42 (s, 2H), 5.32 (s, 2H), 4.53-4.45 (m, 3H), 4.04 (br s, 2H), 3.92 (s, 2H), 3.84 (s, 2H), 3.78-3.71 (m, 5H), 3.69 (br d, J = 7.9 Hz, 1H), 3.64-3.59 (m, 4H), 3.58-3.54 (m, 3H), 3.53-3.45 (m, 28H), 3.39-3.36 (m, 3H), 3.22 (d, J = 7.1 Hz, 2H), 3.15 (q, J = 5.8 Hz, 2H), 3.04 (br dd, J = 13.8, 4.4 Hz, 1H), 2.84 (br dd, J = 6.4, 1.8 Hz, 2H), 2.75 (s, 1H), 2.16-2.07 (m, 2H), 2.05-1.97 (m, 1H), 1.92-1.78 (m, 2H), 1.67 (br |
| | | | | d, J = 12.5 Hz, 2H), 1.61 (br d, J = |
| | | | | 12.8 Hz, 4H), 1.55-1.47 (m, 1H), |
| | | | | 1.40 (br d, J = 5.1 Hz, 3H), 1.31- |
| | | | | 1.19 (m, 2H), 1.17-1.05 (m, 2H), |
| | | | | 0.94-0.83 (m, 5H). |
| 65. | 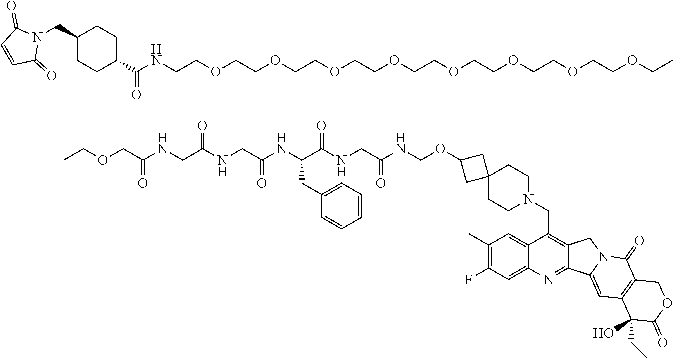 | N-((7S)-7-benzyl-1-((7-((4- ethyl-8-fluoro-4-hydroxy-9- methyl-3,14-dioxo-3,4,12,14- tetrahydro-1H- pyrano[3′,4′:6,7]indolizino[1,2- b]quinolin-11-yl)methyl)-7- azaspiro[3.5]nonan-2-yl)oxy)- 3,6,9,12,15-pentaoxo- 17,20,23,26,29,32,35,38,41- nonaoxa-2,5,8,11,14- pentaazatritetracontan-43-yl)-4- ((2,5-dioxo-2,5-dihydro-1H- pyrrol-1-yl)methyl)cyclohexane- 1-carboxamide | 1554.5 | ¹H NMR (400 MHz, DMSO-d₆) δ 8.46-8.35 (m, 2H), 8.32-8.26 (m, 1H), 8.17-8.04 (m, 2H), 7.93- 7.84 (m, 2H), 7.74-7.66 (m, 1H), 7.32 (s, 1H), 7.28-7.20 (m, 4H), 7.20-7.13 (m, 1H), 7.01 (s, 2H), 6.52 (s, 1H), 5.43 (s, 2H), 5.33 (s, 2H), 4.56-4.40 (m, 3H), 4.08-3.98 (m, 3H), 3.93 (s, 2H), 3.79-3.66 (m, 5H), 3.65-3.59 (m, 3H), 3.59-3.55 (m, 2H), 3.54- 3.45 (m, 31H), 3.40-3.35 (m, 2H), 3.23 (d, J = 7.1 Hz, 2H), 3.16 (q, J = 6.0 Hz, 2H), 3.06 (br dd, J = 13.8, 4.0 Hz, 1H), 2.79 (br dd, J = 13.8, 9.7 Hz, 1H), 2.44-2.37 (m, 3H), 2.18-2.09 (m, 2H), 2.07- 1.97 (m, 1H), 1.94-1.80 (m, 2H), 1.73-1.65 (m, 2H), 1.63- 1.55 (m, 4H), 1.49 (br d, J = 3.4 Hz, 5H), 1.35-1.18 (m, 3H), 0.94- 0.83 (m, 5H) |
| 66. | 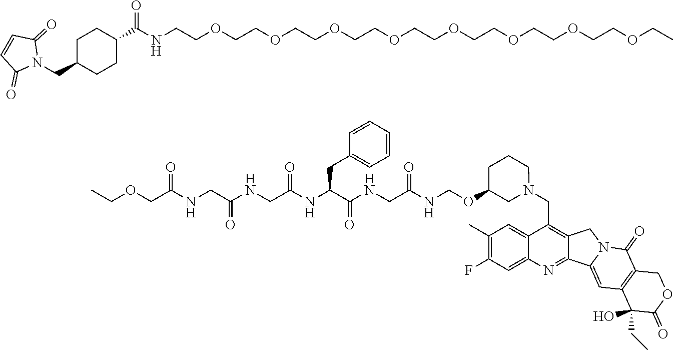 | N-((7S)-7-benzyl-1-(((3S)-1-((4- ethyl-8-fluoro-4-hydroxy-9- methyl-3,14-dioxo-3,4,12,14- tetrahydro-1H- pyrano[3′,4′:6,7]indolizino[1,2- b]quinolin-11- yl)methyl)piperidin-3-yl)oxy)- 3,6,9,12,15-pentaoxo- 17,20,23,26,29,32,35,38,41- nonaoxa-2,5,8,11,14- pentaazatritetracontan-43-yl)-4- ((2,5-dioxo-2,5-dihydro-1H- pyrrol-1-yl)methyl)cyclohexane- 1-carboxamide | 1514.5 | ¹H NMR (400 MHz, DMSO-d₆) δ 8.48-8.35 (m, 2H), 8.31-8.22 (m, 1H), 8.14-8.01 (m, 2H), 7.94- 7.84 (m, 2H), 7.71 (br t, J = 5.5 Hz, 1H), 7.31 (s, 1H), 7.26-7.14 (m, 5H), 7.00 (s, 2H), 6.51 (s, 1H), 5.42 (s, 2H), 5.34 (s, 2H), 4.61- 4.43 (m, 3H), 4.21-4.01 (m, 2H), 3.94-3.88 (m, 2H), 3.76-3.69 (m, 3H), 3.67-3.64 (m, 1H), 3.61- 3.54 (m, 6H), 3.49 (s, 31H), 3.38- 3.34 (m, 2H), 3.22 (d, J = 7.0 Hz, 2H), 3.17-3.13 (m, 2H), 3.01 (br dd, J = 13.7, 4.2 Hz, 1H), 2.94 (br d, J = 8.0 Hz, 1H), 2.76 (br dd, J = 13.5, 9.6 Hz, 1H), 2.60 (br d, J = 11.4 Hz, 1H), 2.22-2.09 (m, 2H), 2.07-1.96 (m, 1H), 1.86 (qd, J = 14.6, 7.2 Hz, 3H), 1.71-1.58 (m, 5H), 1.50 (ddd, J = 11.1, 7.3, 3.5 Hz, 1H), 1.39-1.31 (m, 1H), 1.30- 1.14 (m, 4H), 0.94-0.80 (m, 5H) |

### Example 67: Preparation of Antibody-Drug Conjugates (e.g., ADC-1)

### Antibodies for Exemplary ADCs

Antibodies for the examples' ADC compounds were prepared according to conventional methods, for example, vector construction, eukaryotic cell transfection such as HEK2943 cell (Life Technologies Cat. No. 11625019) transfection, purification, and expression. Antibodies prepared included trastuzumab light chain (SEQ. ID NO. 1), trastuzumab heavy chain (SEQ. ID NO. 2), pertuzumab light chain (SEQ. ID NO. 3), pertuzumab heavy chain (SEQ. ID NO. 4), B7H3 antibody light chain (SEQ. ID NO. 5), and B7H3 antibody heavy chain (SEQ. ID NO. 6).

### General Process of Conjugation

A formulated aqueous solution of tris(2-carboxy-ethyl) phosphine (10 mM, 0.082 mL, 0.82 μmol) was added to a PBS-buffered aqueous solution of antibody (0.05 M PBS-buffered aqueous solution with pH=6.5; 2.5 ml, 9.96 mg/ml, 0.168 umol) at 37° C. The reaction solution was placed in a water bath shaker and shaken at 37° C. for 3 hours before stopping the reaction. The reaction solution was cooled to 25° C. in a water bath and diluted to 5.0 mg/ml. 2.0 ml of the solution was taken for the next reaction.

The linker-camptothecin compound (2.1 mg, 2.02 umol) was dissolved in 0.10 mL of DMSO, and then added to 2.0 ml of the above solution. The reaction solution was placed in a water bath shaker, and shaked at 25° C. for 3 hours before stopping the reaction. The reaction solution was desalted and purified with a Sephadex G25 gel column (elution phase: 0.05 M PBS-buffered aqueous solution with pH=6.5, containing 0.001 M EDTA) to obtain the PBS-buffered solution of the exemplary product ADC, which was stored at 4° C.

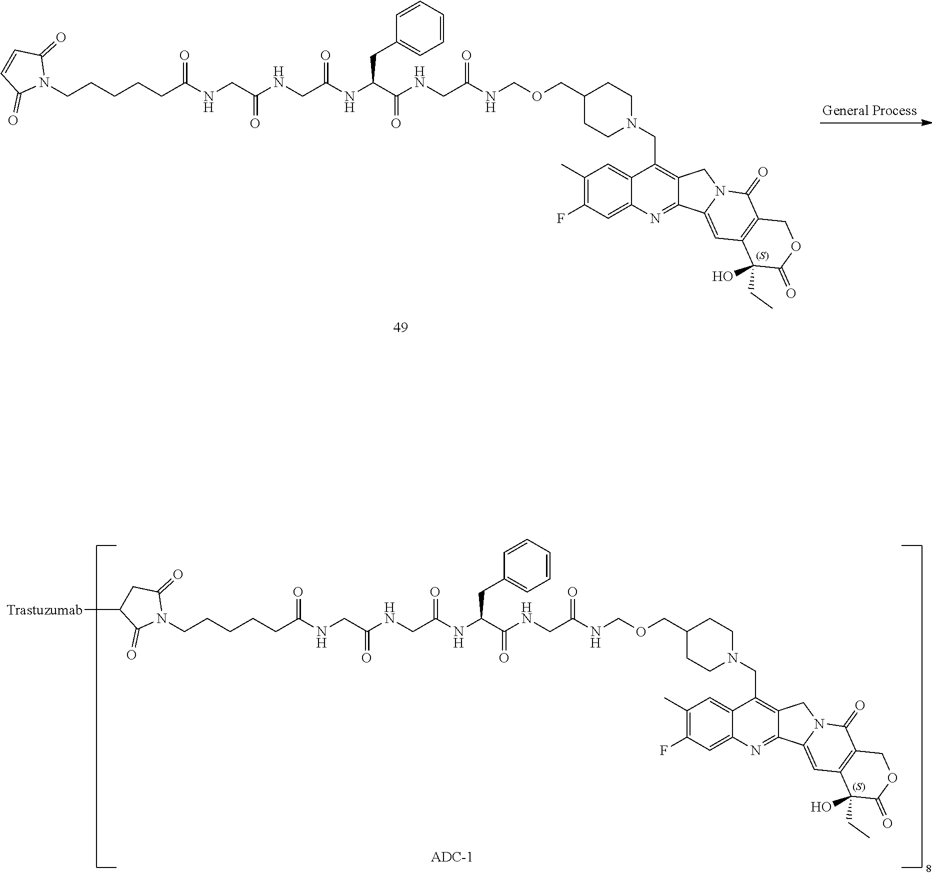

The analysis of drug loading of the ADC (UV method) was carried out according to the method of U.S. Patent Application Publication No. US 2021/0353764 (i.e., paragraphs [0702]ff).

The analysis of drug loading of the ADC (LC-MS method) was carried out according to the method of U.S. Pat. No. 11,572,414 (i.e., col. 2, line 51, to col. 3, line 15).

The ADC aggregation levels was determined by Size Exclusion Chromatography (SEC). All samples were filtered through 0.22 μm filter prior to HPLC-SEC analysis.

The HPLC method was conducted as follows:
Instrument: Thermo Ultimate 3000
Column: Waters, XBridge BEH 200 Å SEC 3.5 μm (7.8×300 mm)
Mobile Phase: PBS with 15% isopropanol, pH 7.4
Flow Rate: 0.5 ml/min, 30 min.

The ADCs below were synthesized following the general procedures described in Example 67.

| | | Payload | | SEC | LC- | |
|---|---|---|---|---|---|---|
| ADC | | Example | Yield | Aggregation | MS- | UV- |
| Name | Structure | # | (%) | (%) | DAR | DAR |
| Com- par- ator ADC-2 | 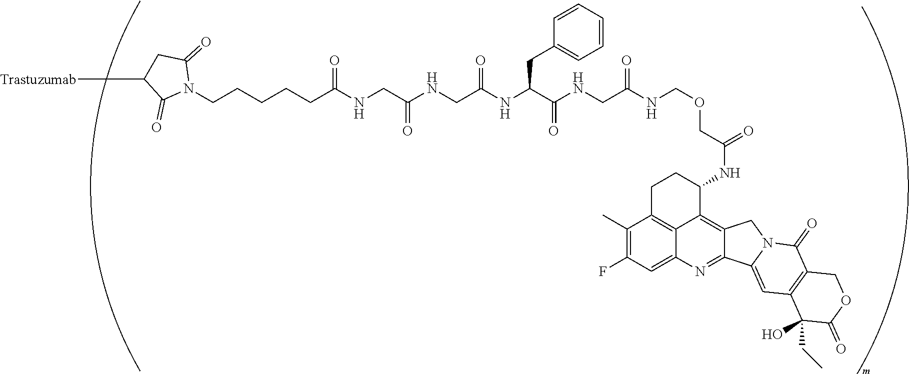 | Dxd | 80.0% | 3.04% | 8.0 | / |
| ADC-3 | 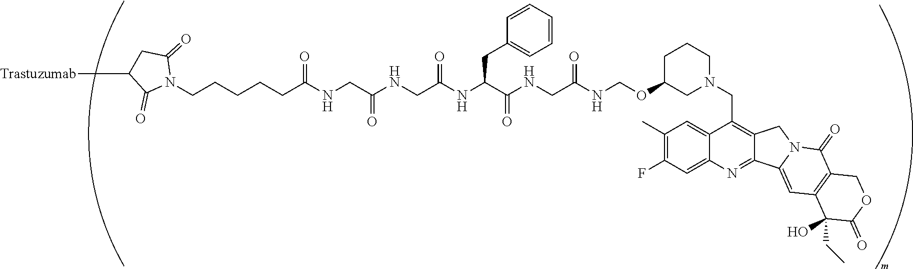 | 19 | 86.4% | 2.55% | 7.5 | / |
| ADC-4 | 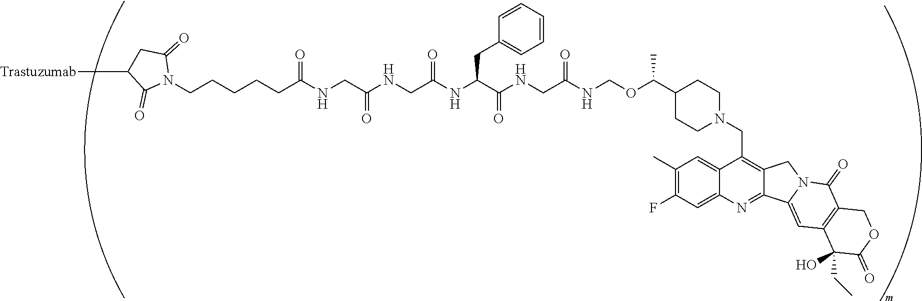 | 15 | 60.7% | 21.23% | 5.2 | / |
| ADC-5 | 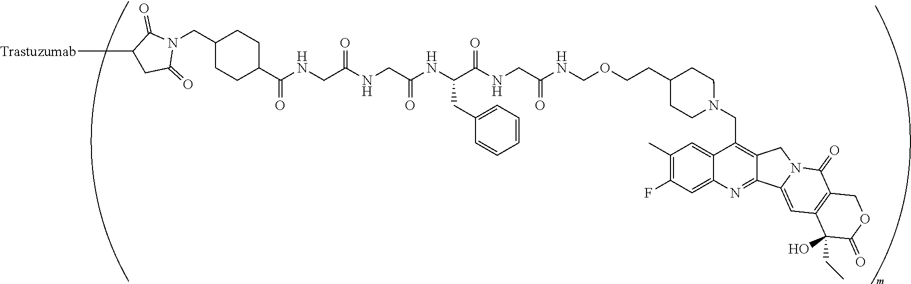 | 27 | 53.0% | 76.01% | / | 6.7 |
| ADC-6 | 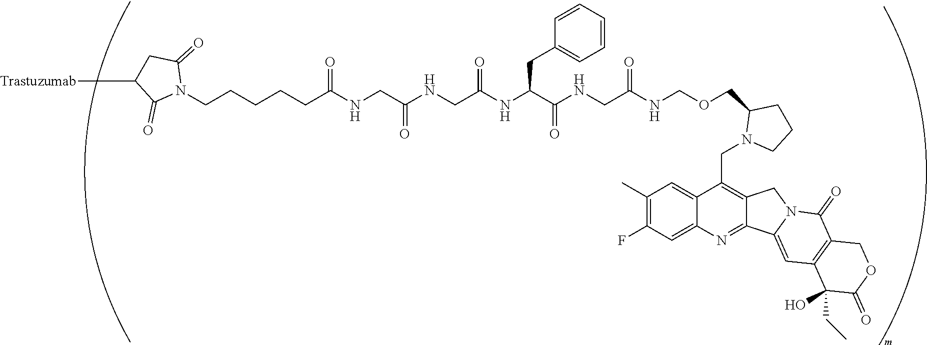 | 7 | 54.1% | 3.25% | 7.3 | / |
| ADC-7 | 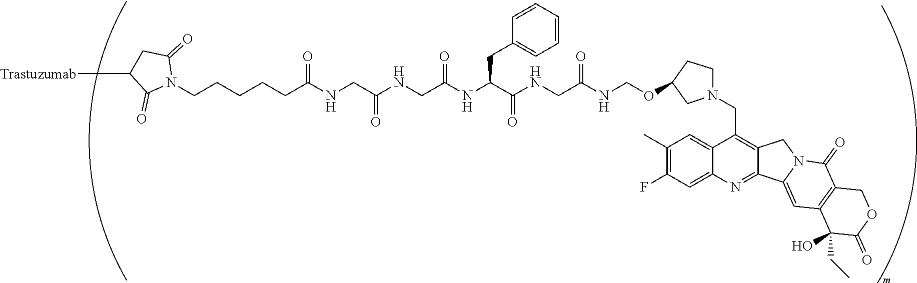 | 3 | 55.9% | 3.76% | 7.5 | / |
| ADC-8 | 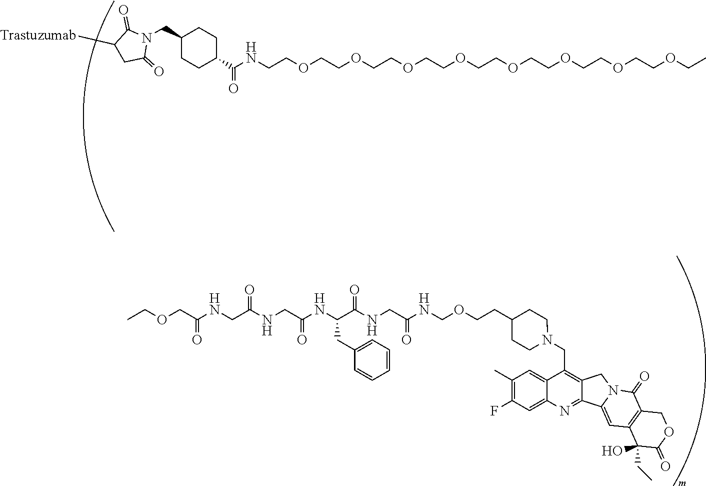 | 27 | 63.4% | 2.87% | 7.9 | / |
| ADC-9 | 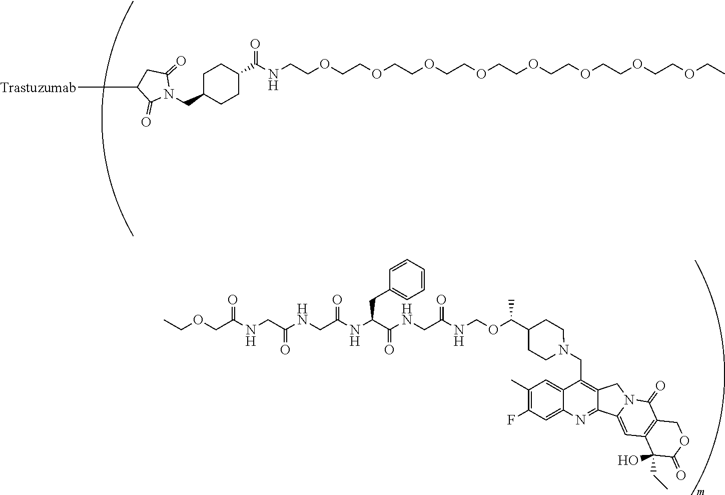 | 15 | 65.3% | 2.93% | 7.3 | / |

### Example 68

### In Vitro Cytotoxicity Test of Camptothecin Payloads

Human lung adenocarcinoma cell line A549 was used to evaluate the cytotoxicity of small molecule fragment of the present invention. A549 cells were seeded to a 96-well plate at 2000 cells per well. After overnight incubation under 5% CO₂ and 37° C., each diluted substance was added. Cell viability was evaluated after 3 days using a CellTiter-Glo luminescent cell viability assay from Promega Corp. and according to the manufacturer's instructions. The results are shown in Table 1 below.

TABLE 1
Cytotoxicity on A549 by Compounds of Formula (VI)

| | A549 Cytotoxicity |
|---|---|
| Example # | IC₅₀ (nM) |
| 1 | 515 |
| 2 | 138 |
| 5 | 65 |
| 8 | 82 |
| 9 | 550 |
| 10 | 67 |
| 11 | 60 |
| 12 | 90 |
| 13 | 33 |
| 15 | 17.6 |
| 16 | 81 |
| 17 | 38 |
| 18 | 19.7 |
| 19 | 15.7 |
| 20 | 24.1 |
| 21 | 55 |
| 22 | 14.3 |
| 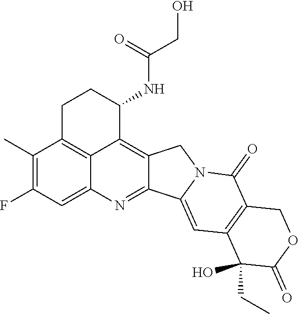 [SMILES: CC[C@@]1(O)C(=O)OCc2c1cc1n(c2=O)Cc2c-1nc1cc(F)c(C)c3c1c2[C@@H](NC(=O)CO)CC3] | 78 |
| Dxd | |
| 23 | 49 |
| 24 | 43 |
| 27 | 16.8 |
| 28 | 74.2 |
| 29 | 29.8 |
| 30 | 28.6 |
| 31 | 12.5 |
| 32 | 12.6 |
| 33 | 12.9 |
| 36 | 38.4 |
| 37 | 29 |
| 38 | 1247 |
| 39 | 128 |
| 40 | 1290 |
| 41 | 31 |
| 44 | 30 |
| 45 | 53 |
| 47 | 28 |

As shown in Table 1, the potency of compounds of Example 15, 19, 22, 27, 31, 32, 33 is 5˜6× higher than the comparator compound (Dxd). Surprisingly, some minor structural changes can significantly impact the cytotoxicity. For example, despite the amide moiety being well tolerated in Dxd, the lactam analog of Example 1 is much less potent than most of the other amine analogs. The 3-OH substituted piperidine compound of Example 17 is the most potent compound among the other four-, five- or six-membered analogs of Example 2, 9 and 16. The piperidine compounds of Example 10, 11 and 12 with —CH₂OH substitution are also less potent than Example 17. Moreover, the S-isomer of Example 17, Example 19, is also more potent than Example 17. Increasing the lipophilicity, which potentially increases the cellular permeability, may also improve the potency, as shown in Example 15, 27, 31, 32 and 33. However, these molecules may also have lower solubility, which may cause increase of aggregation during the conjugation with antibody, as evidenced by the high aggregation and low DAR value in the synthesis of ADC-4 and ADC-5. Although the aggregation issue could be improved by employing a hydrophilic PEG-like linker, this increases the difficulty of manufacturing and may impair the potency. For example, Example ADC-8 and ADC-9 are less potent than Example ADC-3 in Table 2.

### Example 69

### In Vitro Cytotoxicity Test of ADCs

Cancer cell lines with different level of Her2 expression, including NCI-N87, Calu-3, SK-BR-3, CAPAN-1 and CFPAC-1 cells, were used to test the cytotoxicity of ADC of the present invention. These cell lines were seeded to a 96-well plate at 1000-4000 cells per well. After overnight incubation under 5% CO₂ and 37° C., each diluted substance was added. Cell viability was evaluated after 6 days using a CellTiter-Glo luminescent cell viability assay from Promega Corp. and according to the manufacturer's instructions. The results are shown in Table 2 below.

TABLE 2
In vitro cytotoxicity test of ADCs

| | | Cytotoxicity IC₅₀, nM | | | | |
|---|---|---|---|---|---|---|
| | LC-MS- | SK-BR- | Calu- | NCI- | CAPAN- | CFPAC- |
| Example | DAR | 3 | 3 | N87 | 1 | 1 |
| ADC-2 | 8.0 | 1.2 | 2.9 | 3.2 | 40.7 | 122.3 |
| ADC-3 | 7.5 | 1.0 | 2.9 | 1.6 | 10.7 | 24.9 |
| ADC-6 | 7.3 | / | / | 5.7 | / | / |
| ADC-8 | 7.9 | / | / | 8.8 | / | / |
| ADC-9 | 7.3 | / | / | 6.0 | / | / |

Calu-3, NCI-N87 and SK-BR-3 are all Her2 high-expression cancer cell lines, CAPAN-1 and CFPAC-1 are reported as Her2 low-expression cell lines. The ADC of the present invention, such as ADC-3, displays comparable cytotoxicity in the Her2 high-expression cancer cell lines, but surprisingly with significantly higher potency in the Her2 low-expression cell lines. Among the similar analogs, ADC-3 is the most potent one against NCI-N87 cell line.

### Example 70

### In Vitro Bystander Killing of ADC

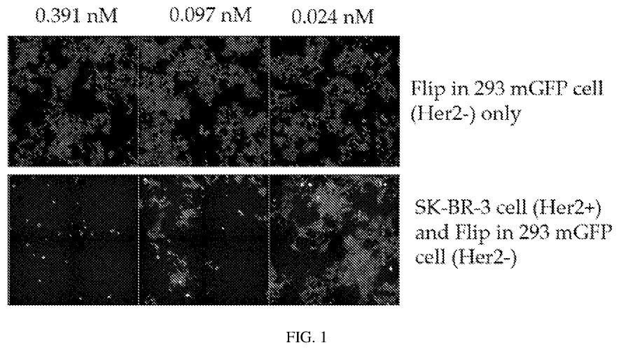

Target cell SK-BR-3 and GFP-labelled tool cell Flip in 293 mGFP, plated either individually or mixed and cultured for 1 day, were treated with 4-fold serial dilution of ADC-3 solution for 5 days. (The mixed cells were prepared by mixing these two cells and seeding 40 μL/well with a final density of 750 cells/well for SK-BR-3 and 250 cells/well for Flip in 293 mGFP, respectively.) At the end of treatment, the number of live Flip in 293 mGFP cells was determined by High-Content Screening (HCS) Assays in DPC and FITC channel. As shown in FIG. 1, the tool cell Flip in 293 mGFP stably expresses green fluorescent protein (GFP). When plated individually, no significant decrease in Flip in 293 mGFP cell count was observed, but when cells were plated in co-culture, cell counts for Flip in 293 mGFP were reduced at the same concentration of ADC, demonstrating a bystander killing effect.

### Example 71

### In Vitro Plasma Stability Study of ADC

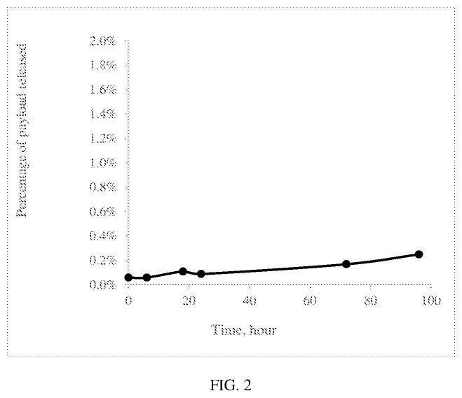

A solution of ADC-3 was prepared at 1.6 mg/mL and added to human plasma, and the mixture was cultured at 37° C. A 40 uL sample was taken at each time point of 0, 6, 18, 24, 72, and 96 hours. After normal processing, the plasma samples were tested for the free payload by a LCMSMS method. As shown in FIG. 2, less than 0.5% of the payload was released from the ADC in plasma up to 4 days, demonstrating good stability of the ADCs of present invention in human plasma.

The LCMSMS method had the following features:
Instrument: Thermo Orbitrap Exploris240
Column: ACQUITY UPLC® BEH C18 1.7 μm (2.1×150 mm)
Mobile Phase: 0.1% FA in ACN
Flow Rate: 0.3 ml/min

### Example 72

### In Vivo Efficacy Study of ADCs

The NCI-N87 cell line was used to create the CDX (Cell Line Derived Xenograft) NCI-N87 xenograft mouse model. Each 6-8 weeks old nu/nu nude mouse was subcutaneously injected into the right flank with 107 cells in 200 μL of a Matrigel-NCI-N87 cell suspension. The injection sites were palpated up to three times weekly until tumors are established to an average size of 300 mm³ as measured via digital calipers. Animals were randomized into treatment groups. Both ADC-2 and ADC-3 were administrated by i.v. injection once at 3 mg/kg. Tumor size was measured and recorded weekly.

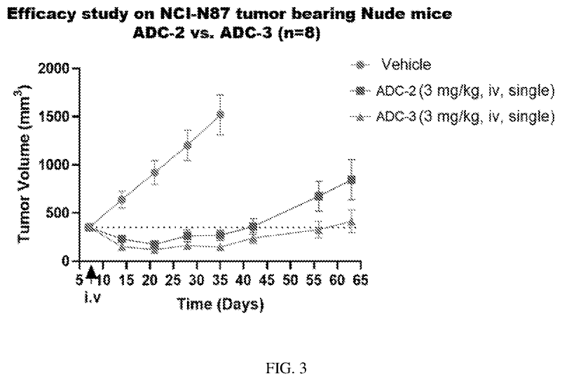

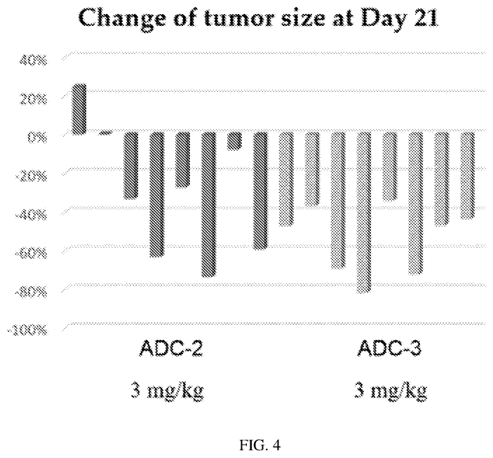

As shown in FIG. 3, treatment with ADC greatly suppresses the tumor growth after treatment. Despite the comparable in vitro cytotoxity, ADC-3 displayed better efficacy in this NCI-N87 mice model than ADC-2. With one single dose of treatment, mice in the ADC-3 group achieve stable disease for almost 2 months, much longer than that in ADC-2 group. Importantly, in FIG. 4, the mice in the ADC-3 group (the last eight bars in the graph) had higher rate of response and better tumor regression at day 21 than the ADC-2 group (the first eight bars in the graph). No progression of tumor was observed in the ADC-3 group. Therefore, this dramatic improvement of in vivo efficacy for the compound of Example ADC-3 is entirely unexpected and very surprising.

The representative data (FIGS. 3 and 4) taken in their entirety reveal a surprisingly superior therapeutic potential for the compounds described herein, with the beneficial unexpected advantages in areas of potency, efficacy, and bystander killing. The dramatic and surprising improvement in in vivo efficacy for ADCs provided herein offers marked benefits for human or mammal therapy, including but not limited to better clinical cure rate, a reduced effective drug dose, and reduced possible adverse effects.

1. A ligand-drug conjugate, or a pharmaceutically acceptable salt or solvate thereof, wherein the ligand-drug conjugate comprises a structure of formula (IIa) 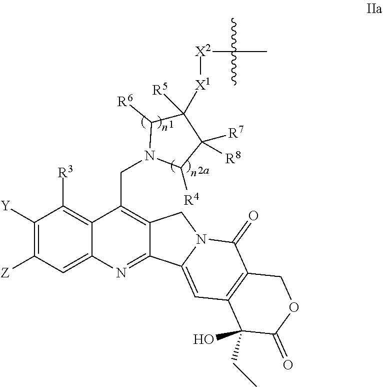
wherein:
Y and Z are independently selected from H halo, C₁-C₈ alkyl, and C₁-C₈ alkoxy;
R³ is selected from H, halo, C₁-C₈ alkyl, and C₁-C₈ alkoxy; 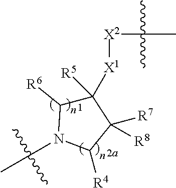
is

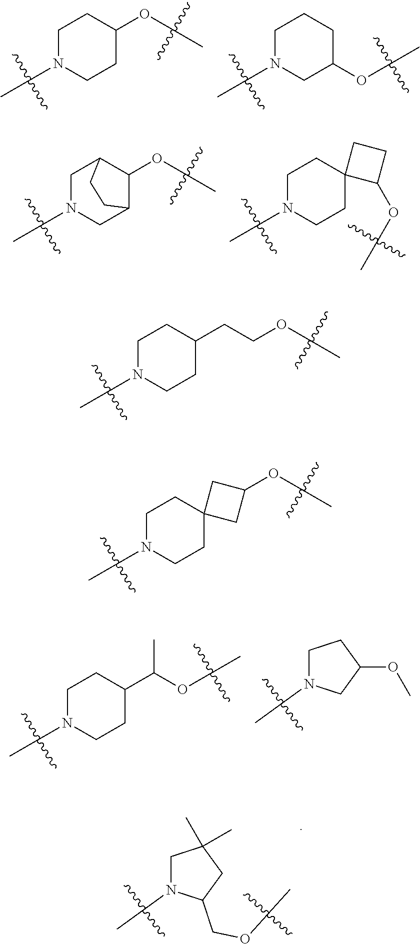

2. (canceled)

3. The ligand-drug conjugate, or a pharmaceutically acceptable salt or solvate thereof, according to claim 1, being a ligand-drug conjugate of formula (IIIa) or a pharmaceutically acceptable salt or solvate thereof: 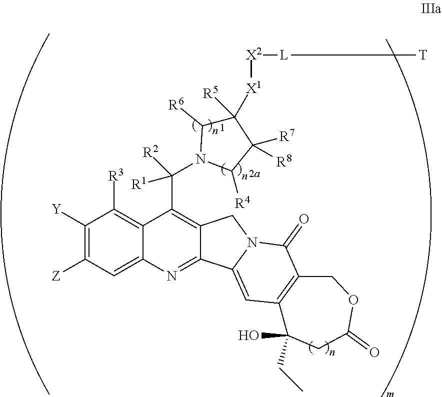
wherein:
R¹ and R² are each hydrogen;
T is a targeting or binding ligand;
L is a releasable linker; and
m is an integer or fraction of integer selected from 1 to 10.

4. The ligand-drug conjugate or the pharmaceutically acceptable salt or solvate thereof according to claim 1, wherein: 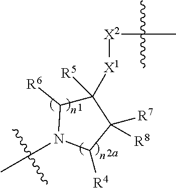
is

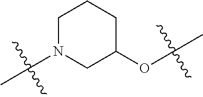 [SMILES: *OC1CCCN(*)C1]

5. The ligand-drug conjugate, or a pharmaceutically acceptable salt or solvate thereof, according to claim 3, wherein m is an integer or fraction of an integer selected from 2 to 8; optionally m is an integer or fraction of an integer selected from 3 to 8.

6. The ligand-drug conjugate, or a pharmaceutically acceptable salt or solvate thereof, according to claim 3, wherein L is -L¹-L²-L³-L⁴-, and optionally wherein L¹ is connected to the ligand, and the L⁴ is connected to X² and X² is —O—;
L¹ is selected from the group consisting of 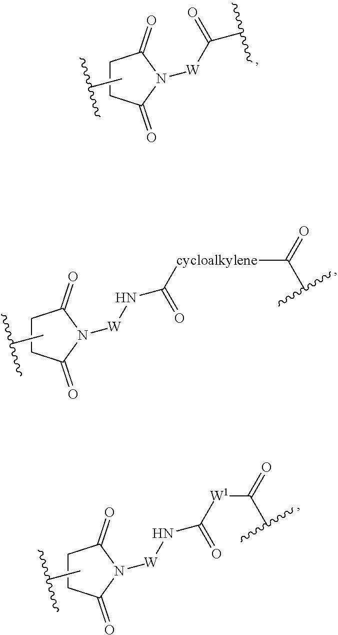
—CH₂—C(O)—NR¹⁰—W—C(O)—, C(O)—W—C(O)—, and —W—, wherein W and W¹ are independently selected from the group consisting of C₁-C₈ alkylene, —(C₁-C₈ alkylene)-cycloalkylene-, arylene, heteroarylene, and linear heteroalkylene, wherein the linear heteroalkylene comprise 1 to 8 carbon atom(s), and 1 to 3 heteroatom(s) selected from the group consisting of N, O, S, SO, and SO₂, and wherein the —(C₁-C₈ alkylene)-cycloalkylene-, linear heteroalkylene, arylene, and heteroarylene are each independently optionally further substituted by one or more substituent(s) selected from the group consisting of halogen, hydroxy, cyano, amino, alkyl, chloroalkyl, deuterated alkyl, alkoxy and cycloalkyl; wherein the left side of each of the L¹ groups provided above is attached to T;
L² is selected from the group consisting of —NR¹¹(CH₂CH₂O)ₚ¹CH₂CH₂C(O)—, —NR¹¹(CH₂CH₂O)ₚ¹CH₂CH₂—, —NR¹¹(CH₂CH₂O)ₚ¹CH₂C(O)—, —S(CH₂)ₚ¹C(O)—, and a chemical bond, wherein p¹ at each occurrence is an integer selected from 1 to 20; and L² is preferably a chemical bond; wherein the left side of each of the L² groups provided above is attached to L¹;
L³ is a peptide residue composed of 2 to 7 amino acids, wherein the amino acids are optionally further substituted by one or more substituent(s) selected from the group consisting of halogen, hydroxy, cyano, amino, alkyl, chloroalkyl, deuterated alkyl, alkoxy and cycloalkyl; wherein the left side of each of the L³ groups provided above is attached to L²;
L⁴ is selected from the group consisting of —NR¹²(CR¹³R¹⁴)—, —C(O)NR¹²—, —C(O)NR¹²(CH)ₜ—, 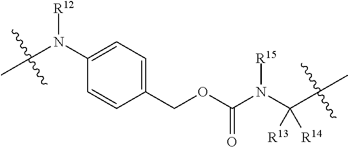
and a chemical bond, wherein t at each occurrence is an integer selected from 1 to 6; and L⁴ is preferably —NR¹²(CR¹³R¹⁴)ₜ⁻; wherein the left side of each of the L⁴ groups provided above is attached to the right side of L³ and the right side of each of the L⁴ groups is attached to X²;
R¹⁰, R¹¹ and R¹² are each independently selected from the group consisting of H, allyl, haloalkyl, deuterated alkyl and hydroxyalkyl;
R¹³ and R¹⁴ are each independently selected from the group consisting of H, halogen, alkyl, haloalkyl, deuterated alkyl, and hydroxyalkyl; and
R¹⁵ is selected from —CH₂CH₂SO₂CH₃, and —CH₂CH₂N(CH₃)₂
optionally wherein L¹ is selected from the group consisting of 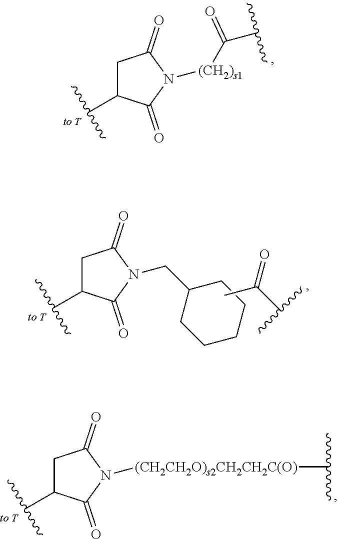
—CH₂—C(O)—NR¹⁰—(CH₂)s³-C(O)— (wherein the left hand side of this group is attached to T), —C(O)—(CH₂)s⁴-C(O)— (wherein the left hand side of this group is attached to T), and —C₆H₄—, wherein s¹ is an integer selected from 2 to 8; s² is an integer selected from 1 to 3; s³ is an integer selected from 1 to 8; and s⁴ is an integer selected from 1 to 8; and/or
optionally wherein L² is selected from the group consisting of —NR¹¹(CH₂CH₂O)ₚ¹CH₂CH₂C(O)—, —NR¹¹(CH₂CH₂O)ₚ¹CH₂CH₂—, —NR¹¹(CH₂CH₂O)ₚ¹CH₂C(O)—, —S(CH₂)ₚ¹C(O)—, and a chemical bond, wherein p¹ is an integer selected from 6 to 12; and/or
optionally L³ is a peptide residue composed of 2 to 7 amino acids, wherein the amino acids are selected from Phenylalanine (F), Glycine (G), Valine (V), Lysine (K), Citrulline, Serine (S), Glutamic acid (E), and Aspartic acid (N); preferably a peptide residue composed of 1, 2 or more Phenylalanine and Glycine; more preferably is a peptide residue composed of 4 amino acids; the most preferably is a peptide residue composed of GGFG; and/or
optionally L⁴ is —NR¹²(CR¹³R¹⁴)t-, R¹² is H or alkyl, R¹³ and R¹⁴ are each independently selected from H and alkyl, t is 1 or 2; L⁴ is optionally —NR¹²CR¹³R¹⁴—; L⁴ is preferably —NHCH₂—.

7. The ligand-drug conjugate, or a pharmaceutically acceptable salt or solvate thereof, according to claim 3, wherein L is -L¹-L²-L³-L⁴-,
L¹ is 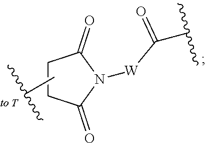
L² is a chemical bond;
L³ is a peptide residue comprising 4 amino acids;
L⁴ is —NR¹²(CR¹³R¹⁴)t-, R¹² is H or alkyl, R¹³ and R¹⁴ are each independently H or alkyl, and
t is 1 or 2.

8. The ligand-drug conjugate, or a pharmaceutically acceptable salt or solvate thereof, according to claim 3, wherein L is -L¹-L²-L³-L⁴-,
L¹ is 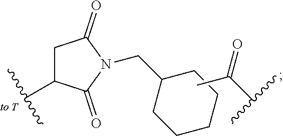
L² is —NR¹¹(CH₂CH₂O)₉CH₂CH₂C(O)—;
L³ is a peptide residue comprising 4 amino acids;
L⁴ is —NR¹²(CR¹³R¹⁴)t-, R¹² is H or alkyl, R¹³ and R¹⁴ are each independently H or alkyl, and
t is 1 or 2.

9. The ligand-drug conjugate, or a pharmaceutically acceptable salt or solvate thereof, according to claim 1, selected from:

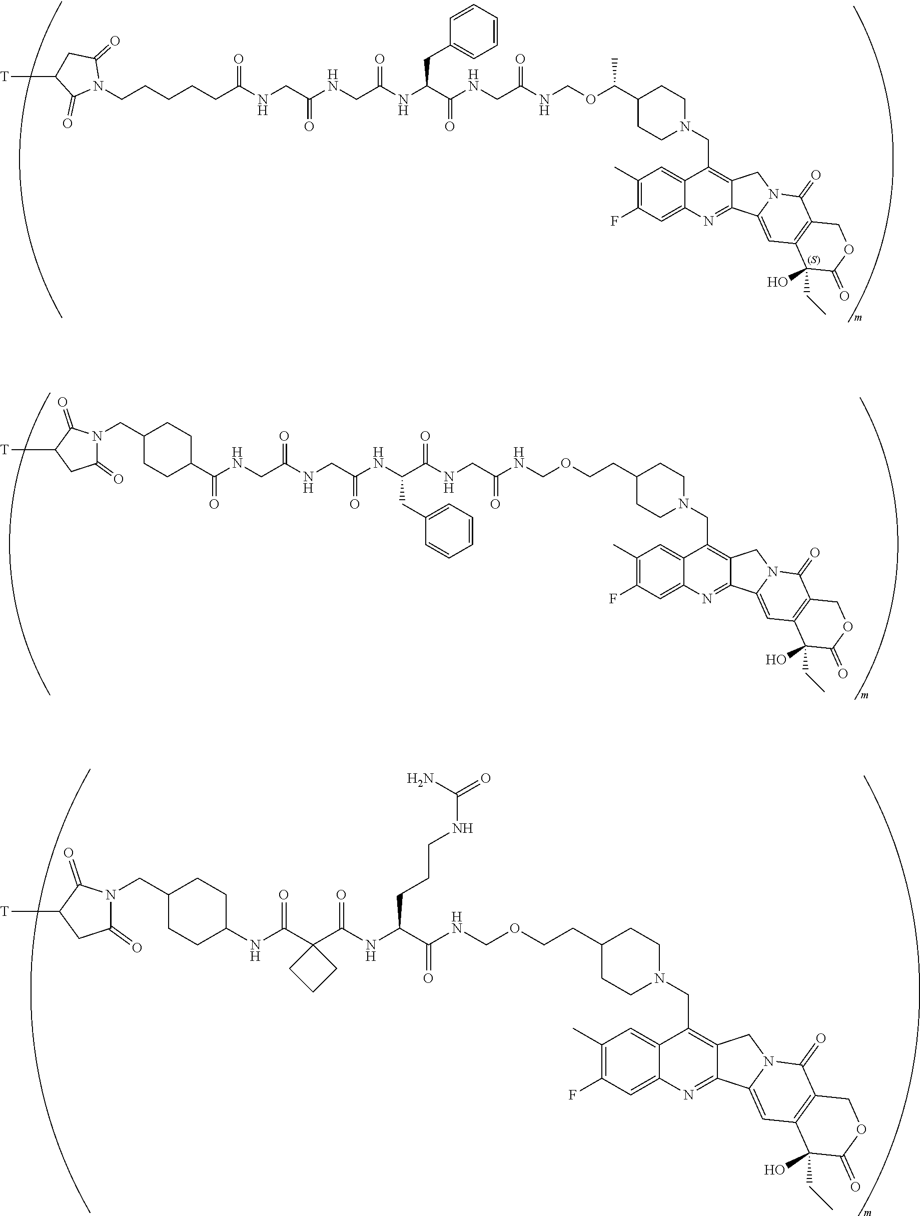

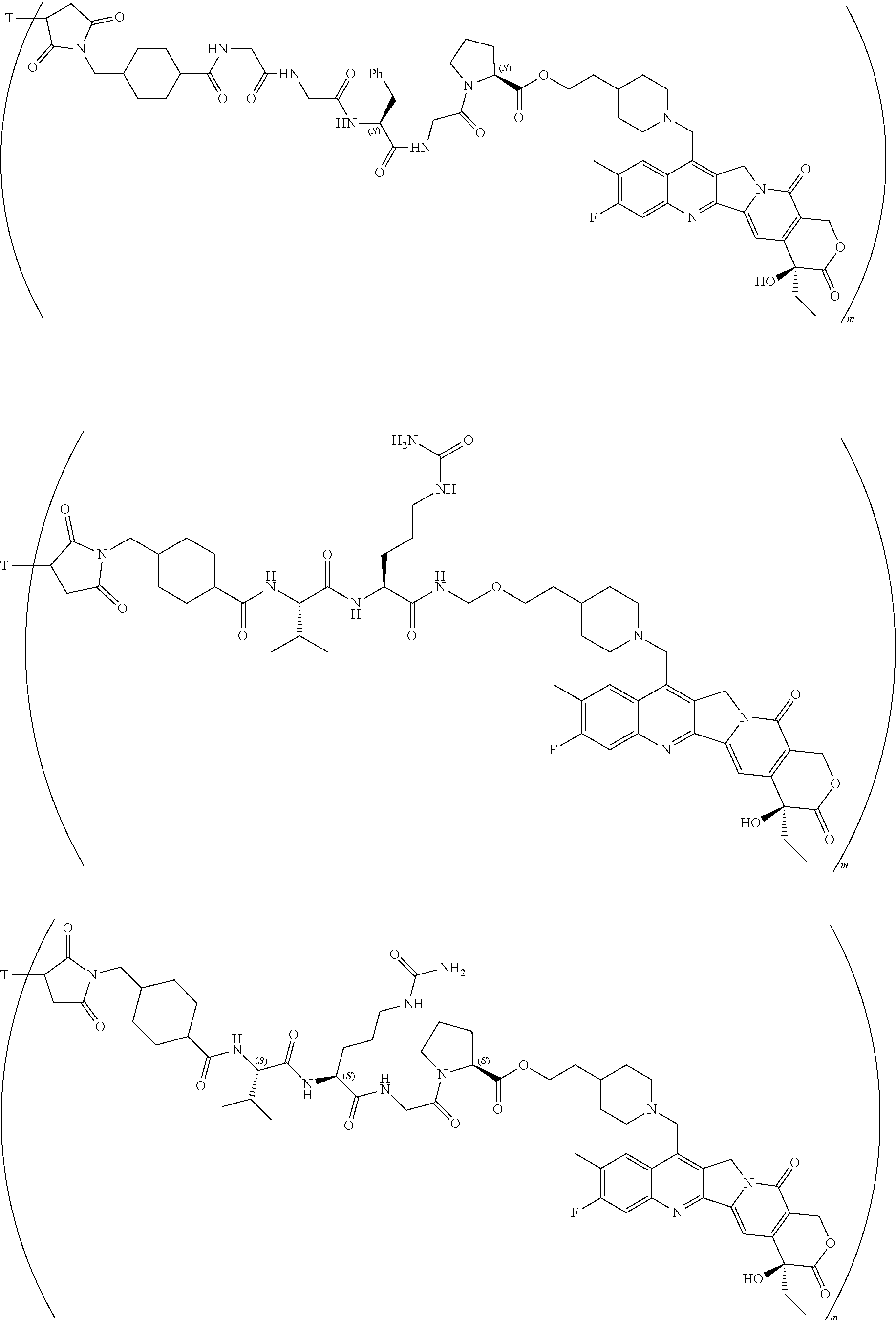

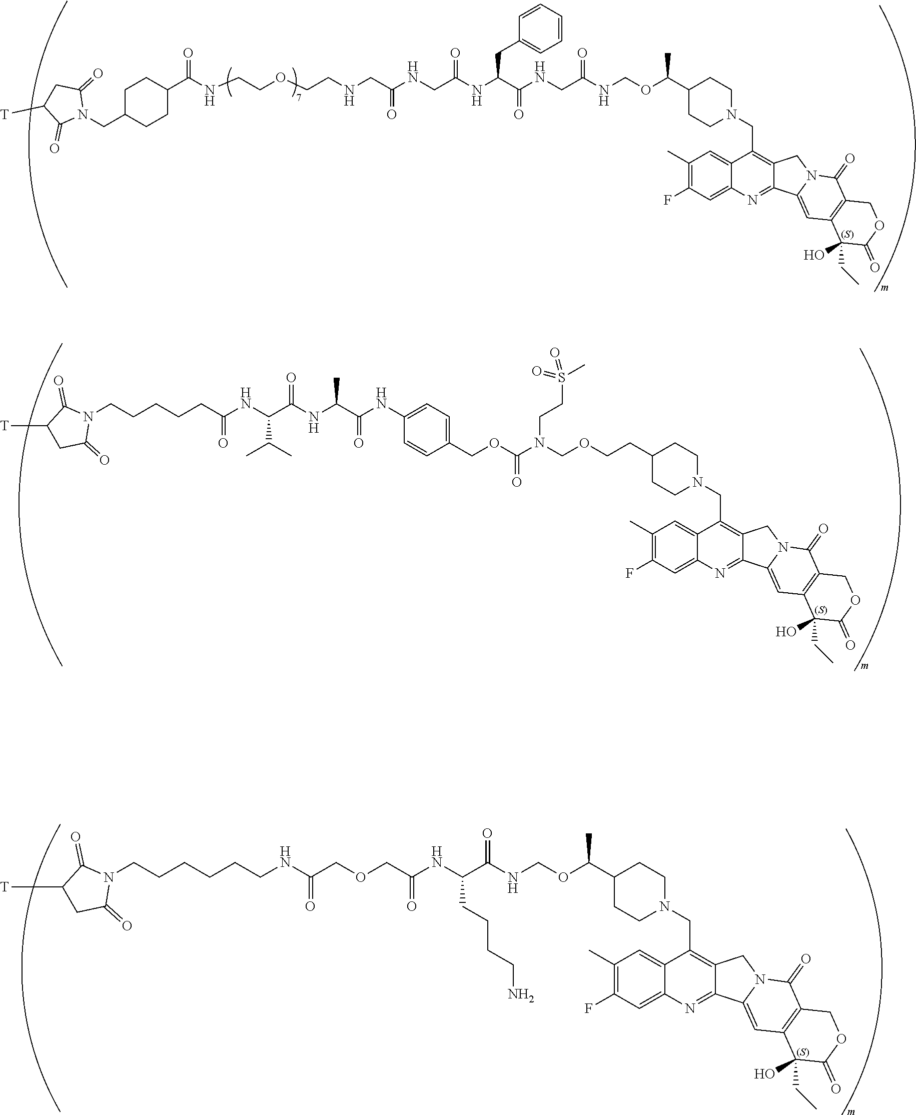

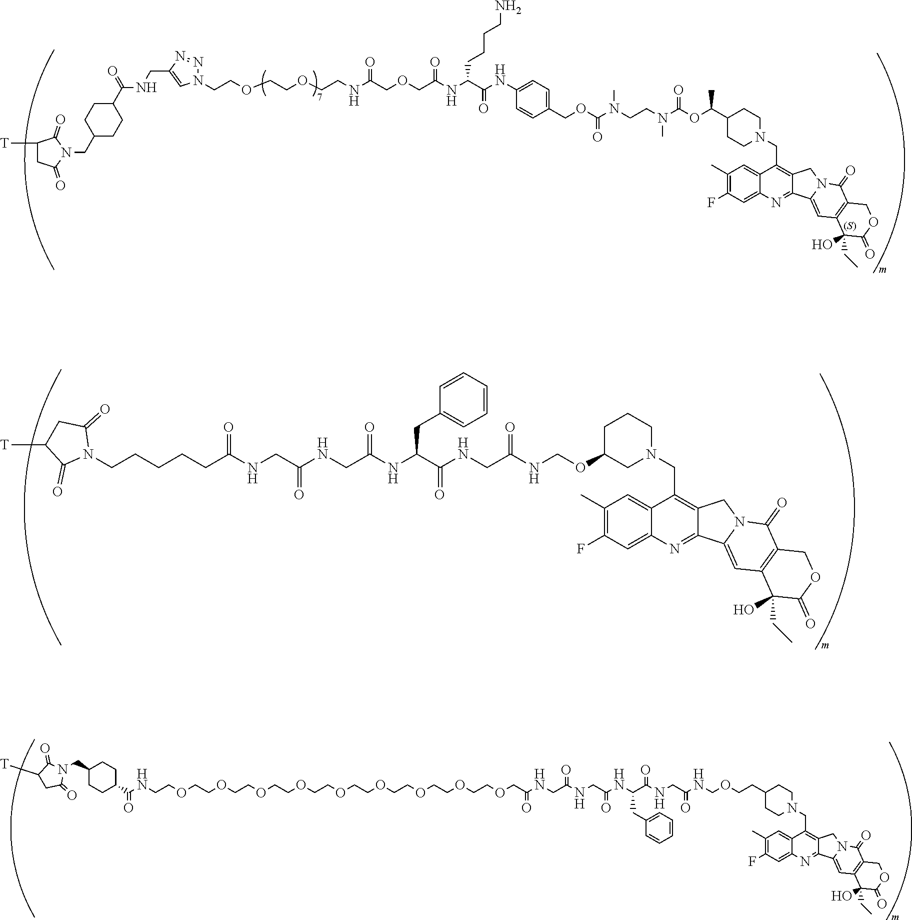

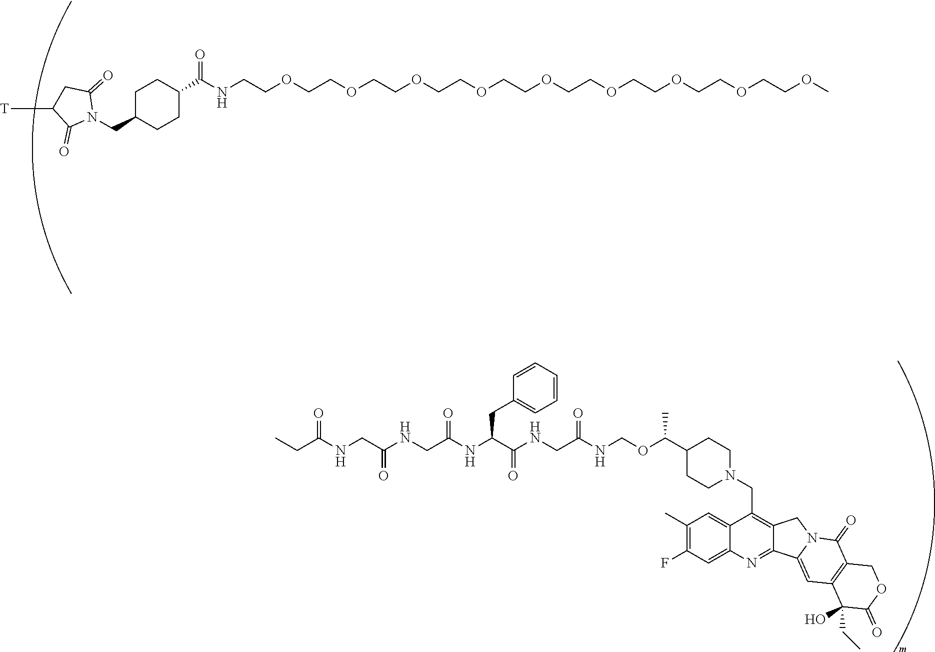

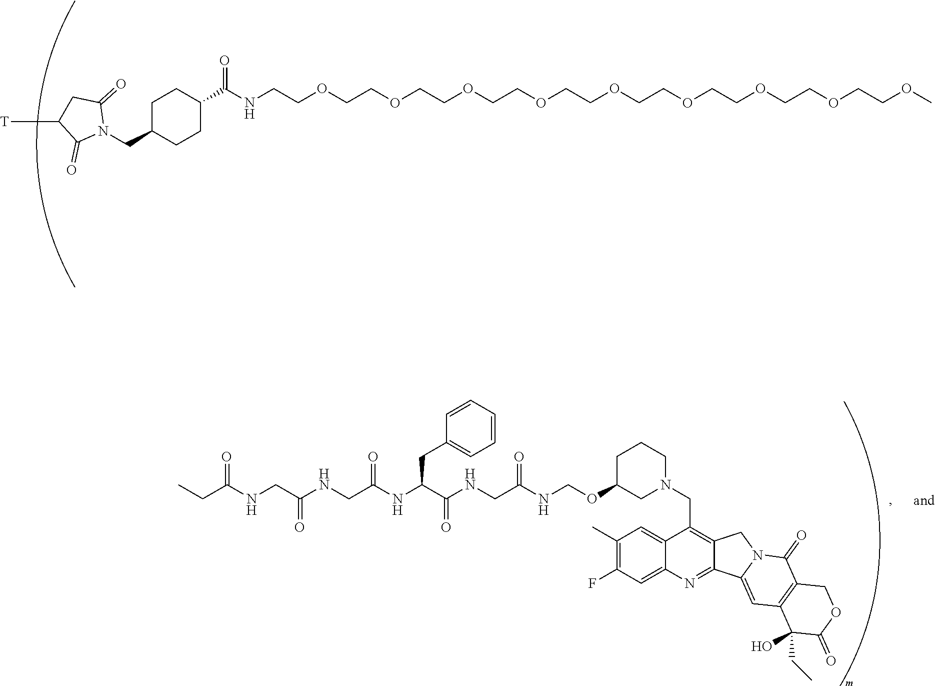

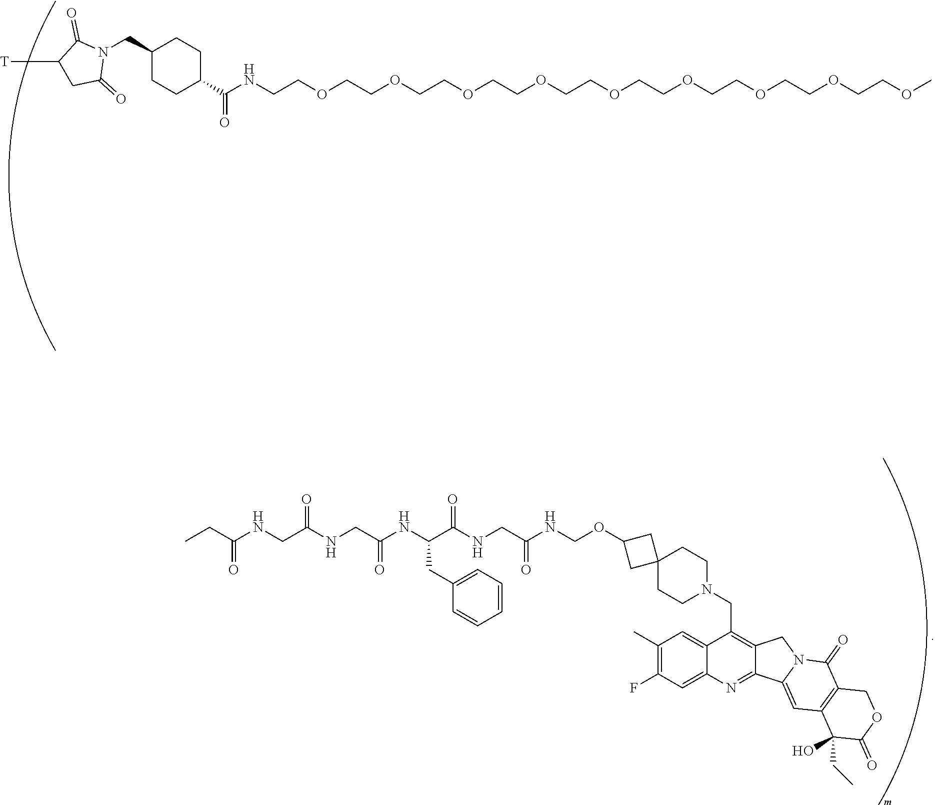

10. The ligand-drug conjugate, or a pharmaceutically acceptable salt or solvate thereof, according to claim 3, wherein the ligand-drug conjugate is according to formula (IIIa), or a pharmaceutically acceptable salt or solvate thereof:
wherein T is a targeting antibody or ligand binding to antigen; wherein the antibody is selected from chimeric antibody, humanized antibody and human antibody; and optionally wherein T is a monoclonal antibody; or
wherein T is selected from anti-Her2(ErbB2) antibody, anti-EGFR antibody, anti-B₇H₃ antibody, anti-c-MET antibody, anti-Her3(ErbB3) antibody, anti-Her4(ErbB4) antibody, anti-CD20 antibody, anti-CD22 antibody, anti-CD30 antibody, anti-CD33 antibody, anti-CD44 antibody, anti-CD56 antibody, anti-CD70 antibody, anti-CD73 antibody, anti-CD105 antibody, anti-CEA antibody, anti-A33 antibody, anti-Cripto antibody, anti-EphA2 antibody, anti-G250 antibody, anti-MICI antibody, anti-Lewis Y antibody, anti-VEGFR antibody, anti-GPNMB antibody, anti-Integrin antibody, anti-PSMA antibody, anti-Tenascin-C antibody, anti-SLC44A4 antibody or anti-Mesothelin antibody, and anti-ROR1 antibody or the fragment binding to the antigen; or
wherein T is selected from Trastuzumab, Pertuzumab, Nimotuzumab, Enoblituzumab, Emibetuzumab, Inotuzumab, Pinatuzumab, Brentuximab, Gemtuzumab, Bivatuzumab, Lorvotuzumab, cBR96 and Glembatumumab or the fragment binding to the antigen.

11. The ligand-drug conjugate, or a pharmaceutically acceptable salt or solvate thereof, according to claim 1, selected from:

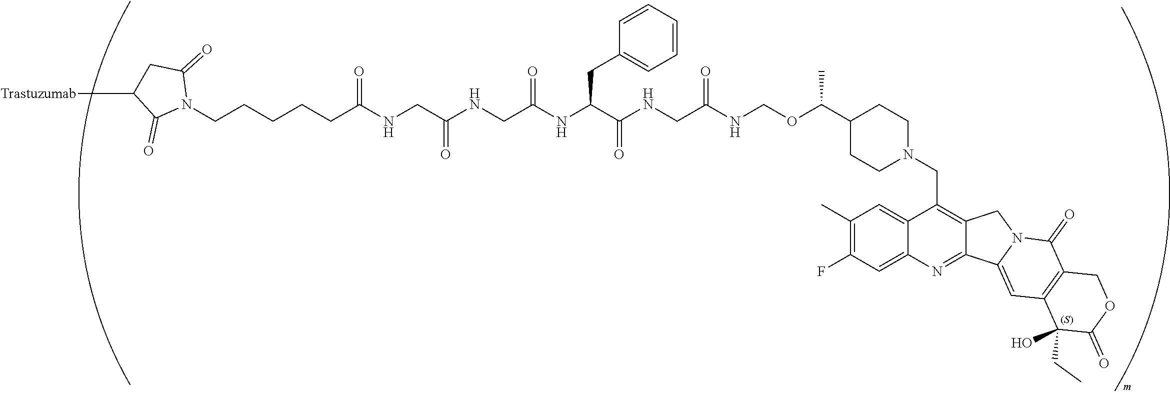

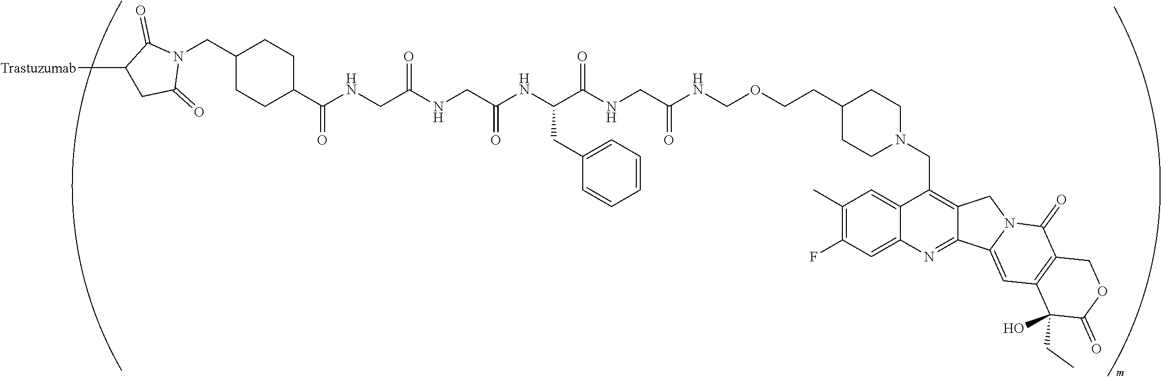

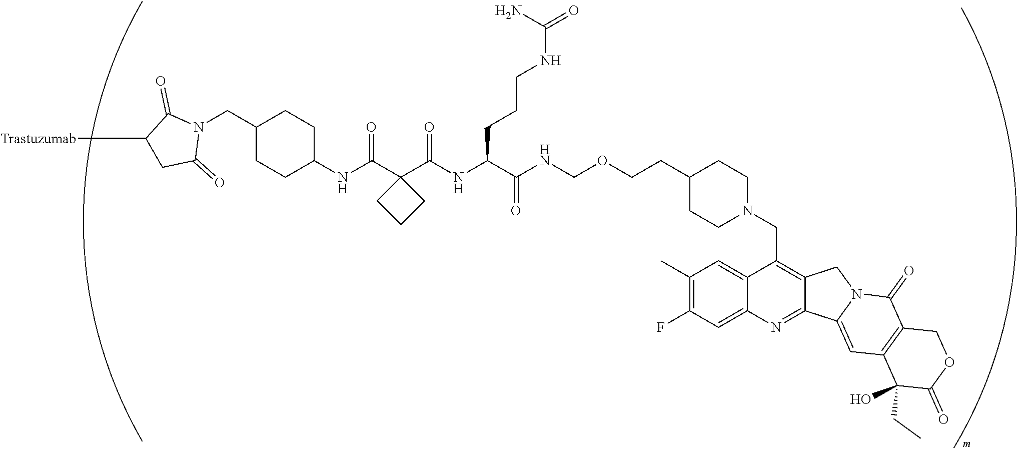

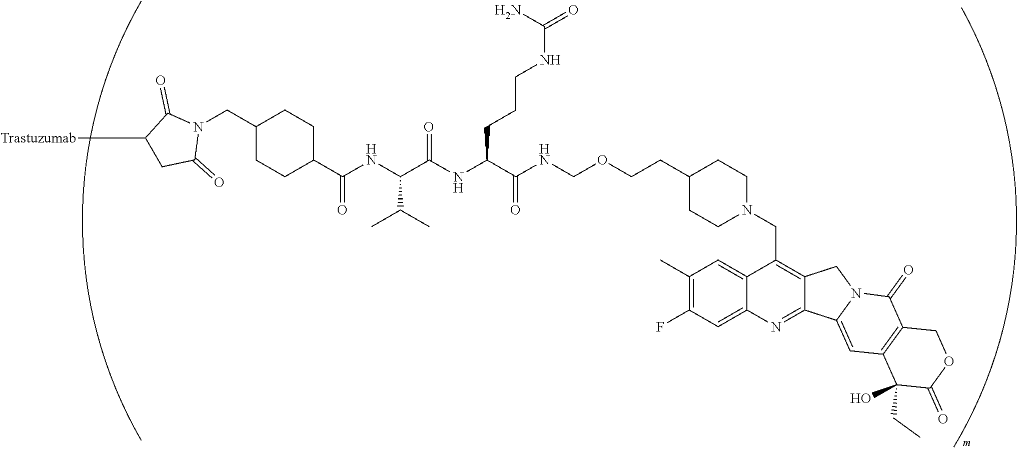

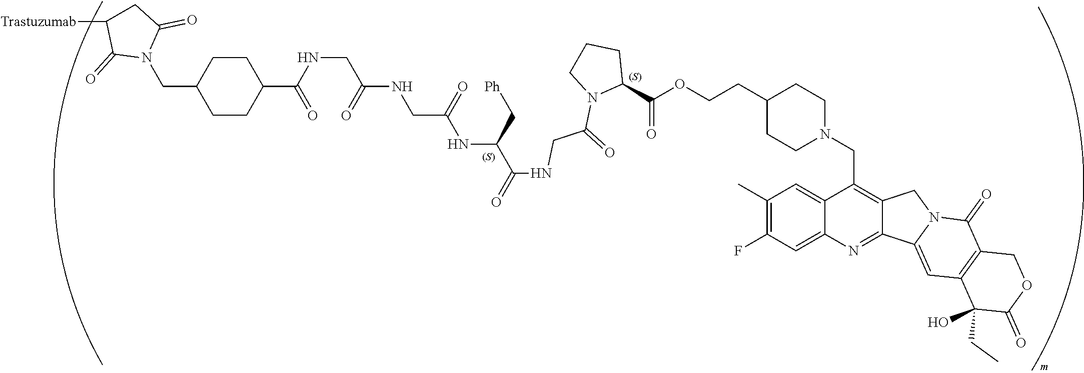

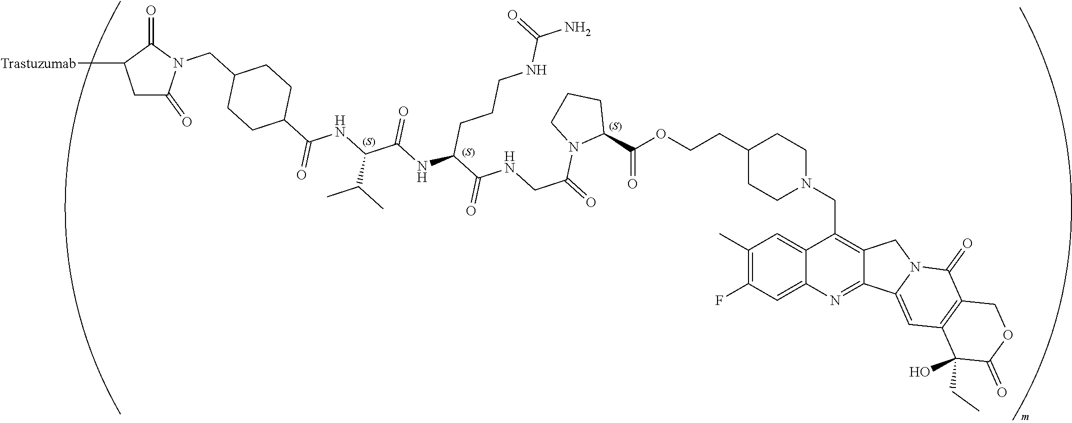

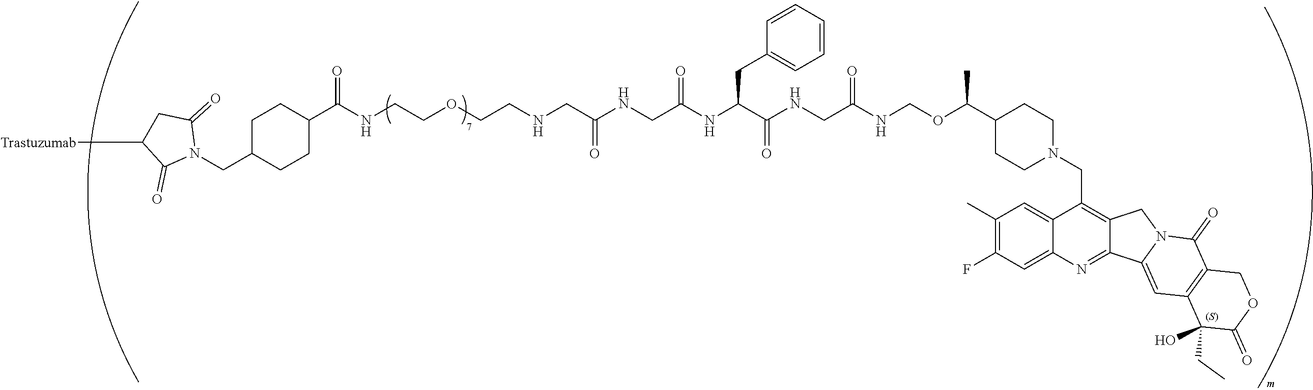

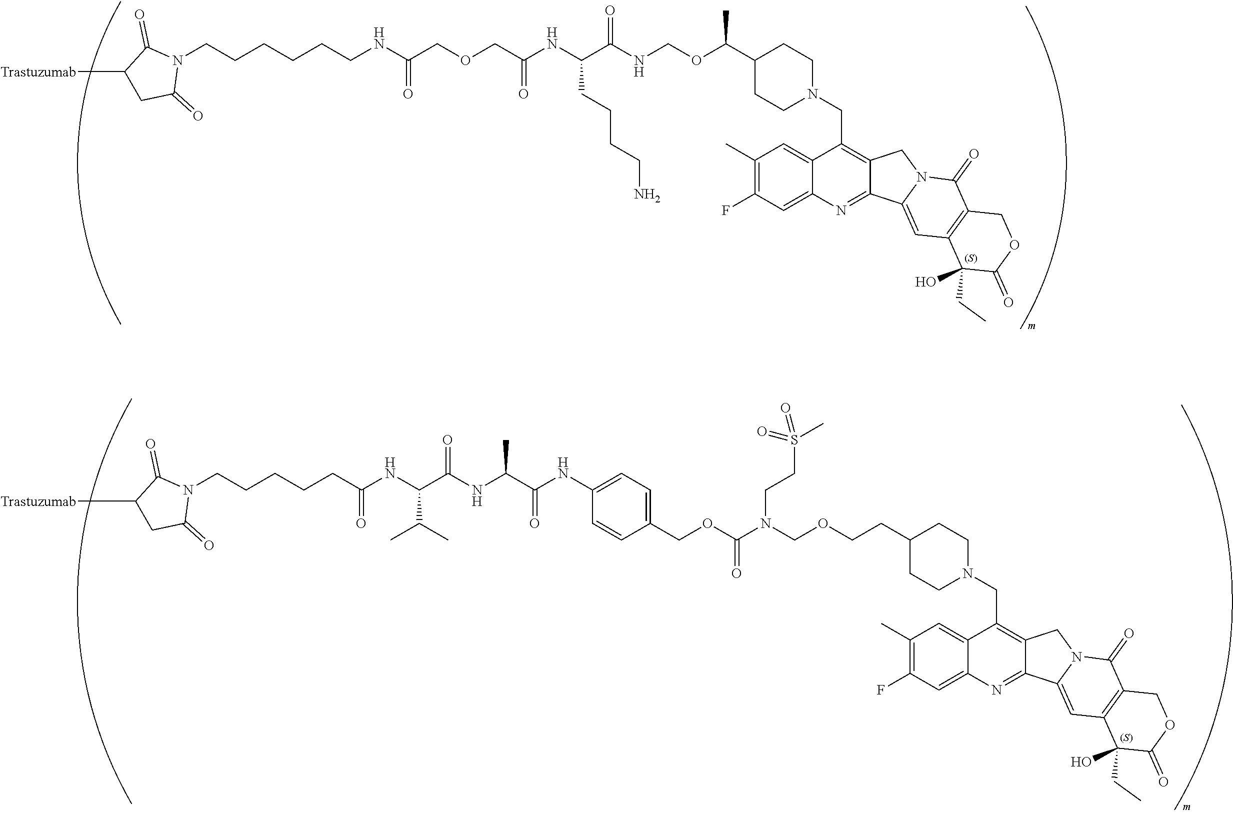

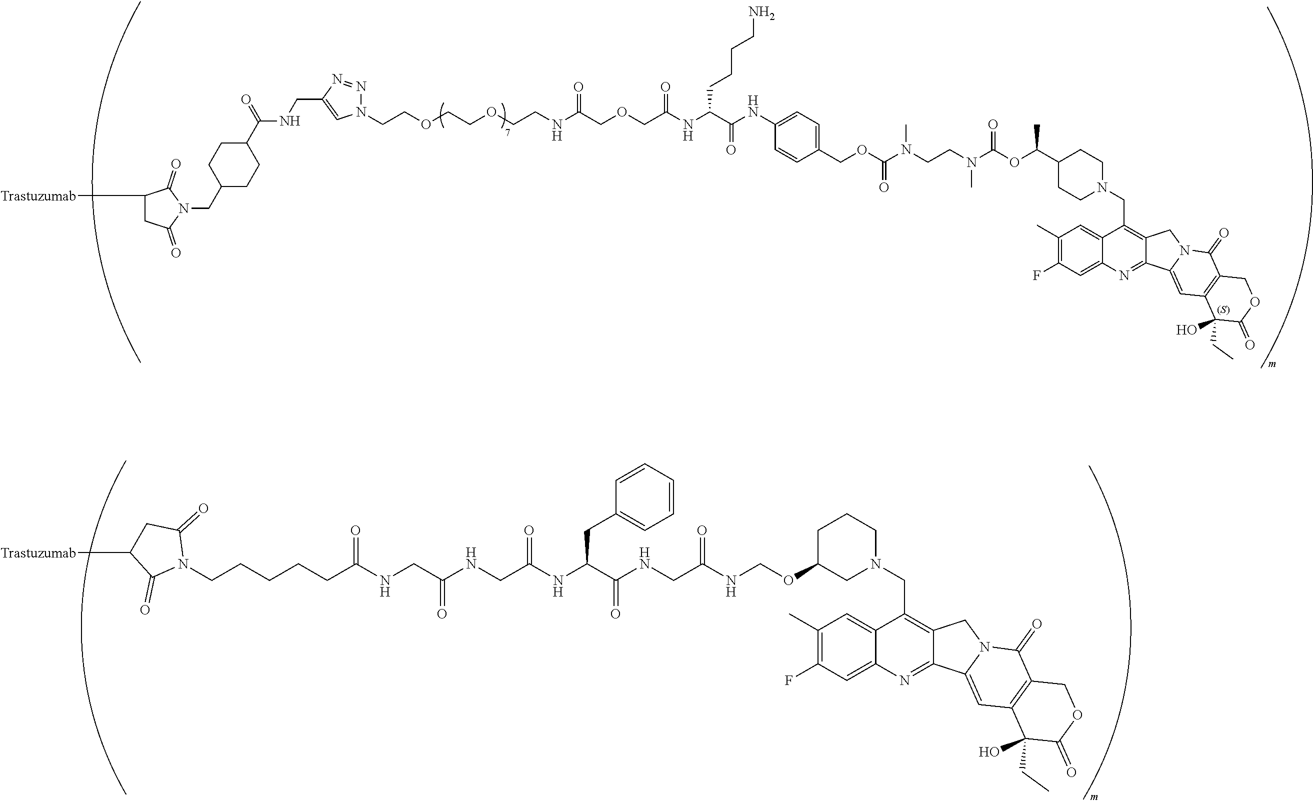

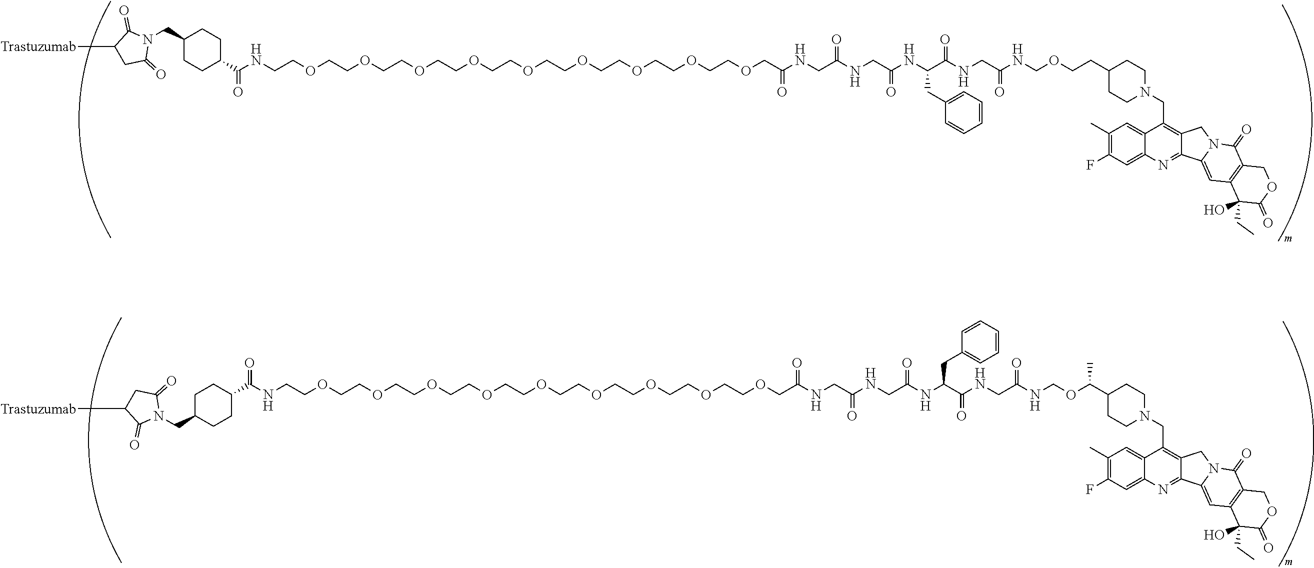

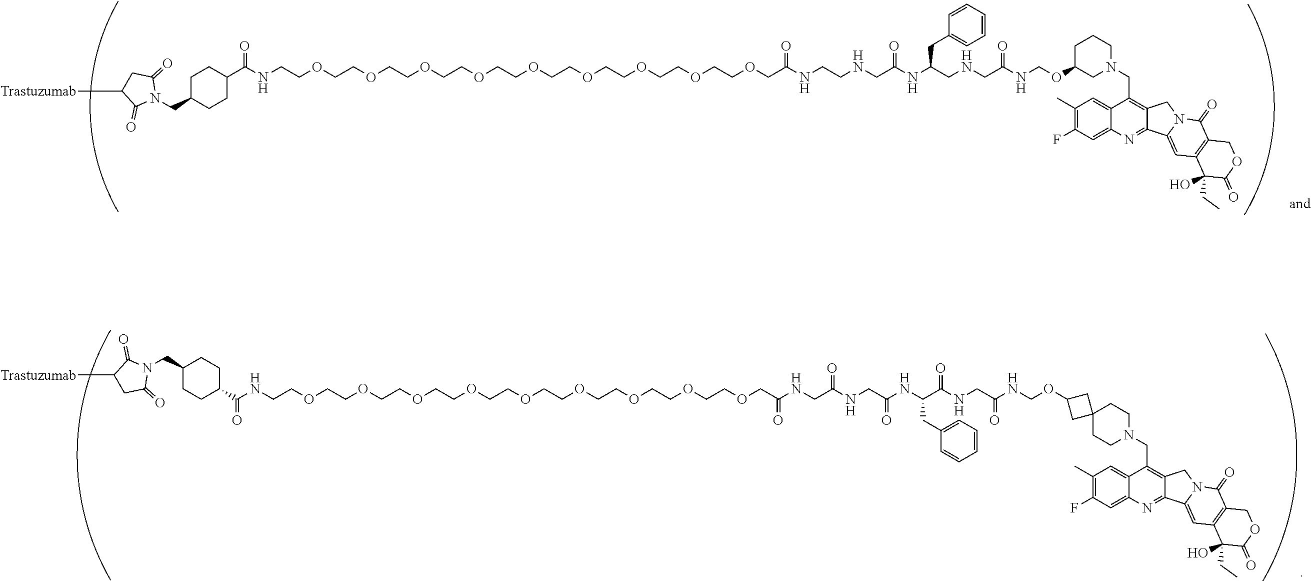

12. A compound of formula (Va), or a pharmaceutically acceptable salt or solvate thereof: 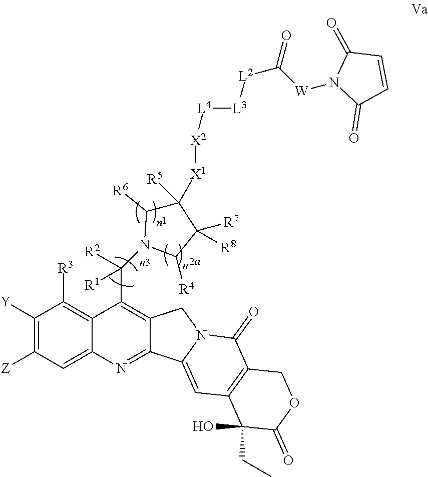
wherein:
Y and Z are independently selected from H, halo, C₁-C₈ alkyl, and C₁-C₈ alkoxy;
R³ is selected from H, halo, C₁-C₈ alkyl, and C₁-C₈ alkoxy; 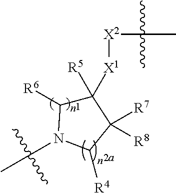
is 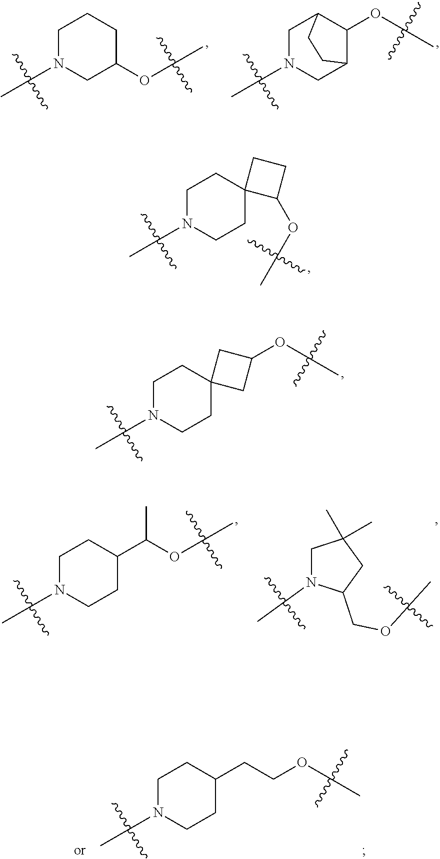
R¹ and R² are each hydrogen;
n³ is 1;
W is selected from the group consisting of C₁-C₈ alkylene, —(C₁-C₈ alkylene)-cycloalkylene-, arylene, heteroarylene, and linear heteroalkylene, wherein the linear heteroalkylene comprise 1 to 8 carbon atom(s), and 1 to 3 heteroatom(s) selected from the group consisting of N, O, S, SO, and SO₂, and wherein the —(C₁-C₈ alkylene)-cycloalkylene-linear heteroalkylene, arylene, and heteroarylene are each independently optionally further substituted by one or more substituent(s) selected from the group consisting of halogen, hydroxy, cyano, amino, alkyl, chloroalkyl, deuterated alkyl, alkoxy and cycloalkyl;
L² is the group consisting of —NR¹¹(CH₂CH₂O)ₚ¹CH₂CH₂C(O)—, —NR¹¹(CH₂CH₂O)ₚ¹CH₂CH₂—, —NR¹¹(CH₂CH₂O)ₚ¹CH₂C(O)—, —S(CH₂)ₚ¹C(O)— and a chemical bond, wherein p¹ is an integer selected from 1 to 20; wherein the left side of each of the L² groups provided above is attached to L¹;
L³ is a peptide residue composed of 2 to 7 amino acids, wherein the amino acids are optionally further substituted by one or more substituent(s) selected from the group consisting of halogen, hydroxy, cyano, amino, alkyl, chloroalkyl, deuterated alkyl; wherein the left side of each of the L³ groups provided above is attached to L²;
L⁴ is selected from the group consisting of —NR¹²(CR¹³R¹⁴)t-, —C(O)NR¹²—, —C(O)NR¹²(CH)ₜ—, 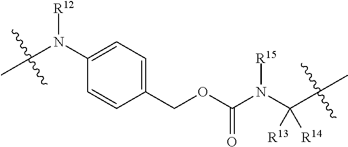
and a chemical bond, wherein t is an integer selected from 1 to 6; wherein the left side of each of the L⁴ groups provided above is attached to the right side of L³ and the right side of each of the L⁴ groups is attached to X²;
R¹⁰, R¹¹ and R¹² are each independently selected from the group consisting of H, allyl, haloalkyl, deuterated alkyl and hydroxyalkyl;
R¹³ and R¹⁴ are each independently selected from the group consisting of H, halogen, alkyl, haloalkyl, deuterated alkyl and hydroxyalkyl; and
R¹⁵ is selected from —CH₂CH₂SO₂CH₃, and —CH₂CH₂N(CH₃)₂.

13. The compound of claim 12, selected from structures below:

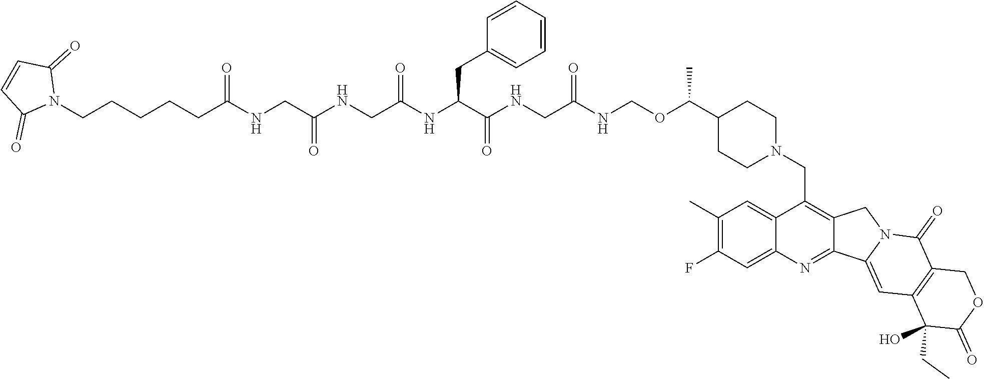 [SMILES: CC[C@@]1(O)C(=O)OCc2c1cc1n(c2=O)Cc2c-1nc1cc(F)c(C)cc1c2CN1CCC([C@@H](C)OCNC(=O)CNC(=O)[C@H](Cc2ccccc2)NC(=O)CNC(=O)CNC(=O)CCCCCN2C(=O)C=CC2=O)CC1]

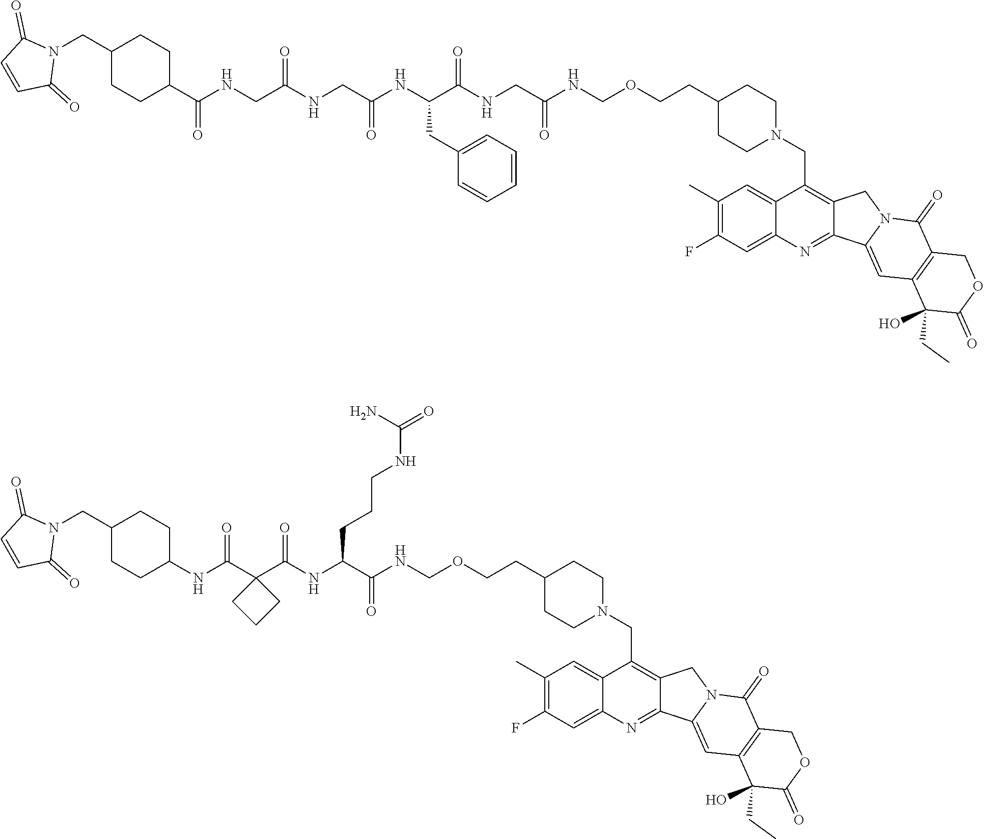 [SMILES: CC[C@@]1(O)C(=O)OCc2c1cc1n(c2=O)Cc2c-1nc1cc(F)c(C)cc1c2CN1CCC(CCOCNC(=O)CNC(=O)[C@H](Cc2ccccc2)NC(=O)CNC(=O)CNC(=O)C2CCC(CN3C(=O)C=CC3=O)CC2)CC1 ; CC[C@@]1(O)C(=O)OCc2c1cc1n(c2=O)Cc2c-1nc1cc(F)c(C)cc1c2CN1CCC(CCOCNC(=O)[C@H](CCCNC(N)=O)NC(=O)C2(C(=O)NC3CCC(CN4C(=O)C=CC4=O)CC3)CCC2)CC1]

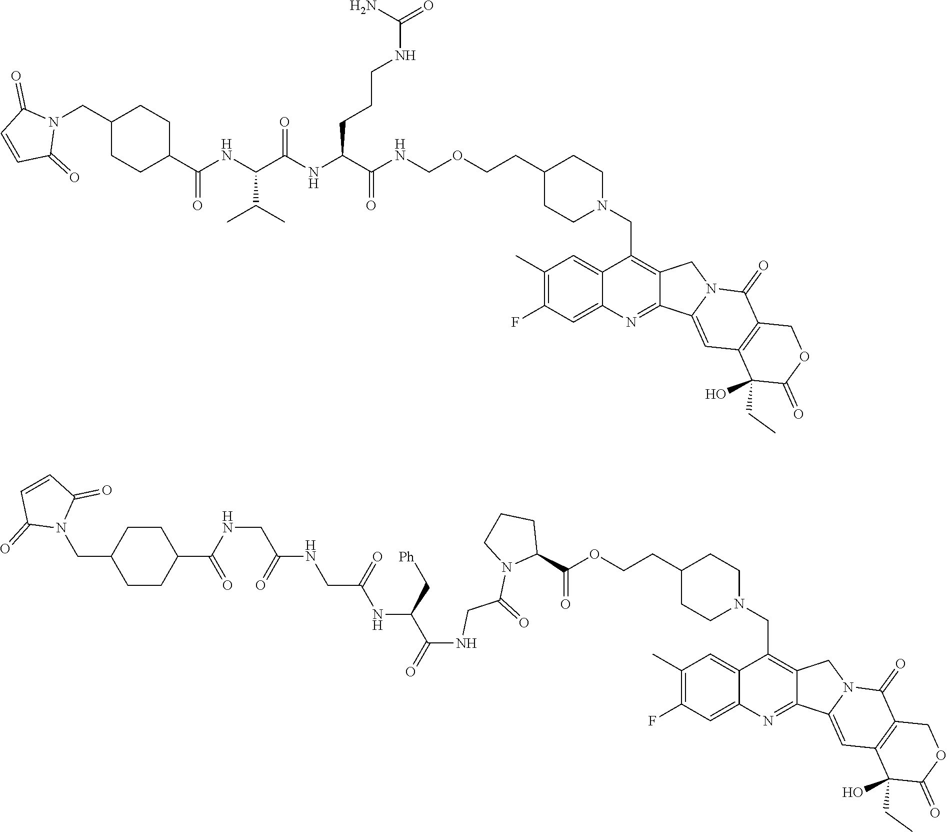 [SMILES: CC[C@@]1(O)C(=O)OCc2c1cc1n(c2=O)Cc2c-1nc1cc(F)c(C)cc1c2CN1CCC(CCOCNC(=O)[C@H](CCCNC(N)=O)NC(=O)[C@@H](NC(=O)C2CCC(CN3C(=O)C=CC3=O)CC2)C(C)C)CC1 ; CC[C@@]1(O)C(=O)OCc2c1cc1n(c2=O)Cc2c-1nc1cc(F)c(C)cc1c2CN1CCC(CCOC(=O)[C@@H]2CCCN2C(=O)CNC(=O)[C@H](Cc2ccccc2)NC(=O)CNC(=O)CNC(=O)C2CCC(CN3C(=O)C=CC3=O)CC2)CC1]

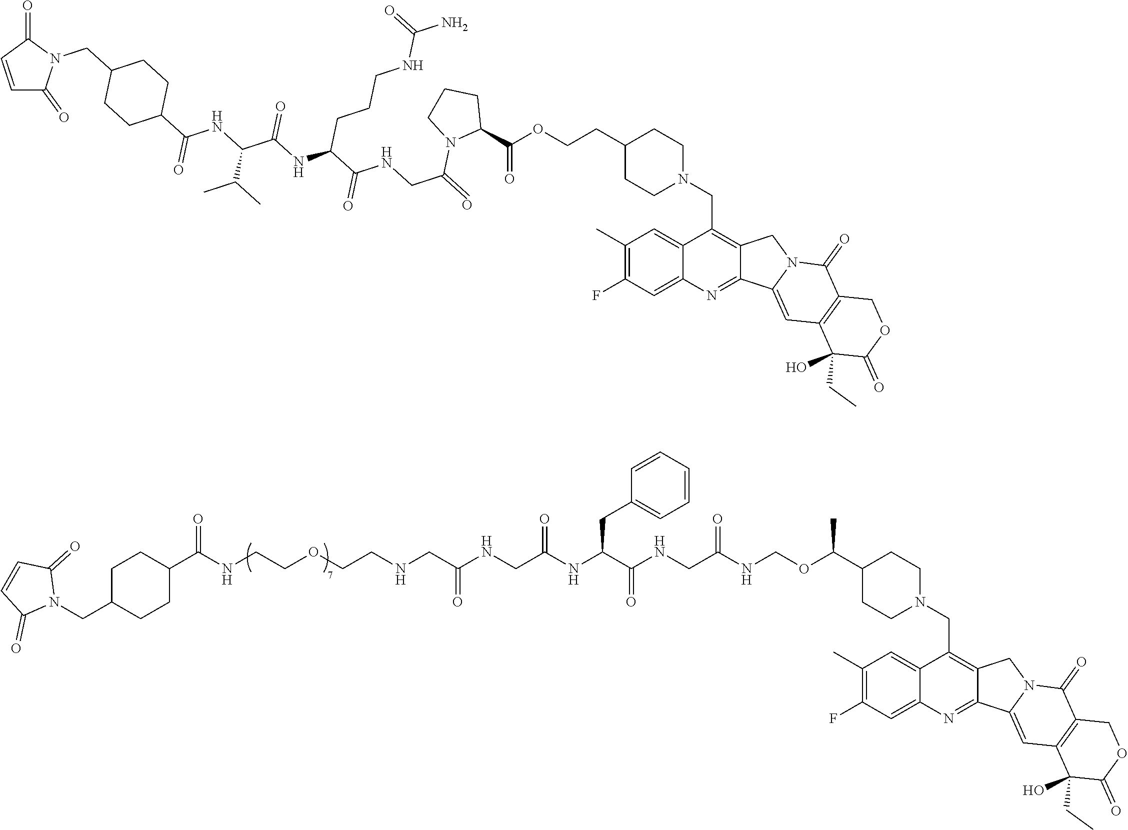

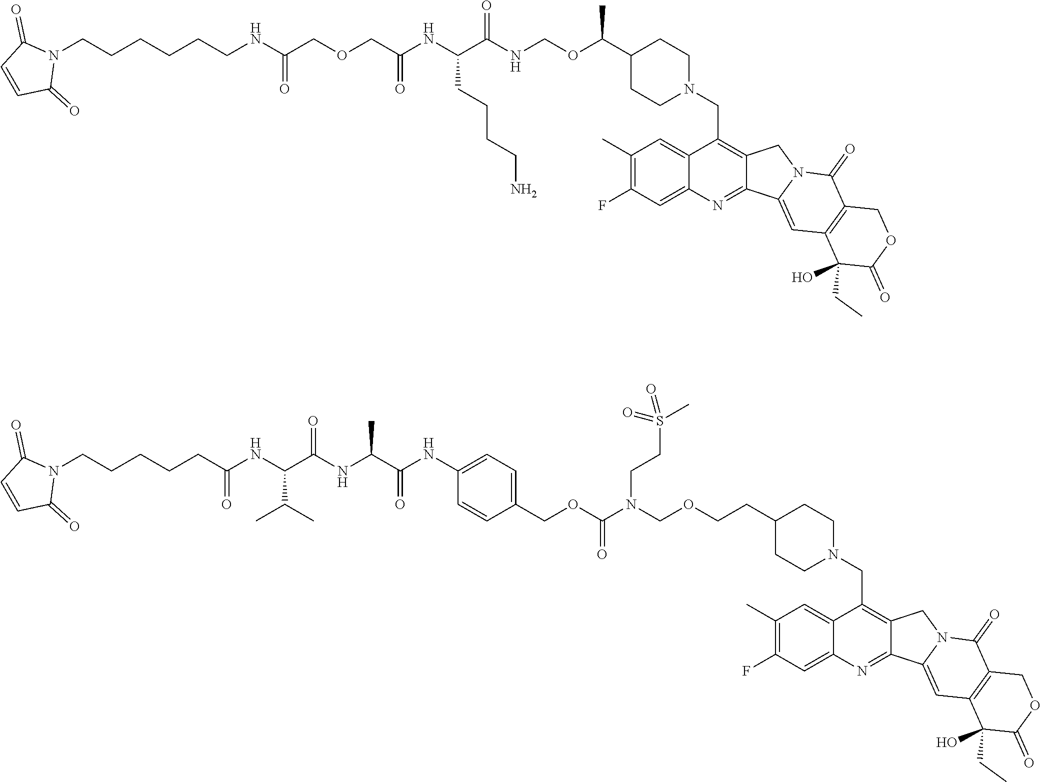 [SMILES: CC[C@@]1(O)C(=O)OCc2c1cc1n(c2=O)Cc2c-1nc1cc(F)c(C)cc1c2CN1CCC([C@H](C)OCNC(=O)[C@H](CCCCN)NC(=O)COCC(=O)NCCCCCCN2C(=O)C=CC2=O)CC1 ; CC[C@@]1(O)C(=O)OCc2c1cc1n(c2=O)Cc2c-1nc1cc(F)c(C)cc1c2CN1CCC(CCOCN(CCS(C)(=O)=O)C(=O)OCc2ccc(NC(=O)[C@H](C)NC(=O)[C@@H](NC(=O)CCCCCN3C(=O)C=CC3=O)C(C)C)cc2)CC1]

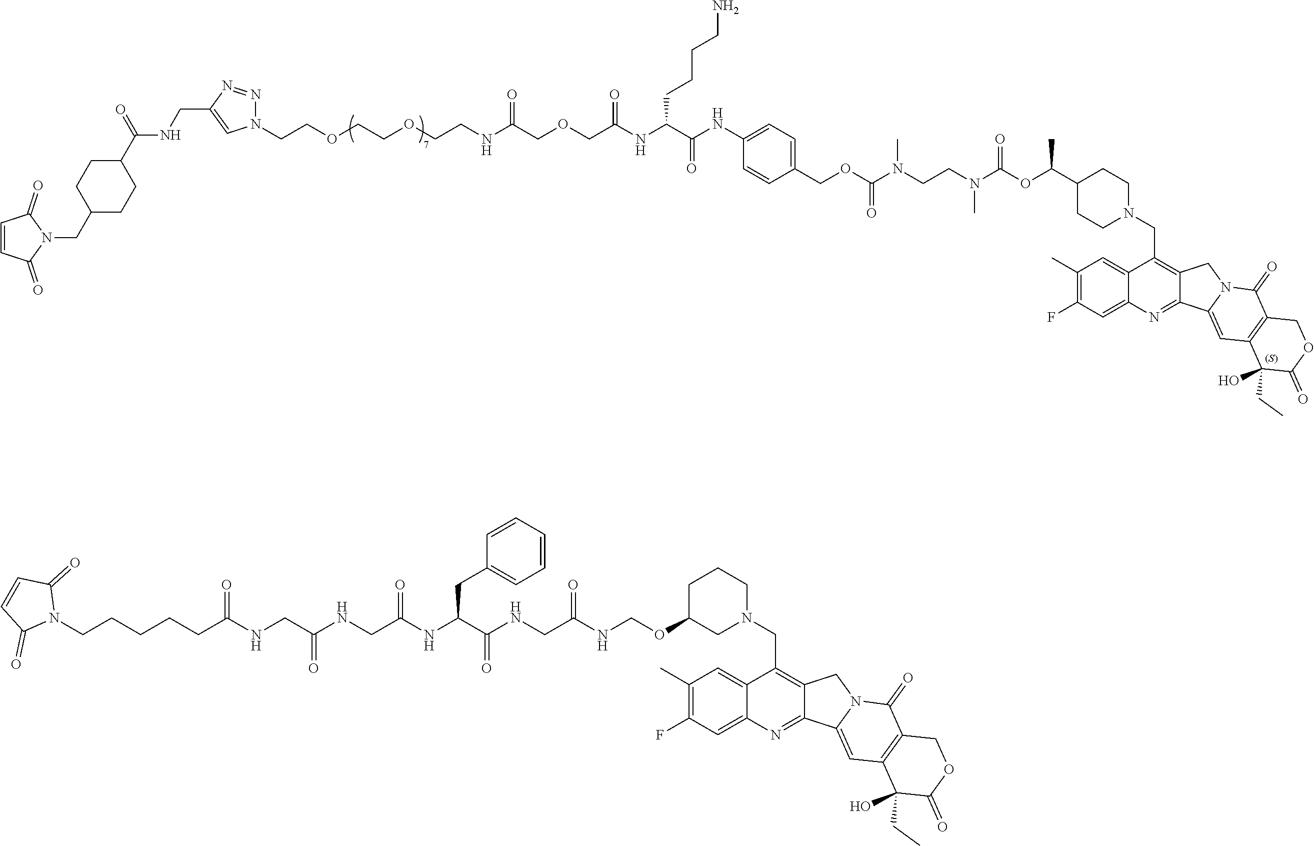

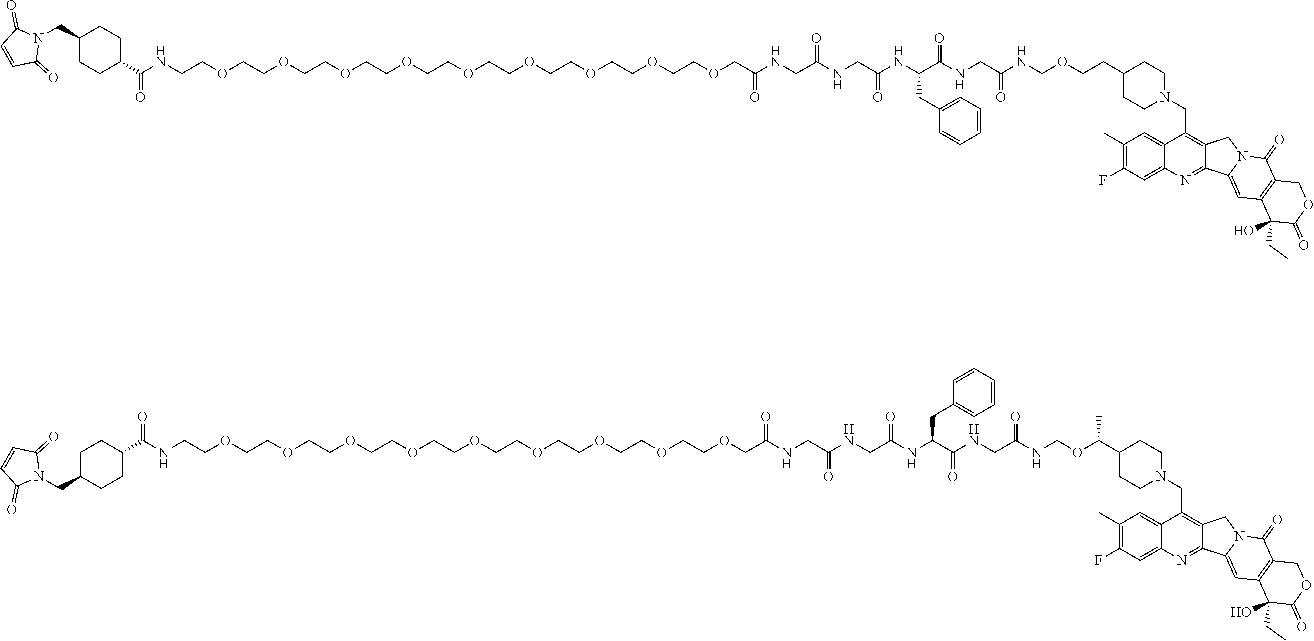 [SMILES: CC[C@@]1(O)C(=O)OCc2c1cc1n(c2=O)Cc2c-1nc1cc(F)c(C)cc1c2CN1CCC(CCOCNC(=O)CNC(=O)[C@H](Cc2ccccc2)NC(=O)CNC(=O)CNC(=O)COCCOCCOCCOCCOCCOCCOCCOCCOCCNC(=O)[C@H]2CC[C@H](CN3C(=O)C=CC3=O)CC2)CC1 ; CC[C@@]1(O)C(=O)OCc2c1cc1n(c2=O)Cc2c-1nc1cc(F)c(C)cc1c2CN1CCC([C@@H](C)OCNC(=O)CNC(=O)[C@H](Cc2ccccc2)NC(=O)CNC(=O)CNC(=O)COCCOCCOCCOCCOCCOCCOCCOCCOCCNC(=O)[C@H]2CC[C@H](CN3C(=O)C=CC3=O)CC2)CC1]

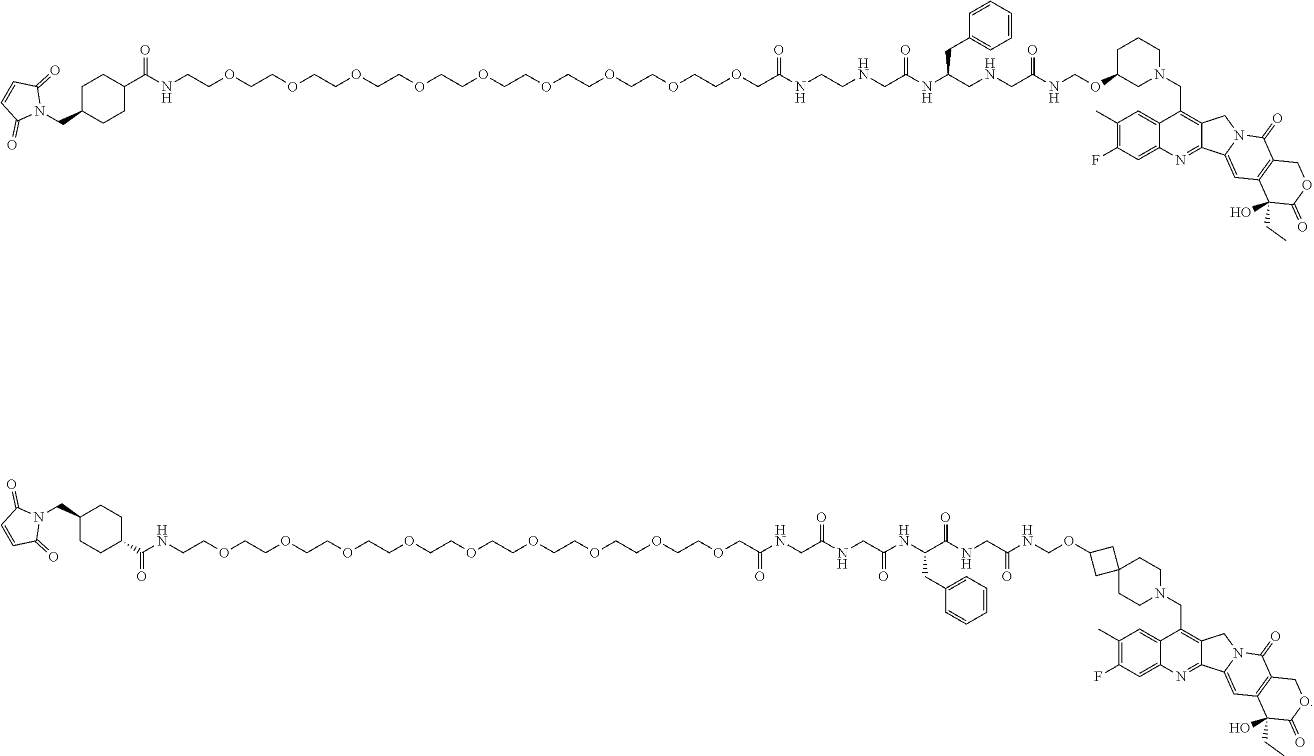

14. A compound of formula (VIIa) or a pharmaceutically acceptable salt thereof: 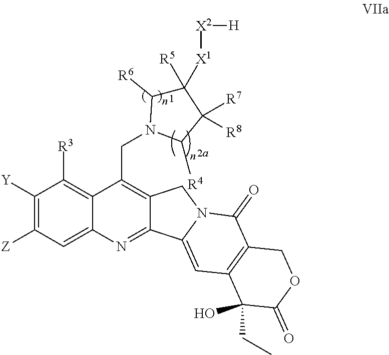
wherein:
Y and Z are independently selected from H, halo, C₁-C₈ alkyl, and C₁-C₈ alkoxy;
R³ is selected from H, halo, C₁-C₈ alkyl, and C₁-C₈ alkoxy; 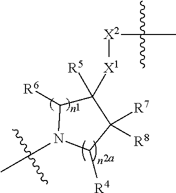
is

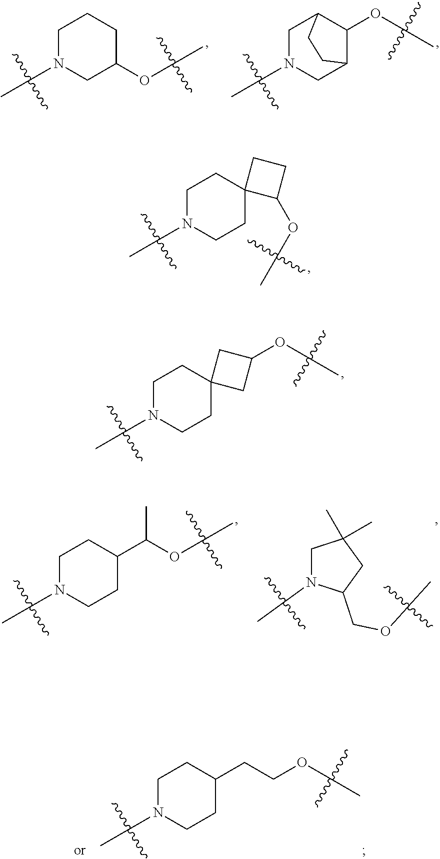

15. The compound according to claim 14, wherein: 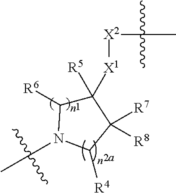
is

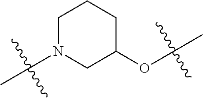 [SMILES: *OC1CCCN(*)C1]

16. The compound according to claim 14, selected from structures below:

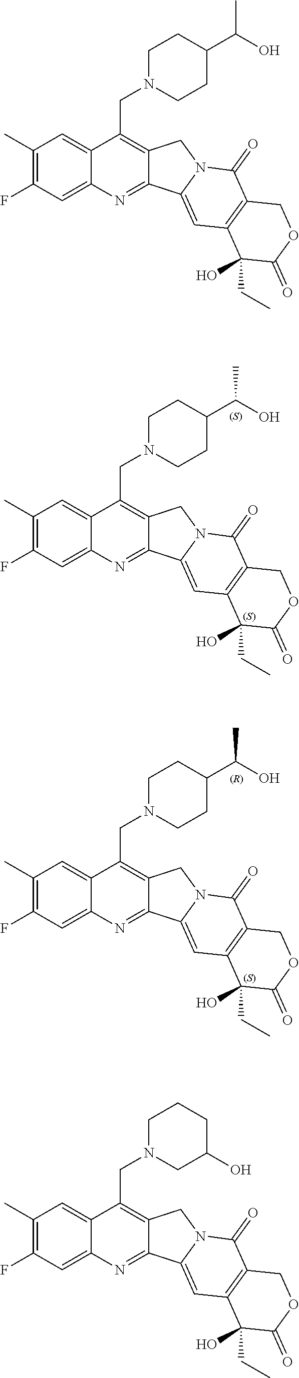

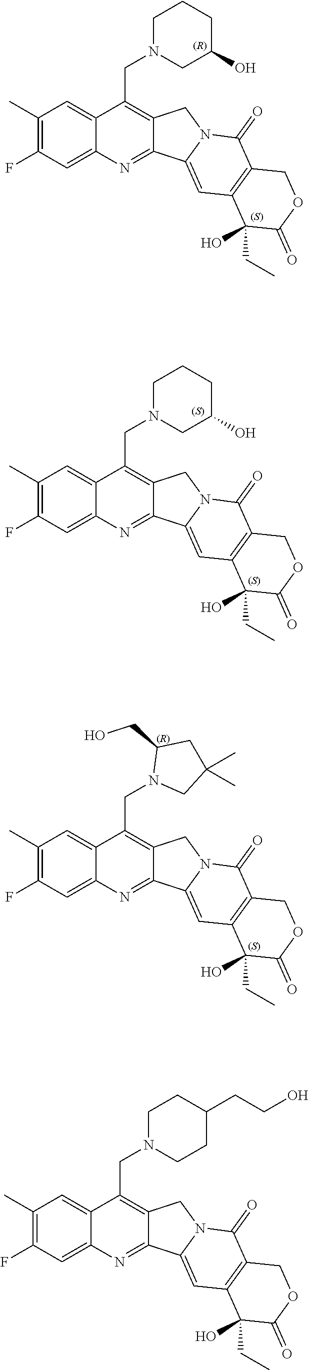

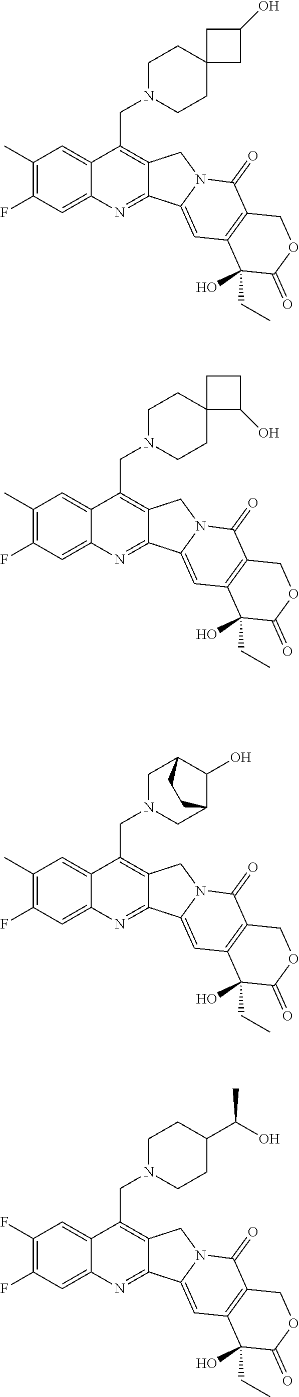 [SMILES: CC[C@@]1(O)C(=O)OCc2c1cc1n(c2=O)Cc2c-1nc1cc(F)c(C)cc1c2CN1CCC2(CC1)CC(O)C2 ; CC[C@@]1(O)C(=O)OCc2c1cc1n(c2=O)Cc2c-1nc1cc(F)c(C)cc1c2CN1CCC2(CCC2O)CC1 ; CC[C@@]1(O)C(=O)OCc2c1cc1n(c2=O)Cc2c-1nc1cc(F)c(C)cc1c2CN1C[C@H]2CC[C@@H](C1)C2O ; CC[C@@]1(O)C(=O)OCc2c1cc1n(c2=O)Cc2c-1nc1cc(F)c(F)cc1c2CN1CCC([C@@H](C)O)CC1]

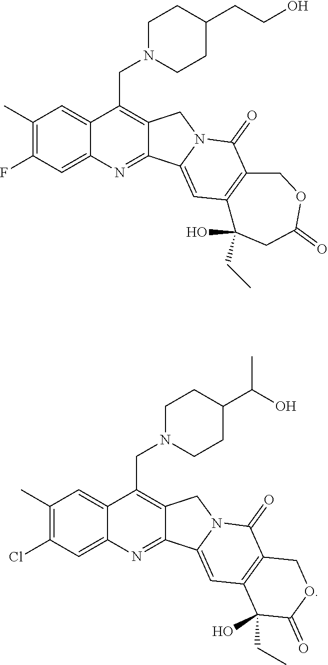 [SMILES: CC[C@@]1(O)CC(=O)OCc2c1cc1n(c2=O)Cc2c-1nc1cc(F)c(C)cc1c2CN1CCC(CCO)CC1 ; CC[C@@]1(O)C(=O)OCc2c1cc1n(c2=O)Cc2c-1nc1cc(Cl)c(C)cc1c2CN1CCC(C(C)O)CC1]

17. A pharmaceutical composition, comprising a therapeutically effective amount of the ligand-drug conjugate, or the pharmaceutically acceptable salt or solvate thereof, according to claim 1, and pharmaceutically acceptable carrier(s), diluent(s), or excipient(s).

18. A method of treating cancer, the method comprising administering to a subject in need thereof a ligand-drug conjugate, or the pharmaceutically acceptable salt or solvate thereof, according to claim 1 wherein the cancer is selected from the group consisting of breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer, colon cancer, rectal cancer, colorectal cancer, leukemia, bone cancer, skin cancer, thyroid cancer, pancreatic cancer, and lymphoma.

19. A pharmaceutical composition, comprising a therapeutically effective amount of the compound, or the pharmaceutically acceptable salt or solvate thereof, according to claim 14, and pharmaceutically acceptable carrier(s), diluent(s), or excipient(s).

20. A method of treating cancer, the method comprising administering to a subject in need thereof the pharmaceutical composition according to claim 14; wherein the cancer is selected from the group consisting of breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer, colon cancer, rectal cancer, colorectal cancer, leukemia, bone cancer, skin cancer, thyroid cancer, pancreatic cancer, and lymphoma.

21. A method of treating cancer, the method comprising administering to a subject in need thereof the pharmaceutical composition according to claim 17; wherein the cancer is selected from the group consisting of breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer, colon cancer, rectal cancer, colorectal cancer, leukemia, bone cancer, skin cancer, thyroid cancer, pancreatic cancer, and lymphoma.

22. A method of treating cancer, the method comprising administering to a subject in need thereof the pharmaceutical composition according to claim 19; wherein the cancer is selected from the group consisting of breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer, colon cancer, rectal cancer, colorectal cancer, leukemia, bone cancer, skin cancer, thyroid cancer, pancreatic cancer, and lymphoma.


SEQUENCE LISTING (6 sequences)

SEQ ID NO: 1 - protein, 214 aa, unidentified
  source 1..214: note = Trastuzumab Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, unidentified
  source 1..450: note = Trastuzumab Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, unidentified
  source 1..214: note = Pertuzumab Light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 449 aa, unidentified
  source 1..449: note = Pertuzumab Heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, unidentified
  source 1..214: note = B7H3 Light chain
DTWTQEPSFSVSPGGTVTLTCGLSSGSVSTSHYPSWYQQTPGQAPRMLIYNTNTRSSGVPDRFSGSILGNKAALTITGAQADDESDYYCAIHVDRDIWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 6 - protein, 448 aa, unidentified
  source 1..448: note = B7H3 Heavy chain
QVQLVQSGGGWQPGTSLRLSCAASGFIFSSSAMHWVRQAPGKGLEWVAVISYDGSNKYYVDSVKGRFTISRDNSKNTLYLQMNSLRAEDTAVYYCARSARLYASFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK